(19) United States
(12) Patent Application Publication
(10) Pub. No.: US 2024/0376109 A1
(43) Pub. Date: Nov. 14, 2024

(54) PYRAZOLO[1,5-A]PYRIMIDINE COMPOUND FOR THE TREATMENT OF DERMAL DISORDERS

(71) Applicant: OTSUKA PHARMACEUTICAL CO., LTD., Tokyo (JP)

(72) Inventors: Yukitaka UEMATSU, Osaka-shi, Osaka (JP); Minoru OKADA, Osaka-shi, Osaka (JP); Akihiro SAITO, Osaka-shi, Osaka (JP); Akira TAKAHASHI, Osaka-shi, Osaka (JP); Yuichi NAKAMURA, Osaka-shi, Osaka (JP); Taiki HANARI, Osaka-shi, Osaka (JP); Yasuhiro MENJO, Osaka-shi, Osaka (JP); Takahito YAMAUCHI, Osaka-shi, Osaka (JP); Atsushi YAMAGUCHI, Osaka-shi, Osaka (JP); Yohei YUKI, Osaka-shi, Osaka (JP); Kenji WATANABE, Osaka-shi, Osaka (JP); Satoshi MATSUDA, Osaka-shi, Osaka (JP)

(73) Assignee: OTSUKA PHARMACEUTICAL CO., LTD., Tokyo (JP)

(21) Appl. No.: 18/561,602

(22) PCT Filed: Jun. 1, 2022
(86) PCT No.: PCT/JP2022/022306

§ 371 Date: Nov. 16, 2023

(30) Foreign Application Priority Data
Jun. 2, 2021 (JP) 2021-092947

(51) Int. Cl. C07D 487/04; A61K 31/519; A61K 31/5377; A61K 31/541; A61K 31/55; A61K 31/551; A61K 31/553; A61P 17/04

(52) U.S. Cl. CPC C07D 487/04; A61K 31/519; A61K 31/5377; A61K 31/541; A61K 31/55; A61K 31/551; A61K 31/553; A61P 17/04

(57) ABSTRACT

Provided is a pyrazolo[1,5-a]pyrimidine compound represented by the general formula [I]:

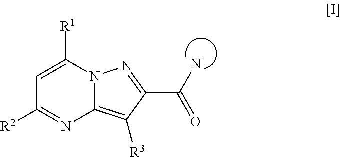

wherein each symbol is as defined in the description, or a salt thereof, having PAR2 inhibitory activity, and a pharmaceutical composition comprising the same.

### TECHNICAL FIELD

The present invention relates to a pyrazolo[1,5-a]pyrimidine compound or a pharmaceutically acceptable salt thereof, which has PAR2-inhibitory activity.

### BACKGROUND ART

Protease-activated receptor 2 (PAR2) is one of G protein-coupled 7-transmembrane receptors encoded by F2RL1 gene, and input signals into cells by proteases. PAR2 is called a tether receptor, and when the N-terminal of PAR2 is digested by proteases, mainly serine protease, the newly exposed N-terminal sequence acts as ligand to activate the receptors. Artificially synthesized peptides with N-terminal sequence occurred by digestion can also activate the receptors (NPLs 1,2).

PAR2 is expressed in a wide area in the body and is known to be involved in pruritus, allergy, inflammation, pain, and cancer. Therefore, PAR2 inhibitors are useful as therapeutic drugs for these diseases (NPL 3).

PAR2 is known to be involved in pruritus, especially in the skin. Exogenous proteases from plants or ticks, proteases secreted from keratinocytes due to skin irritation, and proteases secreted by immune cells such as mast cells activate PAR2 expressed in peripheral nerve endings and induce pruritus via signaling to the brain (NPL 4). It is known that there are several diseases associated with pruritus, some of which are accompanied by skin lesions and others are not. In the former type of pruritus, which is accompanied by inflammation and swelling, proteases derived from immune cells or keratinocytes activate PAR2 as pruritic substances. On the other hand, in the latter type of pruritus, which is not accompanied by skin lesions but results in the formation of permanent dry skin, the threshold of pruritus is lowered by intraepidermal invasion or sprouting of peripheral nerves, and the skin barrier is reduced by scratching, thereby forming an environment in which PAR2 is easily activated (NPL 5). For this reason, PAR2 inhibitors are useful not only for atopic dermatitis and urticaria, but also for pruritus caused by dry skin without skin lesions such as senile xerosis or underlying diseases (e.g., renal or liver failure).

It has also been reported that activation of PAR2 in keratinocytes increases the expression of matrix metalloproteinases, and that mice overexpressing PAR2 in the skin are prone to pruritus and skin inflammation, which are exacerbated by mite antigen sensitization (NPLs 6,7). These findings suggest that PAR2 is involved not only in pruritus but also in skin barrier function and inflammation, and PAR2 inhibitors are useful for restoring the skin barrier and reducing inflammation.

PAR2 is involved in pain signaling as well as pruritus signaling and is a target for hyperalgesia or allodynia (NPL 8). Therefore, PAR2 inhibitors are useful as therapeutic drugs for these diseases.

The PAR2 inhibitory activities of compounds with a pyrazolo[1,5-a]pyrimidine skeleton are described in PTLs 1-5.

### CITATION LIST

### Patent Literature

[PTL 1] JP 2003-286171 A
[PTL 2] JP 2004-170323 A
[PTL 3] WO 2018/043461
[PTL 4] WO 2019/163956
[PTL 5] JP 2020-007262 A

### Non Patent Literature

[NPL 1] Dery O et al., Am J Physiol, 274, C1429-1452, 1998
[NPL 2] Macfarlane S R et al., Pharmacol Rev,53, 245-282, 2001
[NPL 3] Yau M K et al., Expert Opin Ther Pat., 26, 471-483, 2016
[NPL 4] Akiyama T et al., Handb Exp Pharmacol., 226, 219-223, 2015
[NPL 5] Sato et al., Clinical Practice Guidelines for Generalized Pruritus 2012
[NPL 6] Yamada Y et al., Int Arch Allergy Immunol., 173, 84-92, 2017,
[NPL 7] Smith L et al., Exp Dermatol., 28, 1298-1308, 2019
[NPL 8] Dale C et al., NJ Recept Signal Transduct Res., 28, 29-37, 2008

### SUMMARY OF INVENTION

### Technical Problem

An object of the present invention is to provide pyrazolo[1,5-a]pyrimidine compounds or their salts having PAR2 inhibitory activity, and pharmaceutical compositions containing the same. Another object of the present invention is to provide pyrazolo[1,5-a]pyrimidine compounds or their salts suitable as active ingredients for topical transdermal formulation such as ointment, cream, lotion, and the like.

### Solution to Problem

As a result of conducting extensive studies to solve the above-mentioned problems, the inventors of the present invention found that the pyrazolo[1,5-a]pyrimidine compound represented by the following formula [1] has PAR2 inhibitory activity, thereby leading to completion of the present invention.

Namely, the present invention includes the following embodiments. [1-1] A compound represented by general formula [I]:

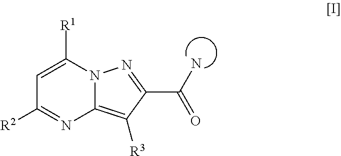

wherein
   R¹ is C₁₋₆ alkyl, C₃₋₈ cycloalkyl, C₁₋₆ haloalkyl, C₁₋₆ alkoxy, C₃₋₈ cycloalkoxy, C₁₋₆ alkylthio, or mono or di C₁₋₆ alkylamino;
   R² is C₃₋₈ cycloalkyl optionally substituted by halogen or C₁₋₆ alkyl, C₄₋₁₀ bicycloalkyl optionally substituted by halogen or C₁₋₆ alkyl, C₅₋₁₃ spiroalkyl, C₆₋₁₂ tricycloalkyl, C₃₋₈ cycloalkyl-C₁₋₆ alkyl optionally substituted by halogen, C₁₋₆ alkyl or C₁₋₆ haloalkyl, C₃₋₈ cycloalkoxy-C₁₋₆ alkyl, C₄₋₁₀ bicycloalkyl-C₁₋₆ alkyl optionally substituted by halogen or C₁₋₆ alkyl, C₆₋₁₂ tricycloalkyl-C₁₋₆ alkyl, C₆₋₁₂ tricycloalkyl-amino or piperidinyl;
   R³ is hydrogen, halogen or C₁₋₆ alkyl;

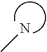

is 5- to 9-membered saturated or partially unsaturated heterocyclic ring or an oxo thereof, containing one nitrogen atom as ring-constituting atom, which may have halogen, C₁₋₆ alkyl, C₁₋₆ alkoxy, C₁₋₆ alkoxy-C₁₋₆ alkyl, hydroxy or methylidene as substituent, wherein the heterocyclic ring may further have one nitrogen atom, one oxygen atom and/or one sulfur atom as ring-constituting atom; or a salt thereof.
[1-2] The compound according to [1-1], wherein in the general formula [I],

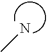

is piperidinyl, azepanyl, azocanyl, azonanyl, azepinyl, 2,3,4,7-tetrahydroazepinyl, 2,3,6,7-tetrahydroazepinyl, diazepanyl, piperazinyl, morpholinyl, thiomorpholinyl, oxazepanyl or an oxo thereof, wherein the heterocyclic ring may have halogen, C₁₋₆ alkyl, C₁₋₆ alkoxy or hydroxy as substituent, or a salt thereof.
[1-3] The compound according to [1-1], wherein in the general formula [I],
R¹ is ethyl, 1-propyl, 2-propyl, 1-butyl, 2-butyl, tert-butyl, 2-methyl-1-propyl, 2-methyl-1-butyl, 1-pentyl, 3-pentyl, 1-hexyl, cyclopropyl, cyclobutyl, cyclopentyl, cyclohexyl, 1-methylcyclohexyl, trifluoromethyl, 1,1-difluoroethyl, propoxy, cyclohexyloxy, ethylthio, methylpropylamino or dipropylamino;
R² is cyclopentyl, cyclohexyl, 1-methylcyclohexyl, 4-butylcyclohexyl, 4,4-difluorocyclohexyl, bicyclo[2.2.1]heptanyl, bicyclo[2.2.1]heptanylmethyl, bicyclo[4.1.0]heptanyl, bicyclo[2.2.2]octanyl, decahydronaphthyl, adamantyl (tricyclo[3.3.1.1]decanyl), spiro[2,5]octanyl, spiro[3,3]heptanylmethyl, 1-cyclohexylcyclopropyl, 1-methylcyclohexylmethyl, 2-methylcyclohexylmethyl, 3-methylcyclohexylmethyl, 3,5-dimethylcyclohexylmethyl, 4-ethylcyclohexylmethyl, 4-butylcyclohexylmethyl, 4-fluorocyclohexylmethyl, 4-methoxycyclohexylmethyl, 4-trifluoromethylcyclohexylmethyl, 4,4-difluorocyclohexylmethyl, 4,4-dimethylcyclohexylmethyl, cyclopropylmethyl, cyclobutylmethyl, cyclopentylmethyl, cyclopentylethyl, cyclohexylmethyl, cyclohexylethyl, cyclohexylpropyl, cyclohexylbutyl, cycloheptylmethyl, 1-cyclohexylethyl, adamantylmethyl, 4-methylcyclohexylmethyl, cyclopentyloxymethyl, cyclohexyloxymethyl, cycloheptyloxymethyl, adamantylamino or piperidinyl;
R³ is hydrogen;

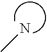

is azepanyl, azocanyl, azonanyl, 2,3,4,7-tetrahydroazepinyl, 2,3,6,7-tetrahydroazepinyl, 1,4-diazepanyl, oxazepanyl, 2,2-dimethylazepanyl, 3-hydroxyazepanyl, 4-hydroxyazepanyl, 4-methylazepanyl, 4,4-difluoroazepanyl, 4-methylpiperidinyl, 2,2-dimethylpiperidinyl, 2,2-dimethyl-3-hydroxypiperidinyl, 2,2-dimethyl-3-methylidene-piperidinyl, 2,2-dimethyl-4-hydroxypiperidinyl, 2,2-dimethyl-3-methoxypiperidinyl, 2,2-dimethyl-4-methoxypiperidinyl, 2,2,4,4-tetramethylpiperidinyl, 2,2,4,4-tetramethyl-3-hydroxypiperidinyl, 2,2,4,4-tetramethyl-4-methoxypiperidinyl, 2,2-dimethyl-4-methoxyethylpiperidinyl, 2,2-dimethyl-3-methylenepiperidinyl, 2,2-dimethylpiperazinyl, 2,2-dimethyl-4-hydroxypiperazinyl, 2,2-dimethylmorpholinyl, 2,2-dimethyl-3-oxopiperidinyl, 2,2,4,4-tetramethyl-3-hydroxypiperidinyl, 2,2,4,4-tetramethyl-3-oxopiperidinyl, 2,2-dimethyl-4-thiomorpholinyl, 3,3-dimethyl-4-thiomorpholinyl or oxazepanyl;
or a salt thereof.
   [1-4] The compound according to [1-1], wherein in the general formula [I],
R¹ is ethyl, 1-propyl, 2-propyl, 1-butyl, 2-butyl, 3-pentyl, cyclohexyl or trifluoromethyl;
R² is cyclopentyl, cyclohexyl, cycloheptyl, adamantyl, cyclobutylmethyl, cyclopentylmethyl, cyclopentylethyl, cyclohexylmethyl, cyclohexylethyl, cyclohexyloxymethyl, 1-cyclohexylethyl, 4-methylcyclohexylmethyl, 4-ethylcyclohexylmethyl, 4-trifluoromethylcyclohexylmethyl, 4,4-dimethylcyclohexylmethyl, bicyclo[2.2.1]heptanylmethyl, spiro[3.3]heptanylmethyl or adamantylamino;
R³ is hydrogen;

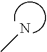

is piperidinyl, azepanyl, azocanyl, 2,3,4,7-tetrahydroazepinyl, 2,2-dimethylpiperidinyl, 2,2-dimthyl-3-hydroxypiperidinyl, 2,2-dimethyl-3-oxopiperidinyl, 2,2,4,4-tetramethyl-3-oxopiperidinyl or 3,3-dimethyl-4-thiomorpholinyl;
or a salt thereof.
   [1-5] The compound according to [1-1], which is selected from the group consisting of the following compounds:

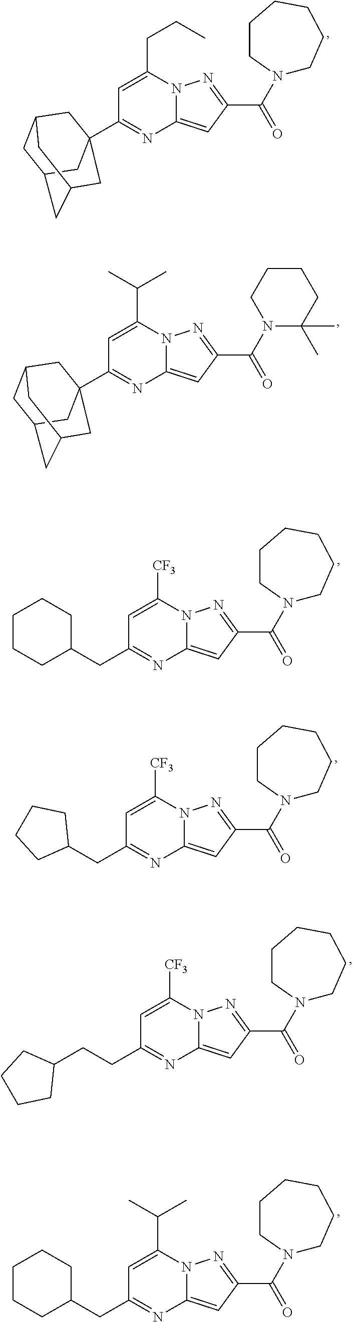 [SMILES: CCCc1cc(C23CC4CC(CC(C4)C2)C3)nc2cc(C(=O)N3CCCCCC3)nn12 ; CC(C)c1cc(C23CC4CC(CC(C4)C2)C3)nc2cc(C(=O)N3CCCCC3(C)C)nn12 ; O=C(c1cc2nc(CC3CCCCC3)cc(C(F)(F)F)n2n1)N1CCCCCC1 ; O=C(c1cc2nc(CC3CCCC3)cc(C(F)(F)F)n2n1)N1CCCCCC1 ; O=C(c1cc2nc(CCC3CCCC3)cc(C(F)(F)F)n2n1)N1CCCCCC1 ; CC(C)c1cc(CC2CCCCC2)nc2cc(C(=O)N3CCCCCC3)nn12]

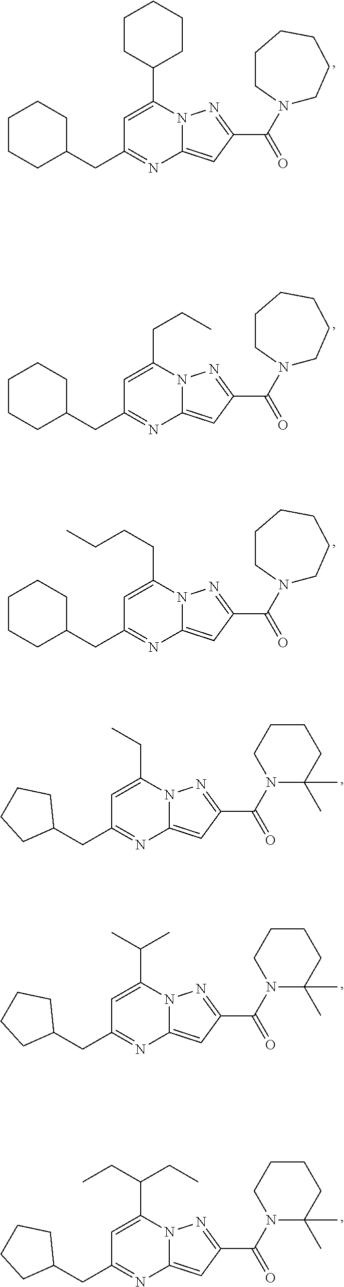 [SMILES: O=C(c1cc2nc(CC3CCCCC3)cc(C3CCCCC3)n2n1)N1CCCCCC1 ; CCCc1cc(CC2CCCCC2)nc2cc(C(=O)N3CCCCCC3)nn12 ; CCCCc1cc(CC2CCCCC2)nc2cc(C(=O)N3CCCCCC3)nn12 ; CCc1cc(CC2CCCC2)nc2cc(C(=O)N3CCCCC3(C)C)nn12 ; CC(C)c1cc(CC2CCCC2)nc2cc(C(=O)N3CCCCC3(C)C)nn12 ; CCC(CC)c1cc(CC2CCCC2)nc2cc(C(=O)N3CCCCC3(C)C)nn12]

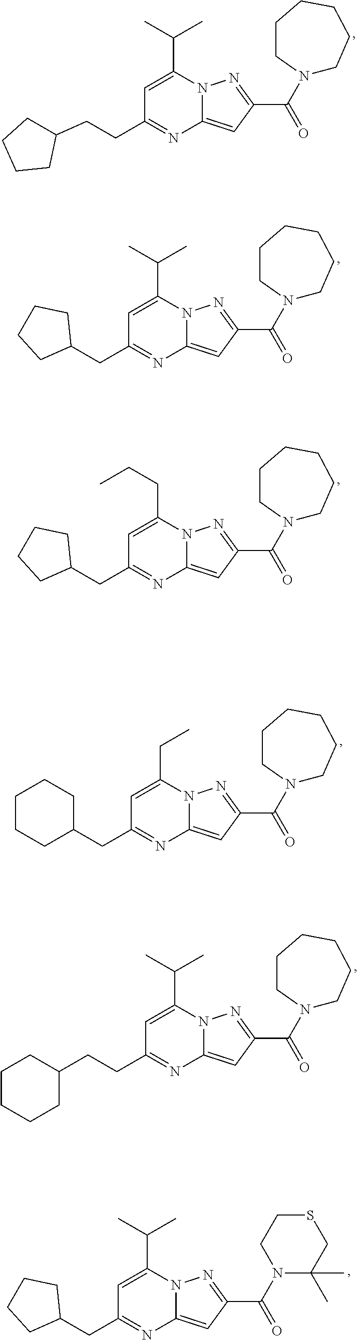 [SMILES: CC(C)c1cc(CCC2CCCC2)nc2cc(C(=O)N3CCCCCC3)nn12 ; CC(C)c1cc(CC2CCCC2)nc2cc(C(=O)N3CCCCCC3)nn12 ; CCCc1cc(CC2CCCC2)nc2cc(C(=O)N3CCCCCC3)nn12 ; CCc1cc(CC2CCCCC2)nc2cc(C(=O)N3CCCCCC3)nn12 ; CC(C)c1cc(CCC2CCCCC2)nc2cc(C(=O)N3CCCCCC3)nn12 ; CC(C)c1cc(CC2CCCC2)nc2cc(C(=O)N3CCSCC3(C)C)nn12]

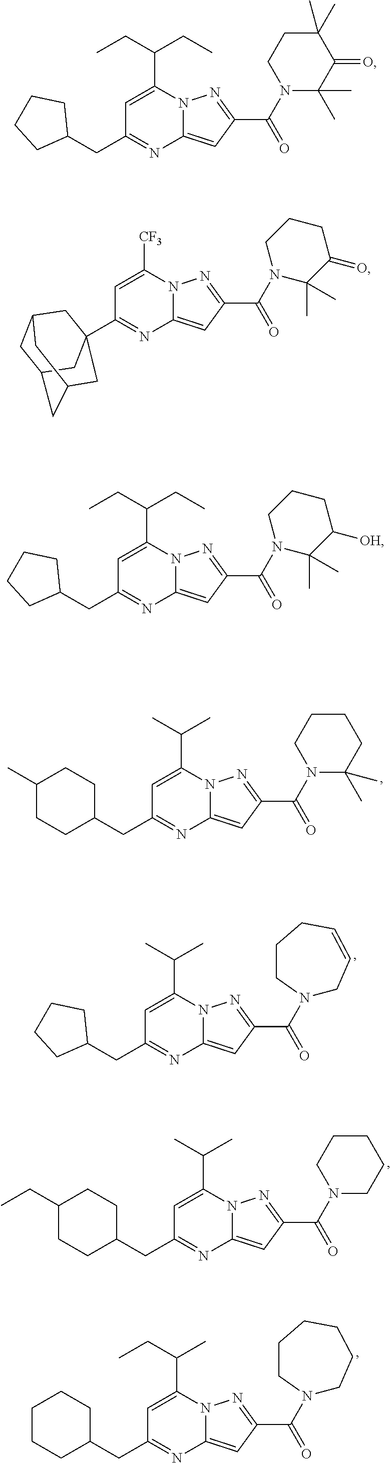

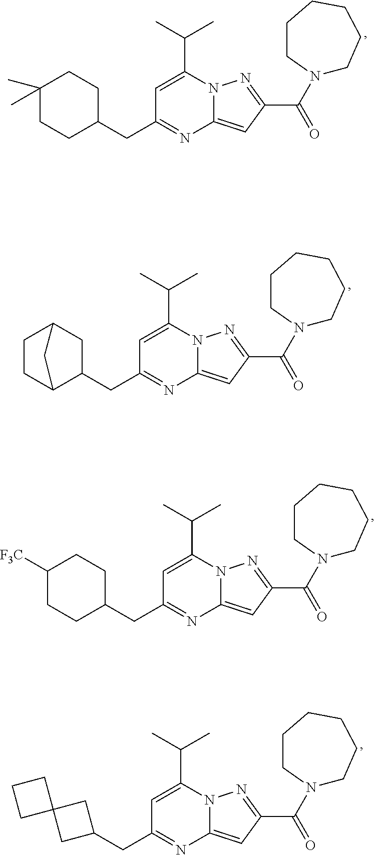 [SMILES: CC(C)c1cc(CC2CCC(C)(C)CC2)nc2cc(C(=O)N3CCCCCC3)nn12 ; CC(C)c1cc(CC2CC3CCC2C3)nc2cc(C(=O)N3CCCCCC3)nn12 ; CC(C)c1cc(CC2CCC(C(F)(F)F)CC2)nc2cc(C(=O)N3CCCCCC3)nn12 ; CC(C)c1cc(CC2CC3(CCC3)C2)nc2cc(C(=O)N3CCCCCC3)nn12]

or a salt thereof.
[2] A pharmaceutical composition comprising the compound according to any one of [1-1] to [1-5] or a salt thereof as active ingredient, and a pharmaceutically acceptable carrier or excipient.
[3-1] A therapeutic, preventative and/or diagnostic agent for a symptom and/or disease caused by PAR2 activation, comprising the compound according to any one of [1-1] to [1-5] or a salt thereof.
[3-2] The therapeutic, preventative and/or diagnostic agent according to [3-1], wherein the symptom caused by PAR2 activation is dermal pruritus.
[3-3] The therapeutic, preventative and/or diagnostic agent according to [3-2], wherein the dermal pruritus is dermal pruritus caused by atopic dermatitis, urticaria, eczema, sebum deficiency, sebum deficiency eczema, senile pruritus, xeroderma, senile xerosis, prurigo, seborrheic dermatitis, psoriasis, contact dermatitis, caterpillar dermatitis, insect bite, photosensitivity, fruit hypersensitivity, neurodermatitis, self-sensitizing dermatitis, pruritus on renal dialysis and/or pruritus associated with chronic liver disease.
[3-4] The therapeutic, preventative and/or diagnostic agent according to [3-1], wherein the disease caused by PAR2 activation is a skin disease.
[3-5] The therapeutic, preventative and/or diagnostic agent according to [3-4], wherein the skin disease is selected from atopic dermatitis, psoriasis, eczema, scleroderma and dermatitis.
[4-1] A therapeutic, preventative and/or diagnostic pharmaceutical composition for a symptom and/or disease caused by PAR2 activation, comprising the compound according to any one of [1-1] to [1-5] or a salt thereof as active ingredient.
[4-2] The therapeutic, preventative and/or diagnostic pharmaceutical composition according to [4-1], wherein the symptom caused by PAR2 activation is dermal pruritus.
[4-3] The therapeutic, preventative and/or diagnostic pharmaceutical composition according to [4-2], wherein the dermal pruritus is dermal pruritus caused by atopic dermatitis, urticaria, eczema, sebum deficiency, sebum deficiency eczema, senile pruritus, xeroderma, senile xerosis, prurigo, seborrheic dermatitis, psoriasis, contact dermatitis, caterpillar dermatitis, insect bite, photosensitivity, fruit hypersensitivity, neurodermatitis, self-sensitizing dermatitis, pruritus on renal dialysis and/or pruritus associated with chronic liver disease.
[4-4] The therapeutic, preventative and/or diagnostic pharmaceutical composition according to [4-1], wherein the disease caused by PAR2 activation is a skin disease.
[4-5] The therapeutic, preventative and/or diagnostic pharmaceutical composition according to [4-4], wherein the skin disease is selected from atopic dermatitis, psoriasis, eczema, scleroderma and dermatitis.
[5-1] A method for treating, preventing and/or diagnosing a symptom and/or disease caused by PAR2 activation, which comprises administering to a human in need thereof an effective amount of the compound or a salt thereof according to any of [1-1] to [1-5].
[5-2] The method according to [5-1], wherein the symptom caused by PAR2 activation is dermal pruritus.
[5-3] The method according to [5-2], wherein the dermal pruritus is dermal pruritus caused by atopic dermatitis, urticaria, eczema, sebum deficiency, sebum deficiency eczema, senile pruritus, xeroderma, senile xerosis, prurigo, seborrheic dermatitis, psoriasis, contact dermatitis, caterpillar dermatitis, insect bite, photosensitivity, fruit hypersensitivity, neurodermatitis, self-sensitizing dermatitis, pruritus on renal dialysis and/or pruritus associated with chronic liver disease.
[5-4] The method according to [5-1], wherein the disease caused by PAR2 activation is a skin disease.
[5-5] The method according to [5-4], wherein the skin disease is selected from atopic dermatitis, psoriasis, eczema, scleroderma and dermatitis.
[6-1] A compound or a salt thereof according to any of [1-1] to [1-5] for use in the treatment, prevention and/or diagnosis of a symptom and/or disease caused by PAR2 activation.
[6-2] The compound or a salt thereof according to [6-1], wherein the symptom caused by PAR2 activation is dermal pruritus.
[6-3] The compound or a salt thereof according to [6-2], wherein the dermal pruritus is dermal pruritus caused by atopic dermatitis, urticaria, eczema, sebum deficiency, sebum deficiency eczema, senile pruritus, xeroderma, senile xerosis, prurigo, seborrheic dermatitis, psoriasis, contact dermatitis, caterpillar dermatitis, insect bite, photosensitivity, fruit hypersensitivity, neurodermatitis, self-sensitizing dermatitis, pruritus on renal dialysis and/or pruritus associated with chronic liver disease.
[6-4] The compound or a salt thereof according to [6-1], wherein the disease caused by PAR2 activation is a skin disease.
[6-5] The compound or a salt thereof according to [6-4], wherein the skin disease is selected from atopic dermatitis, psoriasis, eczema, scleroderma and dermatitis.
[7-1] Use of a compound or a salt thereof according to any of [1-1] to [1-5] in the manufacture of a medicament for treating, preventing and/or diagnosing a symptom and/or disease caused by PAR2 activation.
[7-2] The use according to [7-1], wherein the symptom caused by PAR2 activation is dermal pruritus.
[7-3] The use according to [7-2], wherein the dermal pruritus is dermal pruritus caused by atopic dermatitis, urticaria, eczema, sebum deficiency, sebum deficiency eczema, senile pruritus, xeroderma, senile xerosis, prurigo, seborrheic dermatitis, psoriasis, contact dermatitis, caterpillar dermatitis, insect bite, photosensitivity, fruit hypersensitivity, neurodermatitis, self-sensitizing dermatitis, pruritus on renal dialysis and/or pruritus associated with chronic liver disease.
[7-4] The use according to [7-1], wherein the disease caused by PAR2 activation is a skin disease.
[7-5] The use according to [7-4], wherein the skin disease is selected from atopic dermatitis, psoriasis, eczema, scleroderma and dermatitis.
[8-1] A topical transdermal formulation comprising the compound according to any one of [1-1] to [1-5] or a salt thereof as active ingredient, and a pharmaceutically acceptable carrier or excipient.
[8-2] The topical transdermal formulation according to [8-1], which is in the form selected from ointment, cream, lotion and foam.

### Advantageous Effects of Invention

The compound or a salt thereof of the present invention has an excellent PAR2 inhibitory activity. Moreover, the compound or a salt thereof of the present invention shows few or no skin irritation, and has excellent skin absorption.

### DESCRIPTION OF EMBODIMENTS

The terms and phrases used in the present description will be described in detail below.

In the present description, “halogen” is fluorine, chlorine, bromine, or iodine. It is preferably fluorine, chlorine, or bromine, and more preferably fluorine or chlorine.

In the present description, “C₁₋₆ alkyl” is linear or branched alkyl having 1 to 6 carbon atoms (C₁₋₆), and specific examples thereof include methyl, ethyl, n-propyl, isopropyl, 1-methylpropyl, 2-methylpropyl, n-butyl, isobutyl, sec-butyl, tert-butyl, 3-methylbutyl, n-pentyl, isopentyl, neopentyl, 3-pentyl, n-hexyl, isohexyl, 3-methylpentyl, 1,1-dimethylethyl, 1,2-dimethylethyl, 2,2-dimethylethyl, 1,1-dimethylpropyl, 1,2-dimethylpropyl, 2,2-dimethylpropyl, and the like.

In addition, the “C₁₋₆ alkyl” includes C₁₋₆ alkyl in which 1 to 7 hydrogen atoms are substituted by deuterium atoms.

In the present description, “C₁₋₆ haloalkyl” is linear or branched alkyl having 1 to 6 carbon atoms (C₁₋₆), which is substituted by 1-4 halogens, and specific examples thereof include fluoromethyl, chloromethyl, bromomethyl, iodomethyl, difluoromethyl, dichloromethyl, dibromomethyl, trifluoromethyl, trichloromethyl, 2-fluoroethyl, 2-chloroethyl, 2,2,2-trifluoroethyl, 2,2,2-trichloroethyl, 1,1,2,2-tetrafluoroethyl, 3-chloropropyl, 2,3-dichloropropyl, 4,4,4-trichlorobutyl, 4-fluorobutyl, 5-chloropentyl, 3-chloro-2-methylpropyl, 5-bromohexyl, 5,6-dibromohexyl, and the like.

In the present description, “C₃₋₈ cycloalkyl” is cycloalkyl having 3 to 8 carbon atoms (C₃₋₈), and specific examples thereof include cyclopropyl, cyclobutyl, cyclopentyl, cyclohexyl, cycloheptyl, cyclooctyl, and the like.

In the present description, “C₄₋₁₀ bicycloalkyl” is bicyclic cycloalkyl having 4 to 10 carbon atoms (C₄₋₁₀), and specific examples thereof include bicyclo[2.2.1]heptyl, bicyclo[2.2.2]octyl, and the like.

In the present description, “C₆₋₁₂ tricycloalkyl” is tricyclic cycloalkyl having 6 to 12 carbon atoms (C₆₋₁₂), and specific examples thereof include adamantyl and the like.

In the present description, “C₅₋₁₃ spiroalkyl” includes spiro[2,2]pentanyl, spiro[2,3]hexanyl, spiro[2,4]heptanyl, spiro[2,5]octanyl, spiro[2,6]nonanyl, spiro[2,7]decanyl, spiro[3,3]heptanyl, spiro[3,4]octanyl, spiro[3,5]nonanyl, spiro[3,6]decanyl, and the like.

In the present description, “C₁₋₆ alkoxy” is linear or branched alkoxy having 1 to 6 carbon atoms (C₁₋₆), and specific examples thereof include methoxy, ethoxy, n-propoxy, isopropoxy, 1-methylpropoxy, 2-methylpropoxy, n-butoxy, isobutoxy, sec-butoxy, tert-butoxy, 3-methylbutoxy, n-pentoxy, isopentoxy, neopentoxy, 3-pentoxy, n-hexoxy, isohexoxy, 3-methylpentoxy, 1,1-dimethylethoxy, 1,2-dimethylethoxy, 2,2-dimethylethoxy, 1,1-dimethylpropoxy, 1,2-dimethylpropoxy, 2,2-dimethylpropoxy, and the like.

In the present description, “C₃₋₈ cycloalkoxy” is cycloalkyloxy having 3 to 8 carbon atoms (C₃₋₈), and specific examples thereof include cyclopropyloxy, cyclobutyloxy, cyclopentyloxy, cyclohexyloxy, cycloheptyloxy, cyclooctyloxy, and the like.

In the present description, “C₁₋₆ alkylthio” is linear or branched alkylthio having 1 to 6 carbon atoms (C₁₋₆), and specific examples thereof include methylthio, ethylthio, n-propylthio, isopropylthio, 1-methylpropylthio, 2-methylpropylthio, n-butylthio, isobutylthio, sec-butylthio, tert-butylthio, 3-methylbutylthio, n-pentylthio, isopentylthio, neopentylthio, 3-pentylthio, n-hexylthio, isohexylthio, 3-methylpentylthio, 1,1-dimethylethyl, 1,2-dimethylethylthio, 2,2-dimethylethylthio, 1,1-dimethylpropylthio, 1,2-dimethylpropylthio, 2,2-dimethylpropylthio, and the like.

In the present description, “mono or di C₁₋₆ alkylamino” is amino with one or two linear or branched alkyls having 1 to 6 carbon atoms (C₁₋₆), and specific examples thereof include methylamino, ethylamino, n-propylamino, isopropylamino, 1-methylpropylamino, 2-methylpropylamino, n-butylamino, isobutylamino, sec-butylamino, tert-butylamino, 3-methylbutylamino, dimethylamino, diethylamino, dipropylamino, methylethylamino, methylpropylamino, ethylpropylamino, and the like.

In the present description, “C₃₋₈ cycloalkyl-C₁₋₆ alkyl” is linear or branched alkyl having 1 to 6 carbon atoms (C₁₋₆), which is substituted by cycloalkyl having 3 to 8 carbon atoms (C₃₋₈), and specific examples thereof include cyclopropylmethyl, cyclobutylmethyl, cyclopentylmethyl, cyclohexylmethyl, cycloheptylmethyl, cyclooctylmethyl, cyclopropylethyl, cyclobutylethyl, cyclopentylethyl, cyclohexylethyl, cycloheptylethyl, cyclooctylethyl, cyclohexyl-2-propyl, and the like.

In the present description, “C₄₋₁₀ bicycloalkyl-C₁₋₆ alkyl” is linear or branched alkyl having 1 to 6 carbon atoms (C₁₋₆), which is substituted by bicyclic cycloalkyl having 4 to 10 carbon atoms (C₄₋₁₀), and specific examples thereof include bicyclo[2.2.1]heptylmethyl, bicyclo[2.2.2]octylmethyl, bicyclo[2.2.1]heptylethyl, bicyclo[2.2.2]octylethyl, and the like.

In the present description, “C₆₋₁₂ tricycloalkyl-C₁₋₆ alkyl” is linear or branched alkyl having 1 to 6 carbon atoms (C₁₋₆), which is substituted by tricyclic cycloalkyl having 6 to 12 carbon atoms (C₆₋₁₂), and specific examples thereof include adamantylmethyl, adamantylethyl, adamantylpropyl, and the like.

In the present description, “C₆₋₁₂ tricycloalkyl-amino” is amine substituted by tricyclic cycloalkyl having 6 to 12 carbon atoms (C₆₋₁₂), and specific examples thereof include adamantylamino, and the like.

In the present description, “C₃₋₈ cycloalkoxy-C₁₋₆ alkyl” is linear or branched alkyl having 1 to 6 carbon atoms (C₁₋₆), which is substituted by cycloalkoxy having 3 to 8 carbon atoms (C₃₋₈), and specific examples thereof include cyclopropyloxymethyl, cyclobutyloxymethyl, cyclopentyloxymethyl, cyclohexyloxymethyl, cycloheptyloxymethyl, cyclooctanoxymethyl, cyclopropyloxyethyl, cyclobutyloxyethyl, cyclopentyloxyethyl, cyclohexyloxyethyl, cycloheptyloxyethyl, cyclooctanoxyethyl, and the like.

In the present description, “5- to 9-membered saturated or partially unsaturated heterocyclic ring or an oxo thereof, containing one nitrogen atom as ring-constituting atom, wherein the heterocyclic ring may further have one nitrogen atom, one oxygen atom and/or one sulfur atom as ring-constituting atom” includes pyrrolidinyl, piperidinyl, azepanyl, azocanyl, azonanyl, azepinyl, 2,3,4,7-tetrahydroazepinyl, 2,3,6,7-tetrahydroazepinyl, 1,4-diazepanyl, imidazolidinyl, pyrazolidinyl, piperazinyl, diazepanyl, oxazolidinyl, isoxazolidinyl, morpholinyl, thiazolidinyl, isothiazolidinyl, thiomorpholinyl, and the like.

In the present description, the “condensing agent” is not particularly limited, but specific examples thereof include 1-[3-(dimethylamino)propyl]-3-ethylcarbodiimide hydrochloride (WSC·HCl), N,N′-dicyclohexylcarbodiimide (DCC), N,N′-diisopropylcarbodiimide (DIC), N,N′-carbonyldiimidazole (CDI), 4-(4,6-dimethoxy-1,3,5-triazin-2-yl)-4-methyl morpholinium chloride (DMT-MM), benzotriazol-1-yloxytris(dimethylamino)phosphonium hexafluorophosphate (BOP), benzotriazol-1-yloxytripyrrolidinophosphonium hexafluorophosphate (PyBOP), O-(7-azabenzotriazol-1-yl)-1,1,3,3-tetramethyluronium hexafluorophosphate (HATU), (1-cyano-2-ethoxy-2-oxoethylidenaminooxy)dimethylaminomorpholinocarbenium hexafluorophosphate (COMU), and the like, preferably WSC·HCl, HATU and COMU.

In the present description, “magnesium halide” includes magnesium fluoride, magnesium chloride, magnesium bromide, magnesium iodide, and the like.

In the present description, the “additive” is not particularly limited, but specific examples thereof include 1-hydroxybenzotriazole (HOBt), 1-Hydroxy-7-azabenzotriazole (HOAt), N-Hydroxysuccinimide (HOSu), ethyl (hydroxyimino)cyanoacetate (Oxyma), 4-dimethylaminopyridine (DMAP), triethylamine (TEA), Diisopropylethylamine (DIPEA), N-methylmorpholine, and the like, preferably HOBt, TEA and DIPEA.

In the present description, the “catalyst” to be used in reduction reaction is not particularly limited, but specific examples thereof include palladium-carrying carbon (Pd/C), platinum-carrying carbon (Pt/C), and the like.

In the present description, the “halogenating agent” is not particularly limited, but specific examples thereof include fluorinating agent, chlorinating agent, brominating agent, and iodinating agent, such as potassium fluoride, tetrabutylammonium fluoride, (diethylamino)sulfur trifluoride, phosphorus oxychloride, phosphorus trichloride, phosphorus pentachloride, thionyl chloride, oxalyl chloride, trichlorophosphoric acid, bromine, phosphorus oxybromide, phosphorus tribromide, iodine, sodium iodide, and the like.

In the present description, the “copper compound” is not particularly limited, but specific examples thereof include copper iodide, copper bromide, copper chloride, and the like.

In the present description, the “acid” is not particularly limited, but includes an inorganic acid, an organic acid, and the like. Examples of the “inorganic acid” include hydrochloric acid, sulfuric acid, nitric acid, hydrobromic acid, phosphoric acid, and the like. Examples of “organic acid” include acetic acid, trifluoroacetic acid, oxalic acid, phthalic acid, fumaric acid, tartaric acid, maleic acid, citric acid, succinic acid, methanesulfonic acid, p-toluenesulfonic acid, 10-camphorsulfonic acid, and the like.

These acids are used alone or as a mixture of two or more of them.

In the present description, the “base” is not particularly limited, but includes an inorganic base, an organic base, and the like.

Examples of the “inorganic base” include alkali metal hydroxides (e.g., lithium hydroxide, sodium hydroxide, and potassium hydroxide), alkaline earth metal hydroxides (e.g., magnesium hydroxide, calcium hydroxide, and barium hydroxide), alkali metal carbonates (e.g., sodium carbonate, potassium carbonate, and cesium carbonate), alkaline earth metal carbonates (e.g., magnesium carbonate, calcium carbonate, and barium carbonate), alkali metal hydrogen carbonates (e.g., sodium hydrogen carbonate and potassium hydrogen carbonate), alkali metal phosphates (e.g., sodium phosphate, potassium phosphate, and cerium phosphate), alkaline earth metal phosphates (e.g., magnesium phosphate and calcium phosphate), alkali metal alkoxides (e.g., sodium methoxide, sodium ethoxide, sodium tert-butoxide, and potassium tert-butoxide), alkali metal hydride (e.g., sodium hydride and potassium hydride), and the like.

Examples of the “organic base” include trialkylamines (e.g., trimethylamine, triethylamine, and N,N-diisopropylethylamine (DIPEA)), dialkylamine (e.g., diethylamine and diisopropylamine), 4-dimethylaminopyridine (DMAP), N-methylmorphiline, picoline, 1,5-diazabicyclo[4.3.0]non-5-ene, 1,4-diazabicyclo[2.2.2]octane, 1,8-diazabicyclo[5.4.0]undec-7-ene (DBU), and the like. These bases are used alone or as a mixture of two or more of them.

It is preferably DMAP or TEA. These bases are used alone or as a mixture of two or more of them.

In the present description, the “amine” is not particularly limited, but examples include trialkylamine (e.g., trimethylamine, triethylamine, N,N-diisopropylethylamine (DIPEA)), dialkylamine (e.g., diethylamine, diisopropylamine), dialkylaniline (e.g., N,N-diethylaniline, N,N-dimethylaniline), and the like.

The “palladium compound” to be used in the present description is not particularly limited, and examples thereof include tetravalent palladium catalysts such as sodium hexachloropalladium(IV) acid tetrahydrate and potassium hexachloropalladium(IV) acid; divalent palladium catalysts such as [1,1′-bis(diphenylphosphino)ferrocene]palladium(II) dichloride dichloromethane adduct (Pd(dppf)Cl₂·CH₂Cl₂), (2-dicyclohexylphosphino-2′,4′,6′-triisopropyl-1,1′-biphenyl)[2-(2′-amino-1,1′-biphenyl)]palladium(II) methanesulfonate (XPhos Pd G3), palladium(II) chloride, palladium(II) bromide, palladium(II) acetate, palladium(II) acetylacetonate, dichlorobis(benzonitrile)palladium(II), dichlorobis(acetonitrile)palladium(II), dichlorobis(triphenylphosphine)palladium(II), dichlorotetraammine palladium(II), dichloro(cycloocta-1,5-diene)palladium(II), and palladium(II) trifluoroacetate, and 1,1′-bis(diphenylphosphino)ferrocene dichloropalladium(II) dichloromethane complex; and zerovalent palladium catalysts such as tris(dibenzylideneacetone)dipalladium(0) (Pd₂(dba)₃), tris(dibenzylideneacetone)dipalladium(0)-chloroform complex, and tetrakis(triphenylphosphine)palladium(0) (Pd(PPh3)₄). These palladium compounds are used alone or as a mixture of two or more of them.

Specific examples of the “leaving group” used in the present description include halogen, C₁₋₁₈ alkanesulfonyl, lower alkanesulfonyloxy, arylsulfonyloxy, aralkylsulfonyloxy, perhaloalkanesulfonyloxy, sulfonio, toluenesulfoxy, and the like. A preferable leaving group is halogen.

The “halogen” is fluorine, chlorine, bromine, or iodine.

Examples of the “C₁₋₁₈ alkanesulfonyl” include linear or branched alkanesulfonyl having 1 to 18 carbon atoms (C₁₋₁₈), and specific examples thereof include methanesulfonyl, 1-propanesulfonyl, 2-propanesulfonyl, butanesulfonyl, cyclohexanesulfonyl, dodecanesulfonyl, octadecanesulfonyl, and the like.

Examples of the “lower alkanesulfonyloxy” include linear or branched alkanesulfonyloxy having 1 to 6 carbon atoms (C₁₋₆), and specific examples thereof include methanesulfonyloxy, ethanesulfonyloxy, 1-propanesulfonyloxy, 2-propanesulfonyloxy, 1-butanesulfonyloxy, 3-butanesulfonyloxy, 1-pentanesulfonyloxy, 1-hexanesulfonyloxy, and the like.

Examples of the “arylsulfonyloxy” include phenylsulfonyloxy optionally having 1 to 3 groups selected from the group consisting of linear or branched alkyl having 1 to 6 carbon atoms (C₁₋₆), linear or branched alkoxy having 1 to 6 carbon atoms (C₁₋₆), nitro and halogen, as a substituent on the phenyl ring, naphthylsulfonyloxy, and the like. Specific examples of the “phenylsulfonyloxy optionally having substituent(s)” include phenylsulfonyloxy, 4-methylphenylsulfonyloxy, 2-methylphenylsulfonyloxy, 4-nitrophenylsulfonyloxy, 4-methoxyphenylsulfonyloxy, 2-nitrophenylsulfonyloxy, 3-chlorophenylsulfonyloxy, and the like. Specific examples of the “naphthylsulfonyloxy” include α-naphthylsulfonyloxy, β-naphthylsulfonyloxy, and the like.

Examples of the “aralkylsulfonyloxy” include linear or branched alkanesulfonyloxy having 1 to 6 carbon atoms (C₁₋₆), which is substituted by phenyl optionally having 1 to 3 groups selected from the group consisting of linear or branched alkyl having 1 to 6 carbon atoms (C₁₋₆), linear or branched alkoxy having 1 to 6 carbon atoms (C₁₋₆), nitro and halogen, as a substituent on the phenyl ring, and linear or branched alkanesulfonyloxy having 1 to 6 carbon atoms (C₁₋₆), which is substituted by naphthyl, and the like. Specific examples of the “alkanesulfonyloxy substituted by phenyl” include benzylsulfonyloxy, 2-phenylethylsulfonyloxy, 4-phenylbutylsulfonyloxy, 4-methylbenzylsulfonyloxy, 2-methylbenzylsulfonyloxy, 4-nitrobenzylsulfonyloxy, 4-methoxybenzylsulfonyloxy, 3-chlorobenzylsulfonyloxy, and the like. Specific examples of the “alkanesulfonyloxy substituted by naphthyl” include α-naphthylmethylsulfonyloxy, β-naphthylmethylsulfonyloxy, and the like.

Specific examples of the “perhaloalkanesulfonyloxy” include trifluoromethanesulfonyloxy and the like.

Specific examples of the “sulfonio” include dimethylsulfonio, diethylsulfonio, dipropylsulfonio, di(2-cyanoethyl)sulfonio, di(2-nitroethyl)sulfonio, di-(aminoethyl)sulfonio, di(2-methylaminoethyl)sulfonio, di-(2-dimethylaminoethyl)sulfonio, di-(2-hydroxyethyl)sulfonio, di-(3-hydroxypropyl)sulfonio, di-(2-methoxyethyl)sulfonio, di-(2-carbamoylethyl)sulfonio, di-(2-carbamoylethyl)sulfonio, di-(2-carboxyethyl)sulfonio, di-(2-methoxycarbonylethyl)sulfonio, diphenylsulfonio, and the like.

The “solvent” to be used in the reaction in the present description may be an inert solvent in the reaction, and examples thereof include water, ethers (e.g., dioxane, tetrahydrofuran, diethyl ether, 1,2-dimethoxyethane, cyclopentyl methyl ether, diethylene glycol dimethyl ether, and ethylene glycol dimethyl ether), halohydrocarbons (e.g., methylene chloride, chloroform, 1,2-dichloroethane, and carbon tetrachloride), aromatic hydrocarbons (e.g., benzene, toluene, and xylene), lower alcohols (e.g., methanol, ethanol, and isopropanol), and polar solvents (e.g., N,N-dimethylformamide (DMF), N-methylpyrrolidone (NMP), dimethyl sulfoxide (DMSO), hexamethylphosphoric triamide, and acetonitrile). These solvents are used alone or as a mixture of two or more of them.

The various substituents in the compound represented by general formula [I] of the present invention (hereafter referred to as “compound [1]”) are explained below.

R¹ in the compound [I] is C₁₋₆ alkyl, C₃₋₈ cycloalkyl, C₁₋₆ haloalkyl, C₁₋₆ alkoxy, C₃₋₈ cycloalkoxy, C₁₋₆ alkylthio, or mono or di C₁₋₆ alkylamino, preferably ethyl, 1-propyl, 2-propyl, 1-butyl, 2-butyl, tert-butyl, 2-methyl-1-propyl, 2-methyl-1-butyl, 1-pentyl, 3-pentyl, 1-hexyl, cyclopropyl, cyclobutyl, cyclopentyl, cyclohexyl, 1-methylcyclohexyl, trifluoromethyl, 1,1-difluoroethyl, propoxy, cyclohexyloxy, ethylthio, methylpropylamino or dipropylamino, more preferably ethyl, 1-propyl, 2-propyl, 1-butyl, 2-butyl, 3-pentyl, cyclohexyl, or trifluoromethyl.

R² in the compound [I] is C₃₋₈ cycloalkyl optionally substituted by halogen or C₁₋₆ alkyl, C₄₋₁₀ bicycloalkyl optionally substituted by halogen or C₁₋₆ alkyl, C₅₋₁₃ spiroalkyl, C₆₋₁₂ tricycloalkyl, C₃₋₈ cycloalkyl-C₁₋₆ alkyl optionally substituted by halogen, C₁₋₆ alkyl or C₁₋₆ haloalkyl, C₃₋₈ cycloalkoxy-C₁₋₆ alkyl, C₄₋₁₀ bicycloalkyl-C₁₋₆ alkyl optionally substituted by halogen or C₁₋₆ alkyl, C₆₋₁₂ tricycloalkyl-C₁₋₆ alkyl, C₆₋₁₂ tricycloalkyl-amino or piperidinyl, preferably cyclopentyl, cyclohexyl, 1-methylcyclohexyl, 4-butylcyclohexyl, 4,4-difluorocyclohexyl, bicyclo[2.2.1]heptanyl, bicyclo[2.2.1]heptanylmethyl, bicyclo[4.1.0]heptanyl, bicyclo[2.2.2]octanyl, decahydronaphthyl, adamantyl (tricyclo[3.3.1.1]decanyl), spiro[2,5]octanyl, spiro[3,3]heptanylmethyl, 1-cyclohexylcyclopropyl, 1-methylcyclohexylmethyl, 2-methylcyclohexylmethyl, 3-methylcyclohexylmethyl, 4-methylcyclohexylmethyl, 3,5-dimethylcyclohexylmethyl, 4-ethylcyclohexylmethyl, 4-butylcyclohexylmethyl, 4-fluorocyclohexylmethyl, 4-methoxycyclohexylmethyl, 4-trifluoromethylcyclohexylmethyl, 4,4-difluorocyclohexylmethyl, 4,4-dimethylcyclohexylmethyl, cyclopropylmethyl, cyclobutylmethyl, cyclopentylmethyl, cyclopentylethyl, cyclohexylmethyl, cyclohexylethyl, cyclohexylpropyl, cyclohexylbutyl, cycloheptylmethyl, 1-cyclohexylethyl, adamantylmethyl, 4-methylcyclohexylmethyl, cyclopentyloxymethyl, cyclohexyloxymethyl, cycloheptyloxymethyl, adamantylamino or piperidinyl, more preferably cyclopentyl, cyclohexyl, cycloheptyl, adamantyl, cyclobutylmethyl, cyclopentylmethyl, cyclopentylethyl, cyclohexylmethyl, cyclohexylethyl, cyclopentyloxym ethyl, 1-cyclohexylethyl, 4-methylcyclohexylmethyl, 4-ethylcyclohexylmethyl, 4-trifluoromethylcyclohexylmethyl,4,4-dimethylcyclohexylmethyl, bicyclo[2.2.1]heptanylmethyl, spiro[3.3]heptanylmethyl, or adamantylamino.

R³ in the compound [I] is hydrogen, halogen or C₁₋₆ alkyl, preferably hydrogen, fluorine, chlorine, bromine, or iodine, more preferably hydrogen, or fluorine, further preferably hydrogen.

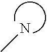

in the compound [I] is 5- to 9-membered saturated or partially unsaturated heterocyclic ring or an oxo thereof, containing one nitrogen atom as ring-constituting atom, which may have halogen, C₁₋₆ alkyl, C₁₋₆ alkoxy, C₁₋₆ alkoxy-C₁₋₆ alkyl, hydroxy or methylidene as substituent, wherein the heterocyclic ring may further have one nitrogen atom, one oxygen atom and/or one sulfur atom as ring-constituting atom, preferably piperidinyl, azepanyl, azocanyl, azonanyl, azepinyl, 2,3,4,7-tetrahydroazepinyl, 2,3,6,7-tetrahydroazepinyl, diazepanyl, piperazinyl, morpholinyl, thiomorpholinyl, oxazepanyl or an oxo thereof, wherein the heterocyclic ring may have halogen, C₁₋₆ alkyl, C₁₋₆ alkoxy, or hydroxy as substituent, more preferably azepanyl, azocanyl, azonanyl, 2,3,4,7-tetrahydroazepinyl, 2,3,6,7-tetrahydroazepinyl, 1,4-diazepanyl, oxazepanyl, 2,2-dimethylazepanyl, 3-hydroxyazepanyl, 4-hydroxyazepanyl, 4-methylazepanyl, 4,4-difluoroazepanyl, 4-methylpiperidinyl, 2,2-dimethylpiperidinyl, 2,2-dimethyl-3-hydroxypiperidinyl, 2,2-dimethyl-3-methylidene-piperidinyl, 2,2-dimethyl-4-hydroxypiperidinyl, 2,2-dimethyl-3-methoxypiperidinyl, 2,2-dimethyl-4-methoxypiperidinyl, 2,2,4,4-tetramethylpiperidinyl, 2,2,4,4-tetramethyl-3-hydroxypiperidinyl, 2,2,4,4-tetramethyl-4-methoxypiperidinyl, 2,2-dimethyl-4-methoxyethylpiperidinyl, 2,2-dimethyl-3-methylenepiperidinyl, 2,2-dimethylpiperazinyl, 2,2-dimethylmorpholinyl, 2,2-dimethyl-3-oxopiperidinyl, 2,2,4,4-tetramethyl-3-hydroxypiperidinyl, 2,2,4,4-tetramethyl-3-oxopiperidinyl, 2,2-dimethyl-4-thiomorpholinyl, 3,3-dimethyl-4-thiomorpholinyl or oxazepanyl, more preferably, piperidinyl, azepanyl, azocanyl, 2,3,4,7-tetrahydroazepinyl, 2,2-dimethylpiperidinyl, 2,2-dimthyl-3-hydroxypiperidinyl, 2,2-dimethyl-3-oxopiperidinyl, 2,2,4,4-tetramethyl-3-oxopiperidinyl, or 3,3-dimethyl-4-thiomorpholinyl.

### Specific Examples of the Compound [1] Include

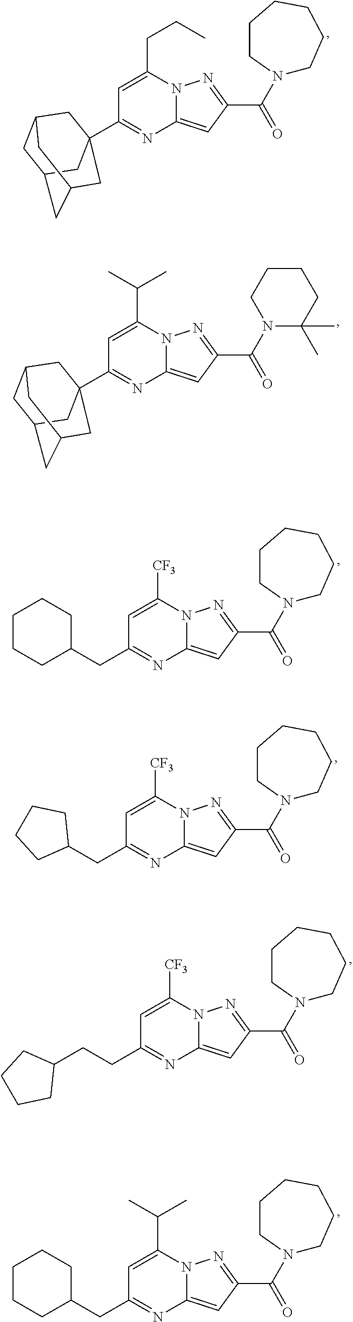 [SMILES: CCCc1cc(C23CC4CC(CC(C4)C2)C3)nc2cc(C(=O)N3CCCCCC3)nn12 ; CC(C)c1cc(C23CC4CC(CC(C4)C2)C3)nc2cc(C(=O)N3CCCCC3(C)C)nn12 ; O=C(c1cc2nc(CC3CCCCC3)cc(C(F)(F)F)n2n1)N1CCCCCC1 ; O=C(c1cc2nc(CC3CCCC3)cc(C(F)(F)F)n2n1)N1CCCCCC1 ; O=C(c1cc2nc(CCC3CCCC3)cc(C(F)(F)F)n2n1)N1CCCCCC1 ; CC(C)c1cc(CC2CCCCC2)nc2cc(C(=O)N3CCCCCC3)nn12]

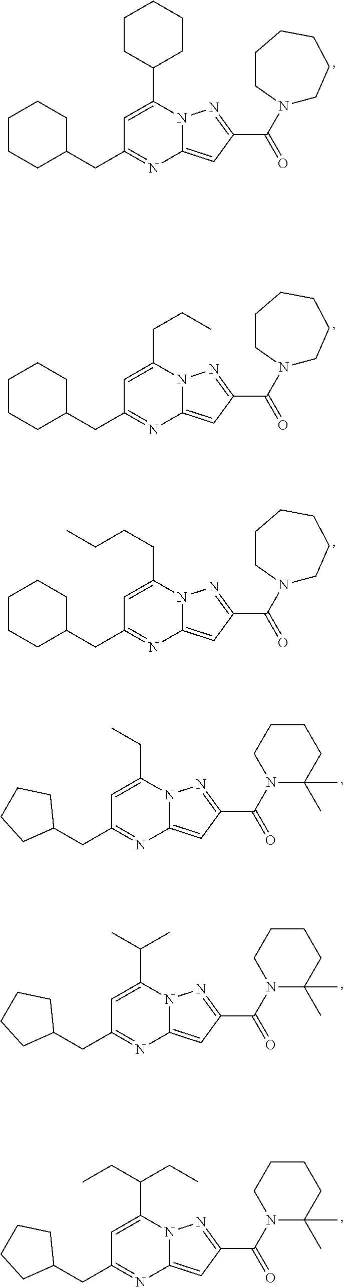 [SMILES: O=C(c1cc2nc(CC3CCCCC3)cc(C3CCCCC3)n2n1)N1CCCCCC1 ; CCCc1cc(CC2CCCCC2)nc2cc(C(=O)N3CCCCCC3)nn12 ; CCCCc1cc(CC2CCCCC2)nc2cc(C(=O)N3CCCCCC3)nn12 ; CCc1cc(CC2CCCC2)nc2cc(C(=O)N3CCCCC3(C)C)nn12 ; CC(C)c1cc(CC2CCCC2)nc2cc(C(=O)N3CCCCC3(C)C)nn12 ; CCC(CC)c1cc(CC2CCCC2)nc2cc(C(=O)N3CCCCC3(C)C)nn12]

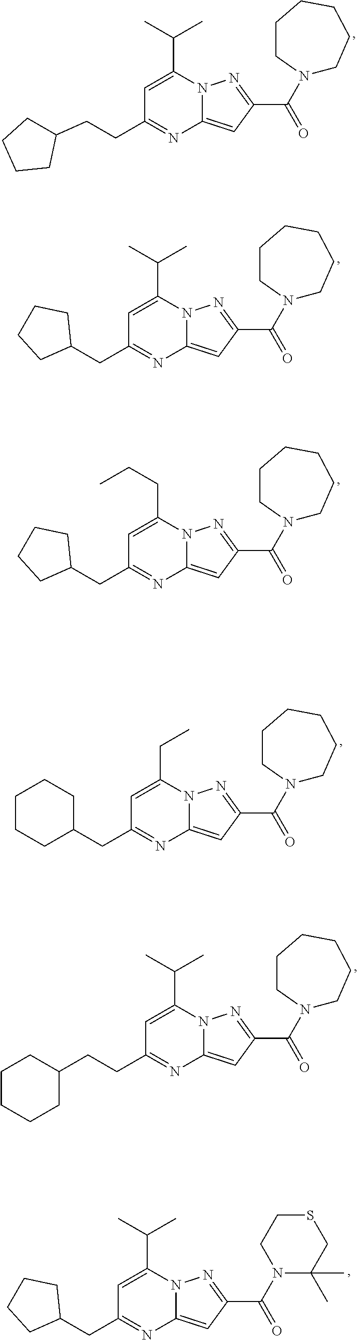 [SMILES: CC(C)c1cc(CCC2CCCC2)nc2cc(C(=O)N3CCCCCC3)nn12 ; CC(C)c1cc(CC2CCCC2)nc2cc(C(=O)N3CCCCCC3)nn12 ; CCCc1cc(CC2CCCC2)nc2cc(C(=O)N3CCCCCC3)nn12 ; CCc1cc(CC2CCCCC2)nc2cc(C(=O)N3CCCCCC3)nn12 ; CC(C)c1cc(CCC2CCCCC2)nc2cc(C(=O)N3CCCCCC3)nn12 ; CC(C)c1cc(CC2CCCC2)nc2cc(C(=O)N3CCSCC3(C)C)nn12]

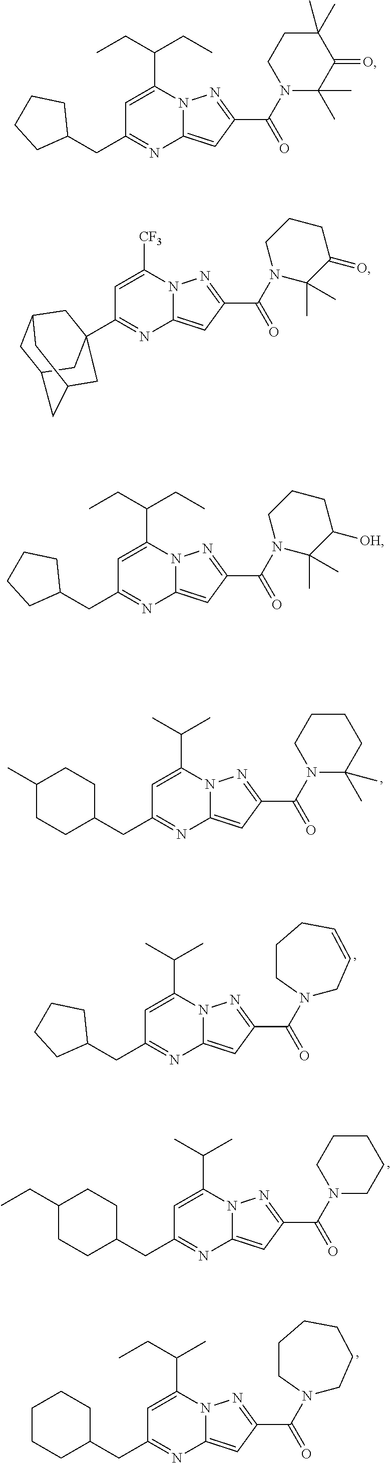

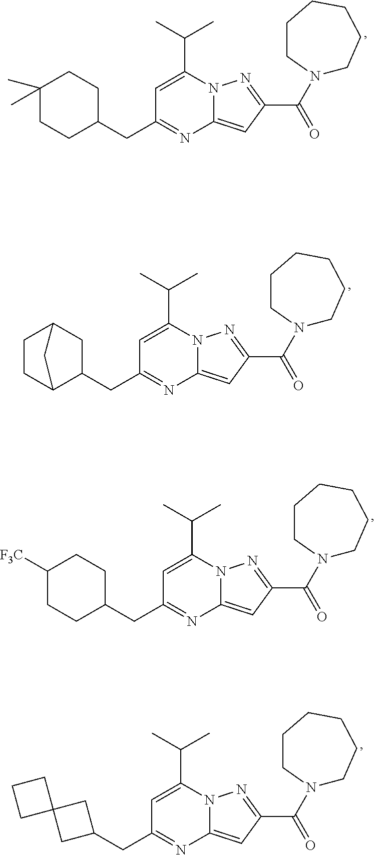 [SMILES: CC(C)c1cc(CC2CCC(C)(C)CC2)nc2cc(C(=O)N3CCCCCC3)nn12 ; CC(C)c1cc(CC2CC3CCC2C3)nc2cc(C(=O)N3CCCCCC3)nn12 ; CC(C)c1cc(CC2CCC(C(F)(F)F)CC2)nc2cc(C(=O)N3CCCCCC3)nn12 ; CC(C)c1cc(CC2CC3(CCC3)C2)nc2cc(C(=O)N3CCCCCC3)nn12]

An embodiment of the present invention relates to a pharmaceutical composition comprising the compound [I] or a salt thereof as active ingredient, and a pharmaceutically acceptable carrier or excipient.

An embodiment of the present invention relates to a therapeutic, preventative and/or diagnostic agent for a symptom and/or disease caused by PAR2 activation, comprising the compound [I] or a salt thereof.

An embodiment of the present invention relates to a therapeutic, preventative and/or diagnostic pharmaceutical composition for a symptom and/or disease caused by PAR2 activation, comprising the compound [I] or a salt thereof as active ingredient.

An embodiment of the present invention relates to a method for treating, preventing and/or diagnosing a symptom and/or disease caused by PAR2 activation, which comprises administering to a human in need thereof an effective amount of the compound [I] or a salt thereof

An embodiment of the present invention relates to the compound [I] or a salt thereof for use in the treatment, prevention and/or diagnosis of a symptom and/or disease caused by PAR2 activation.

An embodiment of the present invention relates to use of the compound [I] or a salt in the manufacture of a medicament for treating, preventing and/or diagnosing a symptom and/or disease caused by PAR2 activation.

An embodiment of the present invention relates to a topical transdermal formulation comprising the compound [I] or a salt thereof as active ingredient, and a pharmaceutically acceptable carrier or excipient.

In the present description, preferred embodiments and alternatives regarding diverse features of the compound [I] or a salt thereof, use, method, and composition of the present invention can be combined, and unless this is incompatible with the nature thereof, the presentation of the combination of preferred embodiments and alternatives regarding the diverse features is also included.

The method for manufacturing the compound [I] will be described below. The compound [I] can be manufactured according to the method for manufacturing described below. These methods for manufacturing are examples and the method for manufacturing the compound [I] is not limited thereto.

In the reaction formulae below, in the case of performing alkylation reaction, hydrolysis reaction, amination reaction, esterification reaction, amidation reaction, etherification reaction, nucleophilic substitution reaction, addition reaction, oxidation reaction, reduction reaction, and the like, these reactions are performed according to methods known per se. Examples of such methods include the methods described in Experimental Chemistry (5th edition, The Chemical Society of Japan ed., Maruzen Co., Ltd.); Organic Functional Group Preparations, 2nd edition, Academic Press, Inc. (1989); Comprehensive Organic Transformations, VCH Publishers Inc. (1989); Greene's Protective Groups in Organic Synthesis, 4th edition, (2006) written by P. G. M. Wuts and T. W. Greene; and the like.

### General Synthetic Pathway (1) of the Compound [I]

### Step 1

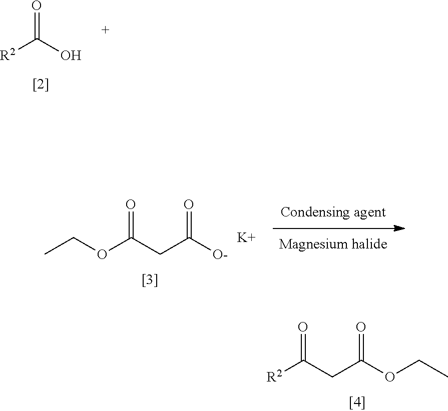

wherein each symbol is as defined above.

The intermediate [4] of the compound [I] can be manufactured by the reaction indicated by the synthetic pathway described above. Specifically, the intermediate [4] can be manufactured by reacting the compound [2] with the compound [3] in an inert solvent for the reaction in the presence of a condensing agent and a magnesium halide.

### Step 2

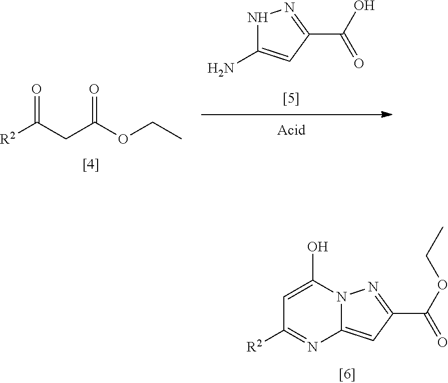

wherein each symbol is as defined above.

The intermediate [6] of the compound [I] can be manufactured by the reaction indicated by the synthetic pathway described above. Specifically, the intermediate [6] can be manufactured by ring-closing reaction of the compound [4] with the compound [5] in ethanol in presence of acid.

The solvent to be used in the reaction is not limited to ethanol, but also includes alcohols such as methanol and propanol. In such cases, ethyl ester of intermediate [6] is not formed, but an alkyl ester of intermediate [6] is formed depending on the solvent used.

### Step 3

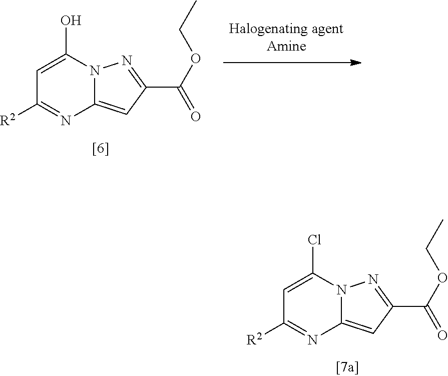

wherein each symbol is as defined above.

The intermediate [7a] of the compound [I] can be manufactured by the reaction indicated by the synthetic pathway described above. Specifically, the intermediate [7a] can be manufactured by reacting the compound [6] with a halogenating agent in an inert solvent for the reaction in the presence of an amine.

### Step 4-1

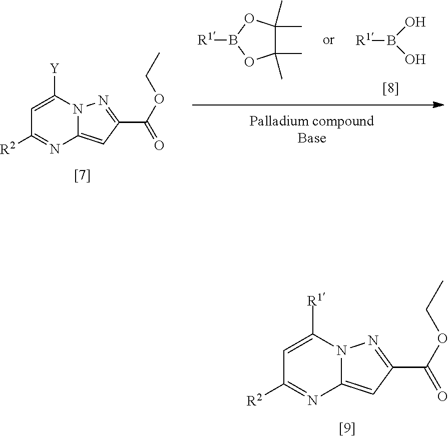

wherein, Y is a leaving group, R^{1′} is R¹ as defined above or partially unsaturated form thereof, and the other symbols are as defined above.

The intermediate [9] of the compound [I] can be manufactured by the reaction indicated by the synthetic pathway described above. Specifically, the intermediate [9] can be manufactured by Suzuki coupling reaction of the compound [7] having a leaving group with a boronic acid compound [8] in an inert solvent for the reaction in the presence of a base and a Palladium compound

The “boronic acid compound” to be used in this reaction may be either a boronic acid ester compound or a boronic acid compound.

### Step 4-2

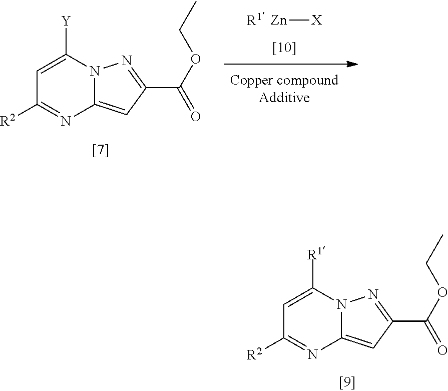

wherein, X and Y are leaving groups, R^{1′} is R¹ as defined above or partially unsaturated form thereof, and the other symbols are as defined above.

The intermediate [9] of the compound [I] can be also manufactured by the reaction indicated by the synthetic pathway described above. Specifically, the intermediate [9] can be manufactured by reacting the compound [7] having a leaving group with an organozinc compound [10] in an inert solvent for the reaction in the presence of a copper compound and an additive.

### Step 5

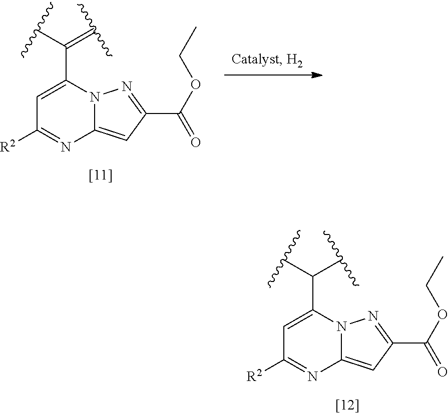

wherein each symbol is as defined above.

The intermediate [12] of the compound [I] can be manufactured by the reaction indicated by the synthetic pathway described above. Specifically, the intermediate [12] can be manufactured by addition of hydrogen to the compound [11] in an inert solvent for the reaction in presence of a catalyst.

### Step 6

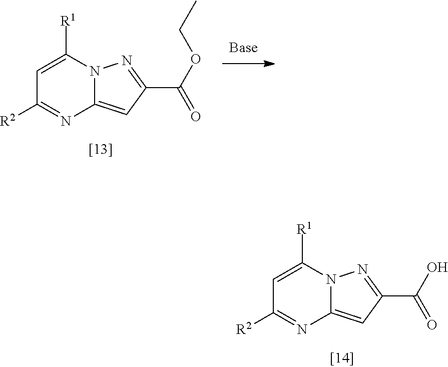

wherein each symbol is as defined above.

The intermediate [14] of the compound [I] can be manufactured by the reaction indicated by the synthetic pathway described above. Specifically, the intermediate [14] can be manufactured by de-esterification of the compound [13] in an inert solvent for the reaction in presence of a base.

### Step 7

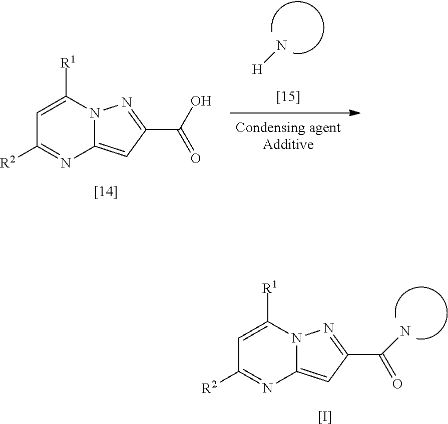

wherein each symbol is as defined above.

The compound [I] can be manufactured by the reaction indicated by the synthetic pathway described above. Specifically, the compound [1] can be manufactured by amidation reaction of the compound [14] with a cyclic amine compound [15] in an inert solvent for the reaction in presence of a condensing agent and an additive.

Other reaction conditions (reaction temperature, reaction time, etc.) can be appropriately determined based on each known reaction.

Each reaction shown in the above synthetic pathway is a general reaction in the present invention, and the order of the reactions may be backward or forward as long as the object compound is obtained.

In each reaction in the above-mentioned equations, the product can be used as a reaction solution or as a crude product thereof in the next reaction. However, the product can be isolated from the reaction mixture in accordance with a conventional method, or easily purified by usual separation means. Examples of the usual separation means include recrystallization, distillation, and chromatography.

The starting material compound, intermediate compound, and objective compound in the above-mentioned steps, and the compound or a salt thereof of the present invention include geometric isomers, stereoisomers, optical isomers, and tautomers. Various isomers can be separated by a general optical resolution method. They can also be manufactured by an appropriate optically active raw material compound.

The compound or a salt thereof of the present invention can be manufactured according to the synthetic methods indicated by the equations described above or methods analogous thereto.

When the specific method of producing the raw material compound used in the manufacturing the compound or a salt thereof of the present invention is not described, the raw material compound may be a commercially available product, or may be a product manufactured according to a method known per se or a method analogous thereto.

The starting material compound and objective compound in the above-mentioned steps can be used in the form of an appropriate salt. Examples of the salt include those similar to the salts exemplified in the following as the salts of the compound [I] of the present invention.

The compound of the present invention includes salt forms thereof including the form of an acid addition salt, or a salt with a base may be formed depending on the kind of the substituent. Examples of the “acid” include an inorganic acid (e.g., hydrochloric acid, hydrobromic acid, nitric acid, sulfuric acid, phosphoric acid, etc.); an organic acid (e.g., methanesulfonic acid, p-toluenesulfonic acid, acetic acid, citric acid, tataric acid, maleic acid, fumaric acid, malic acid, lactic acid, etc.); and the like. Examples of the “base” include an inorganic base (e.g., sodium hydroxide, potassium hydroxide, calcium hydroxide, sodium carbonate, potassium carbonate, sodium hydrogen carbonate, potassium hydrogen carbonate, etc.); an organic base (e.g., methylamine, diethylamine, trimethylamine, triethylamine, ethanolamine, diethanolamine, triethanolamine, ethylenediamine, tris(hydroxymethyl)methylamine, dicyclohexylamine, N,N′-dibenzylethylenediamine, guanidine, pyridine, picoline, choline, etc.); ammonium salts; and the like. In addition, a salt with amino acid such as lysine, arginine, aspartic acid, glutamic acid, and the like may be formed.

The compound of the present invention includes a compound in which one or more atoms are substituted by one or more isotopes. Examples of the isotope include deuterium (²H), tritium (³H), ¹³C, ¹⁵N, ¹⁸O, and the like.

The compound [I] of the present invention includes pharmaceutically acceptable prodrugs. Examples of substituents that can be modified to make prodrugs include reactive functional groups such as —OH, —COOH, amino, and the like. The modifying groups of these functional groups are selected appropriately from the “substituents” in the present description.

The compound represented by the general formula [I] or a pharmaceutically acceptable salt thereof is useful as therapeutic agents, preventative agents, progression preventive agents, or diagnostic agents for symptoms and/or diseases involving hyperfunction of PAR2. Furthermore, the compound represented by the general formula [I] or a salt thereof has PAR2 inhibitory activity, and thus it is useful as research tools to investigate the physiological effects of PAR2.

Examples of symptoms and/or diseases involving hyperfunction of PAR2 include pruritus, skin diseases, allergic diseases, inflammatory diseases, autoimmune diseases, and cancers.

The compound represented by the general formula [I] or a salt thereof shows an excellent antipruritic activity in vivo, and thus useful as antipruritic agents, and therapeutic or preventative agents for various diseases with pruritus. Examples of the disease with pruritus include atopic dermatitis, urticaria, eczema, sebum deficiency, sebum deficiency eczema, senile pruritus, xeroderma, senile xerosis, prurigo, seborrheic dermatitis, psoriasis, contact dermatitis, insect bite, caterpillar dermatitis, photosensitivity, fruit hypersensitivity, neurodermatitis, self-sensitizing dermatitis, pruritus on renal dialysis, pruritus associated with chronic liver disease, lichen amyloidosis, tinea cruris, cutaneous candidiasis, scabies, pruritus due to tick, lice, drug rash, or opioid analgesicdosing, atopic keratoconjunctivitis, allergic keratoconjunctivitis, infectious keratoconjunctivitis, spring catarrh, and the like. Further examples of the disease with pruritus include those caused by internal diseases (malignant tumor, diabetes, liver disease, renal failure, gout, thyroid disease, blood disorder), infection of parasite, fungus, virus, or the like, psychogenic stress, drug hypersensitivity, or pregnancy.

Specific examples of the disease include skin diseases (e.g., atopic dermatitis, psoriasis, eczema, scleroderma and dermatitis), asthma, bronchitis, allergic reactions, allergic contact hypersensitivity, allergic keratoconjunctivitis, arthritis (including osteoarthritis, osteoarthritis, spondyloarthritis, gouty arthritis, systemic lupus erythematosus, juvenile arthritis and chronic rheumatoid arthritis), autoimmune diseases, Huntington's disease, Parkinson's disease, amyotrophic lateral sclerosis, multiple sclerosis, sarcoidosis, Behcet's syndrome, inflammatory bowel disease, Crohn's disease, Alzheimer's disease, organ transplant toxicity, cancers (e.g., solid tumor cancers including colon cancer, breast cancer, lung cancer and prostate cancer; hematopoietic malignant diseases including leukemia and lymphoma; Hodgkin's disease; aplastic anemia, skin cancer and familial adenomatous polyposis), hemophilia, cachexia, tumor invasion, tumor growth, tumor metastasis, and the like.

The following is a description of the dosage and dosage form of a medicament comprising the compound of the present invention as an active ingredient, such as a therapeutic agent, preventive agent, or diagnostic agent for the above diseases.

The compound of the invention can be administered orally or parenterally, and can be used for human or non-human animals in various formulations suitable for oral or parenteral administration as a pharmaceutical composition with appropriate additives, substrates, and carriers. For example, when orally administrated, it can be administrated in a commonly used dosage form such as tablet, capsule, syrup, suspension, etc. When parenterally administered, it can be administrated in the form of injection or eye drop as liquid such as solution, emulsion, suspension, etc., rectally administrated in the form of suppository, or administrated in the form of topical transdermal formulation such as ointment, cream, lotion, spray, etc.

Such dosage forms can be produced according to general methods by blending the active ingredient with auxiliary agents such as carriers, excipients, binders, stabilizers, etc. When used in injection form, they are dissolved or suspended in a physiologically acceptable carrier such as water, physiological saline solution, oil, glucose solution, and the like. If necessary, auxiliary agents such as emulsifiers, stabilizers, osmotic pressure-regulating salts, solubilizers, or buffers may be added thereto.

When administered in topical transdermal formulation, stabilizers, preservatives, emulsifiers, suspension stabilizers, antioxidants, fragrances, fillers, or other transdermal absorption promoting agents can be added as needed, in addition to substrates. Examples of substrate in ointment include fatty oil, lanolin, vaseline, paraffin, plastibase, glycol, higher fatty acid, higher alcohol, etc. Examples of substrate in lotion include ethanol, glycerin, glycol, etc. Examples of substrate in liquid preparation include ethanol, water, glycol, etc.

The dose and the number of doses may vary depending on target diseases, patient's symptom, age, body weight, etc., dosage forms, and the like. When orally administered, the active ingredient can be usually administered to adults in a dose range from about 1 to 1000 mg per day, preferably about 10 to 500 mg per day in single or multiple divided doses. When administered as injection, the active ingredient can be administered in a dose range from about 0.1 to about 500 mg, preferably about 3 to about 100 mg in single or multiple divided doses. When administered as transdermal formulation, a suitable amount of the active ingredient can be applied to the affected area once or several times a day.

The compound of the present invention has excellent transdermal absorbability, and therefore, it is preferably used as topical transdermal formulation such as ointment, cream, and lotion.

The compound of the present invention can be used in combination with steroid (e.g., clobetasol propionate, diflucortolone valerate, betamethasone valerate ester, hydrocortisone butyrate), calcineurin inhibitor (e.g., cyclosporin, tacrolimus), JAK inhibitor (e.g., delgocitinib, baricitinib), PDE4 inhibitor (e.g., crisaborole, apremilast), vitamin D and a derivative thereof (e.g., maxacalcitol), vitamin A derivative (e.g., adapalene), disease-modifying antirheumatic agent (DMARDs, e.g., methotrexate), K-opioid agonist (e.g., nalfurafine hydrochloride), antiallergic agent, antihistamine (e.g., sodium cromoglycate, tranist, suplatast tosylate, chlorpheniramine maleate, fexofenadine hydrochloride, olopatadine hydrochloride, bilastine, rupatadine fumarate), moisturizer (e.g., heparin analogue, urea, zinc oxide), TNFα antibody (e.g., infliximab, adalimumab), IL-4/13R antibody (e.g., dupilumab), IL-12/23p40 antibody (e.g., ustekinumab), IL-13 (e.g., lebrikizumab) antibody, IL-17 antibody (e.g., secukinumab, ixekizumab), IL-17R antibody (e.g., brodalumab), IL-23antibody (e.g., guselkumab), IL-31R antibody (e.g., nemolizumab).

When the compound of the present invention is used in combination with a concomitant drug, each compound may be administered at the same time, separately at about the same time, or separately at different times. The compound and the concomitant drug can also be mixed and administered as a single preparation.

Disclosures of all PTLs and NPLs cited in the present description are incorporated in the present description in their entirety by reference.

### EXAMPLES

The present invention is explained in detail in the following by referring to Test Examples, Reference Examples, and Examples, which are not to be construed as limitative, and the invention may be changed within the scope of the present invention.

In the present description, the following abbreviations may be used.

| Abbreviations | Words |
|---|---|
| REX | Reference Example number |
| EX | Example number |
| STR | Structural formula |
| RProp | Manufacturing method (numbers indicate |
| | that the compound was manufactured using |
| | the corresponding raw materials in the |
| | same way as the reference example |
| | compound having that number as a |
| | reference example number) |
| Prop | Manufacturing method (numbers indicate |
| | that the compound was manufactured using |
| | the corresponding raw materials in the |
| | same way as the example compound |
| | having that number as an example number) |
| Data | Physical property data (NMR1: δ (ppm) |
| | in ¹H-NMR in DMSO-d₆; NMR2: δ (ppm) |
| | in ¹H-NMR in CDCl₃); MS: Mass spectrum) |
| AcOEt | ethyl acetate |
| AcOH | acetic acid |
| AcOK | potassium acetate |
| AcONa | sodium acetate |
| BBrs | boron tribromide |
| n-BuLi | n-butyllithium |
| tBu₃P · HBF₄ | tri-tert-butylphosphonium tetraphenylborate |
| (BPin)₂ | bis(pinacolato)diboron |
| CDI | 1,1′-carbonyldiimidazole |
| COMU | (1-cyano-2-ethoxy-2- |
| | oxoethylidenaminooxy)dimethyl- |
| | aminomorpholinocarbenium |
| | hexafluorophosphate |
| m-CPBA | m-chloroperoxybenzoic acid |
| CPME | cyclopentyl methyl ether |
| Cs₂CO₃ | cesium carbonate |
| DBU | 1,8-diazabicyclo[5.4.0]-7-undecene |
| DCC | dicyclohexylcarbodiimide |
| DCE | 1,2-dichloroethane |
| DCM | dichloromethane |
| DEAD | diethylazodicarboxylate |
| DHP | 3,4-dihydro-2H-pyran |
| DIBAL | diisobutylaluminum hydride |
| DIBOC | di-t-butyl dicarbonate |
| DIPEA | diisopropylethylamine |
| DMA | N,N-dimethylacetamide |
| DMAP | 4-(dimethylamino)pyridine |
| DME | dimethoxyethane |
| DMF | N,N-dimethylformamide |
| DMSO | dimethyl sulfoxide |
| DPPA | diphenylphosphoryl azide |
| Et₂O | diethyl ether |
| EtOH | ethanol |
| HATU | o-(7-azabenzotriazol-1-yl)- |
| | 1,1,3,3-tetramethyluronium |
| | hexafluorophosphate |
| HCl | hydrochloric acid |
| Hexane | n-hexane |
| HOBt | 1-hydroxybenzotriazole |
| IPA | 2-propanol |
| IPE | diisopropyl ether |
| K₂CO₃ | potassium carbonate |
| K₃PO₄ | tripotassium phosphate |
| KHCO₃ | potassium hydrogen carbonate |
| KOH | potassium hydroxide |
| KOtBu | potassium tert-butoxide |
| LAH | lithium aluminum hydride |
| LDA | lithium diisopropylamide |
| LHMDS | lithium hexamethyldisilazide |
| LiOH | lithium hydroxide |
| MeCN | acetonitrile |
| MEK | 2-butanone |
| MeOH | methanol |
| NaBH₄ | sodium borohydride |
| Na₂CO₃ | sodium carbonate |
| NaH | sodium hydride |
| NaHCO₃ | sodium hydrogen carbonate |
| NaOH | sodium hydroxide |
| NaOtBu | sodium t-butoxide |
| NBS | N-bromosuccinimide |
| NCS | N-chlorosuccinimide |
| NHS | N-hydroxysuccinimide |
| NMP | N-methylpyrrolidone |
| Pd/C | palladium-carrying carbon |
| Pd₂(dba)₃ | tris(dibenzylideneacetone)dipalladium(0) |
| Pd(tBu₃P)₂ | bis(tri-t-butylphosphine)palladium |
| PdCl₂(dppf)DCM | [1,1′-bis(diphenylphosphino)ferrocene]palladium(II) |
| | dichloride dichloromethane adduct |
| PdCl₂(PPh₃)₂ | Bis(triphenylphosphine)palladium(II)chloride |
| Pd(OAc)₂ | palladium(II) acetate |
| Pd(PPh₃)₄ | tetrakis(triphenylphosphine)palladium(0) |
| Pt/C | platinum-carrying carbon |
| PEG | polyethylene glycol |
| PPTS | pyridinium p-toluenesulfonate |
| TBAF | tetra-n-butylammonium fluoride |
| TCDI | 1,1′-thiocarbonyldiimidazole |
| TEA | triethylamine |
| TFA | trifluoroacetic acid |
| THF | tetrahydrofuran |
| TosMIC | tosylmethyl isocyanide |
| TPP | triphenylphosphine |
| p-TsOH · H₂O | p-Toluenesulfonic Acid Monohydrate |
| WSC | 3-ethyl-1-(3-dimethylaminopropyl)carbodiimide |
| ZCl | benzyl chloroformate |

In the following Examples, “room temperature” generally means about 10° C. to about 35° C. The ratios indicated for mixed solvents are volume mixing ratios, unless otherwise specified. % means wt %, unless otherwise specified.

1HNMR (proton nuclear magnetic resonance spectrum) was measured by Fourier-transform type NMR (either of BrukerAVANCE III 400 (400 MHz) and Bruker AVANCE III HD (500 MHz)).

Mass spectrum (MS) was measured by LC/MS (ACQUITY UPLC H-Class). As ionization method, ESI method was used. The data indicates actual measured value (found). Generally, molecular ion peaks ([M+H]⁺, [M−H]⁻, etc.) are observed. In the case of a salt, a molecular ion peak or fragment ion peak of free form is generally observed.

In silica gel column chromatography, when denoted as basic, aminopropylsilane-bonded silica gel was used.

The absolute configuration of the compound was determined by known X-ray crystal structure analysis method (e.g., “Basic Course for Chemists 12, X-ray Crystal Structure Analysis” written by Shigeru Ohba and Shigenobu Yano, 1st edition, 1999) or estimated from the empirical rule of Shi asymmetric epoxidation (Waldemar Adam, Rainer T. Fell, Chantu R. Saha-Moller and Cong-Gui Zhao: Tetrahedron: Asymmetry 1998, 9, 397-401; Yuanming Zhu, Yong Tu, Hongwu Yu, Yian Shi: Tetrahedron Lett. 1988, 29, 2437-2440).

### REFERENCE EXAMPLES

### Reference Example 1: Synthesis of 5-(1-adamantyl)-7-propylpyrazolo[1,5-a]pyrimidine-2-carboxylic acid

To a solution of ethyl 5-(1-adamantyl)-7-propylpyrazolo[1,5-a]pyrimidine-2-carboxylate (170 mg) in THE (1.7 ml)-MeOH (1.7 ml) was added 4N LiOH (0.578 ml) at 0° C., and the mixture was stirred at room temperature overnight. The reaction mixture was acidified by the addition of 1N HCl, and then extracted with AcOEt. The organic layer was concentrated to obtain the object compound (157 mg).

### Reference Example 2: Synthesis of ethyl 5-(1-adamantyl)-7-propylpyrazolo[1,5-a]pyrimidine-2-carboxylate

To a solution of ethyl 5-(1-adamantyl)-7-chloropyrazolo[1,5-a]pyrimidine-2-carboxylate (900 mg) and n-propylboronic acid (770 mg) in 1,4-dioxane (18 ml) were added K₂CO₃ (1383 mg) and trans-dichlorobis(triphenylphosphine)palladium(II) (176 mg), and the mixture was stirred at 80° C. overnight. Water was added to the reaction mixture, and then the insoluble material was filtered off. The filtrate was extracted with AcEt, and the organic layer was concentrated. The residue was purified by medium pressure column chromatography (Hexane/AcOEt) to obtain the object compound (615 mg).

### Reference Example 3: Synthesis of 5-(1-adamantyl)-7-propan-2-ylpyrazolo[1,5-a]pyrimidine-2-carboxylic acid

To a solution of ethyl 5-(1-adamantyl)-7-propan-2-ylpyrazolo[1,5-a]pyrimidine-2-carboxylate (98 mg) in EtOH (3 ml) was added 1N NaOH (0.533 ml), and the mixture was stirred at room temperature for 3 hours. The reaction mixture was concentrated, and the residue was acidified by the addition of water and 1 N HCl. The resulting mixture was stirred for 30 minutes, and then the precipitate was collected by filtration to obtain the object compound (81 mg).

### Reference Example 4: Synthesis of ethyl 5-(1-adamantyl)-7-propan-2-ylpyrazolo[1,5-a]pyrimidine-2-carboxylate

To a solution of ethyl 5-(1-adamantyl)-7-prop-1-en-2-ylpyrazolo[1,5-a]pyrimidine-2-carboxylate (100 mg) in AcOEt (5 ml) was added Pd/C (25 mg), and the mixture was stirred under hydrogen atmosphere at room temperature for 1 hour. The reaction mixture was filtered through Celite, and the filtrate was concentrated. The residue was purified by column chromatography (Hexane/AcOEt) to obtain the object compound (99 mg).

### Reference Example 5: Synthesis of ethyl 5-(1-adamantyl)-7-prop-1-en-2-ylpyrazolo[1,5-a]pyrimidine-2-carboxylate

To a solution of ethyl 5-(1-adamantyl)-7-chloropyrazolo[1,5-a]pyrimidine-2-carboxylate (825 mg) and 2-isopropenyl-4,4,5,5-tetramethyl-1,3,2-dioxaborolane (0.517 ml) in 1,4-dioxane (10 ml) were added PdCl₂(dppf)DCM (187 mg) and 2N Na₂CO₃ aq. (3.44 ml), and the mixture was stirred under argon atmosphere at 90° C. for 5 hours. The reaction mixture was concentrated. Water and AcOEt were added to the residue, and then the resulting mixture was filtered through Celite. The filtrate was extracted with AcOEt, and the organic layer was concentrated. The residue was purified by column chromatography (Hexane/AcOEt) to obtain the object compound (747 mg).

### Reference Example 6: Synthesis of ethyl 5-(1-adamantyl)-7-chloropyrazolo[1,5-a]pyrimidine-2-carboxylate

Ethyl 5-(1-adamantyl)-7-oxo-4H-pyrazolo[1,5-a]pyrimidine-2-carboxylate (1.1 g), phosphorus oxychloride (11 ml) and N,N-dimethylaniline (0.408 ml) were mixed and stirred at 90° C. for 8 hours. The reaction mixture was concentrated, and the residue was poured into ice water. Na₂CO₃ aq. was added and neutralized. The resulting mixture was extracted with AcOEt, and the extract was concentrated. The residue was purified by column chromatography (Hexane/AcOEt) to obtain the object compound (825 mg).

### Reference Example 7: Synthesis of ethyl 5-(1-adamantyl)-7-oxo-4H-pyrazolo[1,5-a]pyrimidine-2-carboxylate

Ethyl 3-(1-adamantyl)-3-oxopropanoate (7.2 g), ethyl 5-amino-1H-pyrazole-3-carboxylate (4.46 g), and p-TsOH·H₂O (0.547 g) were added to EtOH (80 ml), and the mixture was heated to reflux overnight. The reaction mixture was concentrated, water was added thereto, and then the precipitate was collected by filtration to obtain the object compound (7.59 g).

### Reference Example 8: Synthesis of 5-(cyclohexylmethyl)-7-(trifluoromethyl)pyrazolo[1,5-a]pyrimidine-2-carboxylic acid

A solution of ethyl 5-(cyclohexylmethyl)-7-(trifluoromethyl)pyrazolo[1,5-a]pyrimidine-2-carboxylate (138 mg) in THE (10 ml) was cooled to −2° C. LiOH (186 mg) aq. (3 ml) was added dropwise to the solution, and the mixture was stirred at −2° C. overnight. To the reaction mixture was added HCl, and the mixture was stirred for 1 hour. Water was added to the mixture, and the resulting mixture was extracted with AcOEt. The organic layer was concentrated to obtain the object compound (133 mg).

### Reference Example 9: Synthesis of ethyl 5-(cyclohexylmethyl)-7-(trifluoromethyl)pyrazolo[1,5-a]pyrimidine-2-carboxylate

Ethyl 5-bromo-7-(trifluoromethyl)pyrazolo[1,5-a]pyrimidine-2-carboxylate (780 mg), (cyclohexylmethyl)zinc bromide solution (5.08 ml), and Pd(Ph₃P)₄ (267 mg) were dissolved in THE (3 ml), and the solution was stirred under argon atmosphere at 50° C. for 4 hours. To the reaction mixture were added water and NH₄Cl aq., and the resulting mixture was extracted with AcOEt. The organic layer was concentrated, and the residue was purified by column chromatography (Hexane/AcOEt) to obtain the object compound (177 mg).

### Reference Example 10: Synthesis of ethyl 5-bromo-7-(trifluoromethyl)pyrazolo[1,5-a]pyrimidine-2-carboxylate

To a solution of ethyl 5-oxo-7-(trifluoromethyl)-4H-pyrazolo[1,5-a]pyrimidine-2-carboxylate (7.0 g) in 1,4-dioxane (70 ml) was added phosphorus oxybromide (14.58 g). The mixture was stirred at 90° C. for 4 hours. After allowing to cool, the reaction mixture was poured into ice water, and the precipitate was collected by filtration to obtain the object compound (8.09 g).

### Reference Example 11: Synthesis of 5-(cyclopentylmethyl)-7-(trifluoromethyl)pyrazolo[1,5-a]pyrimidine-2-carboxylic acid

A solution of 5-cyclopentyl-1,1,1-trifluoropentane-2,4-dione (8.55 g) and 5-amino-1H-pyrazole-3-carboxylic acid (4.89 g) in AcOH (50 ml) was heated to reflux for 2 hours. The reaction mixture was concentrated, and AcOEt was added to the residue. The resulting mixture was extracted with 5N NaOH. The aqueous layer was acidified by the addition of 5N HCl, and the mixture was extracted with AcOEt. The organic layer was concentrated to obtain the object compound (8.80 g).

### Reference Example 12: Synthesis of 5-cyclopentyl-1,1,1-trifluoropentane-2,4-dione

To a solution of 1-cyclopentylpropan-2-one (2.83 g) and ethyl trifluoroacetate (3.20 ml) in THE (30 ml) was added KOtBu (5.03 g) while ice-cooling, and the mixture was stirred at room temperature overnight. To the reaction mixture was added 1N HCl, and the resulting mixture was extracted with Et₂O. The organic layer was concentrated to obtain the object compound (4.39 g).

### Reference Example 15: Synthesis of 5-(cyclohexylmethyl)-7-propan-2-ylpyrazolo[1,5-a]pyrimidine-2-carboxylic acid

To a solution of ethyl 5-(cyclohexylmethyl)-7-propan-2-ylpyrazolo[1,5-a]pyrimidine-2-carboxylate (4.86 g) in EtOH (50 ml) was added 5N NaOH (5.90 ml), and the mixture was stirred at room temperature for 3 hours. The reaction mixture was concentrated. To the residue was added water, and the mixture was acidified by the addition of 5N HCl. The precipitate was collected by filtration to obtain the object compound (4.28 g).

### Reference Example 16: Synthesis of ethyl 5-(cyclohexylmethyl)-7-propan-2-ylpyrazolo[1,5-a]pyrimidine-2-carboxylate

To a solution of ethyl 5-(cyclohexylmethyl)-7-prop-1-en-2-ylpyrazolo[1,5-a]pyrimidine-2-carboxylate (12.9 g) in AcOEt (65 ml) was added 10% Pd/C (1.3 g). The mixture was stirred under hydrogen atmosphere at room temperature for 1 hour. The reaction mixture was filtered through Celite, and the filtrate was concentrated. The residue was purified by column chromatography (Hexane/AcOEt) to obtain the object compound (10.7 g).

### Reference Example 17: Synthesis of ethyl 5-(cyclohexylmethyl)-7-prop-1-en-2-ylpyrazolo[1,5-a]pyrimidine-2-carboxylate

A suspension of ethyl 7-chloro-5-(cyclohexylmethyl)pyrazolo[1,5-a]pyrimidine-2-carboxylate (14 g), 2-isopropenyl-4,4,5,5-tetramethyl-1,3,2-dioxaborolane (9.14 ml), PdCl₂(dppf)DCM (0.355 g), and K₃PO₄ (18.47 g) in 1,4-dioxane (120 ml)-water (30 ml) was heated to reflux under nitrogen atmosphere for 5 hours. The reaction mixture was concentrated. To the residue was added water, and the mixture was extracted with AcOEt. The organic layer was concentrated, and the residue was purified by column chromatography (Hexane/AcOEt) to obtain the object compound (12.9 g).

### Reference Example 18: Synthesis of ethyl 7-chloro-5-(cyclohexylmethyl)pyrazolo[1,5-a]pyrimidine-2-carboxylate

To a solution of ethyl 5-(cyclohexylmethyl)-7-hydroxypyrazolo[1,5-a]pyrimidine-2-carboxylate (17.2 g) in toluene (170 ml) were added phosphorus oxychloride (13.21 ml) and DIPEA (9.90 ml), and the mixture was heated to reflux for 4.5 hours. The reaction mixture was concentrated. To the residue was added ice water, and the mixture was neutralized by the addition of saturated sodium bicarbonate aq. The resulting mixture was extracted with AcOEt, and the organic layer was concentrated to obtain the object compound (18.4 g).

### Reference Example 19: Synthesis of ethyl 5-(cyclohexylmethyl)-7-hydroxypyrazolo[1,5-a]pyrimidine-2-carboxylate

To a suspension of ethyl 4-cyclohexyl-3-oxobutanoate (8.35 g), 5-amino-1H-pyrazole-3-carboxylic acid (5.00 g) in EtOH (50 ml) was added p-TsOH·H₂O (3.74 g), and the mixture was heated to reflux for 5 hours. The reaction mixture was concentrated. To the residue was added water, and the precipitate was collected by filtration to obtain the object compound (13.9 g).

### Reference Example 20: Synthesis of Azepan-1-yl-[7-(cyclohexen-1-yl)-5-(cyclohexylmethyl)pyrazolo[1,5-a]pyrimidin-2-yl]methanone

A solution of azepan-1-yl-[7-chloro-5-(cyclohexylmethyl)pyrazolo[1,5-a]pyrimidin-2-yl]methanone (500 mg), 1-cyclohexen-1-yl-boronic acid pinacol ester (305 mg), PdCl₂(dppf)DCM (109 mg), and K₃PO₄ (566 mg) in 1,4-dioxane (6 ml)-water (3 ml) was heated to reflux under nitrogen atmosphere for 3 hours. Water was added to the mixture, and the resulting mixture was extracted with AcOEt. The organic layer was concentrated, and the residue was purified by column chromatography (Hexane/AcOEt) to obtain the object compound (570 mg).

### Reference Example 21: Synthesis of Azepan-1-yl-[7-chloro-5-(cyclohexylmethyl)pyrazolo[1,5-a]pyrimidin-2-yl]methanone

To a solution of azepan-1-yl-[5-(cyclohexylmethyl)-7-hydroxypyrazolo[1,5-a]pyrimidin-2-yl]methanone (7.4 g) in toluene (40 ml) were added phosphorus oxychloride (5.80 ml) and DIPEA (3.63 ml), and the mixture was heated to reflux for 5 hours. The reaction mixture was concentrated. To the residue was added ice water, and the mixture was neutralized by the addition of saturated sodium bicarbonate aq. The resulting mixture was extracted with AcOEt, and the organic layer was concentrated. The residue was purified by column chromatography (Hexane/AcOEt) to obtain the object compound (5.2 g).

### Reference Example 22: Synthesis of azepan-1-yl-[5-(cyclohexylmethyl)-7-hydroxypyrazolo[1,5-a]pyrimidin-2-yl]methanone

To a solution of 5-(cyclohexylmethyl)-7-hydroxypyrazolo[1,5-a]pyrimidine-2-carboxylic acid (4.78 g) in DMF (50 ml) were added HATU (7.92 g), TEA (2.90 ml), and hexamethyleneimine (2.348 ml), and the mixture was stirred at room temperature overnight. To the reaction mixture were added HCl and water, and the mixture was stirred. The precipitate was collected by filtration to obtain the object compound (5.6 g).

### Reference Example 31: Synthesis of ethyl 5-(cyclopentylmethyl)-7-pentan-3-ylpyrazolo[1,5-a]pyrimidine-2-carboxylate

To a suspension of ethyl 7-chloro-5-(cyclopentylmethyl)pyrazolo[1,5-a]pyrimidine-2-carboxylate (500 mg), copper(I) iodide (30.9 mg), and lithium chloride (68.9 mg) in NMP (5 ml) was added 0.5N 1-ethylpropyl zinc bromide solution (4.87 ml). The mixture was stirred at 50° C. for 5 hours. To the mixture were added water and AcOEt, and the mixture was filtered through Celite. The filtrate was extracted with AcOEt. The organic layer was concentrated, and the residue was purified by medium pressure column chromatography (Hexane/AcOEt) to obtain the object compound (222 mg).

### Reference Example 43: Synthesis of ethyl 5-(2-cyclohexylethyl)-7-hydroxypyrazolo[1,5-a]pyrimidine-2-carboxylate

A solution of diethyl 5-aminopyrazole-1,3-dicarboxylate (3.08 g) in methanesulfonic acid (15 ml) was stirred at 120° C. for 4 hours. To the reaction mixture were added EtOH (30 ml) and ethyl 5-cyclohexyl-3-oxopentanoate (3.22 g), and the mixture was heated to reflux for 3 hours. After allowing to cool to room temperature, water was added to the mixture. The resulting mixture was concentrated. To the residue was added water, and the mixture was stirred. The precipitate was collected by filtration to obtain the object compound (3.07 g).

### Reference Example 44: Synthesis of diethyl 5-aminopyrazole-1,3-dicarboxylate

To a suspension of Potassium (Z)-1-cyano-3-ethoxy-3-oxoprop-1-en-2-olate (109 g) and ethyl carbazate (66.5 g) in MeCN (1000 ml) was added TFA (94 ml), and the mixture was stirred at room temperature for 2 hours. To the reaction mixture was added TEA (339 ml), and the mixture was stirred at room temperature for 2 hours. Then, the resulting mixture was concentrated. To the residue were added IPE and water, and the mixture was stirred. The solid was collected by filtration to obtain the object compound (100.7 g).

### Reference Example 49: Synthesis of ethyl 4-(4-methylcyclohexyl)-3-oxobutanoate

To a solution of 4-methylcyclohexaneacetic acid (2 g) in CPME (40 ml) was added CDI (2.283 g), and the mixture was stirred at room temperature for 1 hour. To the mixture were added monoethyl potassium malonate (2.397 g) and magnesium chloride (1.341 g), and the resulting mixture was stirred at 70° C. for 4 hours. To the reaction mixture was added 1N HCl, and the mixture was stirred for a while, and extracted with AcOEt. The organic layer was concentrated, and the residue was purified by column chromatography (Hexane/AcOEt) to obtain the object compound (2.65 g).

### Reference Example 84: Synthesis of tert-butyl 4-[5-(1-adamantyl)-7-(trifluoromethyl)pyrazolo[1,5-a]pyrimidine-2-carbonyl]-3,3-dimethyl piperazine-1-carboxylate

To a suspension of 5-(1-adamantyl)-7-(trifluoromethyl)pyrazolo[1,5-a]pyrimidine-2-carboxylic acid (200 mg) in DCM (5 ml) were added HATU (312 mg) and TEA (0.114 ml). Ten (10) minutes later, 1-Boc-3,3-dimethyl-piperazine (176 mg) was added to the mixture, and the resulting mixture was stirred at room temperature overnight. To the reaction mixture were added water and Na₂CO₃ aq., and the mixture was extracted with DCM. The organic layer was concentrated, and the residue was purified by column chromatography (Hexane/AcOEt). The product was recrystallized from IPE-Hexane to obtain the object compound (167 mg).

### Reference Example 86: Synthesis of ethyl 5-cyclohexyl-7-(trifluoromethyl)pyrazolo[1,5-a]pyrimidine-2-carboxylate

A solution of 1-cyclohexyl-4,4,4-trifluorobutane-1,3-dione (1.18 g) and ethyl 5-amino-1H-pyrazole-3-carboxylate (0.824 g) in AcOH (15 ml) was heated to reflux overnight. The reaction mixture was concentrated. AcOEt was added to the residue, and the mixture was filtrated. Saturated Na₂CO₃ aq. was added to the filtrate, and the mixture was extracted with AcOEt. The organic layer was concentrated, and the residue was purified by column chromatography (Hexane/AcOEt) to obtain the object compound (1.07 g).

### Reference Example 93: Synthesis of ethyl 5-(1-methylcyclohexyl)-7-(trifluoromethyl)pyrazolo[1,5-a]pyrimidine-2-carboxylate

Ethyl 7-(trifluoromethyl)pyrazolo[1,5-a]pyrimidine-2-carboxylate (100 mg), 1-methylcyclohexanecarboxylic acid (165 mg), ammonium peroxodisulfate (440 mg), and silver nitrate (262 mg) were dissolved in MeCN-water (6 ml), and the solution was stirred at 60° C. for 2 hours. Water was added to the mixture, and the resulting mixture was extracted with AcOEt. The resulting mixture was dried with anhydrous sodium sulfate, and concentrated. The residue was purified by column chromatography (Hexane/AcOEt) to obtain the object compound (114 mg).

### Reference Example 99: Synthesis of 2,2-dimethylazepane hydrochloride

To a solution of 1-benzyl-2,2-dimethylazepane (0.54 g) in EtOH (15 ml) were added palladium hydroxide on carbon (0.122 g) and 2,2-dichloropropane (0.311 ml), and the mixture was stirred under hydrogen atmosphere at 35° C. for 2.5 hours. After nitrogen substitution, the reaction mixture was filtered through Celite, and washed with AcOEt. To the filtrate was added 4N HCl/AcOEt (0.7 ml), and the mixture was subjected to sonication and then concentrated to obtain the object compound (0.33 g).

### Reference Example 100: Synthesis of 2,2,4,4-tetramethylpiperidin-3-one hydrochloride

To a solution of 1-benzyl-2,2,4,4-tetramethylpiperidin-3-one (1.00 g) in AcOEt (10 ml) was added Pd-C(200 mg), and the mixture was stirred under hydrogen atmosphere at room temperature for 30 minutes. Then, the reaction mixture was filtered through Celite. HCl/AcOEt (5.00 ml) was added to the filtrate, and the precipitate was collected by filtration to obtain the object compound (754 mg).

### Reference Example 101: Synthesis of 1-benzyl-2,2,4,4-tetramethylpiperidin-3-one

To a solution of 1-benzyl-2,2-dimethylpiperidin-3-one (100 mg) in THE (1 ml) were added KOtBu (207 mg) and iodomethane (0.086 ml), and the mixture was stirred at room temperature for 30 minutes. Water was added to the mixture, and the resulting mixture was extracted with AcOEt. The organic layer was concentrated, and the residue was purified by column chromatography (Hexane/AcOEt) to obtain the object compound (89 mg).

### Reference Example 108: Synthesis of ethyl 5-(1-adamantylamino)-7-(trifluoromethyl)pyrazolo[1,5-a]pyrimidine-2-carboxylate

Ethyl 5-bromo-7-(trifluoromethyl)pyrazolo[1,5-a]pyrimidine-2-carboxylate (500 mg), 1-adamantanamine (268 mg), and K₂CO₃ (266 mg) were dissolved in DMF, and the solution was stirred at 100° C. for 1 hour. Water was added to the reaction mixture, and the precipitate was collected by filtration to obtain the object compound (614 mg).

### Reference Example 109: Synthesis of 5-(1-adamantyl)-7-propoxypyrazolo[1,5-a]pyrimidine-2-carboxylic acid

To a solution of ethyl 5-(1-adamantyl)-7-chloropyrazolo[1,5-a]pyrimidine-2-carboxylate (200 mg) in THE (2.5 ml) were added 1-propanol (0.831 ml) and 4N LiOH (0.695 ml), and the mixture was stirred at room temperature for 3 hours. The reaction mixture was weakly acidified by the addition of 1N HCl while ice-cooling, and the precipitate was collected by filtration to obtain the object compound (180 mg).

### Reference Example 115: Synthesis of ethyl 5-(cyclopentyloxymethyl)-7-propan-2-ylpyrazolo[1,5-a]pyrimidine-2-carboxylate

To a solution of ethyl 5-(cyclopentyloxymethyl)-7-prop-1-en-2-ylpyrazolo[1,5-a]pyrimidine-2-carboxylate (150 mg) in AcOEt (5 ml) was added Palladium-activated carbon ethylenediamine complex (10 mg). The mixture was stirred under hydrogen atmosphere at room temperature for 5 hours. The reaction mixture was filtered through Celite, and the filtrate was concentrated. The residue was purified by column chromatography (Hexane/AcOEt) to obtain the object compound (110 mg).

### Reference Example 132: Synthesis of 5-piperidin-1-yl-7-(trifluoromethyl)pyrazolo[1,5-a]pyrimidine-2-carboxylic acid

To a solution of ethyl 5-piperidin-1-yl-7-(trifluoromethyl)pyrazolo[1,5-a]pyrimidine-2-carboxylate (471 mg) in THF/EtOH (5 ml) was added 2N LiOH (2 ml). The mixture was stirred at 0° C. for 2 hours, and then HClaq. was added thereto. The reaction mixture was extracted with AcOEt, and concentrated. AcOH (2 ml) was added to the residue, and the mixture was stirred at 100° C. for 3 hours. The resulting mixture was concentrated, and the residue was purified by column chromatography (Hexane/AcOEt) to obtain the object compound (233 mg).

### Reference Example 133: Synthesis of ethyl 5-piperidin-1-yl-7-(trifluoromethyl)pyrazolo[1,5-a]pyrimidine-2-carboxylate

Ethyl 5-bromo-7-(trifluoromethyl)pyrazolo[1,5-a]pyrimidine-2-carboxylate (482 mg), piperidine (0.17 ml), and K₂CO₃ (256 mg) were dissolved in DMF (1.5 ml), and the mixture was stirred at 100° C. for 1 hour. Water was added to the reaction mixture, and the precipitate was collected by filtration to obtain the object compound (411 mg).

### Reference Example 143: Synthesis of 2,2,4,4-tetramethylpiperidin-3-one Hydrochloride

To a solution of 1-benzyl-2,2,4,4-tetramethylpiperidin-3-one (100 mg) in AcOEt (2 ml) was added Pd/C (20 mg), and the mixture was stirred under hydrogen atmosphere at room temperature for 30 minutes. The reaction mixture was filtered through Celite. To the filtrate was added 4N HCl/AcOEt (1.00 ml), and the mixture was stirred. Then, the precipitate was collected to obtain the object compound (58 mg).

### Reference Example 144: Synthesis of 1-benzyl-2,2,4,4-tetramethylpiperidin-3-one

To a solution of 1-benzyl-2,2-dimethylpiperidin-3-one (100 mg) in THE (1 ml) were added KOtBu (207 mg) and iodomethane (0.086 ml), and the mixture was stirred at room temperature for 30 minutes. Water was added to the mixture, and the resulting mixture was extracted with AcOEt. The organic layer was concentrated, and the residue was purified by medium pressure column chromatography (Hexane/AcOEt) to obtain the object compound (89 mg).

### Reference Example 145: Synthesis of 2,2,3-trimethylpiperidin-3-ol Hydrochloride

To a solution of 1-benzyl-2,2,3-trimethylpiperidin-3-ol (948 mg) in EtOH (10 ml) was added Pd/C (200 mg), and the mixture was stirred under hydrogen atmosphere at room temperature for 1 hour. The reaction mixture was filtered through Celite. To the filtrate was added 1N HCl/AcOEt (8.12 ml), and the mixture was concentrated to obtain the object compound (744 mg).

### Reference Example 146: Synthesis of 1-benzyl-2,2,3-trimethylpiperidin-3-ol

To a solution of 1-benzyl-2,2-dimethylpiperidin-3-one (157 mg) in THE (3 ml) was added methylmagnesium bromide (1.350 ml) while ice-cooling, and the mixture was stirred for 1 hour while ice-cooling. Water was added to the mixture, and the resulting mixture was extracted with AcOEt. The organic layer was concentrated, and the residue was purified by medium pressure column chromatography (Hexane/AcOEt) to obtain the object compound (135 mg).

### Reference Example 149: Synthesis of [5-(cyclohexylmethyl)-7-propan-2-ylpyrazolo[1,5-a]pyrimidin-2-yl]-(2,2-dimethylPiperazin-1-yl)methanone

To a solution of tert-butyl 4-[5-(cyclohexylmethyl)-7-propan-2-ylpyrazolo[1,5-a]pyrimidine-2-carbonyl]-3,3-dimethylpiperazine-1-carboxylate (1.37 g) in DCM (13 ml) was added TFA (2.12 ml) while ice-cooling, and the mixture was stirred at room temperature overnight. The reaction mixture was concentrated, and the residue was neutralized by the addition of saturated Na₂CO₃ aq.. The resulting mixture was extracted with AcOEt, and the organic layer was concentrated to obtain the object compound (1.06 g).

### Reference Example 152: Synthesis of 1-benzyl-2,2,4,4-tetramethylpiperidin-3-ol

To a solution of 1-benzyl-2,2,4,4-tetramethylpiperidin-3-one (1.77 g) in MeOH (20 ml) was added NaBH₄ (0.136 g) while ice-cooling. The mixture was stirred at room temperature for 30 minutes, and then water was added thereto. The resulting mixture was extracted with AcOEt. The organic layer was concentrated, and the residue was purified by medium pressure column chromatography (Hexane/AcOEt) to obtain the object compound (1.45 g).

### Reference Example 155: Synthesis of tert-butyl 2,2-dimethyl-3-methylidenepiperidine-1-carboxylate

To a solution of (methyl)triphenylphosphonium bromide (479 mg) in THE (4 ml) was added potassium tert-butoxide (150 mg), and the mixture was stirred at room temperature for 30 minutes. To the mixture was added a solution of tert-butyl 2,2-dimethyl-3-oxopiperidine-1-carboxylate (203 mg) in THE (5 mL), and the mixture was stirred at room temperature for 1 hour. Water was added to the mixture, and the resulting mixture was extracted with AcOEt. The organic layer was concentrated, and the residue was purified by medium pressure column chromatography (Hexane/AcOEt) to obtain the object compound (39 mg).

### Reference Example 156: Synthesis of ethyl 5-(1-adamantyl)-7-pentylpyrazolo[1,5-a]pyrimidine-2-carboxylate

To a solution of 1-(1-adamantyl)-2-(triphenyl-A5-phosphanilidene)ethanone (1.39 g) in toluene (40 ml) was added Hexanal (0.761 ml), and the mixture was heated to reflux for 3 hours. The reaction mixture was concentrated, and the residue was purified by medium pressure column chromatography (Hexane/AcOEt) to obtain intermediate compound (697 mg). The intermediate compound was dissolved in DMF (10.5 ml), and ethyl 5-amino-1H-pyrazole-3-carboxylate (0.492 g) and K₂CO₃ (0.876 g) were added to the solution. Then, the mixture was stirred at 100° C. overnight. To the reaction mixture was added saturated NH₄Cl aq. solution, and the resulting mixture was extracted with AcOEt. The organic layer was concentrated, and the residue was purified by medium pressure column chromatography (Hexane/AcOEt) to obtain the object compound (329 mg).

The compounds of Reference Examples 13, 14, 23-30, 32-42, 45-48, 50-83, 85, 87-92, 94-98, 102-107, 110-114, 116-131, 134-142, 147, 148, 150, 151, 153, 154 and 157-236 were manufactured in the same manner as in Reference Examples 1-12, 15-22, 31, 43, 44, 49, 84, 86, 93, 99-101, 108, 109, 115, 132, 133, 143-146, 149, 152, 155 and 156. Structural formulae and physicochemical data of the compounds of Reference Examples 1 to 236 are shown in Tables 1-1 to 1-32.

TABLE 1-1

| REX | STR | RProp | Data |
|---|---|---|---|
| 1 | 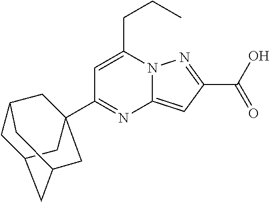 | 1 | NMR1: 13.6-12.7 (brs, 1H), 7.20 (s, 1H), 6.99 (s, 1H), 3.15-3.07 (m, 2H), 2.12- 2.05 (m, 3H), 2.01-1.96 (m, 6H), 1.90- 1.79 (m, 2H), 1.79-1.72 (m, 6H), 0.99 (t, J = 7.4 Hz, 3H). |
| 2 | 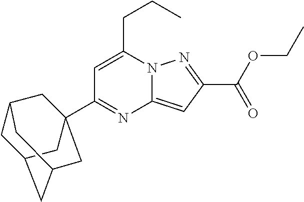 | 2 | NMR2: 7.12 (s, 1H), 6.83 (s, 1H), 4.47 (q, J = 7.1 Hz, 2H), 3.21 (td, J = 7.7, 0.9 Hz, 2H), 2.17-2.12 (m, 3H), 2.04-1.98 (m, 6H), 1.95-1.87 (m, 2H), 1.86-1.75 (m, 6H), 1.44 (t, J = 7.2 Hz, 3H), 1.09 (t, J = 7.4 Hz, 3H). |
| 3 | 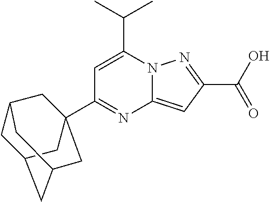 | 3 | NMR1: 13.9-12.7 (brs, 1H), 7.11 (s, 1H), 7.00 (s, 1H), 3.83-3.71 (m, 1H), 2.13- 2.05 (m, 3H), 2.02-1.97 (m, 6H), 1.79- 1.73 (m, 6H), 1.40 (d, J = 6.9 Hz, 6H). |
| 4 | 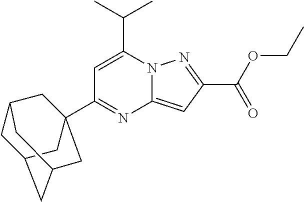 | 4 | NMR2: 7.12 (s, 1H), 6.83 (s, 1H). 4.47 (q, J = 7.1 Hz, 2H), 4.07-3.93 (m, 1H), 2.17- 2.13 (m, 3H), 2.07-2.00 (m, 6H), 1.89- 1.73 (m, 6H), 1.48-1.40 (m, 9H). |
| 5 | 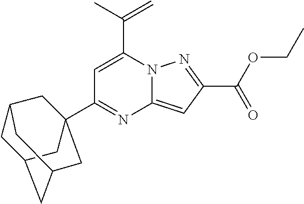 | 5 | NMR2: 7.15 (s, 1H), 6.92 (s, 1H), 6.20- 8.15 (m, 1H), 5.71-5.65 (m, 1H), 4.45 (q, J = 7.1 Hz, 2H), 2.36 (s, 3H), 2.18-2.12 (m, 3H), 2.06-2.01 (m, 6H), 1.90-1.74 (m, 6H), 1.43 (t, J = 7.1 Hz, 3H). |
| 6 | 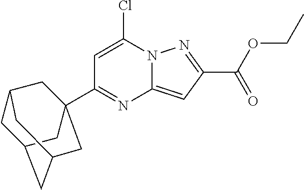 | 6 | NMR2: 7.22 (s, 1H), 7.14 (s, 1H), 4.50 (q, J = 7.1 Hz, 2H), 2.18-2.14 (m, 3H), 2.04- 1.99 (m, 6H), 1.87-1.74 (m, 6H), 1.45 (t, J = 7.1 Hz, 3H). |

TABLE 1-2

| REX | STR | RProp | Data |
|---|---|---|---|
| 7 | 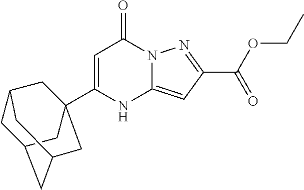 | 7 | NMR2: 9.39 (s, 1H), 6.53 (s, 1H), 5.87 (d, J = 2.2 Hz, 1H), 4.37 (q, J = 7.1 Hz, 2H), 2.18-2.13 (m, 3H), 2.01-1.93 (m, 6H), 1.86-1.69 (m, 6H), 1.33 (t, J = 7.1 Hz, 3H). |
| 8 | 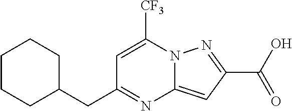 | 8 | NMR1: 13.54 (s, 1H), 7.73 (s, 1H), 7.24 (s, 1H), 2.80 (d, J = 7.1 Hz, 2H), 1.93-1.82 (m, 1H), 1.73-1.53 (m, 5H), 1.30-0.94 (m, 5H). |
| 9 | 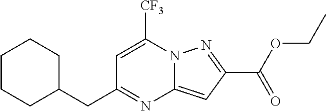 | 9 | NMR2: 7.24 (s, 1H), 7.11 (s, 1H), 4.49 (q, J = 7.1 Hz, 2H), 2.79 (d, J = 7.2 Hz, 2H), 1.92-1.81 (m, 1H), 1.77-1.63 (m, 5H), 1.44 (t, J = 7.1 Hz, 3H), 1.33-1.02 (m, 5H). |
| 10 | 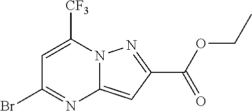 | 10 | NMR2: 7.39 (s, 1H), 7.30 (s, 1H), 4.49 (q, J = 7.1 Hz, 2H), 1.45 (t, J = 7.1 Hz, 3H). |
| 11 | 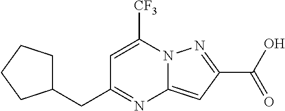 | 11 | NMR1: 13.52 (s, 1H), 7.75 (s, 1H), 7.25 (s, 1H), 2.93 (d, J = 7.5 Hz, 2H), 2.41-2.31 (m, 1H), 1.77-1.68 (m, 2H), 1.68-1.56 (m, 2H), 1.56-1.46 (m, 2H), 1.28-1.18 (m, 2H). |
| 12 | 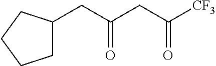 | 12 | NMR2: 5.95-3.69 (m, 2H), 2.62-2.31 (m, 2H), 2.25-2.13 (m, 1H), 1.90-1.51 (m, 6H), 1.19-1.09 (m, 2H). |
| 13 | 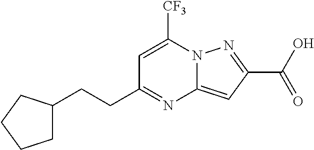 | 11 | NMR1: 13.52 (s, 1H), 7.77 (s, 1H), 7.23 (s, 1H), 2.98-2.89 (m, 2H), 1.90-1.71 (m, 5H), 1.66-1.43 (m, 4H), 1.21-1.08 (m, 2H). |
| 14 | 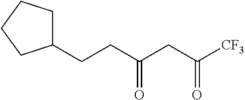 | 12 | NMR2: 6.92 (s, 1H), 2.49-2.41 (m, 3H), 1.84-1.49 (m, 9H), 1.20-1.01 (m, 2H). |

TABLE 1-3

| REX | STR | RProp | Data |
|---|---|---|---|
| 15 | 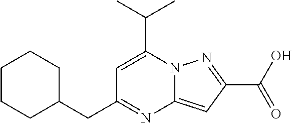 | 15 | NMR1: 13.23 (s, 1H), 7.01-6.96 (m, 2H), 3.82-3.67 (m, 1H), 2.69 (d, J = 7.1 Hz, 2H), 1.92-1.76 (m, 1H), 1.73-1.54 (m, 5H), 1.37 (d, J = 6.9 Hz, 6H), 1.27-0.92 (m, 5H). |
| 16 | 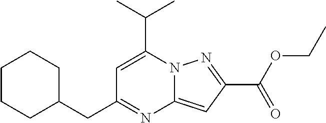 | 16 | NMR2: 7.08 (s, 1H), 6.61 (s, 1H), 4.48 (q, J = 7.1 Hz, 2H), 4.07-3.92 (m, 1H), 2.70 (d, J = 7.2 Hz, 2H), 1.89-1.61 (m, 6H), 1.48-1.39 (m, 9H), 1.31-0.99 (m, 5H). |
| 17 | 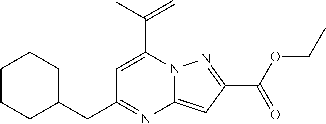 | 17 | NMR2: 7.11 (s, 1H), 6.71 (s, 1H), 6.29- 6.24 (m, 1H), 5.75-5.69 (m, 1H), 4.46 (q, J = 7.1 Hz, 2H), 2.71 (d. J = 7.1 Hz, 2H), 2.36 (s, 3H), 1.89-1.80 (m, 1H), 1.76- 1.63 (m, 5H), 1.43 (t, J = 7 1 Hz, 3H), 1.31-0.99 (m, 5H). |
| 18 | 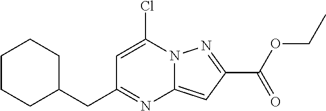 | 18 | NMR2: 7.20 (s, 1H), 6.94 (s, 1H), 4.50 (q, J = 7.1 Hz, 2H), 2.72 (d, J = 7.2 Hz, 2H), 1.91-1.78 (m, 1H), 1.75-1.67 (m, 5H), 1.45 (t, J = 7.1 Hz, 3H), 1.34-0.98 (m, 5H). |
| 19 | 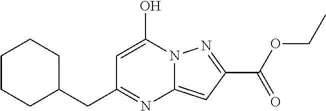 | 19 | NMR2: 12.31 (s, 1H), 6.63 (s, 1H), 5.76 (d, J = 1.9 Hz, 1H), 4.30 (q, J = 7.1 Hz, 2H), 2.66 (d, J = 7.1 Hz, 2H), 1.73-1.62 (m, 6H), 1.21 (t, J = 7.1 Hz. 3H), 1.18-0.88 (m, 5H). |
| 20 | 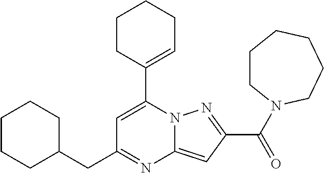 | 20 | NMR1: 7.03-6.96 (m, 1H), 6.92 (s, 1H), 6.78 (s, 1H), 3.67 (t, J = 6.0 Hz, 2H), 3.60 (t, J = 6.0 Hz, 2H), 2.67 (d, J = 7.2 Hz, 2H), 2.58-2.54 (m, 2H), 2.34-2.26 (m, 2H), 1.88-1.51 (m, 14H), 1.32-1.09 (m, 7H), 1.07-0.94 (m, 2H). |
| 21 | 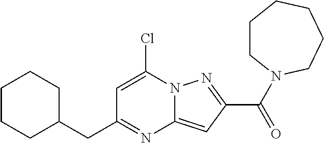 | 21 | NMR2: 6.95 (s, 1H), 6.87 (s, 1H), 3.77- 3.70 (m, 4H), 2.70 (d, J = 7.2 Hz, 2H), 1.92- 1.73 (m, 5H), 1.76-1.61 (m, 9H), 1.33- 0.96 (m, 5H). |

TABLE 1-4

| REX | STR | RProp | Data |
|---|---|---|---|
| 22 | 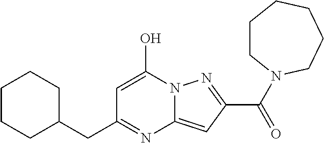 | 22 | NMR2: 11.72 (s, 1H), 6.89 (s, 1H), 5.83 (s, 1H), 3.77 (t, J = 6.0 Hz, 2H), 3.71-3.63 (m, 2H), 2.37 (d, J = 6.8 Hz, 2H), 1.87-1.77 (m, 2H), 1.71-1.47 (m, 12H), 1.22-0.84 (m, 5H). |
| 23 | 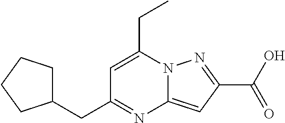 | 1 | NMR1: 13.23 (s, 1H), 7.04-7.01 (m, 1H), 6.99 (s, 1H), 3.13 (q, J = 7 4 Hz, 2H), 2.80 (d, J = 7.5 Hz, 2H), 2.39-2.26 (m, 1H), 1.76- 1.43 (m, 6H), 1.36 (t, J = 7.5 Hz, 3H), 1.29- 1.16 (m, 2H). |
| 24 | 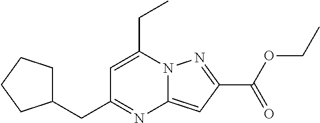 | 2 | NMR2: 7.09 (s, 1H), 6.87 (s, 1H), 4.48 (q, J = 7.1 Hz, 2H), 3.27 (q, J = 7.6 Hz, 2H), 2.83 (d, J = 7.5 Hz, 2H), 2.40-2.24 (m, 1H), 1.84- 1.62 (m, 4H), 1.69-1.51 (m, 2H), 1.44 (t, J = 7.1 Hz, 6H), 1.34-1.18 (m, 2H). |
| 25 | 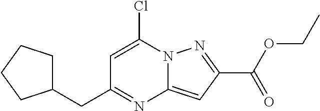 | 6 | NMR2: 7.19 (s, 1H), 6.97 (s, 1H), 4.50 (q, J = 7.1 Hz, 2H), 2.85 (d, J = 7.5 Hz, 2H), 2.40- 2.24 (m, 1H), 1.85-1.51 (m, 6H), 1.45 (t, J = 7.1 Hz, 3H), 1.33-1.20 (m, 2H). |
| 26 | 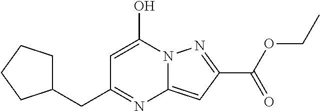 | 7 | NMR2: 12.34 (s, 1H), 6.62 (s, 1H), 5.80 (s, 1H), 4.30 (q, J = 7.1 Hz, 2H), 2.80 (d, J = 7.5 Hz, 2H), 2.36-2.20 (m, 1H), 1.81-1.40 (m, 5H), 1.28-1.15 (m, 5H). |
| 27 | 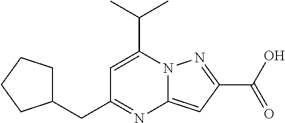 | 1 | NMR1: 13.23 (s, 1H), 7.01 (s, 1H), 6.99 (s, 1H), 3.82-3.67 (m, 1H), 2.81 (d, J = 7.5 Hz, 2H), 2.41-2.25 (m, 1H), 1.75-1.43 (m, 6H), 1.37 (d, J = 6.9 Hz, 6H), 1.29-1.16 (m, 2H). |
| 28 | 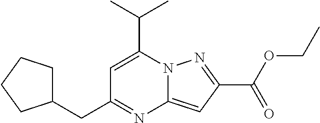 | 4 | NMR2: 7.08 (s, 1H), 6.66 (s, 1H), 4.48 (q, J = 7.1 Hz, 2H), 4.07-3.92 (m, 1H), 2.83 (d, J = 7.5 Hz, 2H), 2.40-2.25 (m, 1H), 1.84- 1.51 (m, 6H), 1.48-1.39 (m, 9H), 1.34- 1.20 (m, 2H). |
| 29 | 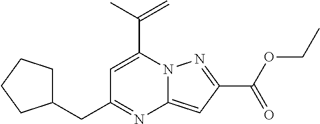 | 2 | NMR2: 7.11 (s, 1H), 6.74 (s, 1H), 6.29-6.24 (m, 1H), 5.75-5.69 (m, 1H), 4.46 (q, J = 7.1 Hz, 2H), 2.84 (d, J = 7.5 Hz, 2H), 2.41- 2.25 (m, 4H), 1.85-1.50 (m, 6H), 1.43 (t, J = 7.1 Hz, 3H), 1.34-1.21 (m, 2H). |

TABLE 1-5

| REX | STR | RProp | Data |
|---|---|---|---|
| 30 | 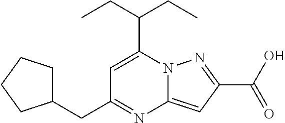 | 1 | NMR2: 7.20 (s, 1H), 6.64 (s, 1H), 3.81-3.69 (m, 1H), 2.86 (d, J = 7.5 Hz, 2H), 2.38-2.26 (m, 1H), 1.98-1.47 (m, 10H), 1.34-1.20 (m, 2H), 0.88 (t, J = 7.4 Hz, 6H). |
| 31 | 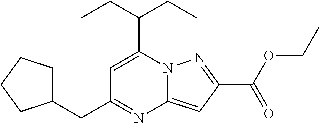 | 31 | NMR2: δ 7.07 (s, 1H), 6.59 (s, 1H), 4.52- 4.42 (m, 2H), 3.86-3.75 (m, 1H), 2.83 (d, J = 7.5 Hz, 2H), 2.37-2.24 (m, 1H), 1.96- 1.49 (m, 10H), 1.42 (t, J = 7.2 Hz, 3H), 1.33- 1.20 (m, 2H), 0.87 (t, J = 7.4 Hz, 6H). |
| 32 | 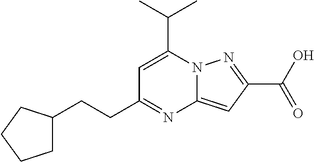 | 1 | NMR1: 13.24 (s, 1H), 7.03 (s, 1H), 6.98 (s, 1H), 3.80-3.69 (m, 1H), 2.86-2.78 (m, 2H), 1.87-1.68 (m, 5H), 1.65-1.44 (m, 4H), 1.37 (d, J = 6.9 Hz, 6H), 1.19-1.10 (m, 2H). |
| 33 | 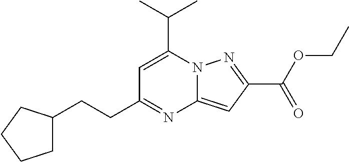 | 4 | NMR2: 7.08 (s, 1H), 6.65 (s, 1H), 4.47 (q, J = 7.1 Hz, 2H), 4.07-3.92 (m, 1H), 2.88- 2.80 (m, 2H), 1.92-1.74 (m, 4H), 1.70- 1.49 (m, 4H), 1.48-1.39 (m, 10H), 1.24- 1.11 (m, 2H). |
| 34 | 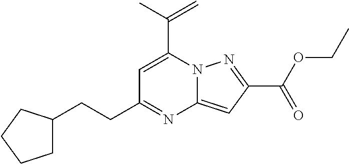 | 2 | NMR2: 7.11 (s, 1H), 6.74 (s, 1H), 6.28-6.22 (m, 1H), 5.74-5.68 (m, 1H), 4.46 (q, J = 7.1 Hz, 2H), 2.90-2.81 (m, 2H), 2.38-2.33 (m, 3H), 1.92-1.75 (m, 3H), 1.70-1.49 (m, 6H), 1.43 (t, J = 7.1 Hz, 3H), 1.24-1.13 (m, 2H). |
| 35 | 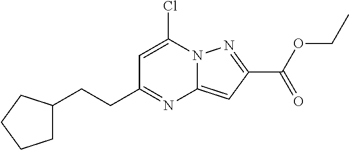 | 6 | NMR2: 7.19 (s, 1H), 6.98 (s, 1H), 4.50 (q, J = 7.1 Hz, 2H), 2.90-2.82 (m, 2H), 1.91- 1.75 (m, 5H), 1.70-1.49 (m, 4H), 1.45 (t, J = 7.1 Hz, 3H), 1.24-1.07 (m, 2H). |
| 36 | 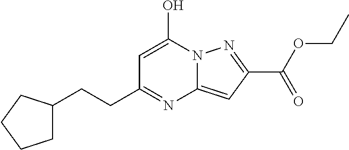 | 7 | NMR1: 12.46 (s, 1H), 6.49 (s, 1H), 5.73 (d, J = 1.8 Hz, 1H), 4.34 (q, J = 7.1 Hz, 2H), 2.63-2.55 (m, 2H), 1.87-1.40 (m, 9H), 1.33 (t, J = 7.1 Hz, 3H), 1.20-1.08 (m, 2H). |

TABLE 1-6

| REX | STR | RProp | Data |
|---|---|---|---|
| 37 | 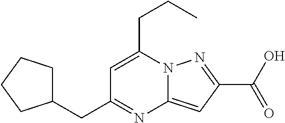 | 7 | NMR2: 7.19 (s, 1H), 6.71 (s, 1H), 3.24-3.15 (m, 2H), 2.86 (d, J = 7.6 Hz, 2H), 2.38-2.28 (m, 1H), 1.99-1.85 (m, 2H), 1.84-1.48 (m, 6H), 1.34-1.21 (m, 2H), 1.10 (t, J = 7 4 Hz, 3H). |
| 38 | 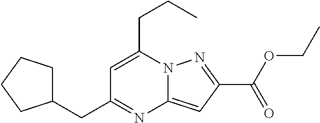 | 2 | NMR2: 7.08 (s, 1H), 6.65 (s, 1H), 4.48 (q, J = 7.1 Hz, 2H), 3.25-3.18 (m, 2H), 2.82 (d, J = 7.5 Hz, 2H), 2.39-2.23 (m, 1H), 1.98- 1.84 (m, 2H), 1.83-1.50 (m, 6H), 1.44 (t, J = 7.1 Hz, 3H), 1.33-1.22 (m, 2H), 1.09 (t, J = 7.4 Hz, 3H). |
| 39 | 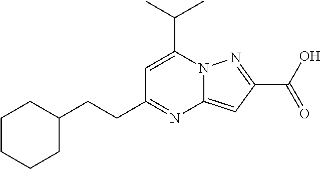 | 1 | NMR1: 13.20 (s, 1H), 7.02 (s, 1H), 6.98 (s, 1H), 3.82-3.67 (m, 1H), 2.86-2.77 (m, 2H), 1.80-1.54 (m, 7H), 1.37 (d, J = 6.9 Hz, 6H), 1.34-1.07 (m, 4H), 1.00-0.86 (m, 2H). |
| 40 | 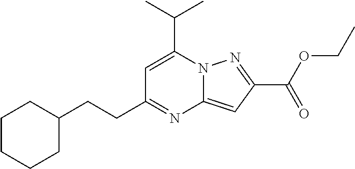 | 4 | NMR1: 7.04 (s, 1H), 7.03 (s, 1H), 4.38 (q, J = 7.1 Hz, 2H), 2.86-2.78 (m, 2H), 1.80- 1.55 (m, 8H), 1.40-1.31 (m, 9H), 1.31- 1.09 (m, 4H), 1.00-0.86 (m, 2H). |
| 41 | 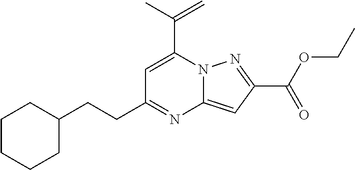 | 2 | NMR1: 7.12 (s, 1H), 7.07 (s, 1H), 6.35-6.30 (m, 1H), 5.82-5.76 (m, 1H), 4.38 (q, J = 7.1 Hz, 2H), 2.87-2.79 (m, 2H), 2.33-2.27 (m, 3H), 1.80-1.54 (m, 7H), 1.35 (t, J = 7.1 Hz, 3H), 1.32-1.04 (m, 4H), 0.99-0.82 (m, 2H). |
| 42 | 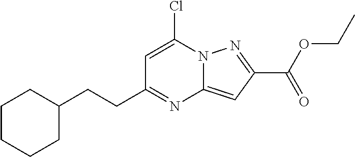 | 6 | NMR1: 7.58 (s, 1H), 7.19 (s, 1H), 4.39 (q, J = 7.1 Hz, 2H), 2.88-2.79 (m, 2H), 1.78- 1.54 (m, 7H), 1.35 (t, J = 7.1 Hz, 3H), 1.35- 1.05 (m, 4H), 0.99-0.82 (m, 2H). |
| 43 | 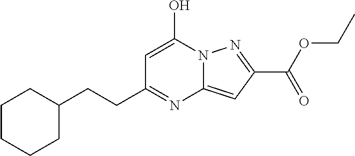 | 43 | NMR1: 12.45 (s, 1H), 6.48 (s, 1H), 5.72 (s, 1H), 4.34 (q, J = 7.1 Hz, 2H), 2.63-2.55 (m, 2H), 1.77-1.49 (m, 7H), 1.33 (t, J = 7.1 Hz, 3H), 1.28-1.04 (m, 4H), 0.99-0.85 (m, 2H). |

TABLE 1-7

| REX | STR | RProp | Data |
|---|---|---|---|
| 44 | 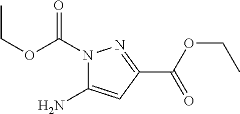 | 44 | NMR1: 6.57 (s, 2H), 5.68 (s, 1H), 4.42 (q, J = 7.1 Hz, 2H), 4.26 (q, J = 7.1 Hz, 2H), 1.35 (t, J = 7.1 Hz, 3H), 1.28 (t, J = 7.1 Hz, 3H). |
| 45 | 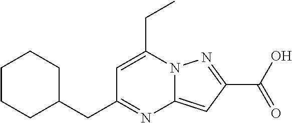 | 1 | NMR1: 13.23 (s, 1H), 7.00 (s, 1H), 6.99 (s, 1H), 3.12 (q, J = 7.5 Hz, 2H), 2.68 (d, J = 7.2 Hz, 2H), 1.88-1.78 (m, 1H), 1.70-1.56 (m, 5H), 1.36 (t, J = 7.5 Hz, 3H), 1.28-0.90 (m, 5H). |
| 46 | 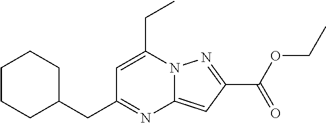 | 2 | NMR2: 7.09 (s, 1H), 6.64 (s, 1H), 4.48 (q, J = 7.1 Hz, 2H), 3.32-3.21 (m, 2H), 2.70 (d, J = 7.2 Hz, 2H), 1.93-1.61 (m, 6H), 1.48- 1.40 (m, 6H), 1.34-0.96 (m, 5H). |
| 47 | 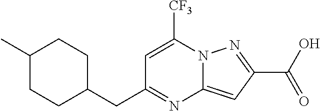 | 11 | NMR2: 7.35 (s, 1H), 7.20-7.14 (m, 1H), 2.98- 2.77 (m, 2H), 2.15-2.07 (m, 1H), 1.89- 1.61 (m, 2H), 1.59-1.27 (m, 5H), 1.19-1.04 (m, 1H), 1.02-0.85 (m, 4H). |
| 48 | 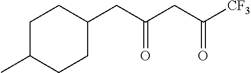 | 12 | NMR2: 5.92-5.86 (m, 1H), 3.79-3.71 (m, 1H), 2.44-2.24 (m, 2H), 2.08-1.17 (m, 8H), 1.09-0.84 (m, 5H). |
| 49 | 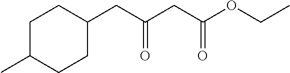 | 49 | NMR2: 4.25-4.14 (m, 2H), 3.41 (d, J = 5.2 Hz, 2H), 2.54-2.36 (m, 2H), 2.19-2.02 (m, 1H), 1.82-1.45 (m, 4H), 1.42-1.09 (m, 6H), 1.06-0.80 (m, 5H). |
| 50 | 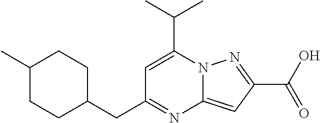 | 1 | NMR2: 7.22-7.17 (m, 1H), 6.70-6.64 (m, 1H), 3.99-3.88 (m, 1H), 2.86-2.69 (m, 2H), 2.13-1.61 (m, 3H), 1.57-1.47 (m, 3H), 1.44 (d, J = 6.9 Hz, 6H), 1.41-1.29 (m, 2H), 1.17- 0.82 (m, 5H). |
| 51 | 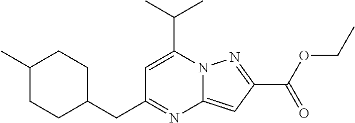 | 4 | NMR2: 7.10-7.06 (m, 1H), 6.65-6.59 (m, 1H), 4.48 (q, J = 7.1 Hz, 2H), 4.05-3.94 (m, 1H), 2.86-2.64 (m, 2H), 1.77-1.61 (m, 2H), 1.56-1.30 (m, 15H), 1.15-0.79 (m, 5H). |
| 52 | 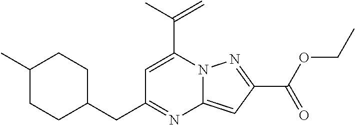 | 2 | NMR2: 7.11 (s, 1H), 6.75-6.69 (m, 1H), 6.30- 6.24 (m, 1H), 5.75-5.69 (m, 1H), 4.46 (q, J = 7.1 Hz, 2H), 2.85-2.64 (m, 2H), 2.38- 2.33 (m, 3H), 2.14-1.62 (m, 3H), 1.56-1.30 (m, 8H), 1.16-0.82 (m, 5H). |

TABLE 1-8

| REX | STR | RProp | Data |
|---|---|---|---|
| 53 | 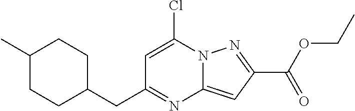 | 6 | NMR2: 7.20 (s, 1H), 6.98-6.92 (m, 1H), 4.50 (q, J = 7.1 Hz, 2H), 2.85-2.67 (m, 2H), 2.09-2.02 (m, 1H), 1.84-1.62 (m, 2H), 1.55-1.28 (m, 8H), 1.16-0 84 (m, 5H). |
| 54 | 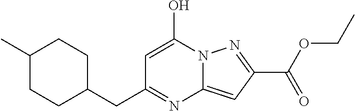 | 7 | NMR2: 12.05 (s, 1H), 6.61 (s, 1H), 5.80- 5.74 (m, 1H), 4.30 (q, J = 7.1 Hz, 2H), 2.80- 2.60 (m, 2H), 2.03-1.98 (m, 1H), 1.78- 1.64 (m, 2H), 1.52-1.16 (m, 8H), 1.07- 0.75 (m, 5H). |
| 55 | 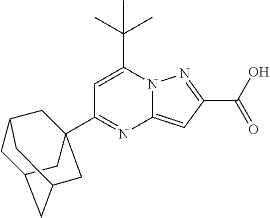 | 1 | NMR2: 7.20 (s, 1H), 6.91 (s, 1H), 2.18-2.13 (m, 3H), 2.05-2.01 (m, 6H), 1.86-1.76 (m, 6H), 1.64 (s, 9H). |
| 56 | 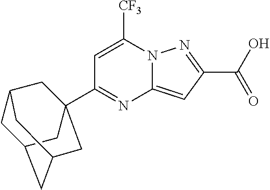 | 1 | NMR1: 13.50 (s, 1H), 7.82 (s, 1H), 7.26 (s, 1H), 2.13-2.07 (m, 3H), 2.04-1.99 (m, 6H), 1.79-1.73 (m, 6H). |
| 57 | 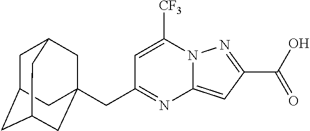 | 11 | NMR2: 7.37 (s, 1H), 7.11 (s, 1H), 2.71 (s, 2H), 2.22-2.09 (m, 1H), 2.04-1.90 (m, 4H), 1.80-1.51 (m, 10H). |
| 58 | 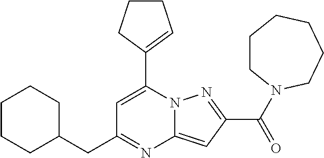 | 20 | NMR1: 7.83 (t, J = 2.4 Hz, 1H), 6.97 (s, 1H), 6.84 (s, 1H), 3.69 (t, J = 6.0 Hz, 2H), 3.65- 3.58 (m, 2H), 2.97-2.82 (m, 2H), 2.70 (d, J = 7.1 Hz, 4H), 2.06-1.94 (m, 2H), 1.92- 1.49 (m, 14H), 1.27-0.89 (m, 6H). |
| 59 | 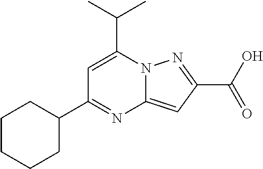 | 1 | NMR1: 13.24 (s, 1H), 7.03 (s, 1H), 6.99 (s, 1H), 3.82-3.67 (m, 1H), 2.84-2.72 (m, 1H), 1.94-1.66 (m, 5H), 1.62-1.48 (m, 2H), 1.46-1.14 (m, 9H). |

TABLE 1-9

| REX | STR | RProp | Data |
|---|---|---|---|
| 60 | 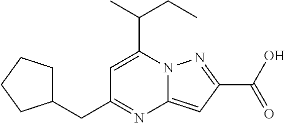 | 1 | NMR2: 7.18 (s, 1H), 6.67 (s, 1H), 3.85-3.73 (m, 1H), 2.86 (d, J = 7.5 Hz, 2H), 2.40-2.24 (m, 1H), 1.99-1.48 (m, 8H), 1.40 (d, J = 7.0 Hz, 3H), 1.36-1.20 (m, 2H), 0.96 (t, J = 7.4 Hz, 3H). |
| 61 | 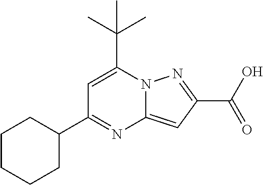 | 1 | NMR1: 13.23 (s, 1H), 6.98 (s, 1H), 6.94 (s, 1H), 2.86-2.74 (m, 1H), 1.95-1.66 (m, 5H), 1.80-1.47 (m, 11H), 1.46-1.16 (m, 3H). |
| 62 | 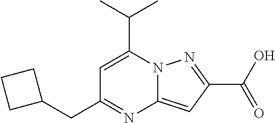 | 1 | NMR2: 7.19 (s, 1H), 6.65 (s, 1H), 3.98-3.87 (m, 1H), 2.95 (d, J = 7.6 Hz, 2H), 2.87-2.72 (m, 1H), 2.18-2.06 (m, 2H), 1.98-1.75 (m, 4H), 1.43 (d, J = 6.9 Hz, 6H). |
| 63 | 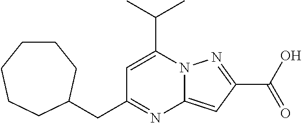 | 1 | NMR2: 7.18 (s, 1H), 6,67 (s, 1H), 3.98-3.86 (m, 1H), 2.75 (d, J = 7.4 Hz, 2H), 2.47-1.48 (m, 11H), 1.44 (d, J = 6.9 Hz, 6H), 1.36- 1.21 (m, 2H). |
| 64 | 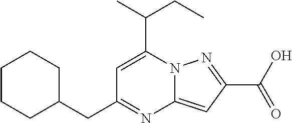 | 1 | NMR2: 7.21 (s, 1H), 6.63 (s, 1H), 3.88-3.78 (m, 1H), 2.73 (d, J = 7.1 Hz, 2H), 2.01-1.57 (m, 8H), 1.40 (d, J = 7.0 Hz, 3H), 1.34-1.00 (m, 5H), 0.97 (t, J = 7.4 Hz, 3H). |
| 65 | 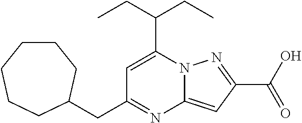 | 1 | NMR2: 7.21 (s, 1H), 6.62 (s, 1H), 3.82-3.70 (m, 1H), 2.77 (d, J = 7.4 Hz, 2H), 2.18-1.20 (m, 17H), 0.88 (t, J = 7.4 Hz, 6H). |
| 66 | 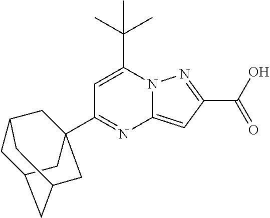 | 31 | NMR2: 7.09 (s, 1H), 6.84 (s, 1H), 4.45 (q, J = 7.1 Hz, 2H), 2.18-2.12 (m, 3H), 2.05- 2.00 (m, 6H), 1.86-1.76 (m, 6H), 1.64 (s, 9H), 1.43 (t, J = 7.1 Hz, 3H). |
| 67 | 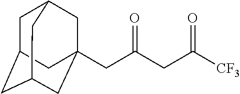 | 12 | NMR2: 5.83-5.51 (m, 1H), 2.19-2.02 (m, 3H), 1.98-1.94 (m, 6H), 1.77-1.47 (m, 9H). |

TABLE 1-10

| REX | STR | RProp | Data |
|---|---|---|---|
| 68 | 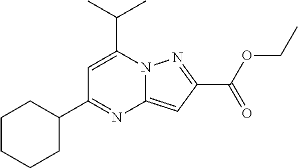 | 4 | NMR2: 7.08 (s, 1H), 6.66 (s, 1H), 4.47 (q, J = 7.1 Hz, 2H), 4.07-3.92 (m, 1H), 2.80- 2.68 (m, 1H), 2.03-1.72 (m, 5H), 1.65- 1.51 (m, 2H), 1.50-1.27 (m, 12H). |
| 69 | 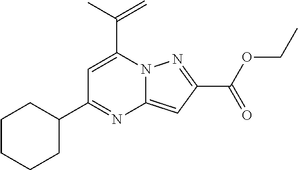 | 2 | NMR2: 7.12 (s, 1H), 6.76 (s, 1H), 6.23 (s, 1H), 5.74-6.67 (m, 1H), 4.46 (q, J = 7.1 Hz, 2H), 2.82-2.70 (m, 1H), 2.36 (s, 3H), 2.05-1.72 (m, 5H), 1.66-1.51 (m, 2H), 1.51-1.23 (m, 6H). |
| 70 | 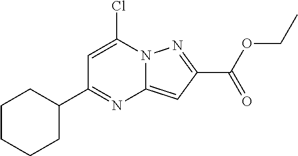 | 6 | NMR2: 7.20 (s, 1H), 6.99 (s, 1H), 4.50 (q, J = 7.1 Hz, 2H), 2.82-2.70 (m, 1H), 2.04- 1.74 (m, 5H), 1.64-1.50 (m, 3H), 1.49- 1.22 (m, 5H). |
| 71 | 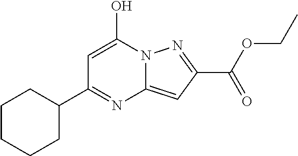 | 7 | NMR1: 12.32 (s, 1H), 6.48 (s, 1H), 5.69 (s, 1H), 4.33 (q, J = 7.1 Hz, 2H), 2.59-2.51 (m, 1H), 1.93-1.64 (m, 5H), 1.54-1.15 (m, 8H). |
| 72 | 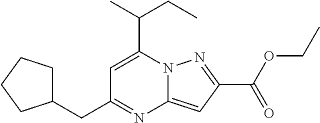 | 31 | NMR2: 7.07 (s, 1H), 6.61 (s, 1H), 4.47 (q, J = 7.1 Hz, 2H), 3.94-3.81 (m, 1H), 2.83 (d, J = 7.5 Hz, 2H), 2.39-2.23 (m, 1H), 1.96- 1.50 (m, 7H), 1.44 (t. J = 7.1 Hz, 3H), 1.38 (d, J = 6.9 Hz, 3H), 1.33-1.19 (m, 3H), 0.95 (t, J = 7.4 Hz, 3H). |
| 73 | 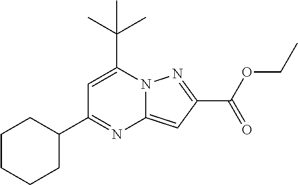 | 31 | NMR2: 7.06 (s, 1H), 6.66 (s, 1H), 4.45 (q, J = 7.1 Hz, 2H), 2.79-2.67 (m, 1H), 2.02- 1.73 (m, 5H), 1.84 (s, 9H), 1.61-1.54 (m, 2H), 1.49-1.28 (m, 6H). |
| 74 | 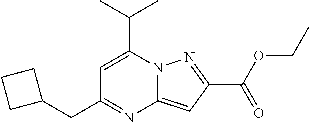 | 4 | NMR1: 7.04 (s, 1H), 6.99 (s, 1H), 4.38 (q, J = 7.1 Hz, 2H), 3.83-3.68 (m, 1H), 2.92 (d, J = 7.6 Hz, 2H), 2.83-2.71 (m, 1H), 2.11- 1.96 (m, 2H), 1.91-1.68 (m, 4H), 1.42- 1.31 (m, 9H) |

TABLE 1-11

| REX | STR | RProp | Data |
|---|---|---|---|
| 75 | 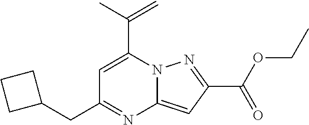 | 2 | NMR1: 7.10-7.04 (m, 2H), 6.38-6.29 (m, 1H), 5.83-5.77 (m, 1H), 4.38 (q, J = 7.1 Hz, 2H), 2.94 (d, J = 7.6 Hz, 2H), 2.85- 2.73 (m, 1H), 2.29 (s, 3H), 2.10-1.96 (m, 2H), 1.92-1.70 (m, 4H), 1.34 (t, J = 7.1 Hz, 3H). |
| 76 | 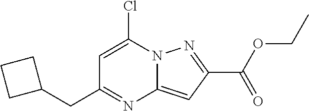 | 6 | NMR2: 7.19 (s, 1H), 6.92 (s, 1H), 4.50 (q, J = 7.1 Hz, 2H), 2.94 (d, J = 7.6 Hz, 2H), 2.88- 2.70 (m, 1H), 2.19-2.06 (m, 2H), 1.99- 1.74 (m, 4H), 1.45 (t, J = 7.1 Hz, 3H). |
| 77 | 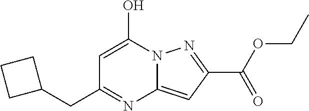 | 43 | NMR1: 12.44 (s, 1H), 6.48 (s, 1H), 5.67 (s, 1H), 4.33 (q, J = 7.1 Hz, 2H), 2.72-2.57 (m, 3H), 2.10-1.98 (m, 2H), 1.91-1.68 (m, 4H), 1.33 (t, J = 7.1 Hz, 3H). |
| 78 | 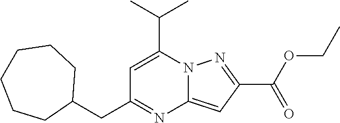 | 4 | NMR2: 7.08 (s, 1H), 6.63 (s, 1H), 4.48 (q, J = 7.1 Hz, 2H), 4.07-3.92 (m, 1H), 2.73 (d, J = 7.4 Hz, 2H), 2.07-2.02 (m, 1H), 1.76- 1.47 (m, 9H), 1.48-1.36 (m, 9H), 1.36- 1.22 (m, 3H). |
| 79 | 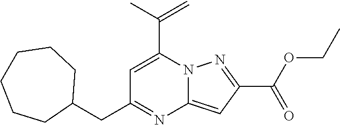 | 2 | NMR2: 7.12 (s, 1H), 6.72 (s, 1H), 6.27 (s, 1H), 5.75-5.69 (m, 1H), 4.46 (q, J = 7.1 Hz, 2H), 2.75 (d, J = 7.4 Hz, 2H), 2.38- 2.33 (m, 3H), 2.13-2.01 (m, 1H), 1.81- 1.36 (m, 12H), 1.36-1.22 (m, 3H). |
| 80 | 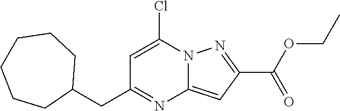 | 6 | NMR2: 7.20 (s, 1H), 6.96 (s, 1H), 4.50 (q, J = 7.1 Hz, 2H), 2.75 (d, J = 7.4 Hz, 2H), 2.14-2.01 (m, 1H), 1.77-1.37 (m, 12H), 1.36- 1.23 (m, 3H). |
| 81 | 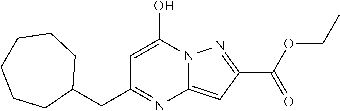 | 7 | NMR2: 12.32 (s, 1H), 6.64 (s, 1H), 5.77 (s, 1H), 4.30 (q, J = 7.1 Hz, 2H), 2.70 (d, J = 7.4 Hz, 2H), 2.07-1.92 (m, 1H), 1.82-1.13 (m, 15H). |
| 82 | 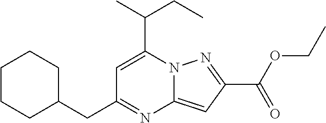 | 31 | NMR2: 7.08 (s, 1H): 6.58 (s, 1H), 4.47 (q, J = 7.1 Hz, 2H), 3.92-3.83 (m, 1H), 2.70 (d, J = 7.2 Hz, 2H), 1.97-1.62 (m, 8H), 1.44 (t, J = 7.1 Hz, 3H), 1.38 (d, J = 6.9 Hz, 3H), 1.31-1.14 (m, 3H), 1.12-0.98 (m, 2H), 0.95 (t, J = 7.5 Hz, 3H). |

TABLE 1-12

| REX | STR | RProp | Data |
|---|---|---|---|
| 83 | 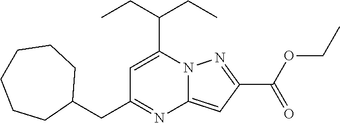 | 31 | NMR2: 7.08 (s, 1H), 8.57 (s, 1H), 4.47 (d, J = 7.2 Hz, 2H), 4.18-4.07 (m, 1H), 3.86- 3.76 (m, 1H), 2.74 (d, J = 7.4 Hz, 2H), 2.22-1.15 (m, 19H), 0.88 (t, J = 7.5 Hz, 6H). |
| 84 | 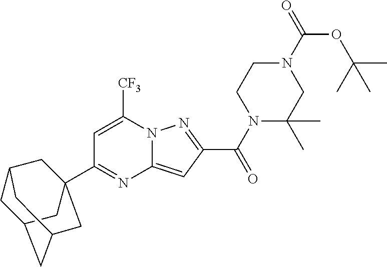 | 84 | NMR2: 7.26 (s, 1H), 7.06-7.01 (m, 1H), 4.01-3.94 (m, 2H), 3.58-3.47 (m, 4H), 2.19-2.15 (m, 3H), 2.02 (d, J = 2.9 Hz, 6H), 1.81 (q, J = 12.6 Hz, 6H), 1.62 (s, 6H), 1.48 (s, 9H). |
| 85 | 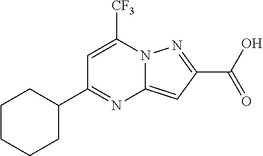 | 1 | NMR1: 13.46 (s, 1H), 7.77 (s, 1H), 7.24 (s, 1H), 2.92 (tt, J = 11.7, 3.4 Hz, 1H), 2.00-0.91 (m, 10H). |
| 86 | 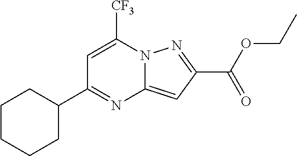 | 86 | NMR2: 7.24 (s, 1H), 7.17 (s, 1H), 4.48 (q, J = 7.1 Hz, 2H), 2.83 (tt, J = 11.8, 3.4 Hz, 1H), 2.07-1.73 (m, 5H), 1.68-1.19 (m, 8H). |
| 87 | 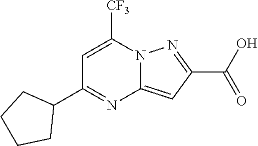 | 1 | NMR1: 13.29 (s, 1H), 7.72 (s, 1H), 7.22 (s, 1H), 3.47-3.36 (m, 1H), 2.14-1.47 (m, 8H). |
| 88 | 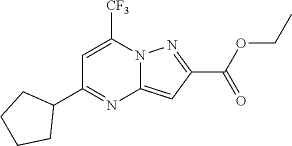 | 86 | NMR2: 7.23 (s, 1H), 7.16 (s, 1H), 4.48 (q, J = 7.1 Hz, 2H), 3.38-3.25 (m, 1H), 2.21- 2.08 (m, 2H), 1.96-1.71 (m, 6H), 1.44 (t, J = 7.1 Hz, 3H). |
| 89 | 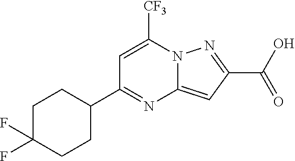 | 1 | NMR1: 13.56 (s, 1H), 7.82 (s, 1H), 7.28 (s, 1H), 3.18-3.07 (m, 1H), 2.22-1.72 (m, 8H). |

TABLE 1-13

| REX | STR | RProp | Data |
|---|---|---|---|
| 90 | 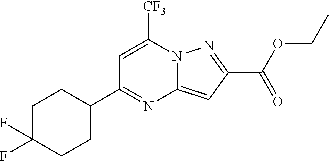 | 86 | NMR2 7.27 (s, 1H), 7.19 (s, 1H), 4.49 (q, J = 7.1 Hz, 2H), 3.01-2.89 (m, 1H), 2.48-2.23 (m, 2H), 2.16-1.70 (m, 6H), 1.45 (t, J = 7.1 Hz, 3H) |
| 91 | 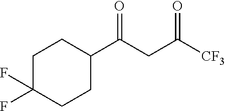 | 12 | NMR2: 5.96 (s, 1H), 2.45-2.40 (m, 2H), 2.27-1.65 (m, 8H). |
| 92 | 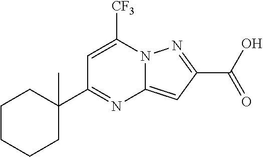 | 1 | NMR1: 13.54 (s, 1H), 7.78 (s, 1H), 7.28 (s, 1H), 2.25-2.16 (m, 2H), 1.64- 1.19 (m, 11H). |
| 93 | 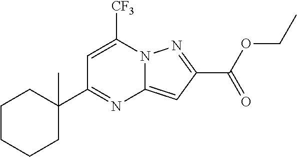 | 93 | NMR2 7.32 (s, 1H), 7.27 (s, 1H), 4.49 (q, J = 7.1 Hz, 2H), 2.23-2.14 (m, 2H), 1.70-1.57 (m, 3H), 1.53-1.38 (m, 8H), 1.30 (s, 3H). |
| 94 | 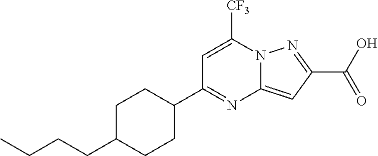 | 1 | NMR2, 7.38-7.32 (m, 1H), 7.27-7.20 (m, 1H), 3.01-2.75 (m, 1H), 2.45- 2.26 (m, 1H), 2.11-1.49 (m, 6H), 1.44- 1.03 (m, 8H), 0.95-0.79 (m, 3H). |
| 95 | 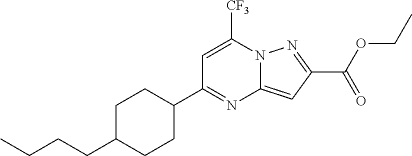 | 93 | NMR2 7.26-7.23 (m, 1H), 7.21-7.15 (m, 1H), 4.48 (q, J = 7.1 Hz, 2H), 3.02- 2.72 (m, 1H), 2.08-1.88 (m, 4H), 1.86-1.73 (m, 1H), 1.71-1 52 (m, 2H), 1.44 (t, J = 7.1 Hz, 3H), 1.41- 1.22 (m, 6H), 1.16-1.01 (m, 2H), 0.93- 0.84 (m, 3H) |
| 96 | 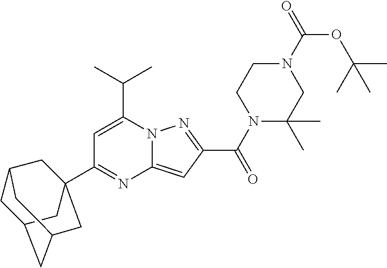 | 84 | NMR2: 6.82 (s, 1H), 6.77 (s, 1H), 3.95- 3.89 (m, 2H), 3.85-3.75 (m, 1H), 3.59-3.47 (m, 4H), 2.17-2.11 (m, 3H), 2.05-1.99 (m, 6H), 1.86-1.74 (m, 6H), 1.65-1.60 (m, 6H), 1.48 (s, 9H), 1.45-1.38 (m, 6H). |

TABLE 1-14

| REX | STR | RProp | Data |
|---|---|---|---|
| 97 | 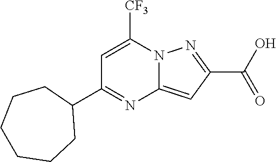 | 1 | NMR2: 7.32 (s, 1H), 7.19 (s, 1H), 3.03 (tt, J = 10.2, 3.8 Hz, 1H), 2.09-1.99 (m, 2H), 1.94-1.62 (m, 10H). |
| 98 | 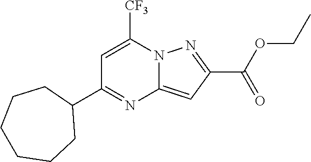 | 93 | NMR2: 7.23 (s, 1H), 7.14 (s, 1H), 4.48 (q, J = 7.1 Hz, 2H), 3.01 (tt, J = 10.2, 3.8 Hz, 1H), 2.08-1.99 (m, 2H), 1.94- 1.57 (m, 10H), 1.44 (t, J = 7.1 Hz, 3H). |
| 99 | 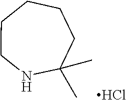 | 99 | NMR1: 8.91 (s, 2H), 3.04-2.97 (m, 2H), 1.85-1.77 (m, 2H), 1.71-1.64 (m, 2H), 1.62-1.52 (m, 4H), 1.30 (s, 6H). |
| 100 | 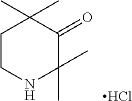 | 100 | NMR2: 10.23 (s, 2H), 3.46-3.39 (m, 2H), 2.23-2.16 (m, 2H), 1.68 (s, 6H), 1.25 (s, 6H). |
| 101 | 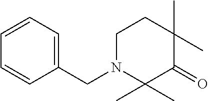 | 101 | NMR2: 7.41-7.19 (m, 5H), 3.61 (s, 2H), 2.64 (t, J = 6.4 Hz, 2H), 1.68 (t, J = 6.4 Hz, 2H), 1.32 (s, 6H), 1.19 (s, 6H). |
| 102 | 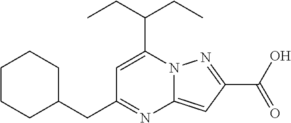 | 1 | NMR2: 7.19 (s, 1H), 6.61 (s, 1H), 3.70 (q, J = 8.8 Hz, 1H), 2.73 (d, J = 7.3 Hz, 2H), 1.97-1.76 (m, 5H), 1.76-1.60 (m, 5H), 1.32-0.97 (m, 5H), 0.88 (t, J = 7.4 Hz, 6H). |
| 103 | 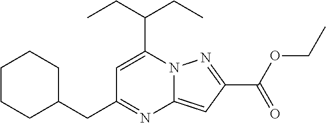 | 31 | NMR2: 7.07 (s, 1H), 6.55 (s, 1H), 4.47 (q, J = 7.1 Hz, 2H), 3.86-3.74 (m, 1H), 2.70 (d, J = 7.2 Hz, 2H), 1.96-1.74 (m, 4H), 1.76-1.62 (m, 6H), 1.43 (t, J = 7.1 Hz, 3H), 1.31-0.96 (m, 5H), 0.87 (t, J = 7.4 Hz, 6H). |
| 104 | 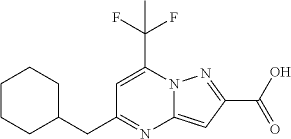 | 1 | NMR2: 7.29 (s, 1H), 7.07 (s, 1H), 2.79 (d, J = 7.2 Hz, 2H), 2.35 (t, J = 19.1 Hz, 3H), 1.94-1.80 (m, 1H), 1.77- 1.64 (m, 5H), 1.34-1.00 (m, 5H). |

TABLE 1-15

| REX | STR | RProp | Data |
|---|---|---|---|
| 105 | 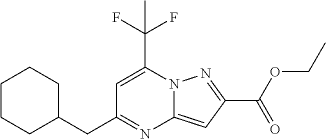 | 86 | NMR2: 7.17 (s, 1H), 7.02 (s, 1H), 4.46 (q, J = 7.1 Hz, 2H), 2.76 (d, J = 7.2 Hz, 2H), 2.36 (t, J = 19.2 Hz, 3H), 1.91- 1.79 (m, 1H), 1.71 (d, J = 12.4 Hz, 5H), 1.43 (t, J = 7.1 Hz, 3H), 1.33-0.99 (m, 5H). |
| 106 | 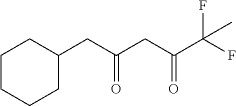 | 12 | NMR2: 14.91 (s, 1H), 5.88 (d, J = 0.9 Hz, 1H), 2.32-2.22 (m, 2H), 1.85- 1.61 (m, 7H), 1.35-0.83 (m, 7H). |
| 107 | 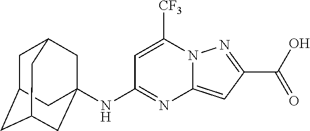 | 1 | NMR1: 13.16 (s, 1H), 7.56 (s, 1H), 7.02 (s, 1H), 6.46 (s, 1H), 2.14-2.06 (m, 9H), 1.70-1.62 (m, 6H). |
| 108 | 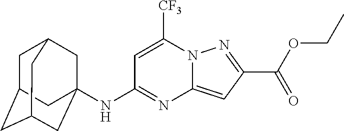 | 108 | NMR1: 7.59 (s, 1H), 7.04 (s, 1H), 6.52 (s, 1H), 4.33 (q, J = 7.1 Hz, 2H), 2.17- 2.04 (m, 9H), 1.76-1.62 (m, 6H), 1.31 (t, J = 7.1 Hz, 3H). |
| 109 | 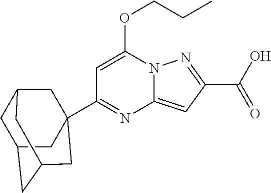 | 109 | NMR1: 6.89 (s, 1H), 6.72 (s, 1H), 4.46 (t, J = 6.7 Hz, 2H), 2.12-2.05 (m, 3H), 2.02-1.97 (m, 6H), 1.97-1.84 (m, 2H), 1.79-1.73 (m, 6H), 1.05 (t, J = 7.4 Hz, 3H). |
| 110 | 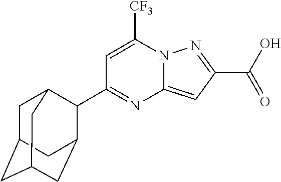 | 1 | NMR2: 7.37 (s, 1H), 7.31 (s, 1H), 3.16 (s, 1H), 2.66-2.61 (m, 2H), 2.12-1.76 (m, 10H), 1.71-1.64 (m, 2H). |
| 111 | 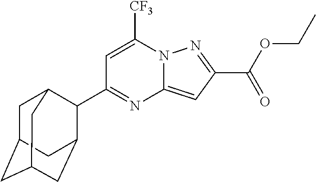 | 31 | NMR2: 7.31-7.22 (m, 2H), 4.48 (q, J = 7.1 Hz, 2H), 3.14 (s, 1H), 2.65-2.60 (m, 2H), 2.08-2.01 (m, 3H), 1.98- 1.82 (m, 5H), 1.80 (s, 2H), 1.70-1.63 (m, 2H), 1.44 (t, J = 7.1 Hz, 3H). |

TABLE 1-16

| REX | STR | RProp | Data |
|---|---|---|---|
| 112 | 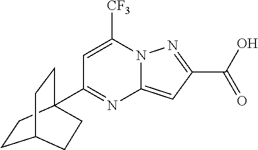 | 1 | NMR2: 7.37-7.31 (m, 2H), 1.95-1.89 (m, 6H), 1.81-1.71 (m, 7H). |
| 113 | 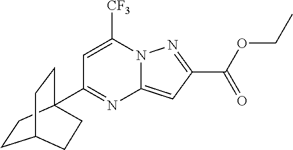 | 93 | NMR2: 7.28 (s, 1H), 7.24 (s, 1H), 4.48 (q, J = 7.1 Hz, 2H), 1.95-1.87 (m, 6H), 1.81- 1.63 (m, 7H), 1.44 (t, J = 7.2 Hz, 3H). |
| 114 | 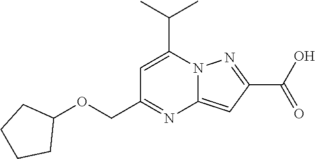 | 1 | NMR2: 7.21 (s, 1H), 7.08 (d, J = 0.7 Hz, 1H), 4.61 (s, 2H), 4.11-4.04 (m, 1H), 4.01-3.87 (m, 1H), 1.87-1.69 (m, 8H), 1.46 (d, J = 6.9 Hz, 6H). |
| 115 | 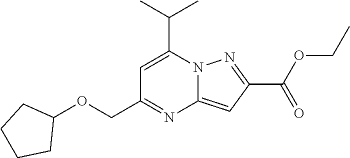 | 115 | NMR2: 7.11 (s, 1H), 7.02 (d, J = 0.8 Hz, 1H), 4.60 (s, 2H), 4.48 (q, J = 7.1 Hz, 2H), 4.11- 3.97 (m, 2H), 1.83-1.72 (m, 8H), 1.50- 1.40 (m, 9H). |
| 116 | 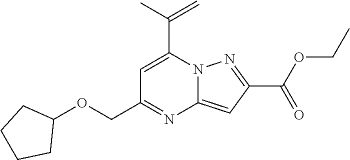 | 2 | NMR2: 7.14 (s, 1H), 7.10 (s, 1H), 6.36-6.31 (m, 1H), 5.78-5.72 (m, 1H), 4.61 (s, 2H), 4.46 (q, J = 7.1 Hz, 2H), 4.08 (s, 1H), 2.39- 2.34 (m, 3H), 1.85-1.73 (m, 6H), 1.61- 1.54 (m, 2H), 1.44 (t, J = 7.1 Hz, 3H). |
| 117 | 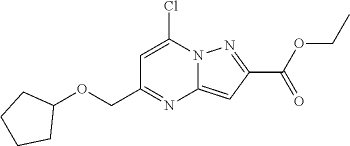 | 6 | NMR2: 7.34 (s, 1H), 7.22 (s, 1H), 4.60 (s, 2H), 4.51 (q, J = 7.1 Hz, 2H), 4.15-4.00 (m, 1H), 1.88-1.68 (m, 6H), 1.64-1.56 (m, 2H), 1.46 (t, J = 7.1 Hz, 3H). |
| 118 | 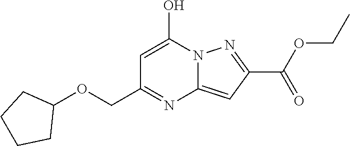 | 7 | NMR2: 9.51 (s, 1H), 6.65 (s, 1H), 5.82-5.77 (m, 1H), 4.52 (s, 2H), 4.41 (q, J = 7.1 Hz, 2H), 4.13-4.05 (m, 1H), 1.90-1.66 (m, 6H), 1.59 (d, J = 8.7 Hz, 2H), 1.37 (t, J = 7.1 Hz, 3H). |

TABLE 1-17

| REX | STR | RProp | Data |
|---|---|---|---|
| 119 | 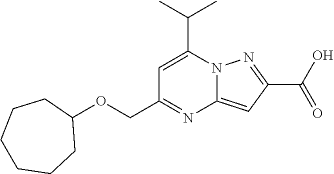 | 1 | NMR2: 7.19 (s, 1H), 7.11 (s, 1H), 4.64 (s, 2H), 4.01-3.88 (m, 1H), 3.61 (tt, J = 8.0, 4.3 Hz, 1H), 2.03-1.91 (m, 2H), 1.81-1.63 (m, 4H), 1.61-1.52 (m, 4H), 1.49-1.37 (m, 8H). |
| 120 | 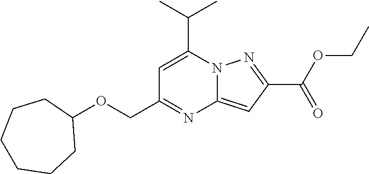 | 115 | NMR2: 7.10 (s, 1H), 7.06 (d, J = 0.6 Hz, 1H), 4.62 (s, 2H), 4.48 (q, J = 7.1 Hz, 2H), 4.10- 3.95 (m, 1H), 3.65-3.55 (m, 1H), 2.02- 1.91 (m, 2H), 1.80-1.64 (m, 4H), 1.60- 1.51 (m, 4H), 1.48-1.38 (m, 11H). |
| 121 | 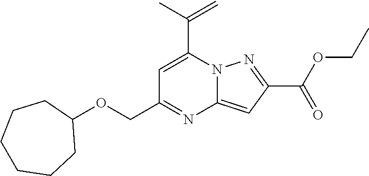 | 2 | NMR2: 7.13 (s, 2H), 6.37-6.32 (m, 1H), 5.78-5.73 (m, 1H), 4.64 (s, 2H), 4.46 (q, J = 7.1 Hz, 2H), 3.62 (tt, J = 8.0, 4.3 Hz, 1H), 2.37 (t, J = 1.2 Hz, 3H), 2.02-1.90 (m, 2H), 1.80-1.67 (m, 4H), 1.63-1.53 (m, 4H), 1.48-1.38 (m, 5H). |
| 122 | 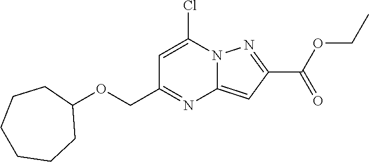 | 6 | NMR2: 7.37 (s, 1H), 7.21 (s, 1H), 4.63 (s, 2H), 4.50 (q, J = 7.1 Hz, 2H), 3.68-3.57 (m, 1H), 2.07-1.90 (m, 2H), 1.80-1.64 (m, 4H), 1.61-1.54 (m, 4H), 1.50-1.39 (m, 5H). |
| 123 | 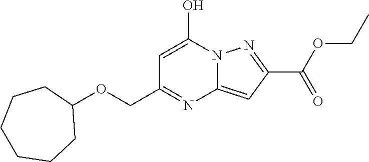 | 7 | NMR2: 10.01 (s, 1H), 6.64 (s, 1H), 5.82 (s, 1H), 4.56 (d, J = 0.9 Hz, 2H), 4.40 (q, J = 7.2 Hz, 2H), 3.63 (tt, J = 8.1, 4.4 Hz, 1H), 2.00-1.89 (m, 2H), 1.74-1.61 (m, 4H), 1.59-1.52 (m, 4H), 1.45-1.25 (m, 5H). |
| 124 | 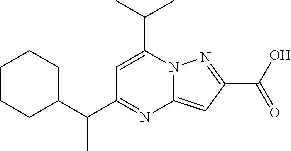 | 1 | NMR1: 13.24 (s, 1H), 7.00 (s, 1H), 6.97 (s, 1H), 3.83-3.68 (m, 1H), 2.80-2.68 (m, 1H), 1.89-1.49 (m, 5H), 1.41-1.35 (m, 7H), 1.28-0.86 (m, 8H). |
| 125 | 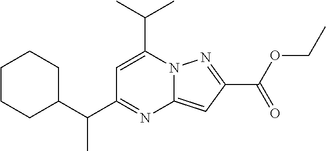 | 4 | NMR2: 7.09 (s, 1H), 6.60 (d, J = 0.7 Hz, 1H), 4.47 (q, J = 7.1 Hz, 2H), 4.07-3.92 (m, 1H), 2.74-2.62 (m, 1H), 1.89 (d, J = 12.5 Hz, 1H), 1.82-1.71 (m, 1H), 1.70-1.61 (m, 3H), 1.47-1.39 (m, 10H), 1.33-0.83 (m, 8H). |

TABLE 1-18

| REX | STR | RProp | Data |
|---|---|---|---|
| 126 | 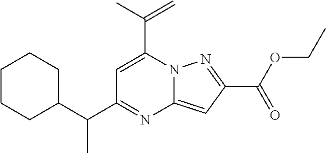 | 2 | NMR2: 7.12 (s, 1H), 8.70 (s, 1H), 6.27-6.22 (m, 1H), 5.74-5.68 (m, 1H), 4.46 (q, J = 7.1 Hz, 2H), 2.76-2.64 (m, 1H), 2.36 (t, J = 1.1 Hz, 3H), 1.88 (d, J = 12.9 Hz, 1H), 1.77 (d, J = 13.0 Hz, 1H), 1.72-1.59 (m, 3H), 1.49-1.39 (m, 4H), 1.35-0.85 (m, 8H). |
| 127 | 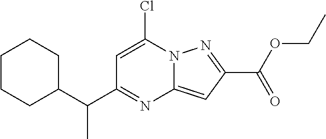 | 6 | NMR2: 7.20 (s, 1H), 6.93 (s, 1H), 4.60 (q, J = 7.1 Hz, 2H), 2.77-2.65 (m, 1H), 1.91- 1.83 (m, 1H), 1.81-1.73 (m, 1H), 1.71- 1.59 (m, 3H), 1.50-1.37 (m, 4H), 1.30 (d, J = 6.9 Hz, 3H), 1.28-0.91 (m, 5H). |
| 128 | 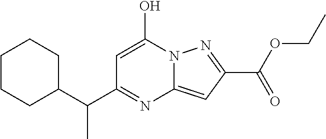 | 7 | NMR2: 11.03 (s, 1H) 6.59 (s, 1H), 5.76 (s, 1H), 4.31 (q, J = 7.1 Hz, 2H), 2.84-2.52 (m, 1H), 1.87-1.79 (m, 1H), 1.77-1.70 (m, 1H), 1.68-1.52 (m, 4H), 1.31 (d, J = 7.0 Hz, 3H), 1.23 (t, J = 7.1 Hz, 3H), 1.19-0.88 (m, 5H). |
| 129 | 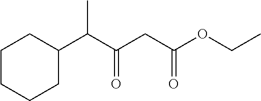 | 49 | NMR2: 4.24-4.07 (m, 3H), 3.46 (s, 2H), 2.53-2.41 (m, 1H), 1.84-1.58 (m, 8H), 1.37-0.82 (m, 8H). |
| 130 | 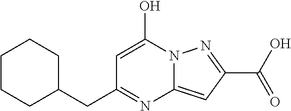 | 1 | NMR1: 13.21 (s, 1H), 12.43 (s, 1H), 6.44 (s, 1H), 5.66 (s, 1H), 2.46 (d, J = 7.1 Hz, 2H), 1.80-1.52 (m, 6H), 1.32-0.86 (m, 5H). |
| 131 | 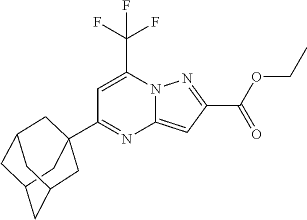 | 86 | NMR2: 7.33 (s, 1H), 7.26 (s, 1H), 4.48 (q, J = 7.1 Hz, 2H), 2.20-2.16 (m, 3H), 2.07- 2.00 (m, 6H), 1.89-1.65 (m, 6H), 1.44 (t, J = 7.1 Hz, 3H). |
| 132 | 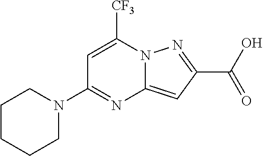 | 132 | NMR1: 13.20 (s, 1H), 7.41 (s, 1H), 6.51 (s, 1H), 3.78-3.71 (m, 4H), 1.70-1.48 (m, 6H). |

TABLE 1-19

| REX | STR | RProp | Data |
|---|---|---|---|
| 133 | 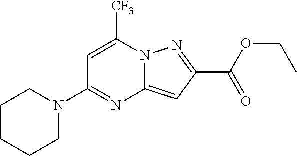 | 133 | NMR2: 6.84 (s, 1H), 6.66 (s, 1H), 4.44 (q, J = 7.1 Hz, 2H), 3.75-3.68 (m, 4H), 1.80- 1.64 (m, 6H), 1.42 (t, J = 7.1 Hz, 3H). |
| 134 | 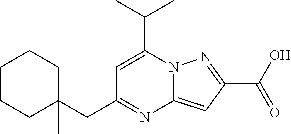 | 3 | NMR2: 7.23-7.18 (m, 1H), 6.68-6.62 (m, 1H), 3.98-3.87 (m, 1H), 2.80-2.73 (m, 2H), 1.79-1.20 (m, 14H), 0.99-0.90 (m, 5H). |
| 135 | 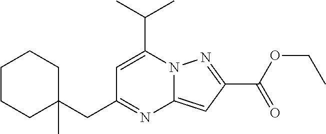 | 4 | NMR2: 7.10 (s, 1H), 6.61 (s, 1H), 4.48 (q, J = 7.1 Hz, 2H), 4.07-3.94 (m, 1H), 2.75 (s, 2H), 1.64-1.25 (m, 19H), 0.96 (s, 3H). |
| 136 | 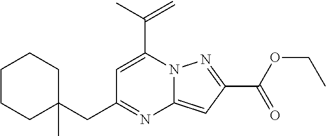 | 5 | NMR2: 7.14 (s, 1H), 6.71 (s, 1H), 6.29- 6.24 (m, 1H), 5.75-5.69 (m, 1H), 4.46 (q, J = 7.1 Hz, 2H), 2.77 (s, 2H), 2.35 (s, 3H), 1.59-1.30 (m, 13H), 0.98 (s, 3H). |
| 137 | 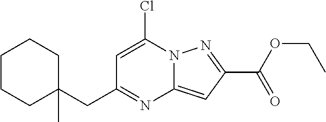 | 6 | NMR2: 7.22 (s, 1H), 6.93 (s, 1H), 4.50 (q, J = 7.1 Hz, 2H), 2.76 (s, 2H), 1.62-1.27 (m, 13H), 0.98 (s, 3H). |
| 138 | 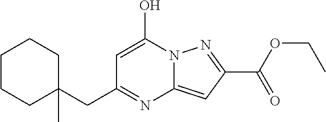 | 7 | NMR2: 11.43 (s, 1H), 6.63 (s, 1H), 5.74 (d, J = 1.8 Hz, 1H), 4.32 (q, J = 7.2 Hz, 2H), 2.70 (s, 2H), 1.55-1.31 (m, 10H), 1.25 (t, J = 7.1 Hz, 3H), 0.98 (s, 3H). |
| 139 | 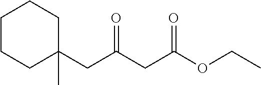 | 49 | NMR2: 4.23-4.14 (m, 2H), 3.42 (s, 2H), 2.46 (s, 2H), 1.50-1.21 (m, 13H), 1.05- 1.00 (m, 3H). |
| 140 | 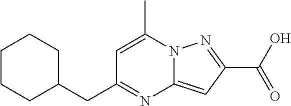 | 3 | NMR1: 13.17 (s, 1H), 7.05 (s, 1H), 6.99 (s, 1H), 2.71 (s, 3H), 2.65 (d, J = 7.2 Hz, 2H), 1.89-1.75 (m, 1H), 1.70-1.60 (m, 5H), 1.28-1.09 (m, 3H), 1.07-0.93 (m, 2H). |
| 141 | 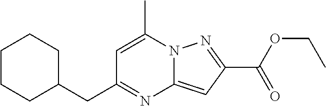 | 2 | NMR2: 7.10 (s, 1H), 6.66 (s, 1H), 4.49 (q, J = 7.1 Hz, 2H), 2.82 (s, 3H), 2.68 (d, J = 7.2 Hz, 2H), 1.90-1.76 (m, 1H), 1.76-1.65 (m, 5H), 1.45 (t, J = 7.1 Hz, 3H), 1.32-1.13 (m, 3H), 1.11-0.98 (m, 2H). |

TABLE 1-20

| REX | STR | RProp | Data |
|---|---|---|---|
| 142 | 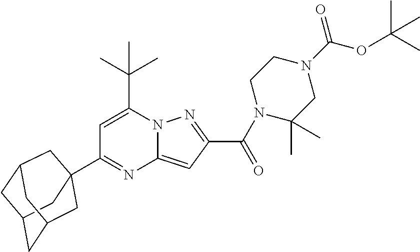 | 84 | NMR2: 6.89 (s, 1H), 6.80 (s, 1H), 4.05- 3.96 (m, 2H), 3.61-3.45 (m, 4H), 2.17- 2.12 (m, 3H), 2.04-2.00 (m, 6H), 1.85- 1.75 (m, 7H), 1.67-1.59 (m, 14H), 1.48 (s, 9H). |
| 143 | 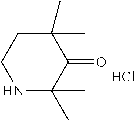 | 143 | NMR2: 3.46-3.38 (m, 2H), 2.22-2.15 (m, 2H), 1.67 (s, 6H), 1.26 (s, 6H). |
| 144 | 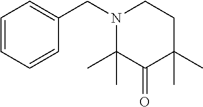 | 144 | NMR2: 7.42-7.28 (m, 4H), 7.26-7.19 (m, 1H), 3.61 (s, 2H), 2.64 (t, J = 6.4 Hz, 2H), 1.68 (t, J = 6.4 Hz, 2H), 1.32 (s, 6H), 1.19 (s, 6H). |
| 145 | 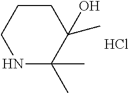 | 145 | NMR1: 9.06 (s, 1H), 8.12 (s, 1H), 4.41 (s, 1H), 3.06-2.78 (m, 2H), 2.03-1.82 (m, 1H), 1.81-1.68 (m, 1H), 1.57-1.45 (m, 2H), 1.28 (s, 3H), 1.27 (s, 3H), 1.09 (s, 3H). |
| 146 | 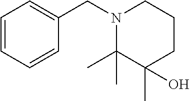 | 146 | NMR2: 7.31 (d, J = 4.4 Hz, 4H), 7.27-7.18 (m, 1H), 3.98 (d, J = 13.8 Hz, 1H), 3.61 (s, 1H), 3.08 (d, J = 13.8 Hz, 1H), 2.52-2.42 (m, 1H), 2.40-2.29 (m, 1H), 1.79-1.61 (m, 2H), 1.56-1.32 (m, 2H), 1.26 (s, 3H), 1.19 (s, 3H), 1.09 (s, 3H). |
| 147 | 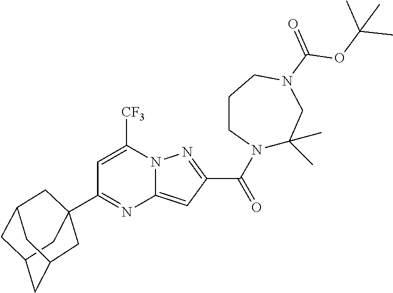 | 84 | NMR2: 7.25 (s, 1H), 7.04 (s, 1H), 3.70- 3.66 (m, 2H), 3.57-3.50 (m, 4H), 2.19- 2.15 (m, 3H), 2.07-1.96 (m, 8H), 1.88- 1.75 (m, 6H), 1.60 (s, 6H), 1.49 (s, 9H). |

TABLE 1-21

| REX | STR | RProp | Data |
|---|---|---|---|
| 148 | 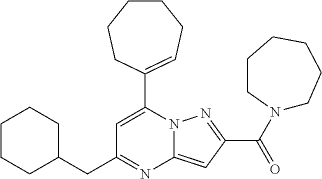 | 20 | NMR2: 6.90 (s, 1H), 6.53 (s, 1H), 6.52 (t, J = 6.6 Hz, 1H), 3.85-3.77 (m, 2H), 3.76-3.69 (m, 2H), 2.70-2.61 (m, 4H), 2.44-2.35 (m, 3H), 1.93-1.58 (m, 19H), 1.32-1.16 (m, 3H), 1.11-0.99 (m, 2H). |
| 149 | 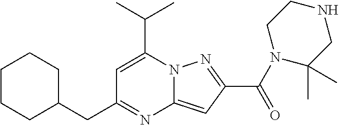 | 149 | NMR1: 6.90 (s, 1H), 8.68 (s, 1H), 3.74-3.60 (m, 1H), 3.50-3.43 (m, 2H), 2.87-2.80 (m, 2H), 2.69 (s, 2H), 2.67 (d, J = 7.1 Hz, 2H), 1.87-1.76 (m, 1H), 1.70-1.57 (m, 6H), 1.47 (s, 6H), 1.36 (d, J = 6.9 Hz, 6H), 1.28-1.07 (m, 3H), 1.07-0.93 (m, 2H). |
| 150 | 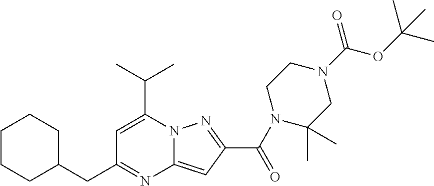 | 84 | NMR1: 6.91 (s, 1H), 6.73 (s, 1H), 3.81-3.77 (m, 2H), 3.73-3.62 (m, 1H), 3.48 (s, 2H), 3.45-3.34 (m, 2H), 2.67 (d, J = 7.1 Hz, 2H), 1.87-1.77 (m, 1H), 1.69-1.60 (m, 6H), 1.50 (s, 6H), 1.42 (s, 9H), 1.37 (d, J = 6.9 Hz, 6H), 1.25-1.10 (m, 3H), 1.07-0.94 (m, 2H). |
| 151 | 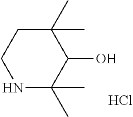 | 145 | NMR1: 8.88 (s, 2H), 5.44 (d, J = 6.4 Hz, 1H), 3.14-3.03 (m, 2H), 2.99-2.90 (m, 1H), 1.62 (td, J = 13.6, 12.9, 4.3 Hz, 1H), 1.52 (dt, J = 14.4, 3.1 Hz, 1H), 1.31 (s, 3H), 1.21 (s, 3H), 0.94 (s, 3H), 0.92 (s, 3H). |
| 152 | 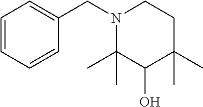 | 152 | NMR2: 7.39-7.27 (m, 4H), 7.26-7.17 (m, 1H), 3.89 (d, J = 14.1 Hz, 1H), 3.21 (d, J = 14.1 Hz, 1H), 3.10 (d, J = 7.3 Hz, 1H), 2.42- 2.31 (m, 2H), 1.73 (d, J = 7.3 Hz, 1H), 1.43- 1.28 (m, 2H), 1.29 (s, 3H), 1.07 (s, 3H), 1.01 (s, 3H), 0.95 (s, 3H). |
| 153 | 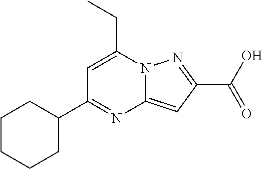 | 3 | NMR1: 13.23 (s, 1H), 7.06 (s, 1H), 6.99 (s, 1H), 3.13 (q, J = 7.4 Hz, 2H), 2.77 (tt, J = 11.7, 3.4 Hz, 1H), 1.95-1.87 (m, 2H), 1.86- 1.77 (m, 2H), 1.75-1.68 (m, 1H), 1.62- 1.48 (m, 2H), 1.46-1.18 (m, 6H). |
| 154 | 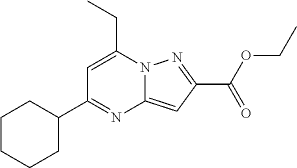 | 2 | NMR2: 7.10 (s, 1H), 6.69 (s, 1H), 4.48 (q, J = 7.1 Hz, 2H), 3.26 (q, J = 7.4 Hz, 2H), 2.75 (tt, J = 11.9, 3.4 Hz, 1H), 2.03-1.85 (m, 4H), 1.79-1.75 (m, 1H), 1.65-1.24 (m, 11H) |

TABLE 1-22

| REX | STR | RProp | Data |
|---|---|---|---|
| 155 | 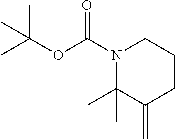 | 155 | NMR2: 4.86 (d, J = 1.1 Hz, 1H), 4.76-4.71 (m, 1H), 3.31 (t, J = 6.5 Hz, 2H), 2.40-2.31 (m, 2H), 1.82-1.70 (m, 2H), 1.54 (s, 6H), 1.47 (s, 9H). |
| 156 | 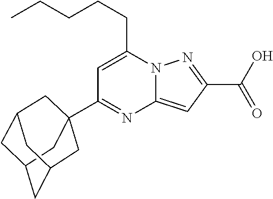 | 156 | NMR2: 7.12 (s, 1H), 6.83 (s, 1H), 4.47 (q, J = 7.1 Hz, 2H), 3.26-3.18 (m, 2H), 2.17-2.13 (m, 3H), 2.05-2.00 (m, 6H), 1.92-1.74 (m, 9H), 1.51-1.34 (m, 7H), 1.31-1.22 (m, 2H). |
| 157 | 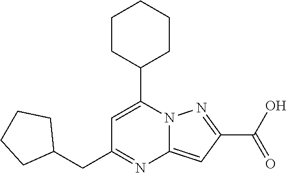 | 3 | NMR1: 13.21 (s, 1H), 6.98 (s, 1H), 6.97 (s, 1H), 3.59-3.44 (m, 1H), 2.79 (d, J = 7.5 Hz, 2H), 2.39-2.24 (m, 1H), 2.09-2.02 (m, 2H), 1.93-1.39 (m, 13H), 1.37-1.15 (m, 3H). |
| 158 | 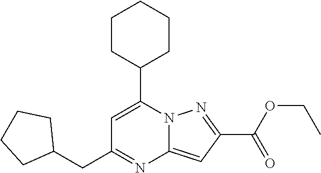 | 4 | NMR2: 7.06 (s, 1H), 6.63 (s, 1H), 4.47 (q, J = 7.1 Hz, 2H), 3.73 (tt, J = 11.8, 3.2 Hz, 1H), 2.82 (d, J = 7.5 Hz, 2H), 2.38-2.22 (m, 1H), 2.21-2.14 (m, 2H), 1.95-1.62 (m, 9H), 1.59- 1.18 (m, 10H). |
| 159 | 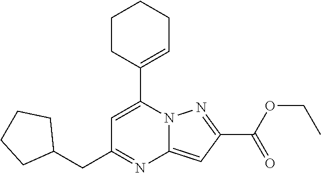 | 5 | NMR2: 7.13-7.08 (m, 1H), 7.08 (s, 1H), 6.69 (s, 1H), 4.45 (q, J = 7.1 Hz, 2H), 2.82 (d, J = 7.6 Hz, 2H), 2.66-2.68 (m, 2H), 2.41-2.27 (m, 2H), 1.90-1.72 (m, 7H), 1.72-1.61 (m, 2H), 1.57-1.49 (m, 2H), 1.44 (t, J = 7.1 Hz, 3H), 1.34-1.20 (m, 2H). |
| 160 | 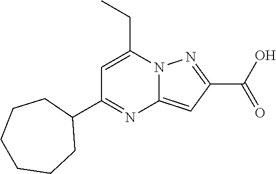 | 3 | NMR1: 13.22 (s, 1H), 7.02 (s, 1H), 6.98 (s, 1H), 3.12 (q, J = 7.4 Hz, 2H), 3.00-2.88 (m, 1H), 1.98-1.87 (m, 2H), 1.84-1.45 (m, 10H), 1.36 (t, J = 7.5 Hz, 3H). |
| 161 | 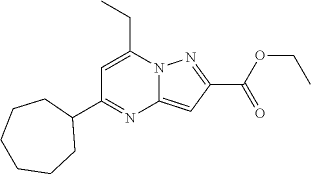 | 2 | NMR2: 7.08 (s, 1H), 6.68-6.63 (m, 1H), 4.48 (q, J = 7.1 Hz, 2H), 3.31-3.21 (m, 2H), 2.97- 2.85 (m, 1H), 2.07-1.96 (m, 2H), 1.93- 1.53 (m, 10H), 1.48-1.40 (m, 6H). |

TABLE 1-23

| REX | STR | RProp | Data |
|---|---|---|---|
| 162 | 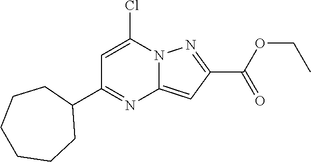 | 6 | NMR2: 7.18 (s, 1H), 8.96 (s, 1H), 4.50 (q, J = 7.1 Hz, 2H), 2.99-2.87 (m, 1H), 2.06- 1.96 (m, 2H), 1.94-1.53 (m, 10H), 1.45 (t, J = 7.1 Hz, 3H). |
| 163 | 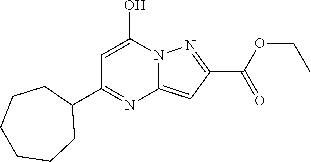 | 7 | NMR1: 12.33 (s, 1H), 6.48 (s, 1H), 5.70 (d, J = 1.9 Hz, 1H), 4.34 (q, J = 7.1 Hz, 2H), 2.73-2.62 (m, 1H), 1.95-1.85 (m, 2H), 1.83-1.42 (m, 10H), 1.33 (t, J = 7.1 Hz, 3H). |
| 164 | 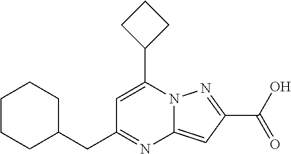 | 3 | NMR1: 13.23 (s, 1H), 7.05 (s, 1H), 6 96 (s, 1H), 4.23-4.10 (m, 1H), 2.70 (d, J = 7.1 Hz, 2H), 2.51-2.42 (m, 1H), 2.34-2.21 (m, 2H), 2.21-2.06 (m, 1H), 1.96-1.80 (m, 2H), 1.70-1.57 (m, 6H), 1.28-1.10 (m, 3H), 1.10-0.94 (m, 2H). |
| 165 | 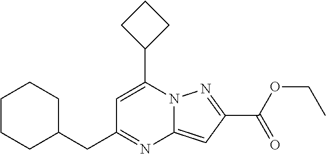 | 31 | NMR2: 7.07 (s, 1H), 6.66 (s, 1H), 4.46 (q, J = 7.1 Hz, 2H), 4.39-4.28 (m, 1H), 2.71 (d, J = 7.2 Hz, 2H), 2.68-2.57 (m, 2H), 2.32- 2.15 (m, 3H), 2.02-1.78 (m, 1H), 1.75- 1.63 (m, 6H), 1.43 (t, J = 7.1 Hz, 3H), 1.29- 1.14 (m, 3H), 1.13-1.00 (m, 2H). |
| 166 | 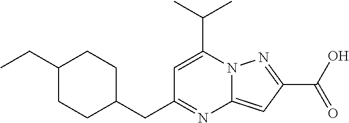 | 3 | NMR2: 7.21 (s, 1H), 6.69-6.64 (m, 1H), 4.03-3.89 (m, 1H), 2.85-2.69 (m, 2H), 2.13-2.03 (m, 1H), 1.85-1.70 (m, 4H), 1.54-1.28 (m, 7H), 1.26-0.99 (m, 4H), 0.96-0.82 (m, 5H). |
| 167 | 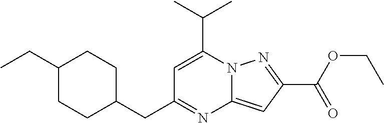 | 4 | NMR2: 7.08 (s, 1H), 6.64-6.59 (m, 1H), 4.48 (q, J = 7.1 Hz, 2H), 4.05-3.94 (m, 1H), 2.82-2.88 (m, 2H), 2.07-2.02 (m, 1H), 1.83-1.68 (m, 4H), 1.54-0.98 (m, 14H), 0.96-0.82 (m, 5H). |
| 168 | 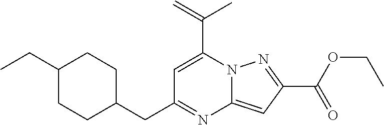 | 5 | NMR2: 7.11 (s, 1H), 6.74-6.69 (m, 1H), 6.30-8.24 (m, 1H), 5.75-5.69 (m, 1H), 4.46 (q, J = 7.1 Hz, 2H), 2.84-2.68 (m, 2H), 2.36 (s, 3H), 1.79-1.71 (m, 4H), 1.54- 0.99 (m, 9H), 0.95-0.80 (m, 5H). |

TABLE 1-24

| REX | STR | RProp | Data |
|---|---|---|---|
| 169 | 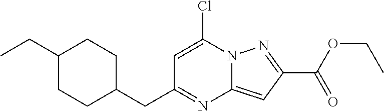 | 6 | NMR2: 7.20 (s, 1H), 6.98-6.92 (m, 1H), 4.50 (q, J = 7.1 Hz, 2H), 2.84-2.69 (m, 2H), 1.85-1.69 (m, 3H), 1.54-0.99 (m, 10H), 0.96-0.79 (m, 5H). |
| 170 | 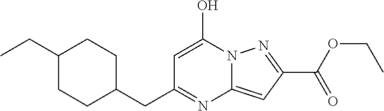 | 7 | NMR2: 12.31 (s, 1H), 6.63 (s, 1H), 5.80- 5.74 (m, 1H), 4.30 (q, J = 7.2 Hz, 2H), 2.81- 2.63 (m, 2H), 1.84-1.52 (m, 4H), 1.47- 1.11 (m, 6H), 1.08-0.91 (m, 3H), 0.87- 0.71 (m, 5H). |
| 171 | 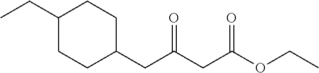 | 49 | NMR2: 4.24-4.14 (m, 2H), 3.47-3.25 (m, 2H), 2.57-2.00 (m, 2H), 1.88-1.40 (m, 7H), 1.38-1.12 (m, 5H), 1.10-0.76 (m, 6H). |
| 172 | 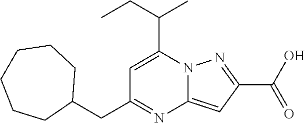 | 3 | NMR2: 7.20 (s, 1H), 6.65 (s, 1H), 3.87- 3.77 (m, 1H), 2.76 (d, J = 7.4 Hz, 2H), 2.15- 2.01 (m, 1H), 1.98-1.42 (m, 15H), 1.37- 1.24 (m, 2H), 0.96 (t, J = 7.4 Hz, 3H). |
| 173 | 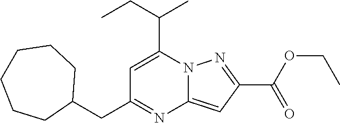 | 31 | NMR2: 7.08 (s, 1H), 6.59 (s, 1H), 4.47 (q, J = 7.1 Hz, 2H), 3.94-3.81 (m, 1H), 2.73 (d, J = 7.4 Hz, 2H), 2.15-1.35 (m, 18H), 1.35- 1.21 (m, 3H), 0.95 (t, J = 7.4 Hz, 3H). |
| 174 | 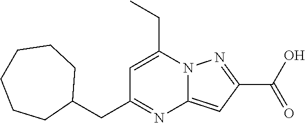 | 3 | NMR2: 7.20 (s, 1H), 6.73-6.68 (m, 1H), 3.24 (qd, J = 7.5, 1.0 Hz, 2H), 2.76 (d, J = 7.4 Hz, 2H), 2.16-2.03 (m, 1H), 1.79-1.38 (m, 13H), 1.37-1.24 (m, 2H). |
| 175 | 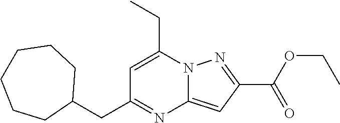 | 2 | NMR2: 7.10 (s, 1H), 6.68-6.62 (m, 1H), 4.48 (q, J = 7.1 Hz, 2H), 3.26 (qd, J = 7.5, 1.1 Hz, 2H), 2.73 (d, J = 7.4 Hz, 2H), 2.14- 2.01 (m, 1H), 1.77-1.36 (m, 14H), 1.36- 1.22 (m, 4H). |
| 176 | 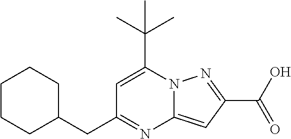 | 3 | NMR2: 7.19 (s, 1H), 6.67 (s, 1H), 2.72 (d, J = 7.2 Hz, 2H), 1.96-1.77 (m, 1H), 1.77- 1.66 (m, 5H), 1.64 (s, 9H), 1.33-1.14 (m, 3H), 1.13-1.00 (m, 2H). |

TABLE 1-25

| REX | STR | RProp | Data |
|---|---|---|---|
| 177 | 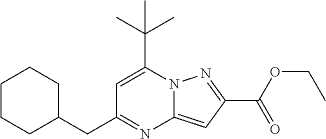 | 31 | NMR2: 7.05 (s, 1H), 8.61 (s, 1H), 4.45 (q, J = 7.1 Hz, 2H), 2.68 (d, J = 7.2 Hz, 2H), 1.87- 1.78 (m, 1H), 1.75-1.67 (m, 5H), 1.64 (s, 9H), 1.43 (t, J = 7.1 Hz, 3H), 1.30-1.14 (m, 3H), 1.12-0.99 (m, 2H). |
| 178 | 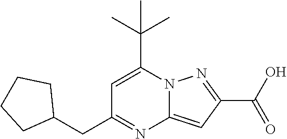 | 3 | NMR2: 7.16 (s, 1H), 6.68 (s, 1H), 2.84 (d, J = 7.5 Hz, 2H), 2.40-2.24 (m, 1H), 1.85- 1.48 (m, 15H), 1.36-1.21 (m, 2H). |
| 179 | 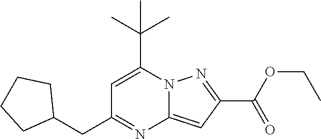 | 31 | NMR2: 7.05 (s, 1H), 6.65 (s, 1H), 4.46 (q, J = 7.1 Hz, 2H), 2.82 (d, J = 7.5 Hz, 2H), 2.37- 2.25 (m, 1H), 1.81-1.65 (m, 6H), 1.64 (s, 9H), 1.43 (t, J = 7.1 Hz, 3H), 1.34-1.20 (m, 2H). |
| 180 | 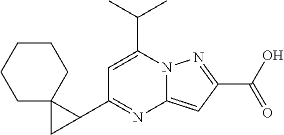 | 3 | NMR2: 7.12 (s, 1H), 6.78 (s, 1H), 3.99- 3.85 (m, 1H), 1.99 (dd, J = 7.8, 5.6 Hz, 1H), 1.72-1.23 (m, 16H), 1.18-1.11 (m, 1H), 0.96 (dd, J = 7.8, 4.5 Hz, 1H). |
| 181 | 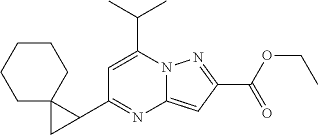 | 4 | NMR2: 7.03 (s, 1H), 5.73 (s, 1H), 4.46 (q, J = 7.2 Hz, 2H), 4.03-3.92 (m, 1H), 1.97 (dd, J = 7.8, 5.6 Hz, 1H), 1.68-1.20 (m, 19H), 1.16-1.10 (m, 1H), 0.98-0.84 (m, 1H). |
| 182 | 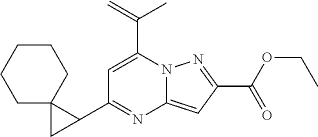 | 5 | NMR2: 7.06 (s, 1H), 6.83 (s, 1H), 6.28- 6.20 (m, 1H), 5.74-5.67 (m, 1H), 4.45 (q, J = 7.1 Hz, 2H), 2.36 (s, 3H), 1.98 (dd, J = 7.8, 5.6 Hz, 1H), 1.68-1.22 (m, 13H), 1.17- 1.10 (m, 1H), 0.99-0.92 (m, 1H). |
| 183 | 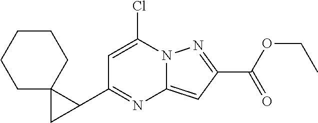 | 6 | NMR2: 7.19 (s, 1H), 6.96 (s, 1H), 5.46- 5.40 (m, 1H), 4.55-4.42 (m, 3H), 2.99- 2.82 (m, 2H), 2.45-2.37 (m, 2H), 2.00- 1.90 (m, 3H), 1.71-1.30 (m, 6H), 1.00 (dd, J = 7.8, 4.5 Hz, 1H). |
| 184 | 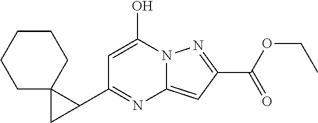 | 7 | NMR2: 11.43 (s, 1H), 6.56 (s, 1H), 5.78 (s, 1H), 4.40-4.26 (m, 2H), 2.89-2.80 (m, 1H), 2.36 (t, J = 7.7 Hz, 1H), 2.02-1.32 (m, 10H), 1.26 (t, J = 7.1 Hz, 3H), 1.03-0.91 (m, 1H). |

TABLE 1-26

| REX | STR | RProp | Data |
|---|---|---|---|
| 185 | 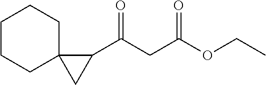 | 49 | NMR2: 4.24-4.16 (m, 2H), 3.56 (s, 2H), 1.91 (dd, J = 7.4, 5.4 Hz, 1H), 1.79-1.39 (m, 8H), 1.37-1.21 (m, 6H), 0.91 (dd, J = 7.4, 4.0 Hz, 1H). |
| 186 | 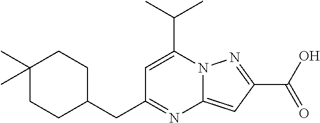 | 3 | NMR2: 7.19 (s, 1H), 6.67 (s, 1H), 4.01-3.86 (m, 1H), 2.75 (d, J = 7.2 Hz, 2H), 1.83-1.69 (m, 1H), 1.57-1.49 (m, 2H), 1.44 (d, J = 6.9 Hz, 6H), 1.41-1.34 (m, 2H), 1.30-1.12 (m, 4H), 0.89 (s, 6H). |
| 187 | 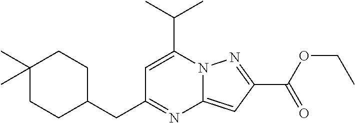 | 4 | NMR2: 7.08 (s, 1H), 6.62 (s, 1H), 4.48 (q, J = 7.1 Hz, 2H), 4.07-3.92 (m, 1H), 2.73 (d, J = 7.3 Hz, 2H), 1.80-1.69 (m, 1H), 1.56-1.48 (m, 2H), 1.48-1.34 (m, 12H), 1.33-1.12 (m, 3H), 0.89 (s, 6H). |
| 188 | 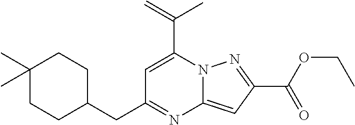 | 5 | NMR2: 7.11 (s, 1H), 6.71 (s, 1H), 6.30-6.25 (m, 1H), 5.75-5.69 (m, 1H), 4.46 (q, J = 7.1 Hz, 2H), 2.74 (d, J = 7.2 Hz, 2H), 2.38-2.33 (m, 3H), 1.84-1.71 (m, 1H), 1.56-1.49 (m, 3H), 1.43 (t, J = 7.1 Hz, 3H), 1.41-1.34 (m, 2H), 1.34-1.12 (m, 3H), 0.89 (s, 6H). |
| 189 | 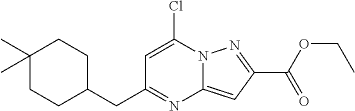 | 6 | NMR2: 7.19 (s, 1H), 6.95 (s, 1H), 4.50 (q, J = 7.1 Hz, 2H), 2.75 (d, J = 7.2 Hz, 2H), 1.83- 1.69 (m, 1H), 1.56-1.48 (m, 2H), 1.45 (t, J = 7.1 Hz, 3H), 1.41-1.35 (m, 2H), 1.34-1.12 (m, 4H), 0.89 (s, 6H). |
| 190 | 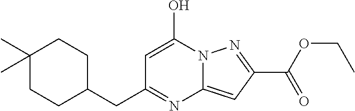 | 7 | NMR2: 11.82 (s, 1H), 6.61 (s, 1H), 5.76 (s, 1H), 4.31 (q, J = 7.1 Hz, 2H), 2.67 (d, J = 7.3 Hz, 2H), 1.75-1.58 (m, 2H), 1.56-1.47 (m, 1H), 1.37-1.29 (m, 2H), 1.26-1.01 (m, 7H), 0.86 (s, 3H), 0.83 (s, 3H). |
| 191 | 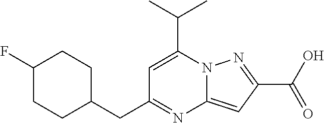 | 3 | NMR2: 7.23-7.18 (m, 1H), 6.69-6.63 (m, 1H), 4.96-4.37 (m, 1H), 4.03-3.88 (m, 1H), 2.83-2.71 (m, 2H), 2.20-1.76 (m, 4H), 1.63- 1.39 (m, 10H), 1.23-1.09 (m, 1H). |

TABLE 1-27

| REX | STR | RProp | Data |
|---|---|---|---|
| 192 | 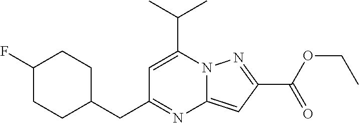 | 4 | NMR2: 7.09 (s, 1H), 6.64-6.57 (m, 1H), 4.96- 4.35 (m, 1H), 4.48 (q, J = 7.1 Hz, 2H), 4.07- 3.93 (m, 1H), 2.78-2.68 (m, 2H), 2.18-1.75 (m, 4H), 1.64-1.38 (m, 13H), 1.30-1.07 (m, 1H). |
| 193 | 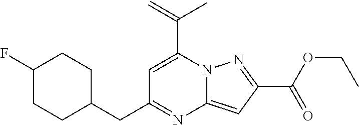 | 5 | NMR2: 7.12 (s, 1H), 6.73-6.67 (m, 1H), [symbol] .31- 6.25 (m, 1H), 5.76-5.70 (m, 1H), 4.96-4.39 (m, 1H), 4.46 (q, J = 7.1 Hz, 2H), 2.79-2.70 (m, 2H), 2.38-2.33 (m, 3H), 2.16-1.77 (m, 3H), 1.67-1.36 (m, 8H), 1.30-1.11 (m, 1H). |
| 194 | 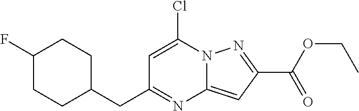 | 6 | NMR2: 7.20 (s, 1H), 6.97-6.91 (m, 1H), 4.95- 4.40 (m, 1H), 4.50 (q, J = 7.1 Hz, 2H), 2.82- 2.69 (m, 2H), 2.16-1.77 (m, 5H), 1.54-1.37 (m, 5H), 1.21-1.05 (m, 2H). |
| 195 | 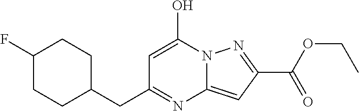 | 7 | NMR2: 12.69-12.45 (m, 1H), 7.30-7.23 (m, 1H), 6.66-6.59 (m, 1H), 5.81-5.73 (m, 1H), 4.89-4.20 (m, 3H), 2.80-2.60 (m, 2H), 2.57- 2.20 (m, 1H), 2.15-0.93 (m, 10H). |
| 196 | 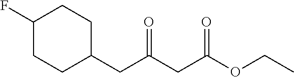 | 49 | NMR2: 4.90-4.34 (m, 1H), 4.25-4.14 (m, 2H), 3.47-3.35 (m, 2H), 2.53-2.37 (m, 1H), 2.32- 2.22 (m, 1H), 2.16-1.22 (m, 11H), 1.15-0.97 (m, 1H). |
| 197 | 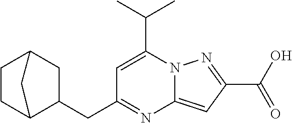 | 3 | NMR2: 7.19 (s, 1H), 6.69 (s, 1H), 4.00-3.85 (m, 1H), 2.86-2.76 (m, 1H), 2.70-2. [symbol] 0 (m, 1H), 2.29-2.24 (m, 1H), 2.07-2.02 (m, 1H), 2.02-1.94 (m, 1H), 1.58-1.37 (m, 10H), 1.25- 1.10 (m, 4H). |
| 198 | 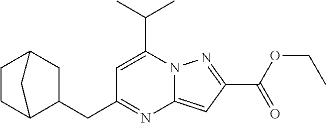 | 4 | NMR2: 7.08 (s, 1H), 6.64 (s, 1H), 4.48 (q, J = 7.1 Hz, 2H), 4.05-3.94 (m, 1H), 2.85-2.74 (m, 1H), 2.67-2.57 (m, 1H), 2.28-2.24 (m, 1H), 2.07-1.91 (m, 2H), 1.5 [symbol] -1.36 (m, 12H), 1.30-1.09 (m, 5H). |
| 199 | 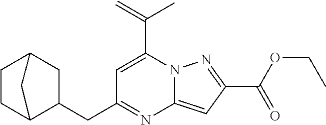 | 5 | NMR2: 7.11 (s, 1H), 6.73 (s, 1H), 6.29-6.24 (m, 1H), 5.76-5.69 (m, 1H), 4.46 (q, J = 7.1 Hz, 2H), 2.85-2.76 (m, 1H), 2.69-2.59 (m, 1H), 2.38-2.33 (m, 3H), 2.29-2.24 (m, 1H), 2.07-2.03 (m, 1H), 2.03-1.93 (m, 1H), 1.55- 1.39 (m, 6H), 1.34-1.12 (m, 5H). |

[symbol]

indicates data missing or illegible when filed

TABLE 1-28

| REX | STR | RProp | Data |
|---|---|---|---|
| 200 | 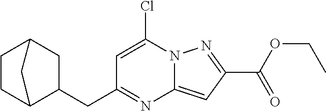 | 6 | NMR2: 7.20 (s, 1H), 6.97 (s, 1H), 4.50 (q, J = 7.1 Hz, 2H), 2.86-2.76 (m, 1H), 2.69- 2.59 (m, 1H), 2.30-2.25 (m, 1H), 2.06- 1.91 (m, 2H), 1.53-1.38 (m, 6H), 1.26- 1.10 (m, 5H). |
| 201 | 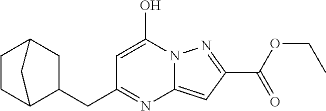 | 7 | NMR2: 11.67 (s, 1H), 6.60 (s, 1H), 5.78 (s, 1H), 4.31 (q, J = 7.1 Hz, 2H), 2.75-2.64 (m, 1H), 2.64-2.54 (m, 1H), 2.24-2.20 (m, 1H), 2.06-2.01 (m, 1H), 1.94-1.84 (m, 1H), 1.51-1.41 (m, 2H), 1.32-1.26 (m, 1H), 1.23 (t, J = 7.1 Hz, 3H), 1.16- 1.06 (m, 5H). |
| 202 | 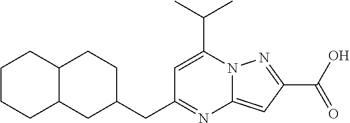 | 3 | NMR2: 7.14 (s, 1H), 6.64 (s, 1H), 3.97- 3.85 (m, 1H), 2.76-2.64 (m, 2H), 1.88- 1.84 (m, 1H), 1.76-0.83 (m, 22H). |
| 203 | 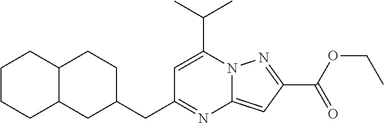 | 4 | NMR2: 7.10-7.05 (m, 1H), 6.85-6.58 (m, 1H), 4.48 (q, J = 7.1 Hz, 2H), 4.05-3.94 (m, 1H), 2.75-2.62 (m, 2H), 1.91-1.79 (m, 1H), 1.76-1.14 (m, 25H). |
| 204 | 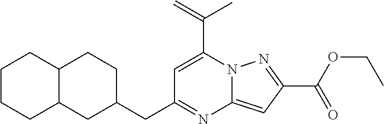 | 5 | NMR2: 7.14-7.09 (m, 1H), 6.75-6.68 (m, 1H), 6.30-6.25 (m, 1H), 5.75-5.69 (m, 1H), 4.46 (q, J = 7.1 Hz, 2H), 2.77-2.65 (m, 2H), 2.38-2.33 (m, 3H), 1.92-1.82 (m, 1H), 1.78-1.03 (m, 19H). |
| 205 | 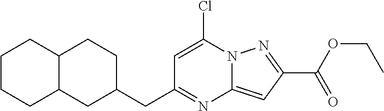 | 6 | NMR2: 7.22-7.19 (m, 1H), 6.98-6.91 (m, 1H), 4.50 (q, J = 7.1 Hz, 2H), 2.81-2.65 (m, 2H), 1.94-1.80 (m, 1H), 1.76-0.88 (m, 19H). |
| 206 | 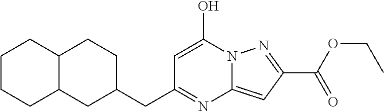 | 7 | NMR2: 11.37 (s, 1H), 6.62-6.57 (m, 1H), 5.78-5.73 (m, 1H), 4.32 (q, J = 7.1 Hz, 2H), 2.67-2.61 (m, 2H), 1.84-0.90 (m, 20H). |
| 207 | 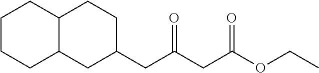 | 49 | NMR2: 4.24-4.15 (m, 2H), 3.46-3.38 (m, 2H), 2.49-2.33 (m, 2H), 2.12-1.99 (m, 1H), 1.91-0.99 (m, 19H). |

TABLE 1-29

| REX | STR | RProp | Data |
|---|---|---|---|
| 208 | 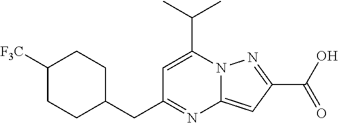 | 3 | NMR2: 7.19 (s, 1H), 6.69-6.63 (m, 1H), 3.98- 3.87 (m, 1H), 2.91-2.72 (m, 2H), 2.32- 2.09 (m, 1H), 2.00-1.82 (m, 3H), 1.76-1.69 (m, 2H), 1.64-1.55 (m, 2H), 1.44 (d, J = 6.9 Hz, 6H), 1.41-1.27 (m, 1H), 1.18-1.08 (m, 1H) |
| 209 | 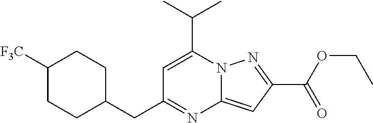 | 4 | NMR2: 7.09 (s, 1H), 6.64-6.58 (m, 1H), 4.53- 4.43 (m, 2H), 4.06-3.95 (m, 1H), 2.89- 2.70 (m, 2H), 2.33-2.08 (m, 1H), 2.00-1.83 (m, 3H), 1.78-1.68 (m, 2H), 1.63-1.57 (m, 2H), 1.48-1.29 (m, 10H), 1.17-1.03 (m, 1H). |
| 210 | 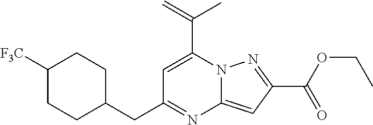 | 5 | NMR2: 7.12 (s, 1H), 6.74-6.68 (m, 1H), 6.31- 6.25 (m, 1H), 5.76-5.70 (m, 1H), 4.46 (q, J = 7.1 Hz, 2H), 2.90-2.71 (m, 2H), 2.36 (s, 3H), 2.33-2.08 (m, 1H), 2.04-1.83 (m, 3H), 1.78-1.68 (m, 2H), 1.68-1.57 (m, 2H), 1.47- 1.40 (m, 3H), 1.38-1.22 (m, 1H), 1.19- 1.04 (m, 1H). |
| 211 | 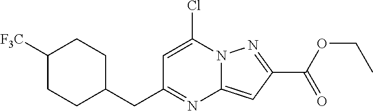 | 6 | NMR2: 7.20 (s, 1H), 6.94 (d, J = 6.8 Hz, 1H), 4.55-4.45 (m, 2H), 2.90-2.71 (m, 2H), 2.32- 2.07 (m, 1H), 2.06-1.80 (m, 3H), 1.77- 1.68 (m, 2H), 1.63-1.54 (m, 2H), 1.45 (t, J = 7.2 Hz, 3H), 1.38-1.27 (m, 1H), 1.18- 1.04 (m, 1H). |
| 212 | 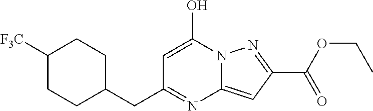 | 7 | NMR2: 12.57 (s, 1H), 6.63-6.57 (m, 1H), 5.82-5.75 (m, 1H), 4.30 (q, J = 7.1 Hz, 2H), 2.93-2.70 (m, 2H), 2.33-1.44 (m, 8H), 1.30- 1.16 (m, 4H), 1.10-0.96 (m, 1H) |
| 213 | 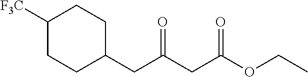 | 49 | NMR2: 4.25-4.14 (m, 2H), 3.46-3.39 (m, 2H), 2.61-2.42 (m, 2H), 2.04-1.79 (m, 3H), 1.77-1.46 (m, 6H), 1.42-1.22 (m, 3H), 1.04- 0.90 (m, 1H). |
| 214 | 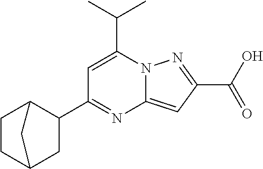 | 3 | NMR2: 7.23-7.17 (m, 1H), 6.70 (s, 1H), 3.98- 3.85 (m, 1H), 2.93-2.85 (m, 1H), 2.69- 2.49 (m, 1H), 2.47-2.35 (m, 1H), 2.26-2.13 (m, 1H), 2.08-1.80 (m, 1H), 1.73-1.52 (m, 3H), 1.52-1.25 (m, 8H), 1.23-1.12 (m, 1H). |

TABLE 1-30

| REX | STR | RProp | Data |
|---|---|---|---|
| 215 | 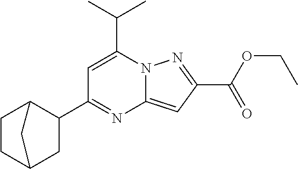 | 4 | NMR2: 7.13-7.06 (m, 1H), 6.65 (s, 1H), 4.52- 4.42 (m, 2H), 4.05-3.91 (m, 1H), 2.92-2.83 (m, 1H), 2.66-2.36 (m, 2H), 2.25-2.14 (m, 1H), 1.72-1.57 (m, 3H), 1.48-1.38 (m, 10H), 1.38-1.22 (m, 2H), 1.22-1.13 (m, 1H). |
| 216 | 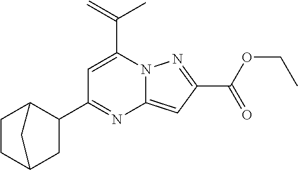 | 5 | NMR2: 7.16-7.09 (m, 1H), 6.77-6.72 (m, 1H), 6.26-6.19 (m, 1H), 5.72-5.66 (m, 1H), 4.51- 4.40 (m, 2H), 2.93-2.85 (m, 1H), 2.70-2.37 (m, 2H), 2.37-2.32 (m, 3H), 2.26-2.15 (m, 1H), 1.73-1.56 (m, 3H), 1.52-1.09 (m, 7H) |
| 217 | 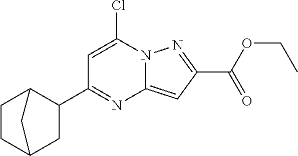 | 6 | NMR2: 7.20 (s, 1H), 7.01-6.95 (m, 1H), 4.57- 4.44 (m, 2H), 2.91-2.83 (m, 1H), 2.68-2.39 (m, 2H), 2.38-2.32 (m, 3H), 2.23-2.12 (m, 1H), 1.72-1.15 (m, 7H). |
| 218 | 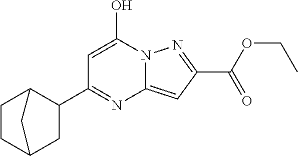 | 7 | NMR1: 12.36-12.16 (m, 1H), 6.49-6.44 (m, 1H), 5.81-5.64 (m, 1H), 4.39-4.29 (m, 2H), 3.19-3.11 (m, 1H), 2.71-2.58 (m, 1H), 2.48- 2.42 (m, 1H), 2.35-2.31 (m, 1H), 1.91-1.11 (m, 10H). |
| 219 | 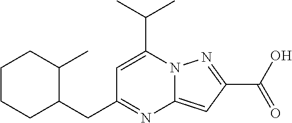 | 3 | NMR2: 7.21-7.16 (m, 1H), 6.70-6.63 (m, 1H), 3.98-3.86 (m, 1H), 3.26-2.36 (m, 2H), 2.18- 1.07 (m, 16H), 1.07-0.94 (m, 3H). |
| 220 | 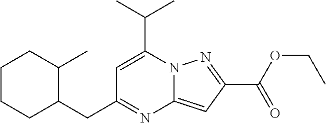 | 4 | NMR2: 7.12-7.06 (m, 1H), 6.65-6.58 (m, 1H), 4.47 (q, J = 7.1 Hz, 2H), 4.07-3.92 (m, 1H), 3.26-2.34 (m, 2H), 2.17-1.07 (m, 19H), 1.06- 0.94 (m, 3H). |
| 221 | 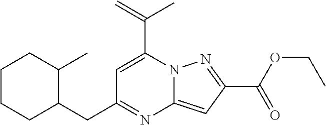 | 5 | NMR2: 7.14-7.09 (m, 1H), 6.76-6.67 (m, 1H), 6.29-6.23 (m, 1H), 5.75-5.69 (m, 1H), 4.46 (q, J = 7.1 Hz, 2H), 3.25-2.39 (m, 2H), 2.38- 2.31 (m, 3H), 2.15-1.07 (m, 13H), 1.07-0.92 (m, 3H) |
| 222 | 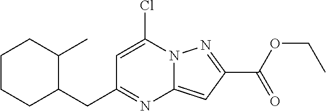 | 6 | NMR2: 7.21-7.18 (m, 1H), 6.99-6.91 (m, 1H), 4.50 (q, J = 7.1 Hz, 2H), 3.25-2.39 (m, 2H), 2.18-1.07 (m, 13H), 1.06-0.93 (m, 3H). |

TABLE 1-31

| REX | STR | RProp | Data |
|---|---|---|---|
| 223 | 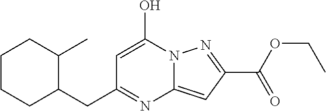 | 7 | NMR2: 12.36-12.26 (m, 1H), 6.66-6.57 (m, 1H), 5.84-5.74 (m, 1H), 4.29 (q, J = 7.1 Hz, 2H), 3.34-2.19 (m, 2H), 2.11-0.84 (m, 13H), 0.82-0.76 (m, 3H). |
| 224 | 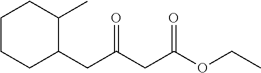 | 49 | NMR2: 4.24-4.13 (m, 2H), 3.46-3.39 (m, 2H), 2.79-2.20 (m, 2H), 2.18-0.91 (m, 13H), 0.90-0.80 (m, 3H). |
| 225 | 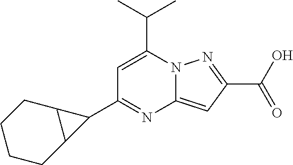 | 3 | NMR2: 7.02 (s, 1H), 6.57 (s, 1H), 3.97-3.82 (m, 1H), 2.06-1.95 (m, 2H), 1.85-1.75 (m, 5H), 1.41 (d, J = 5.9 Hz, 6H), 1.39-1.26 (m, 4H) |
| 226 | 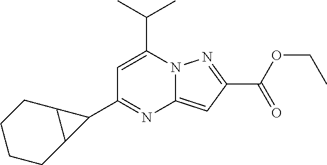 | 31 | NMR2: 6.94 (s, 1H), 6.54 (s, 1H), 4.46 (q, J = 7.1 Hz, 2H), 4.04-3.91 (m, 1H), 2.06-1.96 (m, 2H), 1.86-1.73 (m, 5H), 1.47-1.21 (m, 13H). |
| 227 | 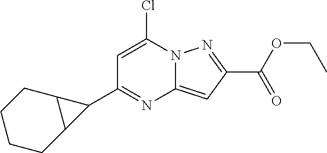 | 6 | NMR2: 7.04 (s, 1H), 6.86 (s, 1H), 4.48 (q, J = 7.1 Hz, 2H), 2.07-1.94 (m, 2H), 1.86-1.73 (m, 5H), 1.44 (t, J = 7.1 Hz, 3H), 1.42-1.22 (m, 4H). |
| 228 | 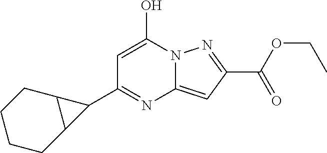 | 7 | NMR2: 11.69-11.39 (m, 1H), 6.48 (s, 1H), 5.49-5.44 (m, 1H), 4.34-4.25 (m, 2H), 2.47- 2.21 (m, 1H), 1.98-1.51 (m, 8H), 1.46- 0.99 (m, 5H). |
| 229 | 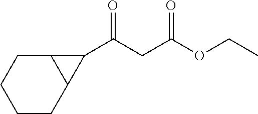 | 49 | NMR2: 4.26-4.15 (m, 2H), 3.52 (s, 2H), 1.96- 1.49 (m, 8H), 1.43-1.14 (m, 6H). |
| 230 | 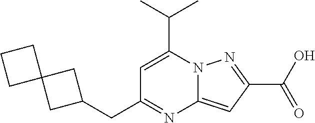 | 3 | NMR2: 7.19 (s, 1H), 6.63 (s, 1H), 4.01-3.86 (m, 1H), 2.90 (d, J = 7.6 Hz, 2H), 2.67-2.50 (m, 1H), 2.21-2.11 (m, 2H), 2.04-1.9 [symbol] (m, 2H), 1.96-1.87 (m, 2H), 1.85-1.73 (m, 4H), 1.43 (d, J = 6.9 Hz, 6H) |

[symbol]

indicates data missing or illegible when filed

TABLE 1-32

| REX | STR | RProp | Data |
|---|---|---|---|
| 231 | 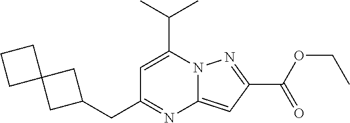 | 4 | NMR2: 7.07 (s, 1H), 6.58 (s, 1H), 4.47 (q, J = 7.1 Hz, 2H), 4.06-3.91 (m, 1H), 2.87 (d, J = 7.6 Hz, 2H), 2.66-2.49 (m, 1H), 2.20-2.10 (m, 2H), 2.03-1.95 (m, 2H), 1.95-1.87 (m, 2H), 1.85-1.72 (m, 4H), 1.48-1.38 (m, 9H). |
| 232 | 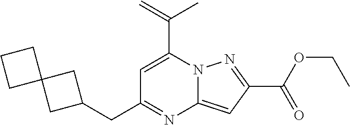 | 5 | NMR2: 7.10 (s, 1H), 6.67 (s, 1H), 6.28-6.23 (m, 1H), 5.74-5.68 (m, 1H), 4.46 (q, J = 7.1 Hz, 2H), 2.89 (d, J = 7.6 Hz, 2H), 2.65-2.51 (m, 1H), 2.37-2.32 (m, 3H), 2.21-2.11 (m, 2H), 2.04-1.96 (m, 2H), 1.96-1.87 (m, 2H), 1.85-1.73 (m, 4H), 1.43 (t, J = 7.1 Hz, 3H). |
| 233 | 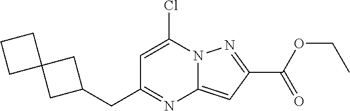 | 6 | NMR2: 7.18 (s, 1H), 6.90 (s, 1H), 4.50 (q, J = 7.1 Hz, 2H), 2.89 (d, J = 7.7 Hz, 2H), 2.66- 2.50 (m, 1H), 2.22-2.12 (m, 2H), 2.07-1.97 (m, 2H), 1.96-1.87 (m, 2H), 1.85-1.72 (m, 4H), 1.45 (t, J = 7.1 Hz, 3H). |
| 234 | 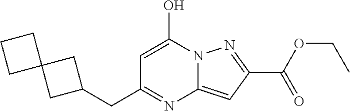 | 7 | NMR2: 11.89 (s, 1H), 6.59 (s, 1H), 5.73 (s, 1H), 4.31 (q, J = 7.1 Hz, 2H), 2.83 (d, J = 7.7 Hz, 2H), 2.61-2.48 (m, 1H), 2.20-2.11 (m, 2H), 2.00-1.92 (m, 2H), 1.90-1.82 (m, 2H), 1.81- 1.66 (m, 4H), 1.23 (t, J = 7.1 Hz, 3H). |
| 235 | 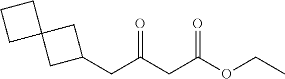 | 49 | NMR2: 4.23-4.13 (m, 2H), 3.37 (s, 2H), 2.64- 2.58 (m, 2H), 2.57-2.45 (m, 1H), 2.30- 2.10 (m, 2H), 2.04-1.96 (m, 2H), 1.91-1.83 (m, 2H), 1.82-1.71 (m, 2H), 1.71-1.47 (m, 2H), 1.31-1.24 (m, 3H). |
| 236 | 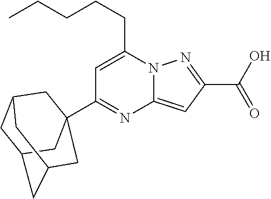 | 1 | NMR2: 13.10 (s, 1H), 7.19 (s, 1H), 6.98 (s, 1H), 3.16-3.08 (m, 2H), 2.12-2.04 (m, 3H), 2.01-1.95 (m, [symbol] H), 1.87-1.63 (m, [symbol] H), 1.44- 1.24 (m, 4H), 0.93-0.84 (m, 3H). |

[symbol]

indicates data missing or illegible when filed

### EXAMPLES

### Example 1: Synthesis of (5-adamantan-1-yl)-7-propylpyrazolo[1,5-a]pyrimidin-2-yl)(azepan-1-yl)methanone

To a solution of 5-(1-adamantyl)-7-propylpyrazolo[1,5-a]pyrimidine-2-carboxylic acid (81.9 mg) in DCM (5 ml) were added hexamethyleneimine (0.041 ml), HATU (138 mg) and TEA (0.067 ml) at 0° C., and the mixture was stirred at room temperature overnight. Saturated sodium bicarbonate was added to the reaction mixture. Then, the resulting mixture was extracted with DCM. The organic layer was concentrated, and the residue was crystallized from AcOEt/Hexane to obtain the object compound (44.4 mg).

### Example 2: Synthesis of (5-adamantan-1-yl)-7-isopropylpyrazolo[1,5-a]pyrimidin-2-yl)(2,2-dimethyl piperidin-1-yl)methanone

To a solution of 5-(1-adamantyl)-7-propan-2-ylpyrazolo[1,5-a]pyrimidine-2-carboxylic acid (300 mg) and HATU (504 mg) in DCM (10 ml) were added TEA (0.370 ml) and 2,2-dimethylpiperidine hydrochloride (159 mg). The mixture was stirred at room temperature overnight. Saturated sodium bicarbonate was added to the reaction mixture. Then, the resulting mixture was extracted with DCM. The organic layer was concentrated, and the residue was purified by column chromatography (Hexane/AcOEt), and recrystallized from EtOH/water to obtain the object compound (320 mg).

### Example 3: Synthesis of azepan-1-yl(5-(cyclohexylmethyl)-7-(trifluoromethyl)pyrazolo[1,5-a]pyrimidin-2-yl)methanone

To a solution of 5-(cyclohexylmethyl)-7-(trifluoromethyl)pyrazolo[1,5-a]pyrimidine-2-carboxylic acid (60 mg) in DCM (3 ml) were added HATU (105 mg), TEA (0.038 ml), and hexamethyleneimine (0.031 ml), and the mixture was stirred at room temperature overnight. Saturated sodium bicarbonate was added to the reaction mixture. Then, the resulting mixture was extracted with DCM. The organic layer was concentrated, and the residue was purified by column chromatography (Hexane/AcOEt) to obtain the object compound (44 mg).

### Example 4: Synthesis of azepan-1-yl(5-(cyclopentylmethyl)-7-(trifluoromethyl)pyrazolo[1,5-a]pyrimidin-2-yl)methanone

To a solution of 5-(cyclopentylmethyl)-7-(trifluoromethyl)pyrazolo[1,5-a]pyrimidine-2-carboxylic acid (250 mg) and hexamethyleneimine (0.135 ml) in DCM (5 ml) were added HATU (455 mg) and TEA (0.334 ml), and the mixture was stirred at room temperature for 2 hours. Water was added to the mixture, and the resulting mixture was extracted with AcOEt. The organic layer was concentrated, and the residue was purified by medium pressure column chromatography (Hexane/AcOEt). The product was recrystallized from EtOH/water to obtain the object compound (232 mg).

### Example 6: Synthesis of azepan-1-yl(5-(2-cyclopentylethyl)-7-(trifluoromethyl)pyrazolo[1,5-a]pyrimidin-2-yl)methanone

To a solution of 5-(2-cyclopentylethyl)-7-(trifluoromethyl)pyrazolo[1,5-a]pyrimidine-2-carboxylic acid (500 mg) in NMP (10 ml) were added hexamethyleneimine (0.21 ml), HATU (871 mg) and TEA (0.32 ml), and the mixture was stirred at room temperature for 3 hours. Saturated Na₂CO₃ aq. was added to the mixture, and the resulting mixture was extracted with AcOEt. The organic layer was concentrated, and the residue was purified by column chromatography (Hexane/AcOEt) to obtain the object compound (530 mg).

### Example 7: Synthesis of azepan-1-yl(5-(cyclohexylmethyl)-7-isopropylpyrazolo[1,5-a]pyrimidin-2-yl)methanone

To a solution of 5-(cyclohexylmethyl)-7-propan-2-ylpyrazolo[1,5-a]pyrimidine-2-carboxylic acid (368 mg) in NMP (4 ml) were added WSC (281 mg) and HOBt (224 mg), and the mixture was stirred at room temperature for 10 minutes. Hexamethyleneimine (0.165 ml) was added to the mixture, and the resulting mixture was stirred overnight. Saturated Na₂CO₃ aq. was added to the mixture, and the resulting mixture was extracted with AcOEt. The organic layer was concentrated, and the residue was purified by column chromatography (Hexane/AcOEt). The product was recrystallized from EtOH/water to obtain the object compound (304 mg).

### Example 8: Synthesis of azepan-1-yl(7-cyclohexyl-5-(cyclohexylmethyl)pyrazolo[1,5-a]pyrimidin-2-yl)methanone

To a solution of azepan-1-yl-[7-(cyclohexen-1-yl)-5-(cyclohexylmethyl)pyrazolo[1,5-a]pyrimidin-2-yl]methanone (440 mg) in AcOEt (4 ml) was added 10% Pd/C (50 mg) under nitrogen gas atmosphere. The mixture was stirred under hydrogen gas atmosphere at room temperature for 1 hour. The reaction mixture was filtered through Celite, and the filtrate was concentrated. The residue was purified by column chromatography (Hexane/AcOEt), and the product was recrystallized from EtOH/water to obtain the object compound (278 mg).

### Example 9: Synthesis of azepan-1-yl(5-(cyclohexylmethyl)-7-propylpyrazolo[1,5-a]pyrimidin-2-yl)methanone

Azepan-1-yl-[7-chloro-5-(cyclohexylmethyl)pyrazolo[1,5-a]pyrimidin-2-yl]methanone (500 mg), n-propylboronic acid (234 mg), PdCl₂(dppf)DCM (109 mg) and K₃PO₄ (849 mg) were dissolved in 1,4-dioxane (5 ml), and the mixture was heated to reflux under nitrogen atmosphere for 6 hours. Water was added to the mixture, and the resulting mixture was extracted with AcOEt. The organic layer was concentrated, and the residue was purified by column chromatography (Hexane/AcOEt) to obtain the object compound (440 mg).

### Example 15: Synthesis of azepan-1-yl-[5-(cyclopentylmethyl)-7-propan-2-ylpyrazolo[1,5-a]pyrimidin-2-yl]methanone

To a solution of 5-(cyclopentylmethyl)-7-propan-2-ylpyrazolo[1,5-a]pyrimidine-2-carboxylic acid (2.00 g) and hexamethyleneimine (1.177 ml) in DCM (20 ml) were added HATU (3.18 g) and TEA (2.91 ml), and the mixture was stirred at room temperature for 1 hour. Water was added to the mixture, and the resulting mixture was extracted with AcOEt. The organic layer was concentrated, and the residue was purified by medium pressure column chromatography (Hexane/AcOEt) to obtain the object compound (2.24 g).

### Example 28: Synthesis of azepan-1-yl-[7-tert-butyl-5-(cyclohexylmethyl)pyrazolo[1,5-a]pyrimidin-2-yl]methanone

To a solution of azepan-1-yl-[7-chloro-5-(cyclohexylmethyl)pyrazolo[1,5-a]pyrimidin-2-yl]methanone (600 mg) in NMP (1.8 ml) were added a solution of 0.5M tert-butylzinc bromide in THE (4.81 ml), copper(I) iodide (91 mg), and lithium chloride (102 mg). The mixture was stirred under nitrogen atmosphere at 50° C. for 6 hours. To the reaction mixture was added 1 N HCl, and the mixture was extracted with AcOEt. The organic layer was concentrated, and the residue was purified by column chromatography (Hexane/AcOEt). Then, the product was recrystallized from EtOH/water to obtain the object compound (363 mg).

### Example 53: Synthesis of [5-(1-adamantyl)-7-(trifluoromethyl)pyrazolo[1,5-a]pyrimidin-2-yl]-(2,2-dimethylpiperazin-1-yl)methanone

To a solution of tert-butyl 4-[5-(1-adamantyl)-7-(trifluoromethyl)pyrazolo[1,5-a]pyrimidine-2-carbonyl]-3,3-dimethylpiperazine-1-carboxylate (3.0 g) in DCM (30 ml) was added TFA (5 ml) while ice-cooling, and the mixture was stirred at room temperature for 5 hours. Water was added to the mixture while ice-cooling, and the resulting mixture was alkalized with Na₂CO₃ aq.. The mixture was extracted with DCM, and the organic layer was concentrated. Then, the residue was purified by column chromatography (MeOH/AcOEt), and the product was recrystallized from EtOH/water to obtain the object compound (1.65 g).

### Example 87: Synthesis of azepan-1-yl-[5-(cyclohexylmethyl)-7-ethylsulfanylpyrazolo[1,5-a]pyrimidin-2-yl]methanone

To a solution of azepan-1-yl-[7-chloro-5-(cyclohexylmethyl)pyrazolo[1,5-a]pyrimidin-2-yl]methanone (200 mg) in THE (2 ml) was added sodium ethanethiol (53.8 mg), and the mixture was stirred at room temperature for 30 minutes. The reaction mixture was concentrated, and the residue was purified by column chromatography (Hexane/AcOEt) to obtain the object compound (187 mg).

### Example 88: Synthesis of azepan-1-yl-[5-(cyclohexylmethyl)-3-fluoro-7-propan-2-ylpyrazolo[1,5-a]pyrimidin-2-yl]methanone

To a solution of azepan-1-yl-[5-(cyclohexylmethyl)-7-propan-2-ylpyrazolo[1,5-a]pyrimidin-2-yl]methanone (419 mg) in CH₃CN (10 ml) was added selectfluor (970 mg), and the mixture was stirred at 40° C. for 3 hours. The reaction mixture was concentrated, and ice was added to the residue. Then, the mixture was neutralized with Na₂CO₃ aq., and extracted with AcOEt. The organic layer was concentrated, and the residue was purified by basic column chromatography (Hexane/AcOEt) to obtain the object compound (95 mg).

### Example 89: Synthesis of azepan-1-yl-[5-(cyclohexylmethyl)-7-(dipropylamino)pyrazolo[1,5-a]pyrimidin-2-yl]methanone

To a solution of azepan-1-yl-[7-chloro-5-(cyclohexylmethyl)pyrazolo[1,5-a]pyrimidin-2-yl]methanone (200 mg) in IPA (2 ml) was added dipropylamine (439 μl), and the mixture was stirred at room temperature for 30 minutes. The reaction mixture was concentrated, and the residue was purified by column chromatography (Hexane/AcOEt) to obtain the object compound (217 mg).

### Example 90: Synthesis of azepan-1-yl-[5-(cyclohexylmethyl)-7-cyclohexyloxypyrazolo[1,5-a]pyrimidin-2-yl]methanone

To a solution of azepan-1-yl-[7-chloro-5-(cyclohexylmethyl)pyrazolo[1,5-a]pyrimidin-2-yl]methanone (200 mg), cyclohexanol (1113 μl) in THE (5 ml) was added KOH (150 mg), and the mixture was stirred at room temperature for 30 minutes. To the reaction mixture were added water and NH₄Cl aq., and the mixture was extracted with AcOEt. The organic layer was concentrated, and the residue was purified by column chromatography (Hexane/AcOEt) to obtain the object compound (215 mg).

### Example 116: Synthesis of [5-(cyclohexylmethyl)-7-propan-2-ylpyrazolo[1,5-a]pyrimidin-2-yl]-[4-(2-methoxyethyl)-2,2-dimethylpiperazin-1-yl]methanone

[5-(cyclohexylmethyl)-7-propan-2-ylpyrazolo[1,5-a]pyrimidin-2-yl]-(2,2-dimethylpiperazin-1-yl)methanone (512 mg), 2-bromoethyl methyl ether (0.147 ml) and K₂CO₃ (267 mg) were dissolved in NMP (5 ml), and the mixture was stirred at room temperature for 3 days. To the mixture was added saturated Na₂CO₃ aq., and the mixture was extracted with toluene. The organic layer was concentrated, and the residue was purified by silica gel column chromatography (hexane/AcOEt) to obtain the object compound (314 mg).

The compounds of Examples 5, 10-14, 16-27, 29-52, 54-86, 91-115, and 117-162 were manufactured in the same manner as in Examples 1-4, 6-9, 15, 28, 53, 87-90 and 116. Structural formulae and physicochemical data of the compounds of Reference Examples 1 to 162 are shown in Tables 2-1 to 2-23.

TABLE 2-1

| EX | STR | Prop | Data |
|---|---|---|---|
| 1 | 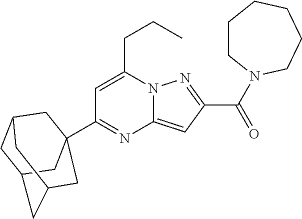 | 1 | NMR1: 7.14 (s, 1H), 6.76 (s, 1H), 3.67-3.56 (m, 4H), 3.11-3.03 (m, 2H), 2.11-2.06 (m, 3H), 2.01-1.95 (m, 7H), 1.89-1.66 (m, 12H), 1.59-1.53 (m, 3H), 0.97 (t, J = 7.4 Hz, 3H). |
| 2 | 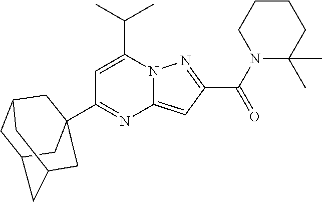 | 2 | NMR: 6.75 (d, J = 2.5 Hz, 2H), 3.91-3.79 (m, 1H), 3.56-3.50 (m, 2H), 2.17-2.12 (m, 3H), 2.02 (d, J = 3.0 Hz, 6H), 1.86-1.75 (m, 5H), 1.72-1.63 (m, 7H), 1.61 (s, 6H), 1.42 (d, J = 6.9 Hz, 6H). |
| 3 | 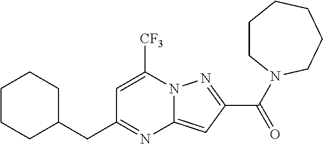 | 3 | NMR2: 7.09 (d, J = 1.2 Hz, 1H), 7.04 (s, 1H), 3.76 (dt, J = 22.0, 6.0 Hz, 4H), 2.78 (d, J = 7.2 Hz, 2H), 1.91-1.78 (m, 6H), 1.77-1.59 (m, 8H), 1.36-0.99 (m, 5H). |
| 4 | 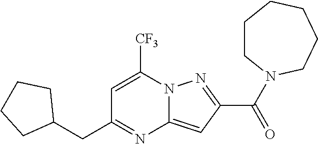 | 4 | NMR2: 7.09 (s, 1H), 7.07 (s, 1H), 3.78 (dt, J = 22.7, 6.1 Hz, 4H), 2.91 (d, J = 7.6 Hz, 2H), 2.40- 2.27 (m, 1H), 1.92-1.75 (m, 6H), 1.75-1. [symbol] 0 (m, 8H), 1.34-1.22 (m, 2H). |
| 5 | 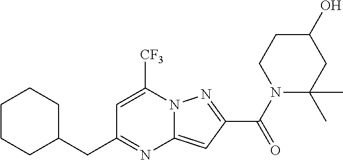 | 1 | NMR2: 7.04 (s, 1H), 6.98 (s, 1H), 4.14-4.05 (m, 1H), 3.97-3.88 (m, 1H), 3.47-3.38 (m, 1H), 2.77 (d, J = 7.2 Hz, 2H), 2.19-2.10 (m, 1H), 1.94-1.60 (m, 10H), 1.57-1.43 (m, 6H), 1.31-1.00 (m, 5H). |
| 6 | 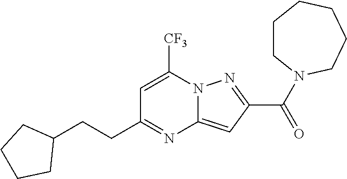 | 6 | NMR1: 7.70 (s, 1H), 7.03 (s, 1H), 3.65-3.56 (m, 4H), 2.97-2.88 (m, 2H), 1.89-1.41 (m, 17H), 1.19-1.10 (m, 2H). |
| 7 | 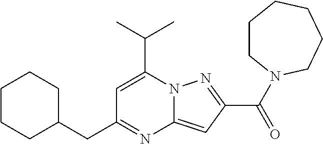 | 7 | NMR2: 6.87 (s, 1H), 6.54 (s, 1H), 3.85-3.69 (m, 5H), 2.68 (d, J = 7.2 Hz, 2H), 1.92-1.75 (m, 5H), 1.75-1.61 (m, 9H), 1.42 (d, J = 6.9 Hz, 6H), 1.31-0.98 (m, 5H). |

[symbol]

indicates data missing or illegible when filed

TABLE 2-2

| EX | STR | Prop | Data |
|---|---|---|---|
| 8 | 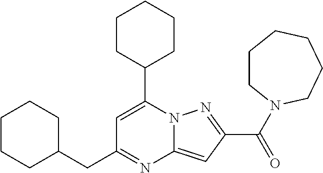 | 8 | NMR1: 6.87 (s, 1H), 6.75 (s, 1H), 3.64 (dt, J = 23.7, 6.0 Hz, 4H), 3.42-3.26 (m, 1H), 2.66 (d, J = 7.1 Hz, 3H), 2.11-2.03 (m, 2H), 1.90-0.93 (m, 26H) |
| 9 | 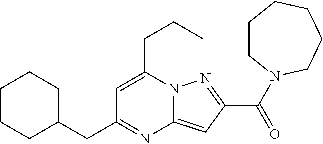 | 9 | NMR1: 6.94 (s, 1H), 6.75 (s, 1H), 3.63 (dt, J = 18.4, 6.1 Hz, 4H), 3.09-3.01 (m, 2H), 2.66 (d, J = 7.1 Hz, 2H), 1.89-1.47 (m, 15H), 1.30- 1.10 (m, 4H), 1.07-0.93 (m, 5H). |
| 10 | 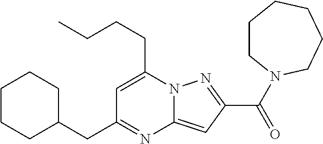 | 9 | NMR1: 6.94 (s, 1H), 6.75 (s, 1H), 3.63 (dt, J = 20.6, 6.0 Hz, 4H), 3.11-3.03 (m, 2H), 2.65 (d, J = 7.2 Hz, 2H), 1.89-1.47 (m, 15H), 1.39 (dq, J = 14.6, 7.3 Hz, 2H), 1.26-0.96 (m, 6H), 0.92 (t, J = 7.4 Hz, 3H). |
| 11 | 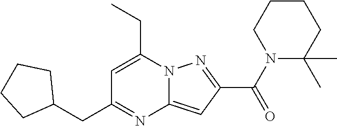 | 1 | NMR2: 6.72 (s, 1H), 6.61-6.56 (m, 1H), 3.53 (t, J = 3.7 Hz, 2H), 3.22-3.12 (m, 2H), 2.80 (d, J = 7.5 Hz, 2H), 2.37-2.23 (m, 1H), 1.82-1.62 (m, 10H), 1.61 (s, 6H), 1.59-1.49 (m, 2H), 1.42 (t, J = 7.5 Hz, 3H), 1.33-1.19 (m, 2H). |
| 12 | 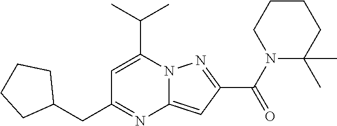 | 1 | NMR2: 6.74 (s, 1H), 6.58-6.54 (m, 1H) 3.92- 3.77 (m, 1H), 3.60-3.50 (m, 2H), 2.80 (d, J = 7.5 Hz, 2H), 2.38-2.22 (m, 1H), 1.83-1.63 (m, 7H), 1.61 (s, 6H), 1.60-1.50 (m, 6H), 1.41 (d, J = 6.9 Hz, 6H), 1.33-1.21 (m, 1H). |
| 13 | 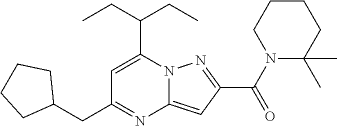 | 1 | NMR2: 6.75 (s, 1H), 6.51 (s, 1H), 3.64-3.51 (m, 3H), 2.81 (d, J = 7.6 Hz, 2H), 2.35-2.23 (m, 1H), 1.92-1.78 (m, 4H), 1.77-1.61 (m, 10H), 1.61 (s, 6H), 1.59-1.50 (m, 2H), 1.32- 1.19 (m, 2H), 0.86 (t, J = 7.4 Hz, 6H). |
| 14 | 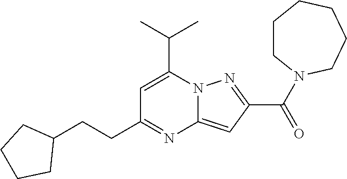 | 1 | NMR1: 6.95 (s, 1H), 6.76 (s, 1H), 3.71-3.57 (m, 5H), 2.85-2.76 (m, 2H), 1.89-1.65 (m, 7H), 1.64-1.42 (m, 9H), 1.37 (d, J = 6.9 Hz, 6H), 1.24-1.05 (m, 3H). |

TABLE 2-3

| EX | STR | Prop | Data |
|---|---|---|---|
| 15 | 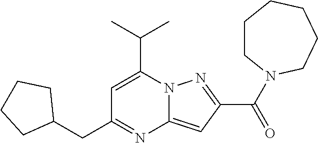 | 1 | NMR2: 6.87 (s, 1H), 6.58 (s, 1H), 3.67-3.70 (m, 5H), 2.81 (d, J = 7.5 Hz, 2H), 2.39-2.23 (m, 1H), 1.93-1.72 (m, 7H), 1.71-1.51 (m, 7H), 1.42 (d, J = 6.9 Hz, 6H), 1.33-1.20 (m, 2H). |
| 16 | 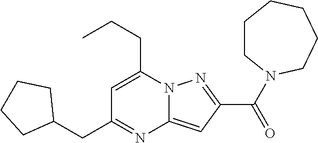 | 1 | NMR2: 6.85 (s, 1H), 6.59 (s, 1H), 3.78-3.70 (m, 4H), 3.14-3.05 (m, 2H), 2.80 (d, J = 7.5 Hz, 2H), 2.37-2.24 (m, 1H), 1.95-1.72 (m, 8H), 1.70-1.51 (m, 8H), 1.26 (ddt, J = 14.0, 12.1, 5.0 Hz, 2H), 1.06 (t, J = 7.4 Hz, 3H). |
| 17 | 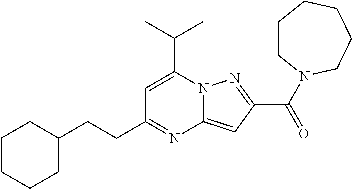 | 1 | NMR1: 6.94 (s, 1H), 6.76 (s, 1H), 3.71-3.57 (m, 5H), 2.84-2.76 (m, 2H), 1.80-1.52 (m, 16H), 1.37 (d, J = 6.9 Hz, 6H), 1.30-1.09 (m, 3H), 0.93 (qd, J = 11.9, 3.3 Hz, 2H). |
| 18 | 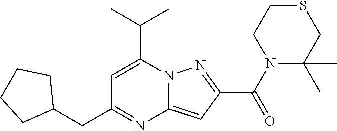 | 1 | NMR2: 6.84 (s, 1H), 6.58 (s, 1H), 3.88 (t, J = 5.8 Hz, 2H), 3.85-3.71 (m, 1H), 3.00 (t, J = 5.8 Hz, 2H), 2.81 (d, J = 7. [symbol] Hz, 2H), 2.75 (s, 2H), 2.36-2.22 (m, 1H), 1.77 (s, 10H), 1.57-1.50 (m, 2H), 1.41 (d, J = 6.9 Hz, 6H), 1.33-1.19 (m, 2H). |
| 19 | 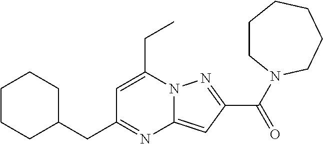 | 1 | NMR1: 6.94 (s, 1H), 6.76 (s, 1H), 3.63 (dt, J = 17.6, 6.0 Hz, 4H), 3.09 (d, J = 7.5 Hz, 2H), 2.66 (d, J = 7.2 Hz, 2H), 1.94-1.46 (m, 14H), 1.35 (t, J = 7.5 Hz, 3H), 1.29-0.89 (m, 5H). |
| 20 | 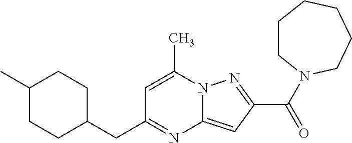 | 1 | NMR2: 7.09 (s, 1H), 7.07-7.01 (m, 1H), 3.82- 3.70 (m, 4H), 2.93-2.73 (m, 2H), 2.16-1.23 (m, 16H), 1.18-1.03 (m, 1H), 1.01-0.83 (m, 4H). |
| 21 | 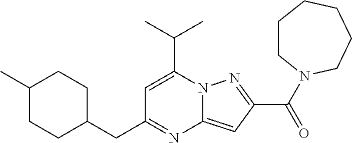 | 1 | NMR2: 6.87 (s, 1H), 6.58-6.52 (m, 1H), 3.86- 3.70 (m, 5H), 2.83-2.64 (m, 2H), 1.92-1.59 (m, 12H), 1.54-1.25 (m, 11H), 1.13-0.83 (m, 4H). |

[symbol]

indicates data missing or illegible when filed

TABLE 2-4

| EX | STR | Prop | Data |
|---|---|---|---|
| 22 | 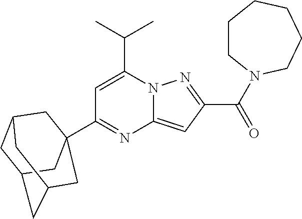 | 1 | NMR2: 6.89 (s, 1H), 6.76 (s, 1H), 3.91-3.66 (m, 5H), 2.22-2.08 (m, 3H), 2.05-2.00 (m, 6H), 1.92-1.73 (m, 10H), 1.69-1.62 (m, 4H), 1.43 (d, J = 6.9 Hz, 6H). |
| 23 | 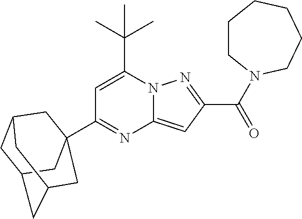 | 1 | NMR2: 6.96 (s, 1H), 6.79 (s, 1H), 3.87-3.81 (m, 2H), 3.78-3.72 (m, 2H), 2.17-2.12 (m, 3H), 2.05-2.00 (m, 6H), 1.92-1.74 (m, 10H), 1.70-1.61 (m, 4H), 1.60 (s, 9H). |
| 24 | 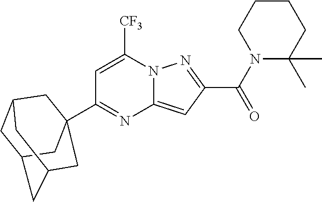 | 1 | NMR2: 7.24 (s, 1H), 6.95 (s, 1H), 3.54 (t, J = 5.7 Hz, 2H), 2.19-2.14 (m, 3H), 2.02 (d, J = 2.9 Hz, 6H), 1.87-1.65 (m, 12H), 1.60 (s, 6H). |
| 25 | 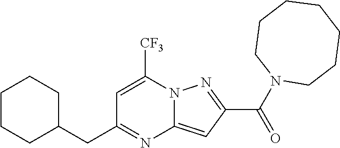 | 1 | NMR1: 7.66 (s, 1H), 7.02 (s, 1H), 3.63-3.54 (m, 4H), 2.79 (d, J = 7.1 Hz, 2H), 1.94-1.45 (m, 16H), 1.28-0.93 (m, 5H). |
| 26 | 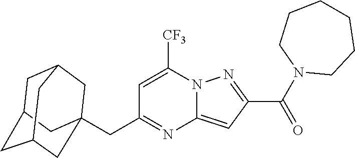 | 1 | NMR2: 7.12 (s, 1H), 6.99 (s, 1H), 3.83-3.71 (m, 4H), 2.68 (s, 2H), 2.01-1.97 (m, 3H), 1.92- 1.79 (m, 6H), 1.74-1.55 (m, 14H). |
| 27 | 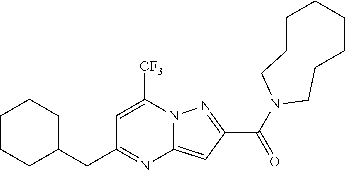 | 1 | NMR1: 7.66 (s, 1H), 7.01 (s, 1H), 3.67-3.60 (m, 2H), 3.60-3.53 (m, 2H), 2.79 (d, J = 7.1 Hz, 2H), 1.94-1.73 (m, 3H), 1.71-1.43 (m, 15H), 1.28-0.93 (m, 5H). |
| 28 | 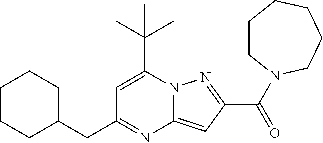 | 28 | NMR1: 6.86 (s, 1H), 6.79 (s, 1H), 3.75 (t, J = 6.0 Hz, 2H), 3.66-3.58 (m, 2H), 2.68 (d, J = 7.1 Hz, 2H), 1.92-1.46 (m, 23H), 1.29-0.93 (m, [symbol] H). |

[symbol]

indicates data missing or illegible when filed

TABLE 2-5

| EX | STR | Prop | Data |
|---|---|---|---|
| 29 | 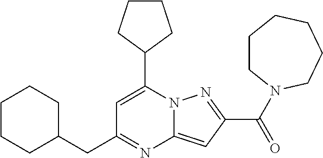 | 1 | NMR1: 6.94 (s, 1H), 6.76 (s, 1H), 3.77-3.57 (m, 5H), 2.67 (d, J = 7.2 Hz, 2H), 2.23-2.13 (m, 2H), 1.87-1.45 (m, 20H), 1.24-0.90 (m, 5H). |
| 30 | 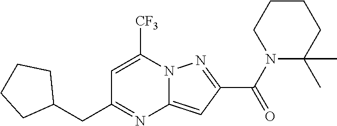 | 1 | NMR2: 7.05 (s, 1H), 6.95 (s, 1H), 3.55 (t, J = 5.7 Hz, 2H), 2.90 (d, J = 7.5 Hz, 2H), 2.39-2.25 (m, 1H), 1.85-1.63 (m, 12H), 1.64-1.54 (m, 6H), 1.33-1.20 (m, 2H). |
| 31 | 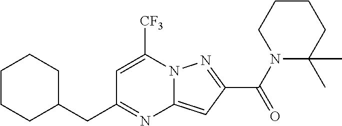 | 1 | NMR2: 7.02 (s, 1H), 6.95 (s, 1H), 3.55 (t, J = 5.7 Hz, 2H), 2.77 (d, J = 7.2 Hz, 2H), 1.93-1.62 (m, 12H), 1.60 (s, 6H), 1.30-0.94 (m, 5H). |
| 32 | 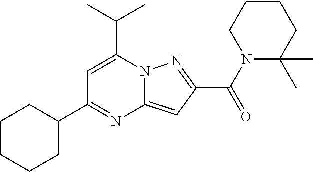 | 1 | NMR2: 6.74 (s, 1H), 6.58 (s, 1H), 3.92-3.77 (m, 1H), 3.58-3.48 (m, 2H), 2.78-2.66 (m, 1H), 2.03-1.85 (m, 5H), 1.82-1.63 (m, 9H), 1.60-1.50 (m, 5H), 1.50-1.24 (m, 9H). |
| 33 | 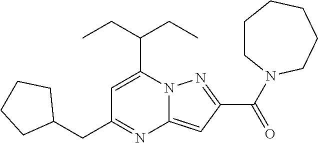 | 1 | NMR2: 6.88 (s, 1H), 6.53 (s, 1H), 3.79-3.70 (m, 4H), 3.59-3.47 (m, 1H), 2.82 (d, J = 7.6 Hz, 2H), 2.38-2.22 (m, 1H), 1.98-1.46 (m, 18H), 1.33-1.19 (m, 2H), 0.86 (t, J = 7.4 Hz, 6H). |
| 34 | 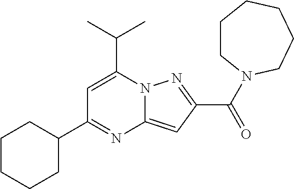 | 1 | NMR2: 6.87 (s, 1H), 6.60 (s, 1H), 3. [symbol] 5-3.68 (m, 5H), 2.79-2.67 (m, 1H), 2.02-1.74 (m, 11H), 1.71-1.51 (m, 5H), 1.50-1.21 (m, 8H). |
| 35 | 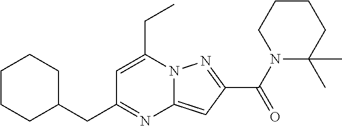 | 1 | NMR2: 6.72 (s, 1H), 6. [symbol] (s, 1H), 3. [symbol] -3. [symbol] 1 (m, 2H), 3.17 (d, J = 7.5 Hz, 2H), 2.67 (d, J = 7.2 Hz, 2H), 1.88-1.64 (m, 14H), 1.61 (s, 6H), 1.42 (t, J = 7.5 Hz, 3H), 1.28-0.95 (m, 3H) |
| 36 | 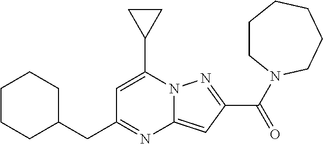 | 9 | NMR2: 6.83 (s, 1H), 6.21 (s, 1H), 3.78-3.70 (m, 5H), 2.86-2.75 (m, 1H), 2.63 (d, J = 7.2 Hz, 2H), 1.93-1.58 (m, 13H), 1.35-0.92 (m, 9H). |

[symbol]

indicates data missing or illegible when filed

TABLE 2-6

| EX | STR | Prop | Data |
|---|---|---|---|
| 37 | 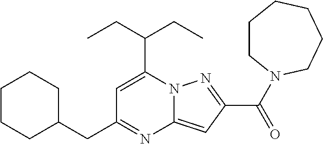 | 1 | NMR2: 6.88 (s, 1H), 6.49 (s, 1H), 3.79-3.70 (m, 4H), 3.58-3.47 (m, 1H), 2.69 (d, J = 7.3 Hz, 2H), 1.92-1.61 (m, 18H), 1.28-0.97 (m, 5H), 0.86 (t, J = 4.7 Hz, 6H). |
| 38 | 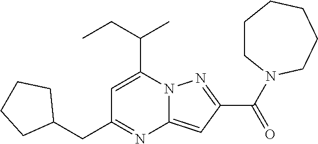 | 1 | NMR2: [symbol] .87 (s, 1H), 6.55 (s, 1H), 3.80-3.70 (m, 4H), 3.69-3.58 (m, 1H), 2.81 (d, J = 7.5 Hz, 2H), 2.36-2.24 (m, 1H), 2.00-1.49 (m, 16H), 1.39 (d, J = 7.0 Hz, 3H), 1.33-1.19 (m, 2H), 0.94 (t, J = 7.4 Hz, 3H). |
| 39 | 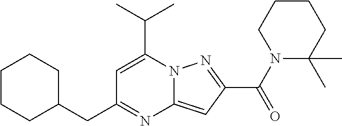 | 1 | NMR2: 6.74 (s, 1H), 6. [symbol] 3 (d, J = 0.6 Hz, 1H), 3.89-3.76 (m, 1H), 3.58-3.51 (m, 2H), 2.67 (d, J = 7.2 Hz, 2H), 1.88-1.75 (m, 1H), 1.75- 1.64 (m, 11H), 1.61 (s, 6H), 1.41 (d, J = 6.9 Hz, 6H), 1.32-1.12 (m, 3H), 1.11-0.97 (m, 2H). |
| 40 | 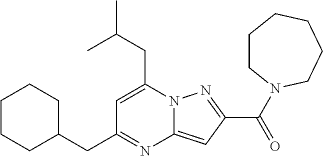 | 9 | NMR2: 6.87 (s, 1H), 6.52 (s, 1H), 3.79-3.70 (m, 4H), 2.97 (d, J = 7.1 Hz, 2H), 2.68 (d, J = 7.2 Hz, 2H), 2.42-2.27 (m, 1H), 1.92-1.62 (m, 14H), 1.31-0.95 (m, 11H). |
| 41 | 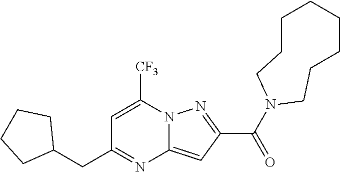 | 1 | NMR1: 7.68 (s, 1H), 7.02 (s, 1H), 3.67-3.60 (m, 2H), 3.60-3.53 (m, 2H), 2.92 (d, J = 7.5 Hz, 2H), 2.44-2.28 (m, 1H), 1.85-1.40 (m, 18H), 1.30-1.17 (m, 2H). |
| 42 | 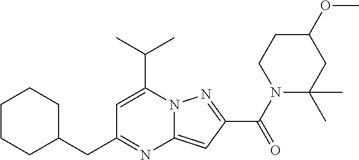 | 1 | NMR2: 6.77 (s, 1H), 6.54 (s, 1H), 3.89-3.75 (m, 2H), 3.65-3. [symbol] 4 (m, 1H), 3.54-3.43 (m, 1H), 3.36 (s, 3H), 2.68 (d, J = 7.2 Hz, 2H), 2.20- 2.04 (m, 1H), 1.96-1.59 (m, 15H), 1.42 (d, J = 6.9 Hz, 6H), 1.31-0.96 (m, 5H). |
| 43 | 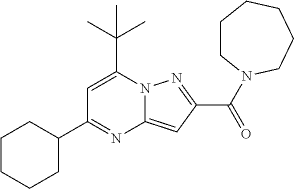 | 1 | NMR1: 6.89 (s, 1H), 6.79 (s, 1H), 3.74 (t, J = 6.0 Hz, 2H), 3 [symbol] .-3.58 (m, 2H), 2.85-2.73 (m, 1H), 1.94-1.66 (m, 10H), 1.63-1.49 (m, 14H), 1.45-1.19 (m, 3H). |

[symbol]

indicates data missing or illegible when filed

TABLE 2-7

| EX | STR | Prop | Data |
|---|---|---|---|
| 44 | 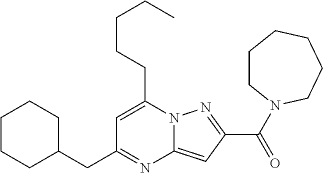 | 9 | NMR1: 6.95 (s, 1H), 6.75 (s, 1H), 3.69-3.57 (m, 4H), 3.06 (t, J = 7.6 Hz, 2H), 2.65 (d, J = 7.1 Hz, 2H), 1.88-1.49 (m, 12H), 1.40-0.81 (m, 16H). |
| 45 | 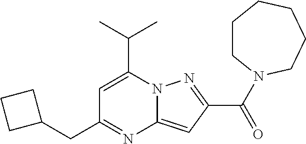 | 1 | NMR1: 6.89 (s, 1H), 6.76 (s, 1H), 3.73-3.57 (m, 6H), 2.90 (d, J = 7.6 Hz, 2H), 2.85-2.70 (m, 1H), 2.09-1.97 (m, 2H), 1.91-1.66 (m, 8H), 1.62-1.50 (m, 4H), 1.37 (d, J = 6.9 Hz, 6H). |
| 46 | 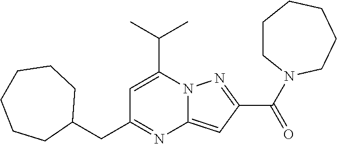 | 1 | NMR2: 6.87 (s, 1H), 6.56 (s, 1H), 3.86-3.68 (m, 5H), 2.72 (d, J = 7.4 Hz, 2H), 2.13-1.99 (m, 1H), 1.95-1.45 (m, 18H), 1.42 (d, J = 6.9 Hz, 6H), 1.35-1.22 (m, 2H). |
| 47 | 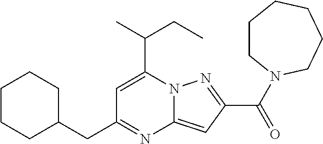 | 1 | NMR2: 6.87 (s, 1H), 6.52 (s, 1H), 3.79-3.70 (m, 4H), 3.70-3.57 (m, 1H), 2.68 (d, J = 7.2 Hz, 2H), 2.00-1.61 (m, 16H), 1.39 (d, J = 6.9 Hz, 3H), 1.28-0.99 (m, 5H), 0.94 (t, J = 7.4 Hz, 3H). |
| 48 | 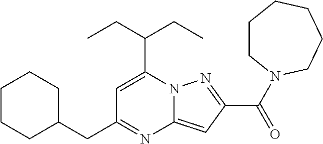 | 1 | NMR2: 6.88 (s, 1H), 6.51 (s, 1H), 3.79-3.69 (m, 4H), 3.57-3.48 (m, 1H), 2.72 (d, J = 7.4 Hz, 2H), 2.12-1.98 (m, 1H), 1.91-1.76 (m, 8H), 1.74-1.36 (m, 14H), 1.34-1.23 (m, 2H), 0.85 (t, J = 7.4 Hz, 6H). |
| 49 | 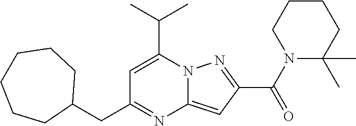 | 1 | NMR2: 6.74 (s, 1H), 6.54 (s, 1H), 3.91-3.76 (m, 1H), 3.59-3.51 (m, 2H), 2.71 (d, J = 7.4 Hz, 2H), 2.11-1.98 (m, 1H), 1.76-1.45 (m, 22H), 1.41 (d, J = 6.9 Hz, 6H), 1.35-1.21 (m, 2H). |
| 50 | 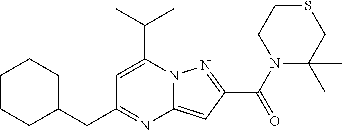 | 1 | NMR2: 6.84 (s, 1H), 6.55 (s, 1H), 3.88 (t, J = 5.8 Hz, 2H), 3.84-3.70 (m, 1H), 3.00 (d, J = 5.8 Hz, 2H), 2.75 (s, 2H), 2.68 (d, J = 7.2 Hz, 2H), 1.91-1.58 (m, 12H), 1.41 (d, J = 6.9 Hz, 6H), 1.32-0.96 (m, 5H). |
| 51 | 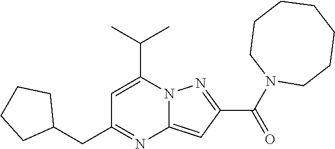 | 1 | NMR1: 6.93 (s, 1H), 6.75 (s, 1H), 3.73-3.62 (m, 3H), 3.59 (t, J = 6.1 Hz, 2H), 2.80 (d, J = 7.5 Hz, 2H), 2.41-2.25 (m, 1H), 1.77-1.46 (m, 16H), 1.38 (d, J = 6.9 Hz, 6H), 1.30-1.19 (m, 2H). |

TABLE 2-8

| EX | STR | Prop | Data |
|---|---|---|---|
| 52 | 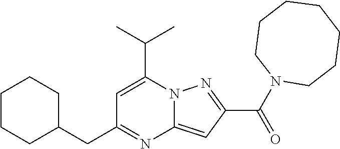 | 1 | NMR1: 6.91 (s, 1H), 6.75 (s, 1H), 3.73-3.55 (m, 5H), 2.67 (d, J = 7.1 Hz, 2H), 1.91-1.47 (m, 16H), 1.38 (d, J = 6.9 Hz, 6H), 1.25-0.92 (m, 5H). |
| 53 | 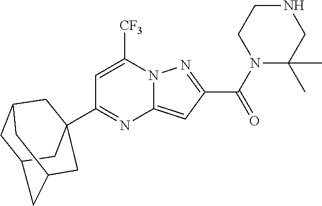 | 53 | NMR2: 7.25 (s, 1H), 6.98 (s, 1H), 3. [symbol] 8-3.62 (m, 2H), 3.03-2.97 (m, 2H), 2.79 (s, 2H), 2.20- 2.14 (m, 3H), 2.04-2.00 (m, 6H), 1.87-1.75 (m, 7H), 1.57 (s, 6H). |
| 54 | 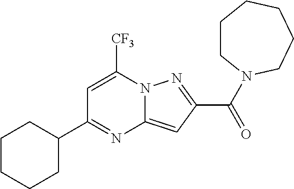 | 1 | NMR2: 7.12-7.07 (m, 2H), 3.82-3.70 (m, 4H), 2.82 (tt, J = 11.8, 3.4 Hz, 1H), 2.05-1.97 (m, 2H), 1.96-1.74 (m, 8H), 1.72-1.58 (m, 4H), 1.55-1.23 (m, 4H) |
| 55 | 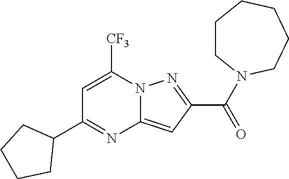 | 1 | NMR2: 7.11-7.05 (m, 2H), 3.82-3.69 (m, 4H), 2.21-2.07 (m, 2H), 1.95-1.71 (m, 10H), 1.67- 1.57 (m, 5H). |
| 56 | 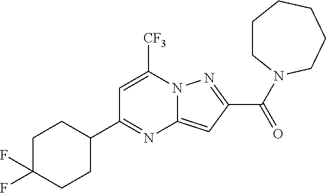 | 1 | NMR2: 7.14-7.08 (m, 2H), 3.82-3.70 (m, 4H), 2.99-2.88 (m, 1H), 2.34-2.22 (m, 3H), 2.15- 1.77 (m, 9H), 1.71-1.59 (m, 4H). |
| 57 | 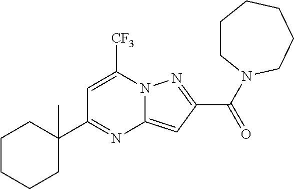 | 1 | NMR2: 7.24 (s, 1H), 7.10 (s, 1H), 3. [symbol] 3-3.70 (m, 4H), 2.22-2.17 (m, 2H), 1.92-1.77 (m, 4H), 1.71-1.58 (m, 12H), 1.29 (s, 3H). |
| 58 | 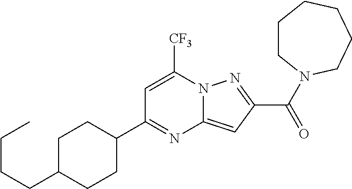 | 1 | NMR2: 7.14-7.05 (m, 2H), 3.84-3.70 (m, 4H), 2.96-2.72 (m, 1H), 2.08-1.72 (m, 7H), 1.71- 1.53 (m, 7H), 1.41-1.21 (m, 7H), 1.16-1.01 (m, 2H), 0.95-0.87 (m, 3H). |

[symbol]

indicates data missing or illegible when filed

TABLE 2-9

| EX | STR | Prop | Data |
|---|---|---|---|
| 59 | 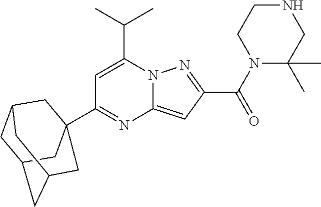 | 53 | NMR2: 6.77 (s, 1H), 6.77-6.74 (m, 1H), 3.91- 3.76 (m, 1H), 3.65-3.58 (m, 2H), 3.01- 2.94 (m, 2H), 2.78 (s, 2H), 2.17-2.12 (m, 3H), 2.02 (d, J = 2.9 Hz, 6H), 1.86-1.75 (m, 7H), 1.58 (s, 6H), 1.42 (d, J = 6.9 Hz, 6H). |
| 60 | 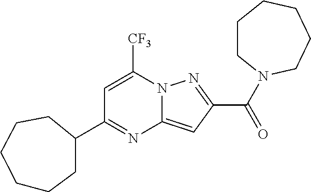 | 1 | NMR2: 7.10-7.03 (m, 2H), 3.85-3.69 (m, 4H), 2.99 (tt, J = 10.2, 3.7 Hz, 1H), 2.08- 1.97 (m, 2H), 1.94-1.54 (m, 18H). |
| 61 | 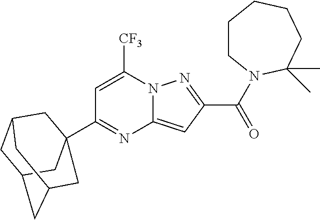 | 1 | NMR2: 7.23 (s, 1H), 7.01 (s, 1H), 3.50-3.43 (m, 2H), 2.20-2.14 (m, 3H), 2.05-1.96 (m, 6H), 1.93-1.70 (m, 11H), 1.66-1.53 (m, 9H). |
| 62 | 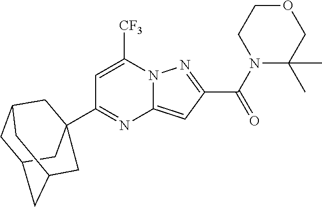 | 1 | NMR2: 7.26 (s, 1H), 7.01 (s, 1H), 3.85-3.77 (m, 4H), 3.50 (s, 2H), 2.19-2.15 (m, 3H), 2.02 (d, J = 2.9 Hz, 6H), 1.87-1.74 (m, 6H), 1.55 (s, 6H). |
| 63 | 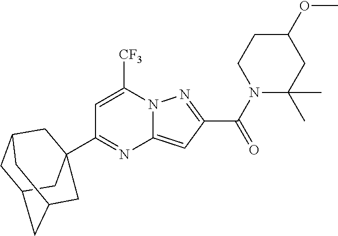 | 1 | NMR2: 7.25 (s, 1H), 6.98 (s, 1H), 3.84-3.75 (m, 1H), 3.65-3.56 (m, 1H), 3.56-3.48 (m, 1H), 3.36 (s, 3H), 2.19-2.12 (m, 4H), 2.04- 2.00 (m, 6H), 1.91 (dd, J = 13.6, 4.7 Hz, 1H), 1.87-1.75 (m, 7H), 1.70-1.58 (m, 7H). |
| 64 | 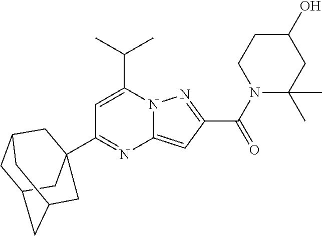 | 1 | NMR2: 6.79 (s, 1H), 6.76 (s, 1H), 4.11-4.04 (m, 1H), 3.99-3.90 (m, 1H), 3.90-3.78 (m, 1H), 3.40-3.31 (m, 1H), 2.20-2.11 (m, 4H), 2.11-2.00 (m, 6H), 1.93-1.86 (m, 1H), 1.85- 1.74 (m, 7H), 1.71 (s, 3H), 1.68-1.49 (m, 5H), 1.45-1.40 (m, 6H). |

TABLE 2-10

| EX | STR | Prop | Data |
|---|---|---|---|
| 65 | 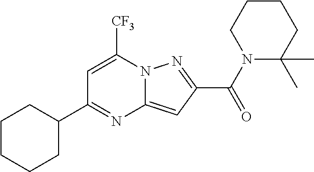 | 1 | NMR2: 7.08 (s, 1H), 6.94 (s, 1H), 3.54 (t, J = 5.7 Hz, 2H), 2.86-2.76 (m, 1H), 2.04-1.97 (m, 2H), 1.95-1.87 (m, 2H), 1.83-1.75 (m, 1H), 1.75-1.65 (m, 2H), 1.63-1.52 (m, 11H), 1.49-1.24 (m, 3H). |
| 66 | 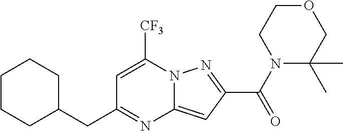 | 1 | NMR2: 7.05 (s, 1H), 7.01 (s, 1H), 3.86-3.78 (m, 4H), 3.51 (s, 2H), 2.78 (d, J = 7.2 Hz, 2H), 1.91-1.80 (m, 1H), 1.76-1.64 (m, 5H), 1.55 (s, 6H), 1.32-0.95 (m, 5H). |
| 67 | 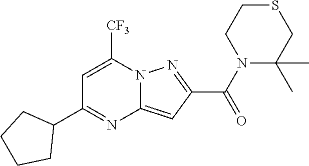 | 1 | NMR2: 7.09 (s, 1H), 7.03 (s, 1H), 3.87 (t, J = 5.9 Hz, 2H), 3.36-3.23 (m, 1H), 3.03 (t, J = 5.8 Hz, 2H), 2.74 (s, 2H), 2.21-2.08 (m, 2H), 1.94-1.81 (m, 4H), 1.77-1.73 (m, 8H). |
| 68 | 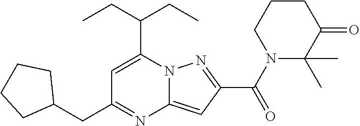 | 1 | NMR2: 6.91 (s, 1H), 6.56 (s, 1H), 3.63 (t, J = 6.3 Hz, 2H), 3.60-3.49 (m, 1H), 2.83 (d, J = 7.5 Hz, 2H), 2.79-2.70 (m, 2H), 2.37-2.22 (m, 3H), 1.92-1.80 (m, 4H), 1.73 (s, 10H), 1.60- 1.52 (m, 2H), 1.33-1.21 (m, 2H), 0.87 (t, J = 7.4 Hz, 6H). |
| 69 | 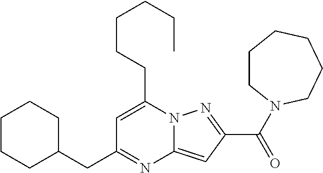 | 9 | NMR1: 6.94 (s, 1H), 6.75 (s, 1H), 3.63 (dt, J = 20.2, 6.1 Hz, 4H), 3.06 (t, J = 7.6 Hz, 2H), 2.65 (d, J = 7.1 Hz, 2H), 1.89-1.49 (m, 17H), 1.42- 0.92 (m, 10H), 0.89-0.81 (m, 3H). |
| 70 | 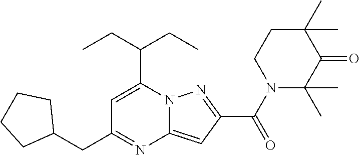 | 1 | NMR2: 6.86 (s, 1H), 6.56 (s, 1H), 3.75-3.67 (m, 2H), 3.61-3.49 (m, 1H), 2.83 (d, J = 7.5 Hz, 2H), 2.36-2.24 (m, 1H), 2.07-1.98 (m, 2H), 1.93-1.81 (m, 4H), 1.75-1.50 (m, 12H), 1.33-1.20 (m, 8H), 0.88 (t, J = 7.4 Hz, 6H). |
| 71 | 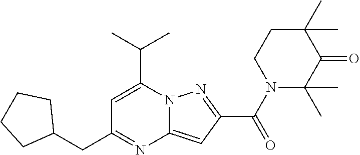 | 1 | NMR2: 6.86 (s, 1H), 6.63-6.58 (m, 1H), 3.86- 3.75 (m, 1H), 3.75-3.68 (m, 2H), 2.82 (d, J = 7.5 Hz, 2H), 2.37-2.25 (m, 1H), 2.06-1.99 (m, 2H), 1.82-1.62 (m, 9H), 1.59-1.49 (m, 3H), 1.44 (d, J = 6.9 Hz, 6H), 1.33-1.20 (m, 8H). |

TABLE 2-11

| EX | STR | Prop | Data |
|---|---|---|---|
| 72 | 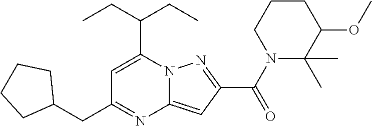 | 1 | NMR2: 6.75 (s, 1H), 6.51 (s, 1H), 3.77-3.68 (m, 1H), 3.66-3.55 (m, 1H), 3.45-3.33 (m, 4H), 3.15-3.07 (m, 1H), 2.80 (d, J = 7.6 Hz, 2H), 2.35-2.23 (m, 1H), 2.02-1.92 (m, 1H), 1.92-1.63 (m, 9H), 1.61-1.49 (m, 10H), 1.32- 1.19 (m, 2H), 0.85 (td, J = 7.4, 2.1 Hz, 6H). |
| 73 | 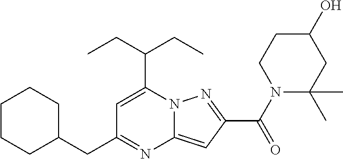 | 1 | NMR2: 6.78 (s, 1H), 6.50 (s, 1H), 4.10-4.05 (m, 1H), 4.00-3.90 (m, 1H), 3.64-3.52 (m, 1H), 3.43-3.32 (m, 1H), 2.68 (d, J = 7.2 Hz, 2H), 2.16-2.04 (m, 1H), 1.94-1.67 (m, 17H), 1.54 (s, 3H), 1.31-0.96 (m, 5H), 0.94-0.81 (m, 6H). |
| 74 | 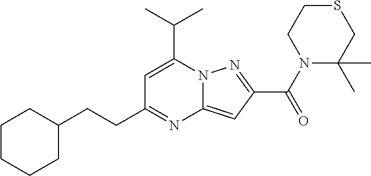 | 1 | NMR2: 6.83 (s, 1H), 6.58 (d, J = 0.7 Hz, 1H), 3.87 (t, J = 5.8 Hz, 2H), 3.83-3.73 (m, 1H), 3.03-2.96 (m, 2H), 2.85-2.77 (m, 2H), 2.75 (s, 2H), 1.84-1.60 (m, 12H), 1.41 (d, J = 6.9 Hz, 6H), 1.38-1.09 (m, 5H), 1.04-0.88 (m, 2H). |
| 75 | 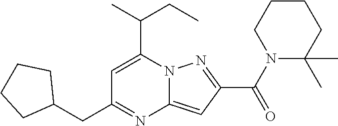 | 1 | NMR2: 6.74 (s, 1H), 6.53 (s, 1H), 3.77-3.64 (m, 1H), 3.60-3.51 (m, 2H), 2.80 (d, J = 7.5 Hz, 2H), 2.38-2.21 (m, 1H), 1.98-1.83 (m, 1H), 1.80-1.63 (m, 11H), 1.61 (d, J = 2.4 Hz, 8H), 1.38 (d, J = 7.0 Hz, 3H), 1.32-1.19 (m, 2H), 0.94 (t, J = 7.4 Hz, 3H) |
| 76 | 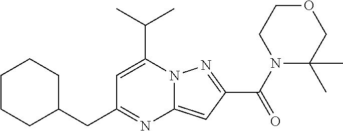 | 1 | NMR2: 6.79 (s, 1H), 6.56 (s, 1H), 3.88-3.73 (m, 5H), 3.50 (s, 2H), 2.68 (d, J = 7.2 Hz, 2H), 1.88-1.59 (m, 6H), 1.56 (s, 6H), 1.41 (d, J = 6.9 Hz, 6H), 1.32-0.95 (m, 5H). |
| 77 | 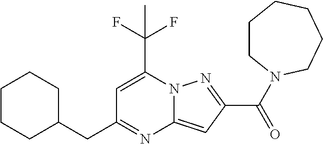 | 1 | NMR2: 7.01 (s, 1H), 6.94 (s, 1H), 3.79-3.70 (m, 4H), 2.75 (d, J = 7.2 Hz, 2H), 2.29 (t, J = 19.1 Hz, 3H), 1.92-1.59 (m, 14H), 1.33-0.98 (m, 5H). |
| 78 | 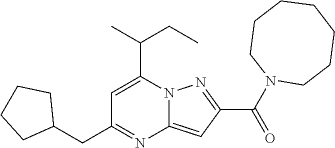 | 1 | NMR1: 6.94 (s, 1H), 6.74 (s, 1H), 3.68-3.46 (m, 5H), 2.79 (d, J = 7.5 Hz, 2H), 2.38-2.24 (m, 1H), 1.97-1.83 (m, 1H), 1.80-1.44 (m, 17H), 1.36 (d, J = 6.9 Hz, 3H), 1.29-1.16 (m, 2H), 0.86 (t, J = 7.4 Hz, 3H). |

TABLE 2-12

| EX | STR | Prop | Data |
|---|---|---|---|
| 79 | 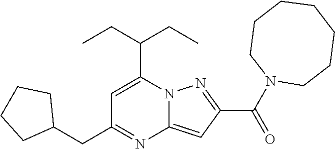 | 1 | NMR1: 6.95 (s, 1H), 6.74 (s, 1H), 3.65 (t, J = 5.4 Hz, 2H), 3.59 (t, J = 6.1 Hz, 2H), 3.48-3.37 (m, 1H), 2.80 (d, J = 7.5 Hz, 2H), 2.31 (p, J = 7.7 Hz, 1H), 1.95-1.43 (m, 19H), 1.30-1.16 (m, 3H), 0.78 (t, J = 7.4 Hz, 6H). |
| 80 | 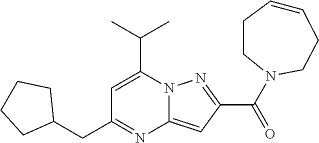 | 1 | NMR2: 6.89 (s, 1H), 6.59 (d, J = 0.8 Hz, 1H), 5.90-5.72 (m, 2H), 3.88-3.71 (m, 5H), 2.82 (d, J = 7.5 Hz, 2H), 2.52-2.40 (m, 4H), 2.37- 2.25 (m, 1H), 1.83-1.63 (m, 4H), 1.60-1.51 (m, 2H), 1.42 (d, J = 6.9 Hz, 6H), 1.35-1.20 (m, 2H). |
| 81 | 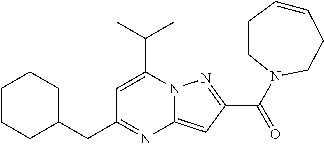 | 1 | NMR2: 6.89 (s, 1H), 6.55 (d, J = 0.8 Hz, 1H), 5.90-5.72 (m, 2H), 3.87-3.73 (m, 5H), 2.69 (d, J = 7.2 Hz, 2H), 2.52-2.40 (m, 5H), 1.89- 1.62 (m, 5H), 1.42 (d, J = 6.9 Hz, 6H), 1.32- 0.98 (m, 5H). |
| 82 | 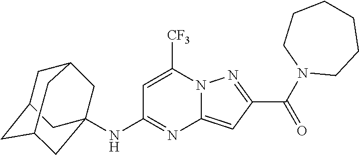 | 1 | NMR1: 7.49 (s, 1H), 6.95 (s, 1H), 6.26 (s, 1H), 3.58 (dt, J = 14.3, 6.0 Hz, 4H), 2.18-2.10 (m, 6H), 1.78-1.60 (m, 12H), 1.60-1.47 (m, 5H). |
| 83 | 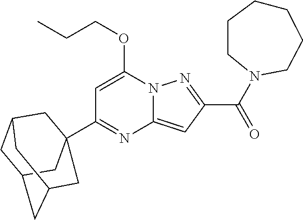 | 1 | NMR1: 6.65 (s, 2H), 4.45 (t, J = 6.5 Hz, 2H), 3.58 (dt, J = 9.0, 6.0 Hz, 4H), 2.11-2.06 (m, 3H), 2.02-1.97 (m, 6H), 1.94-1.81 (m, 2H), 1.79-1.69 (m, 8H), 1.68-1.48 (m, 6H), 1.04 (t, J = 7.4 Hz, 3H). |
| 84 | 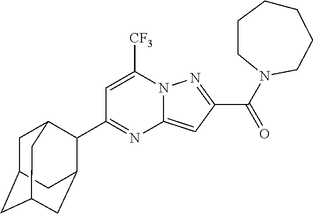 | 1 | NMR2: 7.19 (s, 1H), 7.10 (s, 1H), 3.83-3.69 (m, 4H), 3.13 (s, 1H), 2.63 (s, 2H), 2.09-1.76 (m, 14H), 1.71-1.59 (m, 6H). |
| 85 | 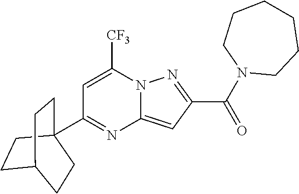 | 1 | NMR2: 7.21 (s, 1H), 7.08 (s, 1H), 3.81-3.70 (m, 4H), 1.95-1.59 (m, 21H). |

TABLE 2-13

| EX | STR | Prop | Data |
|---|---|---|---|
| 86 | 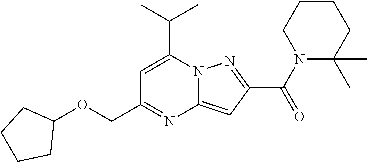 | 1 | NMR2: 6.93 (s, 1H), 6.78 (s, 1H), 4.58 (s, 2H), 4.15-3.98 (m, 1H), 3.95-3.74 (m, 1H), 3.59- 3.49 (m, 2H), 1.81-1.64 (m, 14H), 1.61 (s, 6H), 1.43 (d, J = 6.9 Hz, 6H). |
| 87 | 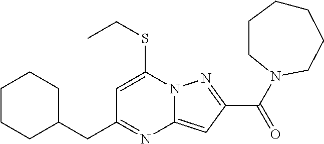 | 87 | NMR2: 6.86 (s, 1H), 6.50 (s, 1H), 3.80-3.69 (m, 4H), 3.14 (q, J = 7.4 Hz, 2H), 2.68 (d, J = 7.2 Hz, 2H), 1.91-1.76 (m, 5H), 1.75-1.59 (m, 9H), 1.52 (t, J = 7.4 Hz, 3H), 1.28-0.98 (m, 5H). |
| 88 | 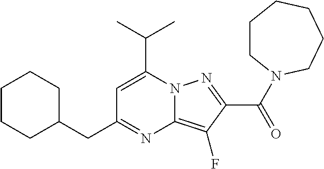 | 88 | NMR2: 6.54 (d, J = 0.7 Hz, 1H), 3.87-3.71 (m, 3H), 3.63 (t, J = 6.0 Hz, 2H), 2.70 (d, J = 7.2 Hz, 2H), 1.92-1.57 (m, 14H), 1.40 (d, J = 6.9 Hz, 6H), 1.33-0.98 (m, 5H). |
| 89 | 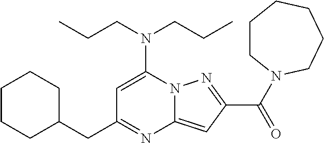 | 89 | NMR2: 6.65 (s, 1H), 5.81 (s, 1H), 3.78-3.66 (m, 8H), 2.55 (d, J = 7.1 Hz, 2H), 1.90-1.52 (m, 16H), 1.31-0.96 (m, 7H), 0.92 (t, J = 7.4 Hz, 6H). |
| 90 | 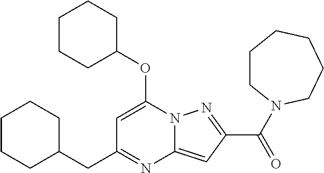 | 90 | NMR2: 6.72 (s, 1H), 6.03 (s, 1H), 4.72-4.61 (m, 1H), 3.74-3.58 (m, 4H), 2.65 (d, J = 7.2 Hz, 2H), 2.17-2.06 (m, 2H), 2.00-0.95 (m, 27H). |
| 91 | 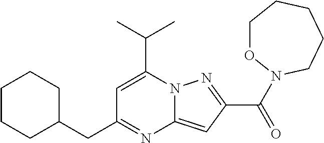 | 1 | NMR2: 7.04 (s, 1H), 6.57 (d, J = 0.8 Hz, 1H), 4.37-3.69 (m, 5H), 2.69 (d, J = 7.2 Hz, 2H), 1.95-1.77 (m, 7H), 1.75-1.62 (m, 5H), 1.43 (d, J = 6.9 Hz, 5H), 1.35-0.97 (m, 6H). |
| 92 | 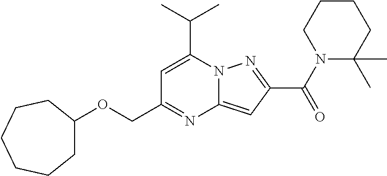 | 1 | NMR2: 6.97 (d, J = 0.6 Hz, 1H), 6.77 (s, 1H), 4.61 (s, 2H), 3.94-3.80 (m, 1H), 3.63-3.51 (m, 3H), 2.02-1.90 (m, 2H), 1.78-1.63 (m, 11H), 1.61 (s, 6H), 1.60-1.50 (m, 4H), 1.43 (d, J = 6.9 Hz, 7H). |
| 93 | 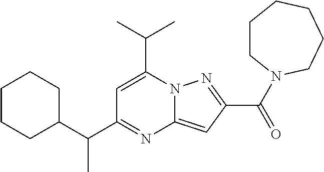 | 1 | NMR2: 6.88 (s, 1H), 6.53 (d, J = 0.7 Hz, 1H), 3.86-3.70 (m, 5H), 2.72-2.61 (m, 1H), 1.93- 1.72 (m, 6H), 1.72-1.57 (m, 8H), 1.43 (dd, J = 6.9, 0.7 Hz, 6H), 1.29 (d, J = 7.0 Hz, 3H), 1.26- 0.91 (m, 5H). |

TABLE 2-14

| EX | STR | Prop | Data |
|---|---|---|---|
| 94 | 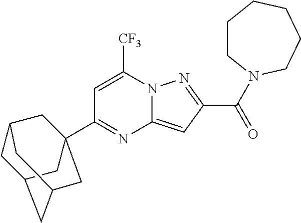 | 1 | NMR2: 7.25 (s, 1H), 7.10 (s, 1H), 3.82-3.70 (m, 4H), 2.19-2.15 (m, 3H), 2.05-2.00 (m, 6H), 1.92-1.75 (m, 10H), 1.68-1.60 (m, 4H). |
| 95 | 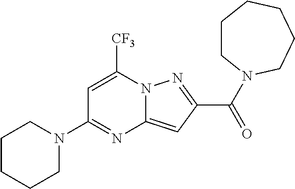 | 1 | NMR2: 6.76 (s, 1H), 6.50 (s, 1H), 3.80-3.73 (m, 2H), 3.73-3.66 (m, 5H), 1.88-1.57 (m, 14H). |
| 96 | 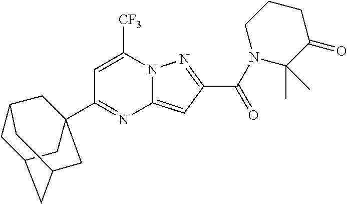 | 1 | NMR2: 7.29 (s, 1H), 7.12 (s, 1H), 3.61 (t, J = 6.3 Hz, 2H), 2.78-2.70 (m, 2H), 2.37-2.25 (m, 2H), 2.21-2.15 (m, 3H), 2.03 (d, J = 2.9 Hz, 6H), 1.89-1.74 (m, 6H), 1.71 (s, 6H). |
| 97 | 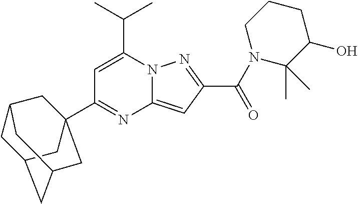 | 1 | NMR2: 6.79 (s, 1H), 6.77 (s, 1H), 3.88-3.79 (m, 1H), 3.68-3.50 (m, 3H), 2.17-2.12 (m, 3H), 2.06-1.92 (m, 8H), 1.86-1.66 (m, 9H), 1.66-1.60 (m, 6H), 1.42 (d, J = 6.9 Hz, 6H). |
| 98 | 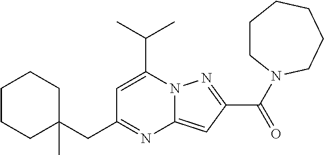 | 1 | NMR2: 6.89 (s, 1H), 6.54 (s, 1H), 3.84-3.71 (m, 5H), 2.74 (s, 2H), 1.94-1.73 (m, H), 1.71-1.54 (m, 7H), 1.53-1.27 (m, 12H), 0.96 (s, 3H). |
| 99 | 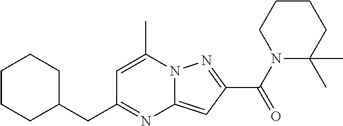 | 1 | NMR2: 6.71 (s, 1H), 6.60-6.54 (m, 1H), 3.56- 3.49 (m, 2H), 2.75 (d, J = 1.0 Hz, 3H), 2.66 (d, J = 7.2 Hz, 2H), 1.88-1.75 (m, 1H), 1.75- 1.65 (m, 1H), 1.61 (s, 6H), 1.30-1.13 (m, 3H), 1.10-0.97 (m, 2H). |
| 100 | 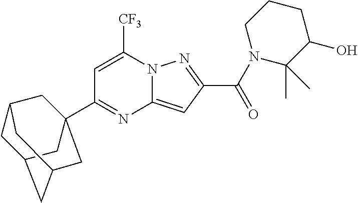 | 1 | NMR2: 7.25 (s, 1H), 6.98 (s, 1H), 3.70-3.48 (m, 3H), 2.20-2.13 (m, 3H), 2.05-1.91 (m, 9H), 1.89-1.68 (m, 8H), 1.66 (s, 3H), 1.58 (s, 3H). |

TABLE 2-15

| EX | STR | Prop | Data |
|---|---|---|---|
| 101 | 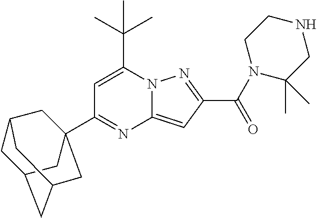 | 53 | NMR2: 6.84 (s, 1H), 6.78 (s, 1H), 3.78-3.61 (m, 2H), 3.06-32.93 (m, 2H), 2.79 (s, 2H), 2.22-2.08 (m, 3H), 2.07-1.97 (m, 6H), 1.87- 1.73 (m, 6H), 1.66-1.32 (m, 16H). |
| 102 | 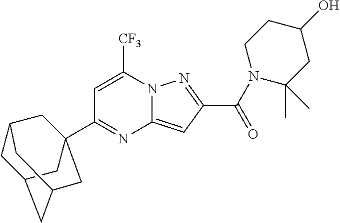 | 1 | NMR2: 7.25 (s, 1H), 6.98 (s, 1H), 4.13-4.04 (m, 1H), 3.96-3.88 (m, 1H), 3.45-3.37 (m, 1H), 2.20-2.09 (m, 4H), 2.04-2.00 (m, 6H), 1.93-1.73 (m, 8H), 1.70 (s, 3H), 1.68-1.59 (m, 1H), 1.55 (s, 3H), 1.53-1.50 (m, 1H). |
| 103 | 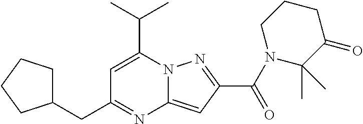 | 1 | NMR2: 6.91 (s, 1H), 6.61 (s, 1H), 3.88-3.75 (m, 1H), 3.64 (t, J = 6.3 Hz, 2H), 2.83 (d, J = 7.5 Hz, 2H), 2.78-2.70 (m, 2H), 2.39-2.22 (m, 3H), 1.60-1.54 (m, 10H), 1.57-1.52 (m, 2H), 1.44 (d, J = 6.9 Hz, 6H), 1.34-1.20 (m, 2H). |
| 104 | 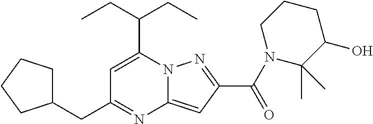 | 1 | NMR2: 6.78 (s, 1H), 6.53 (s, 1H), 3.68-3.49 (m, 4H), 2.81 (d, J = 7.5 Hz, 2H), 2.36-2.21 (m, 1H), 2.17-2.09 (m, 1H), 2.03-1.93 (m, 1H), 1.92-1.62 (m, 13H), 1.61-1.47 (m, 6H), 1.32-1.19 (m, 2H), 0.86 (t, J = 7.4 Hz, 6H). |
| 105 | 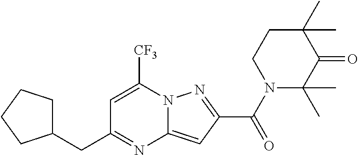 | 1 | NMR2: 7.10 (s, 1H), 7.07 (s, 1H), 3.75-3.68 (m, 2H), 2.92 (d, J = 7.6 Hz, 2H), 2.41-2.28 (m, 1H), 2.09-2.02 (m, 2H), 1.86-1.65 (m, 10H), 1.61-1.56 (m, 2H), 1.34-1.21 (m, 8H). |
| 106 | 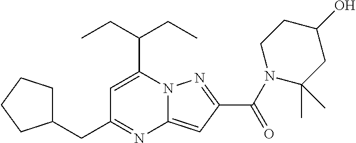 | 1 | NMR2: 6.78 (s, 1H), 6.53 (s, 1H), 4.10-4.06 (m, 1H), 3.95 (ddd, J = 14.2, 6.9, 4.0 Hz, 1H), 3.64-3.53 (m, 1H), 3.38 (ddd, J = 14.2, 9.0, 3.5 Hz, 1H), 2.81 (d, J = 7.6 Hz, 2H), 2.36- 2.21 (m, 1H), 2.13-2.05 (m, 1H), 1.94-1.58 (m, 20H), 1.32-1.19 (m, 2H), 0.86 (td, J = 7.4, 2.8 Hz, 6H). |
| 107 | 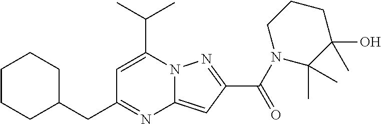 | 1 | NMR2; 6.81 (s, 1H), 6.56 (s, 1H), 3.91-3.76 (m, 2H), 3.57-3.45 (m, 1H), 2.68 (d, J = 7.2 Hz, 2H), 1.93-1.60 (m, 17H), 1.42 (dd, J = 6.9, 0.9 Hz, 6H), 1.30-1.13 (m, 6H), 1.11- 0.98 (m, 2H). |

TABLE 2-16

| EX | STR | Prop | Data |
|---|---|---|---|
| 108 | 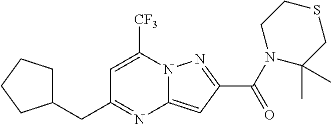 | 1 | NMR2: 7.07 (s, 1H), 7.05 (s, 1H), 3.88 (t, J = 5.9 Hz, 2H), 3.04 (t, J = 5.8 Hz, 2H), 2.91 (d, J = 7.5 Hz, 2H), 2.74 (s, 2H), 2.41-2.25 (m, 1H), 1.85-1.58 (m, 12H), 1.33-1.20 (m, 2H). |
| 109 | 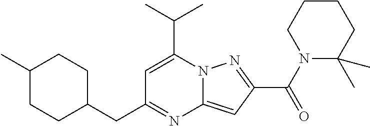 | 1 | NMR2: 6.74 (s, 1H), 6.56-6.50 (m, 1H), 3.89- 3.78 (m, 1H), 3.61-3.46 (m, 2H), 2.81- 2.57 (m, 2H), 2.06-2.02 (m, 1H), 1.77-1.65 (m, 8H), 1.61 (s, 6H), 1.55-1.24 (m, 11H), 1.14-1.01 (m, 1H), 0.98-0.83 (m, 4H). |
| 110 | 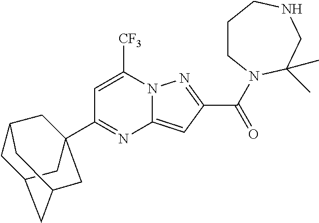 | 53 | NMR2: 7.25 (s, 1H), 7.04 (s, 1H), 3.67-3.58 (m, 2H), 3.01-2.96 (m, 2H), 2.94 (s, 2H), 2.21- 2.13 (m, 4H), 2.11-1.94 (m, 13H), 1.89- 1.74 (m, 7H). |
| 111 | 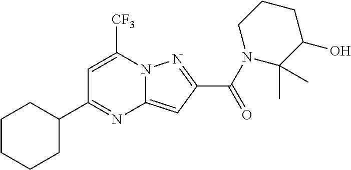 | 1 | NMR1: 7.66 (s, 1H), 6.94 (s, 1H), 5.02 (d, J = 5.0 Hz, 1H), 3.50-3.41 (m, 2H), 3.29-3.21 (m, 1H), 2.90 (tt, J = 11.7, 3.4 Hz, 1H), 1.97- 1.91 (m, 2H), 1.86-1.49 (m, 12H), 1.44-1.32 (m, 5H), 1.32-1.19 (m, 1H). |
| 112 | 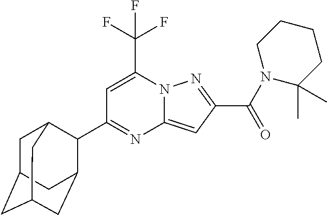 | 1 | NMR2: 7.17 (s, 1H), 6.95 (s, 1H), 3.56 (t, J = 5.7 Hz, 2H), 3.14-3.10 (m, 1H), 2.64-2.60 (m, 2H), 2.07-2.00 (m, 3H), 1.98-1.82 (m, 6H), 1.82-1.77 (m, 2H), 1.77-1.63 (m, 7H), 1.60 (s, 6H). |
| 113 | 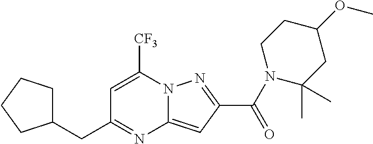 | 1 | NMR2: 7.06 (s, 1H), 6.97 (s, 1H), 3.81 (ddd, J = 14.0, 8.6, 3.9 Hz, 1H), 3.67-3.50 (m, 2H), 3.36 (s, 3H), 2.90 (d, J = 7.5 Hz, 2H), 2.39- 2.27 (m, 1H), 2.20-2.10 (m, 1H), 1.91 (ddd, J = 13.7, 4.8, 1.0 Hz, 1H), 1.85-1.61 (m, 8H), 1.61-1.54 (m, 6H), 1.33-1.20 (m, 2H). |

TABLE 2-17

| EX | STR | Prop | Daata |
|---|---|---|---|
| 114 | 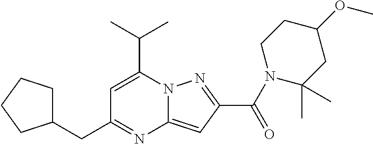 | 1 | NMR2: 6.76 (s, 1H), 6.57 (d, J = 0.7 Hz, 1H), 3.89-3.75 (m, 2H), 3.65-3.54 (m, 1H), 3.49 (ddd, J = 14.0, 7.8, 3.9 Hz, 1H), 3.36 (s, 3H), 2.81 (d, J = 7.5 Hz, 2H), 2.37-2.25 (m, 1H), 2.18-2.06 (m, 1H), 1.92 (ddd, J = 13.6, 4.8, 1.1 Hz, 1H), 1.83-1.72 (m, 2H), 1.71-1.61 (m, 6H), 1.60-1.51 (m, 6H), 1.41 (dd, J = 6.9, 0.9 Hz, 6H), 1.33-1.21 (m, 2H). |
| 115 | 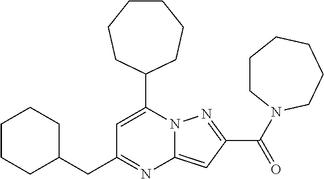 | 8 | NMR1: 6.90 (s, 1H), 6.76 (s, 1H), 3.69 (t, J = 6.0 Hz, 2H), 3.61 (t, J = 6.1 Hz, 2H), 3.57- 3.48 (m, 1H), 2.66 (d, J = 7.1 Hz, 2H), 2.06- 1.96 (m, 2H), 1.86-1.70 (m, 10H), 1.69-1.53 (m, 14H), 1.25-1.10 (m, 3H), 1.07-0.93 (m, 2H). |
| 116 | 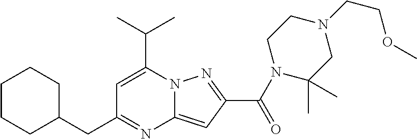 | 116 | NMR1: 6.90 (s, 1H), 6.68 (s, 1H), 3.72-3.61 (m, 1H), 3.56-3.39 (m, 4H), 3.24 (s, 3H), 2.67 (d, J = 7.1 Hz, 2H), 2.49-2.43 (m, 4H), 2.34 (s, 2H), 1.90-1.75 (m, 1H), 1.70-1.56 (m, 5H), 1.46 (s, 6H), 1.36 (d, J = 6.9 Hz, 6H), 1.25-0.94 (m, 5H). |
| 117 | 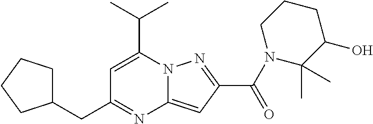 | 1 | NMR2: 6.77 (s, 1H), 6.58 (s, 1H), 3.89-3.76 (m, 1H), 3.69-3.49 (m, 3H), 2.81 (d, J = 7.5 Hz, 2H), 2.37-2.24 (m, 1H), 2.11-2.06 (m, 1H), 2.03-1.91 (m, 1H), 1.87-1.62 (m, 12H), 1.61-1.49 (m, 3H), 1.41 (d, J = 6.9 Hz, 6H), 1.33-1.19 (m, 2H). |
| 118 | 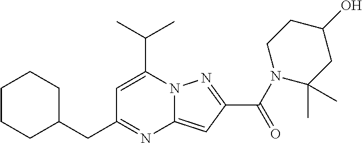 | 1 | NMR2: 6.77 (s, 1H), 6.55 (s, 1H), 4.12-4.03 (m, 1H), 4.00-3.90 (m, 1H), 3.90-3.75 (m, 1H), 3.38 (ddd, J = 14.1, 9.0, 3.5 Hz, 1H), 2.68 (d, J = 7.2 Hz, 2H), 2.17-2.02 (m, 1H), 1.90 (ddd, J = 13.3, 4.8, 1.4 Hz, 1H), 1.85-1.75 (m, 1H), 1.74-1.58 (m, 14H), 1.42 (dd, J = 6.9, 1.4 Hz, 6H), 1.31-1.13 (m, 3H), 1.11- 0.98 (m, 2H). |
| 119 | 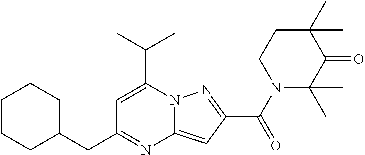 | 1 | NMR2: 6.86 (s, 1H), 6.57 (d, J = 0.7 Hz, 1H), 3.86-3.73 (m, 1H), 3.76-3.68 (m, 2H), 2.69 (d, J = 7.2 Hz, 2H), 2.06-1.98 (m, 2H), 1.89- 1.75 (m, 7H), 1.75-1.63 (m, 6H), 1.44 (d, J = 6.9 Hz, 6H), 1.28 (s, 6H), 1.26-1.13 (m, 2H), 1.12-0.98 (m, 2H). |

TABLE 2-18

| EX | STR | Prop | Data |
|---|---|---|---|
| 120 | 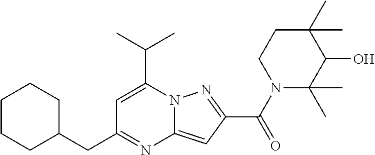 | 1 | NMR2: 6.77 (s, 1H), 6.54 (s, 1H), 3.91-3.73 (m, 2H), 3.45-3.32 (m, 2H), 2.68 (d, J = 7.2 Hz, 2H), 1.89-1.61 (m, 10H), 1.57-1.52 (m, 5H), 1.42 (dd, J = 6.9, 1.6 Hz, 6H), 1.31-1.13 (m, 3H), 1.13-0.98 (m, 8H). |
| 121 | 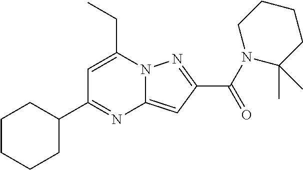 | 1 | NMR2: 6.72 (s, 1H), 6.63-6.58 (m, 1H), 3.55- 3.48 (m, 2H), 3.23-3.12 (m, 2H), 2.78- 2.66 (m, 1H), 2.02-1.94 (m, 2H), 1.94-1.84 (m, 2H), 1.82-1.73 (m, 1H), 1.70-1.51 (m, 14H), 1.49-1.22 (m, 6H). |
| 122 | 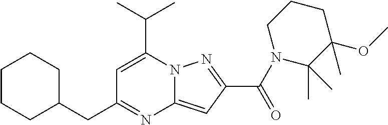 | 1 | NMR2: 6.74 (s, 1H), 6.52 (s, 1H), 3.90-3.76 (m, 1H), 3.69 (dt, J = 13.9, 4.8 Hz, 1H), 3.56- 3.44 (m, 1H), 3.24 (s, 3H), 2.67 (d, J = 7.2 Hz, 2H), 1.88-1.75 (m, 3H), 1.74-1.59 (m, 10H), 1.55 (s, 3H), 1.41 (d, J = 6.9 Hz, 6H), 1.32- 1.13 (m, 6H), 1.11-0.97 (m, 2H). |
| 123 | 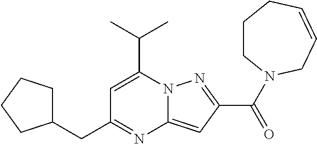 | 1 | NMR2: 6.89 (d, J = 3.8 Hz, 1H), 6.61-6.56 (m, 1H), 5.95-5.66 (m, 2H), 4.36-4.30 (m, 1H), 4.30-4.23 (m, 1H), 3.93 (dt, J = 13.7, 6.1 Hz, 2H), 3.87-3.75 (m, 1H), 2.82 (d, J = 7.5 Hz, 2H), 2.39-2.23 (m, 3H), 2.07-1.95 (m, 1H), 1.95-1.85 (m, 1H), 1.83-1.63 (m, 4H), 1.61-1.49 (m, 2H), 1.42 (dd, J = 6.9, 3.0 Hz, 6H), 1.33-1.20 (m, 2H). |
| 124 | 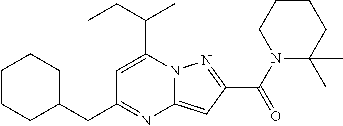 | 1 | NMR2: 6.75 (s, 1H), 6.50 (s, 1H), 3.76-3.63 (m, 1H), 3.63-3.46 (m, 2H), 2.67 (d, J = 7.2 Hz, 2H), 2.02-1.58 (m, 19H), 1.38 (d, J = 7.0 Hz, 3H), 1.33-1.12 (m, 4H), 1.10-0.98 (m, 2H), 0.94 (t, J = 7.4 Hz, 3H). |
| 125 | 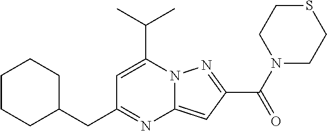 | 1 | NMR2: 6.87 (s, 1H), 6.57 (s, 1H), 4.16-4.05 (m, 4H), 3.84-3.71 (m, 1H), 2.76 (dt, J = 21.3, 5.1 Hz, 4H), 2.69 (d, J = 7.2 Hz, 2H), 1.88-1.76 (m, 1H), 1.74-1.61 (m, 5H), 1.43 (d, J = 6.9 Hz, 6H), 1.31-1.13 (m, 3H), 1.11- 0.98 (m, 2H). |

TABLE 2-19

| EX | STR | Prop | Data |
|---|---|---|---|
| 126 | 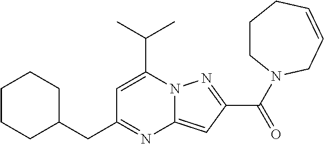 | 1 | NMR2: 6.89 (d, J = 3.4 Hz, 1H), 6.58-6.53 (m, 1H), 5.94-5.68 (m, 2H), 4.36-4.30 (m, 1H), 4.30-4.24 (m, 1H), 3.93 (dt, J = 13.5, 6.1 Hz, 2H), 3.87-3.75 (m, 1H), 2.68 (d, J = 7.2 Hz, 2H), 2.37-2.26 (m, 2H), 2.07-1.95 (m, 1H), 1.95-1.77 (m, 2H), 1.74-1.67 (m, 5H), 1.43 (dd, J = 6.9, 2.9 Hz, 6H), 1.31-1.12 (m, 3H), 1.11-0.98 (m, 2H). |
| 127 | 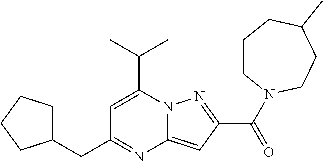 | 1 | NMR2: 6.87 (d, J = 4.0 Hz, 1H), 6.60-6.55 (m, 1H), 4.05-3.46 (m, 5H), 2.85-2.78 (m, 2H), 2.37-2.25 (m, 1H), 2.07-1.48 (m, 12H), 1.42 (dd, J = 6.9, 2.0 Hz, 6H), 1.36-1.20 (m, 3H), 0.98 (dd, J = 13.2, 6.7 Hz, 3H). |
| 128 | 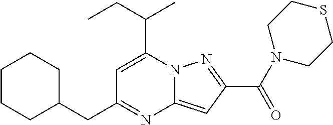 | 1 | NMR2: 6.87 (s, 1H), 6.55 (s, 1H), 4.14-4.08 (m, 4H), 3.66-3.56 (m, 1H), 2.82-2.71 (m, 4H), 2.69 (d, J = 7.2 Hz, 2H), 2.00-1.62 (m, 8H), 1.39 (d, J = 7.0 Hz, 3H), 1.28-1.13 (m, 3H), 1.11-0.98 (m, 2H), 0.95 (t, J = 7.4 Hz, 3H). |
| 129 | 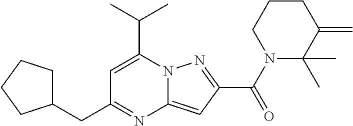 | 1 | NMR2: 6.82 (s, 1H), 6.57 (s, 1H), 4.97-4.92 (m, 1H), 4.86-4.61 (m, 1H), 3.89-3.75 (m, 1H), 3.44 (t, J = 6.4 Hz, 2H), 2.81 (d, J = 7.5 Hz, 2H), 2.53-2.45 (m, 2H), 2.37-2.22 (m, 1H), 2.02-1.90 (m, 2H), 1.82-1.49 (m, 12H), 1.42 (d, J = 6.9 Hz, 6H), 1.33-1.20 (m, 2H). |
| 130 | 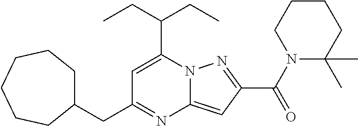 | 1 | NMR2: 6.75 (s, 1H), 6.49 (s, 1H), 3.65-3.46 (m, 3H), 2.71 (d, J = 7.4 Hz, 2H), 2.12-1.98 (m, 1H), 1.92-1.76 (m, 4H), 1.76-1.54 (m, 13H), 1.54-1.35 (m, 5H), 1.34-1.21 (m, 4H), 0.96-0.81 (m, 8H). |
| 131 | 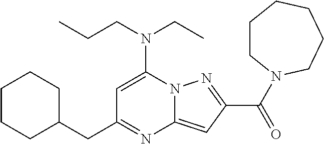 | 1 | NMR2: 6.66 (s, 1H), 5.81 (s, 1H), 3.82 (q, J = 7.0 Hz, 2H), 3.78-3.56 (m, 6H), 2.55 (d, J = 7.1 Hz, 2H), 1.91-1.50 (m, 11H), 1.33-1.10 (m, 10H), 1.08-0.78 (m, 6H). |
| 132 | 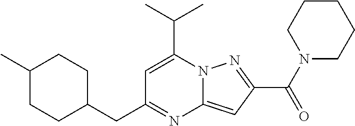 | 1 | NMR2: 6.81 (s, 1H), 6.58-6.52 (m, 1H), 3.88- 3.68 (m, 5H), 2.79-2.67 (m, 2H), 2.13-1.95 (m, 1H), 1.74-1.57 (m, 7H), 1.56-1.24 (m, 12H), 1.15-1.00 (m, 1H), 0.98-0.84 (m, 4H). |

TABLE 2-20

| EX | STR | Prop | Data |
|---|---|---|---|
| 133 | 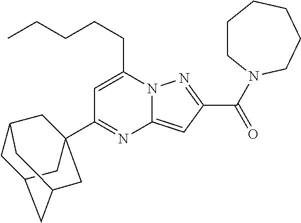 | 1 | NMR1; 7.15 (s, 1H), 6.76 (s, 1H), 3.62 (dt, J = 13.9, 6.0 Hz, 4H), 3.12-3.04 (m, 2H), 2.11- 2.06 (m, 3H), 2.00-1.95 (m, 6H), 1.85-1.65 (m, 12H), 1.62-1.49 (m, 4H), 1.40-1.28 (m, 4H), 0.91-0.83 (m, 3H). |
| 134 | 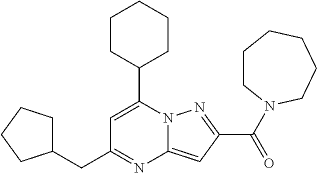 | 1 | NMR1: 6.90 (s, 1H), 6.75 (s, 1H), 3.67 (t, J = 6.0 Hz, 2H), 3.61 (t, J = 6.1 Hz, 2H), 2.78 (d, J = 7.5 Hz, 2H), 2.37-2.25 (m, 1H), 2.11-2.03 (m, 2H), 1.89-1.81 (m, 2H), 1.79-1.14 (m, 23H). |
| 135 | 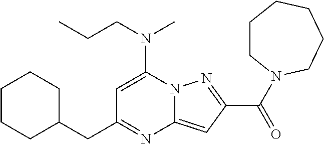 | 89 | NMR1: 6.50 (s, 1H), 6.12 (s, 1H), 3.93-3.85 (m, 2H), 3.69 (t, J = 6.0 Hz, 2H), 3.63-3.56 (m, 2H), 3.17 (s, 3H), 2.53 (d, J = 7.2 Hz, 2H), 1.85-1.48 (m, 16H), 1.26-1.09 (m, 3H), 1.05- 0.92 (m, 2H), 0.81 (t, J = 7.4 Hz, 3H). |
| 136 | 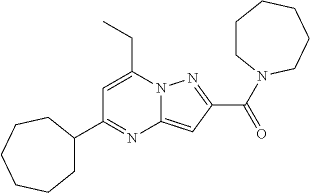 | 1 | NMR1; 6.96 (s, 1H), 6.75 (s, 1H), 3.62 (dt, J = 14.9, 6.0 Hz, 4H), 3.08 (q, J = 7.4 Hz, 2H), 2.92 (tt, J = 9.8, 7.5, 3.2 Hz, 1H), 1.97-1.86 (m, 2H), 1.84-1.46 (m, 18H), 1.35 (t, J = 7.4 Hz, 3H). |
| 137 | 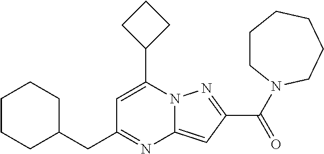 | 1 | NMR2: 6.87 (s, 1H), 6.57 (s, 1H), 4.23-4.10 (m, 1H), 3.74 (dt, J = 10.4, 6.0 Hz, 4H), 2.70 (d, J = 7.2 Hz, 2H), 2.63-2.51 (m, 2H), 2.34- 2.12 (m, 3H), 2.02-1.91 (m, 1H), 1.91-1.74 (m, 4H), 1.74-1.56 (m, 10H), 1.29-1.14 (m, 3H), 1.12-0.99 (m, 2H). |
| 138 | 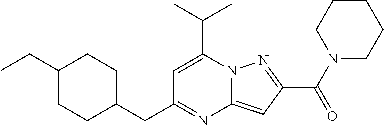 | 1 | NMR2: 6.81 (s, 1H), 6.58-6.52 (m, 1H), 3.88- 3.68 (m, 5H), 2.81-2.65 (m, 2H), 1.83- 1.00 (m, 22H), 0.97-0.82 (m, 5H). |
| 139 | 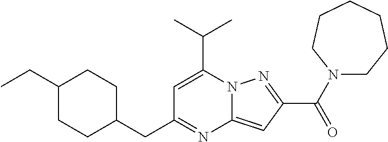 | 1 | NMR2: 6.87 (s, 1H), 6.58-6.52 (m, 1H), 3.86- 3.67 (m, 5H), 2.81-2.65 (m, 2H), 1.92- 1.40 (m, 18H), 1.39-0.76 (m, 11H). |

TABLE 2-21

| EX | STR | Prop | Data |
|---|---|---|---|
| 140 | 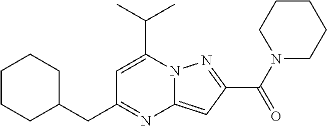 | 1 | NMR2: 6.81 (s, 1H), 6.54 (s, 1H), 3.88-3.64 (m, 5H), 2.68 (d, J = 7.2 Hz, 2H), 1.87-1.77 (m, 1H), 1.74-1.67 (m, 11H), 1.42 (d, J = 6.9 Hz, 6H), 1.32-1.11 (m, 3H), 1.11-0.98 (m, 2H). |
| 141 | 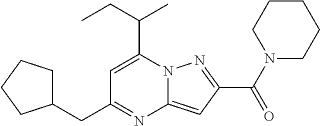 | 1 | NMR2: 6.82 (s, 1H), 6.55 (s, 1H), 3.80-3.60 (m, 5H), 2.81 (d, J = 7.5 Hz, 2H), 2.36-2.24 (m, 1H), 2.00-1.85 (m, 1H), 1.80-1.48 (m, 13H), 1.39 (d, J = 7.0 Hz, 3H), 1.33-1.19 (m, 2H), 0.95 (t, J = 7.4 Hz, 3H). |
| 142 | 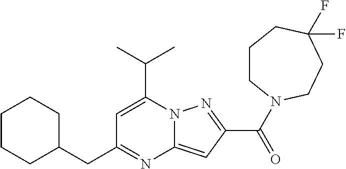 | 1 | NMR2: 6.98 (s, 1H), 6.60-6.55 (m, 1H), 3.97- 3.67 (m, 5H), 2.69 (dd, J = 7.3, 1.2 Hz, 2H), 2.48-2.29 (m, 1H), 2.22-1.61 (m, 10H), 1.43 (dd, J = 6.9, 1.8 Hz, 6H), 1.32-1.14 (m, 3H), 1.12-0.99 (m, 2H). |
| 143 | 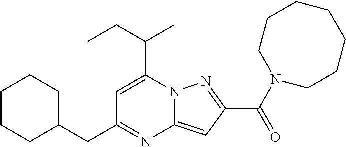 | 1 | NMR2: 6.88 (s, 1H), 6.51 (s, 1H), 3.79-3.57 (m, 5H), 2.68 (d, J = 7.2 Hz, 2H), 2.01-1.86 (m, 3H), 1.81-1.61 (m, 15H), 1.39 (d, J = 6.9 Hz, 3H), 1.31-1.13 (m, 3H), 1.11-0.99 (m, 2H), 0.95 (t, J = 7.4 Hz, 3H). |
| 144 | 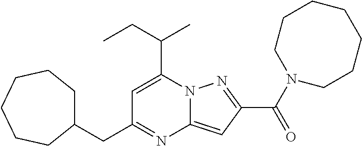 | 1 | NMR2: 6.88 (s, 1H), 6.53 (s, 1H), 3.79-3.57 (m, 5H), 2.72 (d, J = 7.5 Hz, 2H), 2.13-2.00 (m, 1H), 2.01-1.86 (m, 3H), 1.81-1.61 (m, 14H), 1.54-1.36 (m, 7H), 1.34-1.22 (m, 3H), 0.94 (t, J = 7.4 Hz, 3H). |
| 145 | 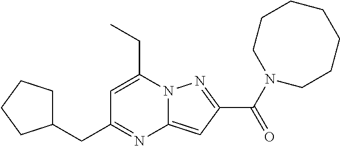 | 1 | NMR2: 6.85 (s, 1H), 6.60 (s, 1H), 3.76-3.65 (m, 4H), 3.15 (q, J = 7.4 Hz, 2H), 2.81 (d, J = 7.5 Hz, 2H), 2.38-2.26 (m, 1H), 1.92-1.86 (m, 2H), 1.83-1.49 (m, 14H), 1.43 (t, J = 7.5 Hz, 3H), 1.33-1.20 (m, 2H). |
| 146 | 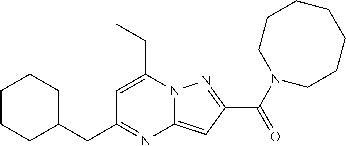 | 1 | NMR2: 6.86 (s, 1H), 6.57 (s, 1H), 3.76-3.65 (m, 4H), 3.15 (q, J = 7.5 Hz, 2H), 2.68 (d, J = 7.2 Hz, 2H), 1.95-1.60 (m, 16H), 1.43 (t, J = 7.5 Hz, 3H), 1.30-1.13 (m, 3H), 1.11-0.98 (m, 2H). |

TABLE 2-22

| EX | STR | Prop | Data |
|---|---|---|---|
| 147 | 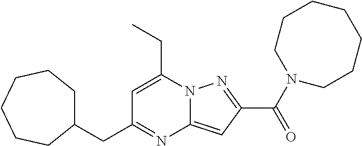 | 1 | NMR2: 6.86 (s, 1H), 6.58 (3, 1H). 3.76-3.65 (m, 4H). 3.15 (q. J = 7.5 Hz, 2H), 2.72 (d, J = 7.4 Hz, 2H). 2.1 -2.00 (m, 1H), 1.94-1.84 (m, 2H), 1.78-1.37 (m, 21H), 1.35-1.22 (m, 2H). |
| 148 | 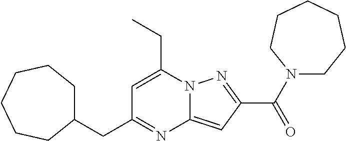 | 1 | NMR2: 6.86 (s, 1H), 6.58 (s. 1H), 3.74 (td, J = 6.0, 1.2 Hz, 4H), 3.16 (qd, J = 7.4, 0,9 Hz, 2H). 2.72 (d. J = 7.4 Hz, 2H), 2.13-1.99 (m, 1H), 1.92 - 1.83 (m, 2H), 1.83 - 1.36 (m, 18H), 1.35-1.22 (m. 3H). |
| 149 | 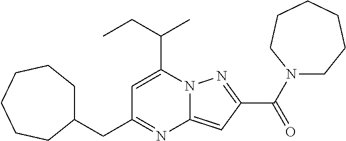 | 1 | NMR2: 6.88 (s. 1H), 6.53 (s, 1H), 3.80-3.71 (m, 4H). 3.71-3.58 (m, 1H). 2.72 (d. J = 7.4 Hz, 2H), 2.12-2.01 (m, 1H), 2.00-1.36 (m, 23H), 1.35-1.22 (m. 2H), 0.94 (t. J = 7.4 Hz, 3H) |
| 150 | 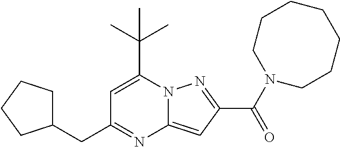 | 1 | NMR2: 6.93 (s, 1H), 6.59 (s, 1H). 3.87-3.80 (m. 2H), 3.74-3.67 (m, 2H). 2.81 (d, J = 7.5 Hz. 2H), 2.37-2.25 (m. 1H), 1.92-1.87 (m, 2H), 1.82-1.74 (m, 4H), 1.72-1.50 (m, 18H), 1.31-1.20 (m, 3H). |
| 151 | 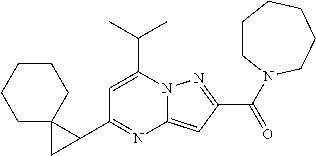 | 1 | NMR2: 6.80 (s, 1H), 6.66 (s, 1H), 3,83-3.70 (m. 5H), 2.00 - 1.92 (m, 1H), 1.92-1.74 (m. 4H), 1.69 - 1.59 (m. 5H), 1.56-1.21 (m, 14H). 1.16 - 1.09 (m, 1H), 0.95-0.84 (m, 2H) |
| 152 | 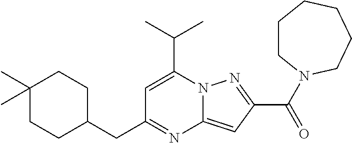 | 1 | NMR2: 6.87 (s. 1H), 6.55 (s, 1H). 3.85-3.70 (m, 5H), 2.71 (d. J = 7.3 Hz, 2H), 1.92-1.60 (m, 9H), 1.58-1.48 (m, 2H), 1.42 (d, J = 6.9 Hz, 6H), 1.40-1.11 (m, 6H), 0.89 (s, 6H) |
| 153 | 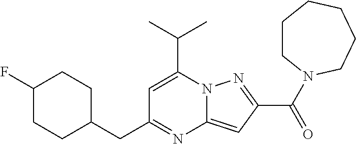 | 1 | NMR2: 6.88 (s, 1H), 6.57-6.51 (m, 1H), 3.84- 3.71 (m, 5H), 2.76-2.67 (m, 2H). 2.14- 1 75 (m, 8H), 1.71-1.40 (m, 15H), 1.21-1.07 (m, 1H). |
| 154 | 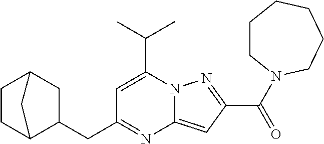 | 1 | NMR2: 6.87 (s, 1H), 6.57 (s, 1H), 3.85-3.70 (m, 6H), 2.83-2.73 (m, 1H), 2.66-2.56 (m, 1H), 2.27-2.23 (m. 1H), 2.02-1.88 (m. 1H), 1.91-1.82 (m, 2H), 1.85 - 1.75 (m, 2H), 1.72- 1.56 (m, 5H), 1.55-1.40 (m, 10H), 1.23- 1.09 (m, 4H). |

TABLE 2-23

| EX | STR | Prop | Data |
|---|---|---|---|
| 155 | 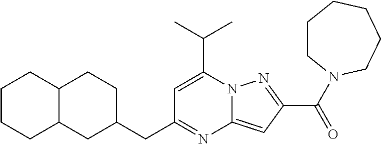 | 1 | NMR2: 6.90-6.85 (m, 1H), 6.58-6.51 (m, 1H), 3.85-3.70 (m. 5H). 2.74-2.62 (m, 2H). 1.92-1.59 (m, 13H), 1.55-1.11 (m, 18H). |
| 156 | 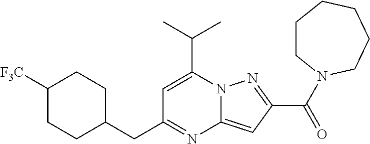 | 1 | NMR2: 6.91-6.86 (m. 1H), 6.57-6.51 (m, 1H), 3.87-3.71 (m, 5H), 2.87 - 2.65 (m, 2H), 2.33-1.55 (m, 13H), 1.46-1.24 (m, 10H). 1.17-1.02 (m, 1H). |
| 157 | 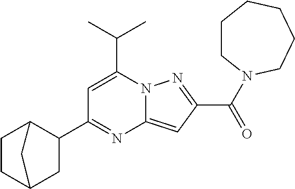 | 1 | NMR2: 6.90-6.84 (m. 1H), 6.61-6.56 (m. 1H), 3.83-3.70 (m, 5H), 2.91-2.82 (m, 1H), 2.66-2.33 (m, 2H), 2.23-2.13 (m, 1H), 1.91- 1.74 (m, 5H), 1.71-1.56 (m, 7H), 1.47- 1.38 (m, 7H), 1.36 - 1.12 (m, 2H). |
| 158 | 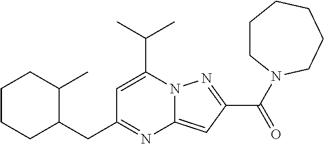 | 1 | NMR2: 6.89-6.85 (m, 1H), 6.58-6.51 (m, 1H), 3.86-3.66 (m, 5H), 3.23 - 2.33 (m, 2H), 2.16-2.06 (m, 1H), 1.97-1.75 (m, 5H), 1.75- 1.59 (m, 6H), 1.59-1.45 (m, 2H), 1.43- 1.07 (m, 9H), 1.04-0.93 (m, 4H). |
| 159 | 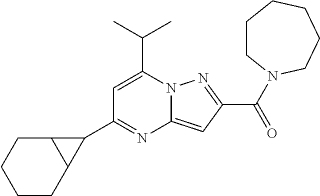 | 1 | NMR2: 5.73 (s, 1H). 6.46 (s, 1H), 3.82-3.69 (m, 5H), 2.07-1.96 (m, 2H), 1.91-1.71 (m, 9H), 1.71-1.58 (m, 4H), 1.44-1.26 (m, 10H). |
| 160 | 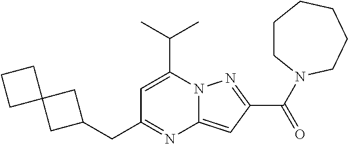 | 1 | NMR2: 6.86 (s, 1H), 6.51 (s, 1H), 3 84-3.70 (m, 5H), 2.86 (d, J = 7.7 Hz, 2H), 2.65-2.49 (m, 1H), 2.18-2.09 (m, 2H), 2.03-1.95 (m. 2H), 1.95-1.71 (m, 10H), 1.70-1.60 (m, 4H), 1.41 (d, J = 6.9 Hz, 6H). |
| 161 | 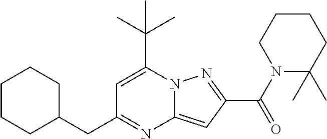 | 1 | NMR2: 6.82 (s, 1H), 6.54 (s, 1H), 3.63 (t, J = 5.6 Hz, 2H), 2.66 (d, J = 7.2 Hz, 2H), 1.88 - 1.64 (m, 12H), 1.63-1.58 (m, 15H), 1.29 - 1.18 (m, 3H), 1.11-0.97 (m, 2H). |
| 162 | 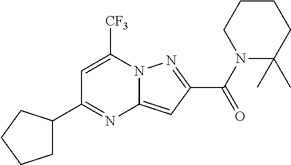 | 1 | NMR2: 7.07 (s, 1H). 6.93 (s, 1H), 3.58-3.50 (m, 2H), 3.35-3.22 (m, 1H), 2.17-2.08 (m, 2H), 1.94-1.80 (m, 4H), 1.79-1.64 (m, 8H), 1.60 (s, 6H). |

### TEST EXAMPLES

### Test Example 1: Intracellular Calcium Concentration Measurement

HEK293 cell lines derived from human embryonic kidney cultured in a MEM culture medium supplemented with 10% fetal bovine serum (10% FBS) (Invitrogen) were adjusted to 4×10⁵ cells/mL by using a MEM culture medium supplemented with 1% FBS, and then seeded at 25 μL/well onto Poly-D-Lysine-coated 384-well black plates (transparent bottom) (Greiner). The seeded cells were cultured in a carbon dioxide incubator for 2 days. Cells were treated with 20 μL of Fluo-8 No Wash Calcium Assay Kit (AAT Bioquest) adjusted with a Hanks-10 mM Hepes buffer containing 0.1% bovine serum albumin (0.1% BSA-HHBS) and 5 μL of a test compound solution adjusted with a 0.1% BSA-HHBS. Then, the cells were cultured in a carbon dioxide incubator for 30 minutes. SLIGKV-NH2 (Sigma-Aldrich) diluted with 0.1% BSA-HHBS buffer solution was added to 384-well polypropylene plates (Greiner) to obtain agonist plates. The cell plates treated with the test compounds and the agonist plates were set in FDSS/μCELL (Hamamatsu Photonics K.K.). Ten (10) microlitter of the SLIGKV-NH2 solution from the agonist plates was added to the cell plate (final concentration: 10 μM) using the built-in automated pipetting system. Fluorescence changes were detected by a CCD camera in FDSS/μCELL at 37° C. for 180 seconds immediately after the addition of the SLIGKV-NH2 solution to determine changes in intracellular calcium.

IC₅₀ values (nM) are shown in Tables 3-1 and 3-2.

TABLE 3-1

| Example | IC₅₀ (nM) |
|---|---|
| 1 | 173 |
| 2 | 31 |
| 3 | 23 |
| 4 | 109 |
| 5 | 130 |
| 6 | 149 |
| 7 | 113 |
| 8 | 95 |
| 9 | 74 |
| 10 | 178 |
| 11 | 252 |
| 12 | 76 |
| 13 | 69 |
| 14 | 186 |
| 15 | 150 |
| 16 | 259 |
| 17 | 232 |
| 18 | 36 |
| 19 | 131 |
| 20 | 24 |
| 21 | 298 |
| 22 | 137 |
| 23 | 30 |
| 24 | 9 |
| 25 | 49 |
| 26 | 80 |
| 27 | 138 |
| 28 | 60 |
| 29 | 467 |
| 30 | 102 |
| 31 | 81 |
| 32 | 118 |
| 33 | 51 |
| 34 | 265 |
| 35 | 130 |
| 36 | 369 |
| 37 | 88 |
| 38 | 102 |
| 39 | 64 |
| 40 | 184 |
| 41 | 246 |
| 42 | 40 |
| 43 | 185 |
| 44 | 208 |
| 45 | 632 |
| 46 | 99 |
| 47 | 146 |
| 48 | 105 |
| 49 | 119 |
| 50 | 43 |
| 51 | 209 |
| 52 | 107 |
| 53 | 404 |
| 54 | 355 |
| 55 | 569 |
| 56 | 283 |
| 57 | 308 |
| 58 | 464 |
| 59 | 357 |
| 60 | 234 |
| 61 | 96 |
| 62 | 52 |
| 63 | 12 |
| 64 | 17 |
| 65 | 107 |
| 66 | 241 |
| 67 | 174 |
| 68 | 15 |
| 69 | 286 |
| 70 | 51 |
| 71 | 115 |
| 72 | 41 |
| 73 | 33 |
| 74 | 386 |
| 75 | 89 |
| 76 | 121 |
| 77 | 273 |
| 78 | 247 |
| 79 | 145 |
| 80 | 250 |
| 81 | 246 |
| 82 | 76 |
| 84 | 274 |
| 85 | 310 |
| 86 | 338 |
| 89 | 243 |
| 91 | 522 |
| 93 | 236 |

TABLE 3-2

| Example | IC₅₀ (nM) |
|---|---|
| 94 | 141 |
| 95 | 742 |
| 96 | 10 |
| 97 | 20 |
| 98 | 270 |
| 99 | 336 |
| 100 | 23 |
| 101 | 70 |
| 102 | 37 |
| 103 | 63 |
| 104 | 46 |
| 105 | 83 |
| 106 | 96 |
| 107 | 67 |
| 108 | 78 |
| 109 | 85 |
| 110 | 228 |
| 111 | 250 |
| 112 | 113 |
| 113 | 263 |
| 114 | 234 |
| 115 | 236 |
| 116 | 233 |
| 117 | 169 |
| 118 | 253 |
| 119 | 185 |
| 120 | 298 |
| 121 | 245 |
| 122 | 178 |
| 123 | 135 |
| 124 | 144 |
| 125 | 256 |
| 126 | 207 |
| 127 | 229 |
| 128 | 151 |
| 129 | 208 |
| 130 | 297 |
| 131 | 225 |
| 132 | 197 |
| 133 | 360 |
| 134 | 382 |
| 135 | 386 |
| 136 | 452 |
| 137 | 482 |
| 138 | 72 |
| 139 | 95 |
| 140 | 266 |
| 141 | 304 |
| 142 | 171 |
| 143 | 91 |
| 144 | 257 |
| 145 | 252 |
| 146 | 136 |
| 147 | 163 |
| 148 | 144 |
| 149 | 208 |
| 150 | 180 |
| 151 | 149 |
| 152 | 132 |
| 153 | 282 |
| 154 | 47 |
| 155 | 218 |
| 156 | 103 |
| 157 | 319 |
| 158 | 117 |
| 159 | 394 |
| 160 | 147 |
| 161 | 296 |
| 162 | 304 |

### Test Example 2: Scratching Behavior Test with PAR2 Agonist Peptide Administration

Magnets for measuring scratching behavior(Neuroscience, Inc.) were implanted in both legs of 6- to 7-week-old female ICR mice under 3.5% isoflurane inhalation anesthesia. After about one week, the mice were acclimatized overnight in a cylindrical cage for a scratching behavior measuring device (Microact: Neuroscience Co., Ltd.).

Under isoflurane inhalation anesthesia, the upper back was shaved about 2×3 cm with a shaver and 40 μL of a 6% of test compound solution was applied with a micropipette. The animals were then kept in a dedicated cage for 1 hour. Solvents used were a 1:1 mixture of acetone and methanol (acetone/methanol in Table 4 below), 100% ethanol or 70% ethanol. Then, under isoflurane inhalation anesthesia, 10 μL of a 25 mg/mL solution of PAR2 agonist peptide (SLIGRL-NH2) in distilled water was administered intradermally by using a needle attached to a Hamilton syringe. The animals were returned to the cage and the scratching frequency was measured by the device for 30 minutes from 10 to 40 minutes after the administration.

The inhibitory effect of the test compounds on scratching behavior was calculated as a percentage of the number of scratches in the solvent-applied and PAR2 agonist peptide-treated group, and is shown in Table 4 as a percentage.

TABLE 4

| | | scratching-behavior |
|---|---|---|
| Example | solvent | inhibition rate |
| 1 | 100% ethanol | 35% |
| 2 | 100% ethanol | 46% |
| 3 | 70% ethanol | 33% |
| 5 | 100% ethanol | 36% |
| 7 | 100% ethanol | 32% |
| 8 | 100% ethanol | 30% |
| 10 | 100% ethanol | 41% |
| 12 | 100% ethanol | 51% |
| 13 | 100% ethanol | 37% |
| 15 | 100% ethanol | 44% |
| 16 | 100% ethanol | 47% |
| 18 | 100% ethanol | 39% |
| 19 | 100% ethanol | 42% |
| 32 | 100% ethanol | 41% |
| 37 | 100% ethanol | 30% |
| 53 | Acetone/methanol | 44% |
| 54 | Acetone/methanol | 25% |
| 55 | Acetone/methanol | 47% |
| 59 | Acetone/methanol | 28% |
| 65 | 100% ethanol | 27% |
| 70 | 100% ethanol | 33% |
| 71 | 100% ethanol | 27% |
| 94 | Acetone/methanol | 44% |
| 105 | 100% ethanol | 27% |
| 110 | Acetone/methanol | 25% |
| 117 | 100% ethanol | 34% |

### Test Example 3: Scratching Behavior Test Using Atopic Dermatitis Model

Magnets for measuring scratching behavior were implanted in both legs of 7-week-old female NC/Nga mice under anesthesia. After about one week, an area of 2×3 cm on the upper back was shaved with a shaver and dehaired with a depilatory cream under isoflurane anesthesia. Then, 100 μL of 4% SDS was applied on the hair removal site under anesthesia. Two hours later, a suitable amount (about 100 μg) of mite antigen ointment (Biota AD: Biota Co., Ltd.) was applied. This sensitization by SDS and mite antigen ointment application was performed a total of 6 times over 14 days.

Before the last sensitization, the skin symptoms of the animals were scored in terms of redness (7-point scale) and edema (7-point scale) using the score criteria shown below. Animals with a total score (dermatitis score) of 2 or higher were selected as test candidates. Transepidermal water loss (TEWL) values of these animals were measured using Tewameter TM300 (Courage+Khazaka). The animals were then divided into groups by using the dermatitis score and TEWL value as indices, and a final sensitization was performed. After grouping, animals were acclimatized overnight in a cylindrical cage for a scratching behavior measuring device (Microact: Neuroscience Inc.). The next morning, 60 μL of each solution of the test compound (1%, 3%, or 6%) in a solvent was applied under anesthesia, and the number of scraches was measured 7 hours after the application by using the device. The solvents used were a 1:1 mixture of acetone and methanol (acetone/methanol in Table 5 below), 100% ethanol, and 70% ethanol.

The number of scratching in non-sensitized animals to which the solvent had been applied was converted to a 100% inhibition rate, and the number of scratching in sensitized animals to which the solvent had been applied was converted to a 0% inhibition rate, and then the inhibition effects of the example compounds on scratching are shown in Table 5 as percentages.

| | Redness score | Edema score |
|---|---|---|
| 0 | No redness | No edema |
| 0.5 | Redness milder | Edema area is less |
| | than score 1 | than 15% |
| 1 | Slight redness | Edema area is not |
| | | less than 15% but |
| | | less than 30% |
| 1.5 | Redness between | Edema area is not |
| | score 1 and | less than 30% but |
| | score 2 | less than 45% |
| 2 | Obvious redness | Edema area is not |
| | | less than 45% but |
| | | less than 60% |
| 2.5 | Redness between | Edema area is not |
| | score 2 and | less than 60% but |
| | score 3 | less than 75% |
| 3 | Remarkable redness | Edema area is not |
| | | less than 75% |

TABLE 5

| | | | Scratching |
|---|---|---|---|
| | Assessed | | behavior |
| Example | concentration | Solvent | inhibition rate |
| 1 | 6% | 100% ethanol | 76% |
| 3 | 6% | 70% ethanol | 71% |
| 4 | 6% | 70% ethanol | 56% |
| 6 | 6% | 100% ethanol | 60% |
| 7 | 6% | 100% ethanol | 70% |
| 8 | 6% | 100% ethanol | 50% |
| 10 | 1% | 100% ethanol | 48% |
| 11 | 6% | 100% ethanol | 80% |
| 12 | 6% | 100% ethanol | 102% |
| 14 | 6% | 100% ethanol | 95% |
| 15 | 3% | 100% ethanol | 52% |
| 17 | 6% | 100% ethanol | 60% |
| 19 | 1% | 100% ethanol | 70% |
| 55 | 6% | Acetone/methanol | 54% |

### Test Example 4: Rabbit Skin Cumulative Irritation Test

18-20 week-old female NZW rabbits shaved on the back with a shaver were fitted with a collar (Natsume Seisakusho Co., Ltd.). A 2.5 cm×2.5 cm frame was placed on the back of the rabbits, and 50 μL of a 3% solution of each compound in 70% ethanol was applied to the place in 2 or 3 rabbits. The next day (approximately 24 hours later), the compound applied on the previous day was wiped off with a cotton pad soaked in lukewarm water, and approximately 30 minutes later, erythema (5-point scale) and edema (5-point scale) were evaluated according to the score criteria shown below.

After the score evaluation, the compound was applied again. This process was conducted for 7 days, and irritancy was evaluated by the average of the total score of erythema and edema on the last day of evaluation (Day 7) and the maximum (total) scores during the test period.

The score evaluation criteria were as follows.

| | Erythema score | Edema score |
|---|---|---|
| 0 | No erythema | No edema |
| 1 | Very mild erythema | Very mild edema |
| | (barely discernible) | (barely discernible) |
| 2 | Clear erythema | Mild edema (defined |
| | | by obvious elevation |
| | | of the edges of the area) |
| 3 | Moderate to severe | Moderate edema (about |
| | erythema | 1 mm elevation) |
| 4 | Severe erythema (meat's | Severe edema (elevation |
| | red color) to crust | greater than 1 mm and |
| | formation that prevents | beyond the exposure area) |
| | erythema classification | |

TABLE 6

Irritant score

| Example | Day 7 | Maximum |
|---|---|---|
| 3 | 1.5 | 1.6 |
| 7 | 0.3 | 1.3 |
| 12 | 0.0 | 0.0 |
| 14 | 2.0 | 2.0 |
| 15 | 0.0 | 0.7 |
| 17 | 0.7 | 1.3 |

The invention claimed is:

1. A compound represented by general formula [I]: 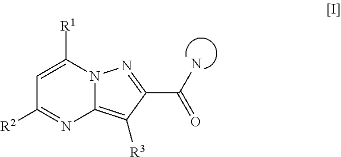
wherein
R¹ is C₁₋₆ alkyl, C₃₋₈ cycloalkyl, C₁₋₆ haloalkyl, C₁₋₆ alkoxy, C₃₋₈ cycloalkoxy, C₁₋₆ alkylthio, or mono or di C₁₋₆ alkylamino;
R² is C₃₋₈ cycloalkyl optionally substituted by halogen or C₁₋₆ alkyl, C₄₋₁₀ bicycloalkyl optionally substituted by halogen or C₁₋₆ alkyl, C₅₋₁₃ spiroalkyl, C₆₋₁₂ tricycloalkyl, C₃₋₈ cycloalkyl-C₁₋₆ alkyl optionally substituted by halogen, C₁₋₆ alkyl or C₁₋₆ haloalkyl, C₃₋₈ cycloalkoxy-C₁₋₆ alkyl, C₄₋₁₀ bicycloalkyl-C₁₋₆ alkyl optionally substituted by halogen or C₁₋₆ alkyl, C₆₋₁₂ tricycloalkyl-C₁₋₆ alkyl, C₆₋₁₂ tricycloalkyl-amino or piperidinyl;
R³ is hydrogen, halogen or C₁₋₆ alkyl; 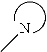
is 5- to 9-membered saturated or partially unsaturated heterocyclic ring or an oxo thereof, containing one nitrogen atom as ring-constituting atom, which may have halogen, C₁₋₆ alkyl, C₁₋₆ alkoxy, C₁₋₆ alkoxy-C₁₋₆ alkyl, hydroxy or methylidene as substituent, wherein the heterocyclic ring may further have one nitrogen atom, one oxygen atom and/or one sulfur atom as ring-constituting atom;
or a salt thereof.

2. The compound according to claim 1, wherein in the general formula [I], 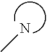
is piperidinyl, azepanyl, azocanyl, azonanyl, azepinyl, 2,3,4,7-tetrahydroazepinyl, 2,3,6,7-tetrahydroazepinyl, diazepanyl, piperazinyl, morpholinyl, thiomorpholinyl, oxazepanyl or an oxo thereof, wherein the heterocyclic ring may have halogen, C₁₋₆ alkyl, C₁₋₆ alkoxy or hydroxy as substituent,
or a salt thereof.

3. The compound according to claim 1, wherein in the general formula [I],
R¹ is ethyl, 1-propyl, 2-propyl, 1-butyl, 2-butyl, tert-butyl, 2-methyl-1-propyl, 2-methyl-1-butyl, 1-pentyl, 3-pentyl, 1-hexyl, cyclopropyl, cyclobutyl, cyclopentyl, cyclohexyl, 1-methylcyclohexyl, trifluoromethyl, 1,1-difluoroethyl, propoxy, cyclohexyloxy, ethylthio, methylpropylamino or dipropylamino;
R² is cyclopentyl, cyclohexyl, 1-methylcyclohexyl, 4-butylcyclohexyl, 4,4-difluorocyclohexyl, bicyclo[2.2.1]heptanyl, bicyclo[2.2.1]heptanylmethyl, bicyclo[4.1.0]heptanyl, bicyclo[2.2.2]octanyl, decahydronaphthyl, adamantyl (tricyclo[3.3.1.1]decanyl), spiro[2,5]octanyl, spiro[3,3]heptanylmethyl, 1-cyclohexylcyclopropyl, 1-methylcyclohexylmethyl, 2-methylcyclohexylmethyl, 3-methylcyclohexylmethyl, 4-methylcyclohexylmethyl, 3,5-dimethylcyclohexylmethyl, 4-ethylcyclohexylmethyl, 4-butylcyclohexylmethyl, 4-fluorocyclohexylmethyl, 4-methoxycyclohexylmethyl, 4-trifluoromethylcyclohexylmethyl, 4,4-difluorocyclohexylmethyl, 4,4-dimethylcyclohexylmethyl, cyclopropylmethyl, cyclobutylmethyl, cyclopentylmethyl, cyclopentylethyl, cyclohexylmethyl, cyclohexylethyl, cyclohexylpropyl, cyclohexylbutyl, cycloheptylmethyl, 1-cyclohexylethyl, adamantylmethyl, 4-methylcyclohexylmethyl, cyclopentyloxymethyl, cyclohexyloxymethyl, cycloheptyloxymethyl, adamantylamino or piperidinyl;
R³ is hydrogen; 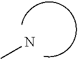
is azepanyl, azocanyl, azonanyl, 2,3,4,7-tetrahydroazepinyl, 2,3,6,7-tetrahydroazepinyl, 1,4-diazepanyl, oxazepanyl, 2,2-dimethylazepanyl, 3-hydroxyazepanyl, 4-hydroxyazepanyl, 4-methylazepanyl, 4,4-difluoroazepanyl, 4-methylpiperidinyl, 2,2-dimethylpiperidinyl, 2,2-dimethyl-3-hydroxypiperidinyl, 2,2-dimethyl-3-methylidene-piperidinyl, 2,2-dimethyl-4-hydroxypiperidinyl, 2,2-dimethyl-3-methoxypiperidinyl, 2,2-dimethyl-4-methoxypiperidinyl, 2,2,4,4-tetramethylpiperidinyl, 2,2,4,4-tetramethyl-3-hydroxypiperidinyl, 2,2,4,4-tetramethyl-4-methoxypiperidinyl, 2,2-dimethyl-4-methoxyethylpiperidinyl, 2,2-dimethyl-3-methylenepiperidinyl, 2,2-dimethylpiperazinyl, 2,2-dimethyl-4-hydroxypiperazinyl, 2,2-dimethylmorpholinyl, 2,2-dimethyl-3-oxopiperidinyl, 2,2,4,4-tetramethyl-3-hydroxypiperidinyl, 2,2,4,4-tetramethyl-3-oxopiperidinyl, 2,2-dimethyl-4-thiomorpholinyl, 3,3-dimethyl-4-thiomorpholinyl or oxazepanyl;
or a salt thereof.

4. The compound according to claim 1, wherein in the general formula [I],
R¹ is ethyl, 1-propyl, 2-propyl, 1-butyl, 2-butyl, 3-pentyl, cyclohexyl or trifluoromethyl;
R² is cyclopentyl, cyclohexyl, cycloheptyl, adamantyl, cyclobutylmethyl, cyclopentylmethyl, cyclopentylethyl, cyclohexylmethyl, cyclohexylethyl, cyclohexyloxymethyl, 1-cyclohexylethyl, 4-methylcyclohexylmethyl, 4-ethylcyclohexylmethyl, 4-trifluoromethylcyclohexylmethyl,4,4-dimethylcyclohexylmethyl, bicyclo[2.2.1]heptanylmethyl, spiro[3.3]heptanylmethyl or adamantylamino;
R³ is hydrogen; 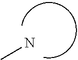
is piperidinyl, azepanyl, azocanyl, 2,3,4,7-tetrahydroazepinyl, 2,2-dimethylpiperidinyl, 2,2-dimthyl-3-hydroxypiperidinyl, 2,2-dimethyl-3-oxopiperidinyl, 2,2,4,4-tetramethyl-3-oxopiperidinyl or 3,3-dimethyl-4-thiomorpholinyl;
or a salt thereof.

5. The compound according to claim 1, which is selected from the group consisting of the following compounds:

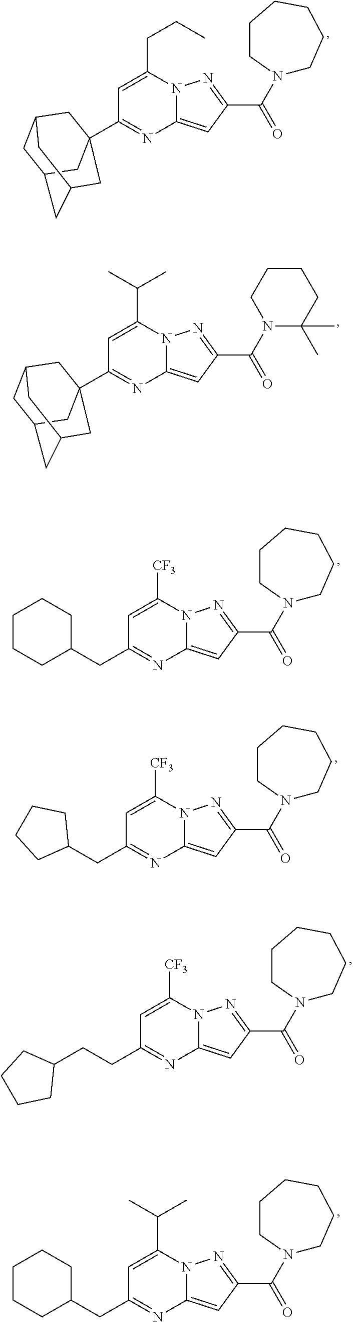 [SMILES: CCCc1cc(C23CC4CC(CC(C4)C2)C3)nc2cc(C(=O)N3CCCCCC3)nn12 ; CC(C)c1cc(C23CC4CC(CC(C4)C2)C3)nc2cc(C(=O)N3CCCCC3(C)C)nn12 ; O=C(c1cc2nc(CC3CCCCC3)cc(C(F)(F)F)n2n1)N1CCCCCC1 ; O=C(c1cc2nc(CC3CCCC3)cc(C(F)(F)F)n2n1)N1CCCCCC1 ; O=C(c1cc2nc(CCC3CCCC3)cc(C(F)(F)F)n2n1)N1CCCCCC1 ; CC(C)c1cc(CC2CCCCC2)nc2cc(C(=O)N3CCCCCC3)nn12]

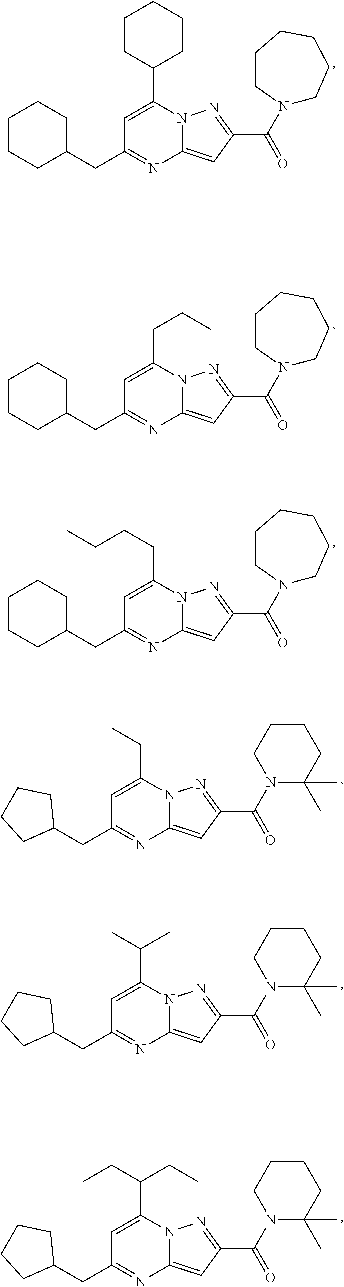 [SMILES: O=C(c1cc2nc(CC3CCCCC3)cc(C3CCCCC3)n2n1)N1CCCCCC1 ; CCCc1cc(CC2CCCCC2)nc2cc(C(=O)N3CCCCCC3)nn12 ; CCCCc1cc(CC2CCCCC2)nc2cc(C(=O)N3CCCCCC3)nn12 ; CCc1cc(CC2CCCC2)nc2cc(C(=O)N3CCCCC3(C)C)nn12 ; CC(C)c1cc(CC2CCCC2)nc2cc(C(=O)N3CCCCC3(C)C)nn12 ; CCC(CC)c1cc(CC2CCCC2)nc2cc(C(=O)N3CCCCC3(C)C)nn12]

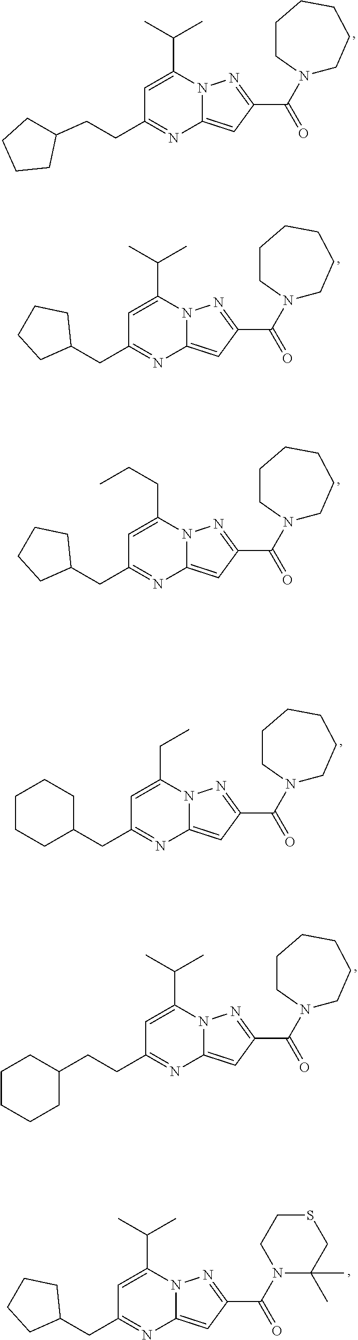 [SMILES: CC(C)c1cc(CCC2CCCC2)nc2cc(C(=O)N3CCCCCC3)nn12 ; CC(C)c1cc(CC2CCCC2)nc2cc(C(=O)N3CCCCCC3)nn12 ; CCCc1cc(CC2CCCC2)nc2cc(C(=O)N3CCCCCC3)nn12 ; CCc1cc(CC2CCCCC2)nc2cc(C(=O)N3CCCCCC3)nn12 ; CC(C)c1cc(CCC2CCCCC2)nc2cc(C(=O)N3CCCCCC3)nn12 ; CC(C)c1cc(CC2CCCC2)nc2cc(C(=O)N3CCSCC3(C)C)nn12]

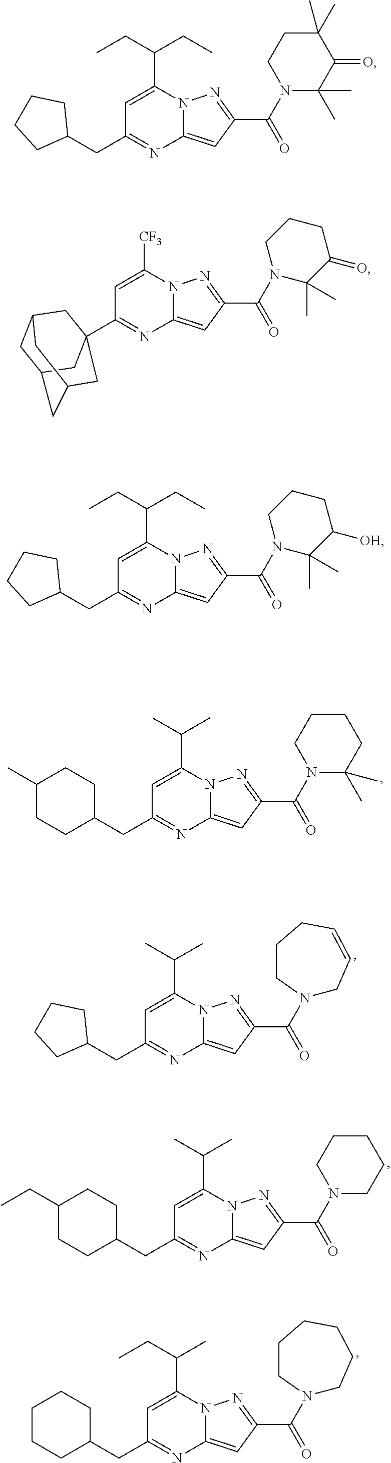

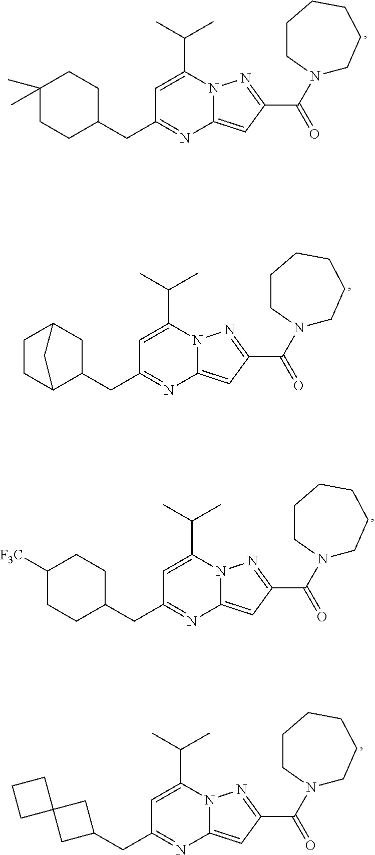 [SMILES: CC(C)c1cc(CC2CCC(C)(C)CC2)nc2cc(C(=O)N3CCCCCC3)nn12 ; CC(C)c1cc(CC2CC3CCC2C3)nc2cc(C(=O)N3CCCCCC3)nn12 ; CC(C)c1cc(CC2CCC(C(F)(F)F)CC2)nc2cc(C(=O)N3CCCCCC3)nn12 ; CC(C)c1cc(CC2CC3(CCC3)C2)nc2cc(C(=O)N3CCCCCC3)nn12]

6. A pharmaceutical composition comprising the compound according to claim 1 or a salt thereof as active ingredient, and a pharmaceutically acceptable carrier or excipient.

7. A therapeutic, preventative and/or diagnostic agent for a symptom and/or disease caused by PAR2 activation, comprising the compound according to claim 1 or a salt thereof.

8. The therapeutic, preventative and/or diagnostic agent according to claim 7, wherein the symptom caused by PAR2 activation is dermal pruritus.

9. The therapeutic, preventative and/or diagnostic agent according to claim 8, wherein the dermal pruritus is dermal pruritus caused by atopic dermatitis, urticaria, eczema, sebum deficiency, sebum deficiency eczema, senile pruritus, xeroderma, senile xerosis, prurigo, seborrheic dermatitis, psoriasis, contact dermatitis, caterpillar dermatitis, insect bite, photosensitivity, fruit hypersensitivity, neurodermatitis, self-sensitizing dermatitis, pruritus on renal dialysis and/or pruritus associated with chronic liver disease.

10. The therapeutic, preventative and/or diagnostic agent according to claim 7, wherein the disease caused by PAR2 activation is a skin disease.

11. The therapeutic, preventative and/or diagnostic agent according to claim 10, wherein the skin disease is selected from atopic dermatitis, psoriasis, eczema, scleroderma and dermatitis.

12. A therapeutic, preventative and/or diagnostic pharmaceutical composition for a symptom and/or disease caused by PAR2 activation, comprising the compound according to claim 1 or a salt thereof as active ingredient.

13. The therapeutic, preventative and/or diagnostic pharmaceutical composition according to claim 12, wherein the symptom caused by PAR2 activation is dermal pruritus.

14. The therapeutic, preventative and/or diagnostic pharmaceutical composition according to claim 13, wherein the dermal pruritus is dermal pruritus caused by atopic dermatitis, urticaria, eczema, sebum deficiency, sebum deficiency eczema, senile pruritus, xeroderma, senile xerosis, prurigo, seborrheic dermatitis, psoriasis, contact dermatitis, caterpillar dermatitis, insect bite, photosensitivity, fruit hypersensitivity, neurodermatitis, self-sensitizing dermatitis, pruritus on renal dialysis and/or pruritus associated with chronic liver disease.

15. The therapeutic, preventative and/or diagnostic pharmaceutical composition according to claim 12, wherein the disease caused by PAR2 activation is a skin disease.

16. The therapeutic, preventative and/or diagnostic pharmaceutical composition according to claim 15, wherein the skin disease is selected from atopic dermatitis, psoriasis, eczema, scleroderma and dermatitis.

17. A method for treating, preventing and/or diagnosing a symptom and/or disease caused by PAR2 activation, which comprises administering to a human in need thereof an effective amount of the compound or a salt thereof according to claim 1.

18. The method according to claim 17, wherein the symptom caused by PAR2 activation is dermal pruritus.

19. The method according to claim 18, wherein the dermal pruritus is dermal pruritus caused by atopic dermatitis, urticaria, eczema, sebum deficiency, sebum deficiency eczema, senile pruritus, xeroderma, senile xerosis, prurigo, seborrheic dermatitis, psoriasis, contact dermatitis, caterpillar dermatitis, insect bite, photosensitivity, fruit hypersensitivity, neurodermatitis, self-sensitizing dermatitis, pruritus on renal dialysis and/or pruritus associated with chronic liver disease.

20. The method according to claim 17, wherein the disease caused by PAR2 activation is a skin disease.

21. The method according to claim 20, wherein the skin disease is selected from atopic dermatitis, psoriasis, eczema, scleroderma and dermatitis.

22. A compound or a salt thereof according to claim 1 for use in the treatment, prevention and/or diagnosis of a symptom and/or disease caused by PAR2 activation.

23. The compound or a salt thereof according to claim 22, wherein the symptom caused by PAR2 activation is dermal pruritus.

24. The compound or a salt thereof according to claim 23, wherein the dermal pruritus is dermal pruritus caused by atopic dermatitis, urticaria, eczema, sebum deficiency, sebum deficiency eczema, senile pruritus, xeroderma, senile xerosis, prurigo, seborrheic dermatitis, psoriasis, contact dermatitis, caterpillar dermatitis, insect bite, photosensitivity, fruit hypersensitivity, neurodermatitis, self-sensitizing dermatitis, pruritus on renal dialysis and/or pruritus associated with chronic liver disease.

25. The compound or a salt thereof according to claim 22, wherein the disease caused by PAR2 activation is a skin disease.

26. The compound or a salt thereof according to claim 25, wherein the skin disease is selected from atopic dermatitis, psoriasis, eczema, scleroderma and dermatitis.

27. Use of a compound or a salt thereof according to claim 1 in the manufacture of a medicament for treating, preventing and/or diagnosing a symptom and/or disease caused by PAR2 activation.

28. The use according to claim 27, wherein the symptom caused by PAR2 activation is dermal pruritus.

29. The use according to claim 28, wherein the dermal pruritus is dermal pruritus caused by atopic dermatitis, urticaria, eczema, sebum deficiency, sebum deficiency eczema, senile pruritus, xeroderma, senile xerosis, prurigo, seborrheic dermatitis, psoriasis, contact dermatitis, caterpillar dermatitis, insect bite, photosensitivity, fruit hypersensitivity, neurodermatitis, self-sensitizing dermatitis, pruritus on renal dialysis and/or pruritus associated with chronic liver disease.

30. The use according to claim 27, wherein the disease caused by PAR2 activation is a skin disease.

31. The use according to claim 30, wherein the skin disease is selected from atopic dermatitis, psoriasis, eczema, scleroderma and dermatitis.

32. A topical transdermal formulation comprising the compound according to claim 1 or a salt thereof as active ingredient, and a pharmaceutically acceptable carrier or excipient.

33. The topical transdermal formulation according to claim 32, which is in the form selected from ointment, cream, lotion and foam.

34. The compound according to claim 1, which is 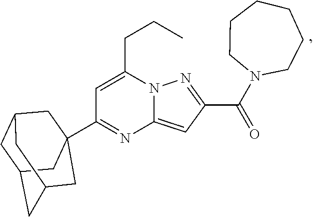 [SMILES: CCCc1cc(C23CC4CC(CC(C4)C2)C3)nc2cc(C(=O)N3CCCCCC3)nn12]
or a salt thereof.

35. The compound according to claim 1, which is 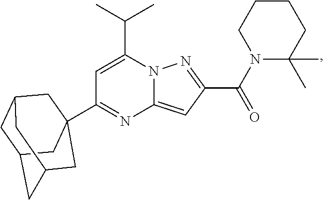
or a salt thereof.

36. The compound according to claim 1, which is 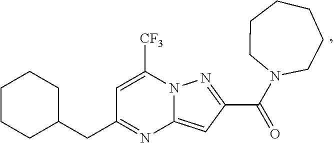 [SMILES: O=C(c1cc2nc(CC3CCCCC3)cc(C(F)(F)F)n2n1)N1CCCCCC1]
or a salt thereof.

37. The compound according to claim 1, which is 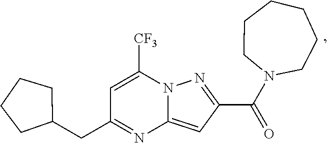 [SMILES: O=C(c1cc2nc(CC3CCCC3)cc(C(F)(F)F)n2n1)N1CCCCCC1]
or a salt thereof.

38. The compound according to claim 1, which is 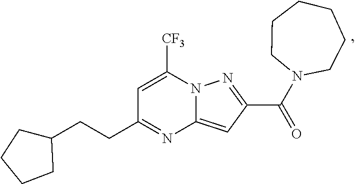 [SMILES: O=C(c1cc2nc(CCC3CCCC3)cc(C(F)(F)F)n2n1)N1CCCCCC1]
or a salt thereof.

39. The compound according to claim 1, which is 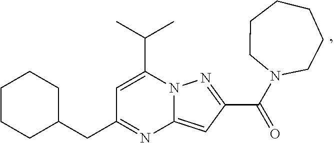 [SMILES: CC(C)c1cc(CC2CCCCC2)nc2cc(C(=O)N3CCCCCC3)nn12]
or a salt thereof.

40. The compound according to claim 1, which is 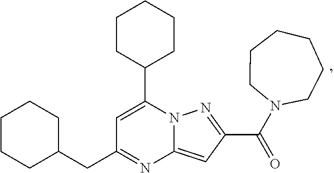 [SMILES: O=C(c1cc2nc(CC3CCCCC3)cc(C3CCCCC3)n2n1)N1CCCCCC1]
or a salt thereof.

41. The compound according to claim 1, which is 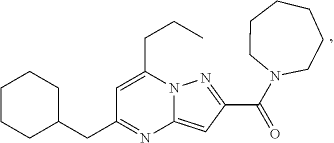 [SMILES: CCCc1cc(CC2CCCCC2)nc2cc(C(=O)N3CCCCCC3)nn12]
or a salt thereof.

42. The compound according to claim 1, which is 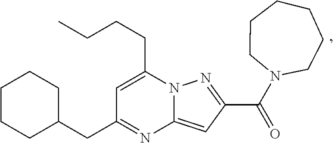 [SMILES: CCCCc1cc(CC2CCCCC2)nc2cc(C(=O)N3CCCCCC3)nn12]
or a salt thereof.

43. The compound according to claim 1, which is 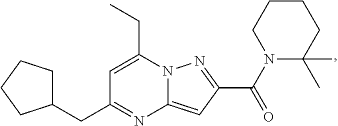
or a salt thereof.

44. The compound according to claim 1, which is 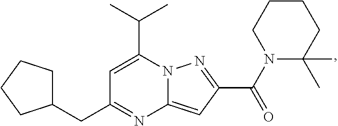
or a salt thereof.

45. The compound according to claim 1, which is 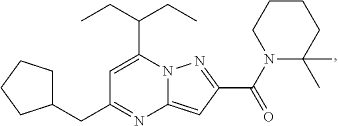
or a salt thereof.

46. The compound according to claim 1, which is 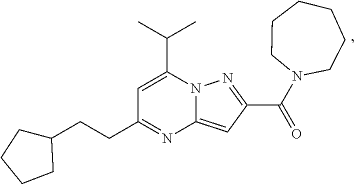 [SMILES: CC(C)c1cc(CCC2CCCC2)nc2cc(C(=O)N3CCCCCC3)nn12]
or a salt thereof.

47. The compound according to claim 1, which is 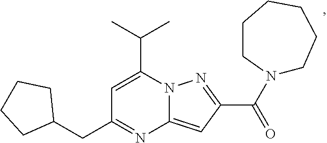 [SMILES: CC(C)c1cc(CC2CCCC2)nc2cc(C(=O)N3CCCCCC3)nn12]
or a salt thereof.

48. The compound according to claim 1, which is 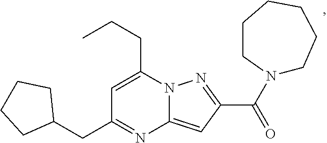 [SMILES: CCCc1cc(CC2CCCC2)nc2cc(C(=O)N3CCCCCC3)nn12]
or a salt thereof.

49. The compound according to claim 1, which is 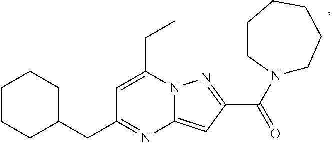 [SMILES: CCc1cc(CC2CCCCC2)nc2cc(C(=O)N3CCCCCC3)nn12]
or a salt thereof.

50. The compound according to claim 1, which is 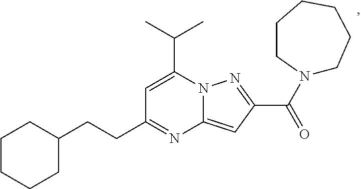 [SMILES: CC(C)c1cc(CCC2CCCCC2)nc2cc(C(=O)N3CCCCCC3)nn12]
or a salt thereof.

51. The compound according to claim 1, which is 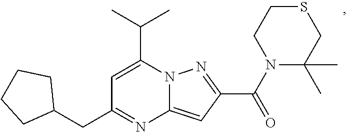 [SMILES: CC(C)c1cc(CC2CCCC2)nc2cc(C(=O)N3CCSCC3(C)C)nn12]
or a salt thereof.

52. The compound according to claim 1, which is 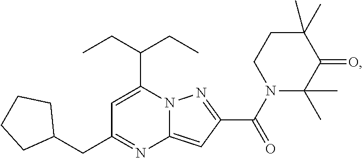 [SMILES: CCC(CC)c1cc(CC2CCCC2)nc2cc(C(=O)N3CCC(C)(C)C(=O)C3(C)C)nn12]
or a salt thereof.

53. The compound according to claim 1, which is 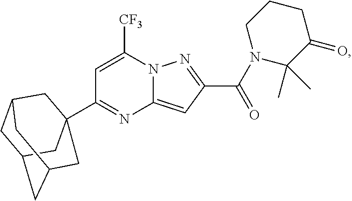 [SMILES: CC1(C)C(=O)CCCN1C(=O)c1cc2nc(C34CC5CC(CC(C5)C3)C4)cc(C(F)(F)F)n2n1]
or a salt thereof.

54. The compound according to claim 1, which is 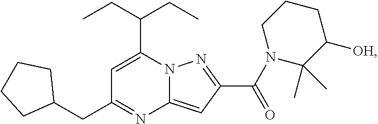 [SMILES: CCC(CC)c1cc(CC2CCCC2)nc2cc(C(=O)N3CCCC(O)C3(C)C)nn12]
or a salt thereof.

55. The compound according to claim 1, which is 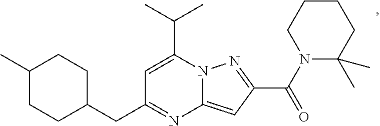
or a salt thereof.

56. The compound according to claim 1, which is 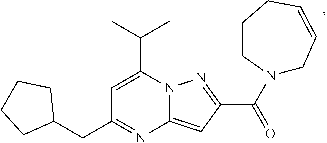 [SMILES: CC(C)c1cc(CC2CCCC2)nc2cc(C(=O)N3CC=CCCC3)nn12]
or a salt thereof.

57. The compound according to claim 1, which is 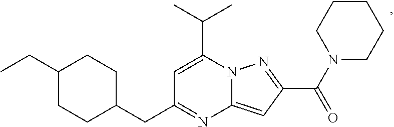 [SMILES: CCC1CCC(Cc2cc(C(C)C)n3nc(C(=O)N4CCCCC4)cc3n2)CC1]
or a salt thereof.

58. The compound according to claim 1, which is 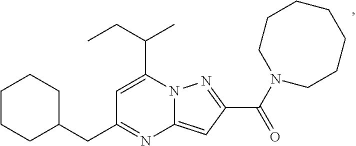 [SMILES: CCC(C)c1cc(CC2CCCCC2)nc2cc(C(=O)N3CCCCCCC3)nn12]
or a salt thereof.

59. The compound according to claim 1, which is 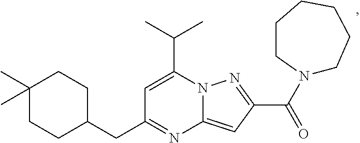 [SMILES: CC(C)c1cc(CC2CCC(C)(C)CC2)nc2cc(C(=O)N3CCCCCC3)nn12]
or a salt thereof.

60. The compound according to claim 1, which is 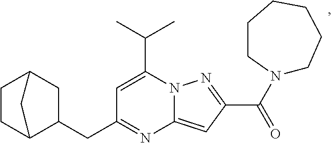 [SMILES: CC(C)c1cc(CC2CC3CCC2C3)nc2cc(C(=O)N3CCCCCC3)nn12]
or a salt thereof.

61. The compound according to claim 1, which is 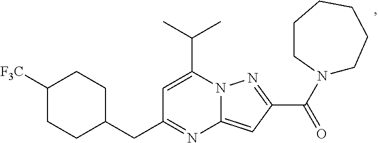 [SMILES: CC(C)c1cc(CC2CCC(C(F)(F)F)CC2)nc2cc(C(=O)N3CCCCCC3)nn12]
or a salt thereof.

62. The compound according to claim 1, which is 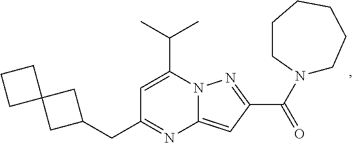 [SMILES: CC(C)c1cc(CC2CC3(CCC3)C2)nc2cc(C(=O)N3CCCCCC3)nn12]
or a salt thereof.

63. The compound according to claim 1, which is

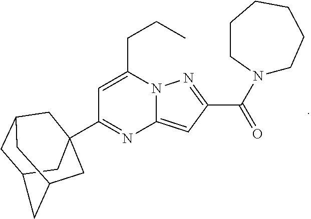 [SMILES: CCCc1cc(C23CC4CC(CC(C4)C2)C3)nc2cc(C(=O)N3CCCCCC3)nn12]

64. The compound according to claim 1, which is

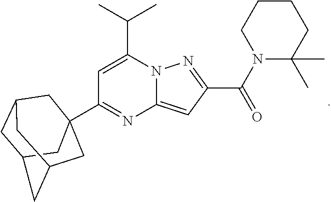 [SMILES: CC(C)c1cc(C23CC4CC(CC(C4)C2)C3)nc2cc(C(=O)N3CCCCC3(C)C)nn12]

65. The compound according to claim 1, which is

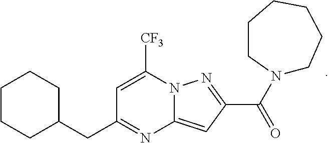 [SMILES: O=C(c1cc2nc(CC3CCCCC3)cc(C(F)(F)F)n2n1)N1CCCCCC1]

66. The compound according to claim 1, which is

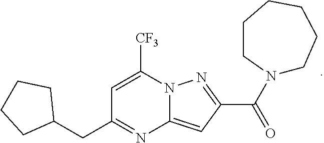 [SMILES: O=C(c1cc2nc(CC3CCCC3)cc(C(F)(F)F)n2n1)N1CCCCCC1]

67. The compound according to claim 1, which is

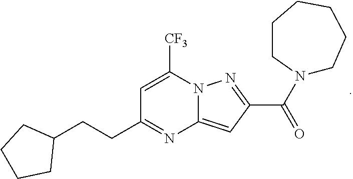 [SMILES: O=C(c1cc2nc(CCC3CCCC3)cc(C(F)(F)F)n2n1)N1CCCCCC1]

68. The compound according to claim 1, which is

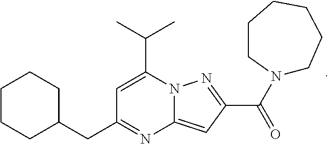 [SMILES: CC(C)c1cc(CC2CCCCC2)nc2cc(C(=O)N3CCCCCC3)nn12]

69. The compound according to claim 1, which is

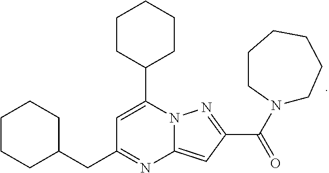 [SMILES: O=C(c1cc2nc(CC3CCCCC3)cc(C3CCCCC3)n2n1)N1CCCCCC1]

70. The compound according to claim 1, which is

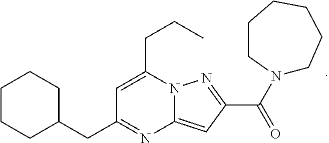 [SMILES: CCCc1cc(CC2CCCCC2)nc2cc(C(=O)N3CCCCCC3)nn12]

71. The compound according to claim 1, which is

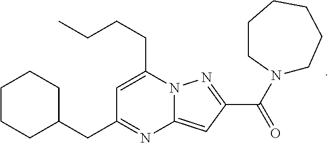 [SMILES: CCCCc1cc(CC2CCCCC2)nc2cc(C(=O)N3CCCCCC3)nn12]

72. The compound according to claim 1, which is

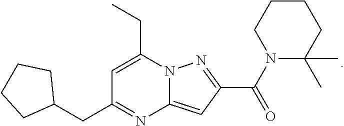 [SMILES: CCc1cc(CC2CCCC2)nc2cc(C(=O)N3CCCCC3(C)C)nn12]

73. The compound according to claim 1, which is

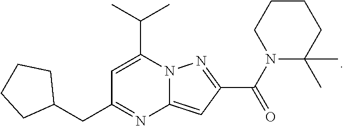 [SMILES: CC(C)c1cc(CC2CCCC2)nc2cc(C(=O)N3CCCCC3(C)C)nn12]

74. The compound according to claim 1, which is

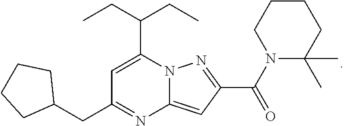 [SMILES: CCC(CC)c1cc(CC2CCCC2)nc2cc(C(=O)N3CCCCC3(C)C)nn12]

75. The compound according to claim 1, which is

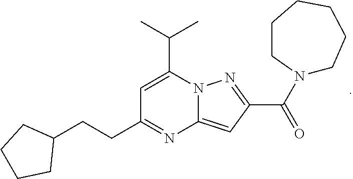 [SMILES: CC(C)c1cc(CCC2CCCC2)nc2cc(C(=O)N3CCCCCC3)nn12]

76. The compound according to claim 1, which is

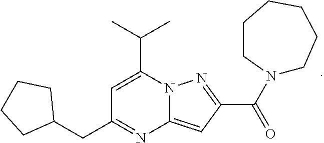 [SMILES: CC(C)c1cc(CC2CCCC2)nc2cc(C(=O)N3CCCCCC3)nn12]

77. The compound according to claim 1, which is

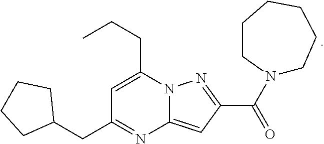 [SMILES: CCCc1cc(CC2CCCC2)nc2cc(C(=O)N3CCCCCC3)nn12]

78. The compound according to claim 1, which is

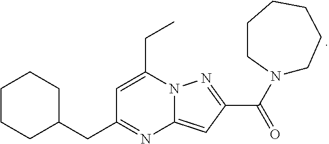 [SMILES: CCc1cc(CC2CCCCC2)nc2cc(C(=O)N3CCCCCC3)nn12]

79. The compound according to claim 1, which is

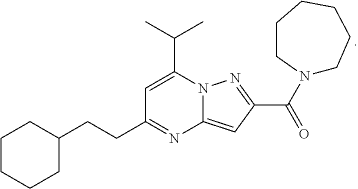 [SMILES: CC(C)c1cc(CCC2CCCCC2)nc2cc(C(=O)N3CCCCCC3)nn12]

80. The compound according to claim 1, which is

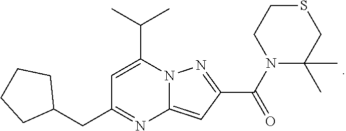 [SMILES: CC(C)c1cc(CC2CCCC2)nc2cc(C(=O)N3CCSCC3(C)C)nn12]

81. The compound according to claim 1, which is

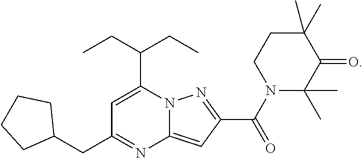 [SMILES: CCC(CC)c1cc(CC2CCCC2)nc2cc(C(=O)N3CCC(C)(C)C(=O)C3(C)C)nn12]

82. The compound according to claim 1, which is

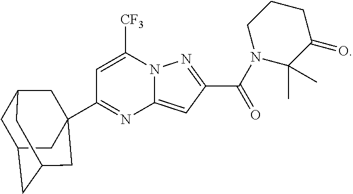 [SMILES: CC1(C)C(=O)CCCN1C(=O)c1cc2nc(C34CC5CC(CC(C5)C3)C4)cc(C(F)(F)F)n2n1]

83. The compound according to claim 1, which is

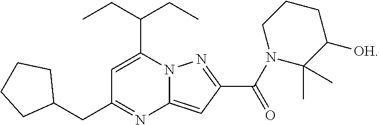 [SMILES: CCC(CC)c1cc(CC2CCCC2)nc2cc(C(=O)N3CCCC(O)C3(C)C)nn12]

84. The compound according to claim 1, which is

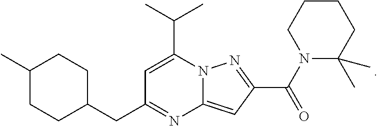

85. The compound according to claim 1, which is

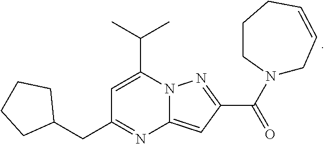 [SMILES: CC(C)c1cc(CC2CCCC2)nc2cc(C(=O)N3CC=CCCC3)nn12]

86. The compound according to claim 1, which is

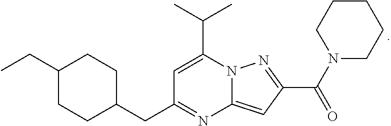 [SMILES: CCC1CCC(Cc2cc(C(C)C)n3nc(C(=O)N4CCCCC4)cc3n2)CC1]

87. The compound according to claim 1, which is

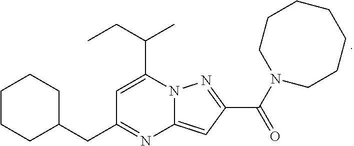 [SMILES: CCC(C)c1cc(CC2CCCCC2)nc2cc(C(=O)N3CCCCCCC3)nn12]

88. The compound according to claim 1, which is

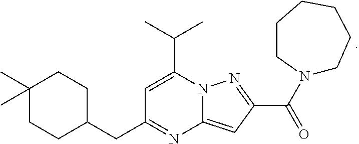 [SMILES: CC(C)c1cc(CC2CCC(C)(C)CC2)nc2cc(C(=O)N3CCCCCC3)nn12]

89. The compound according to claim 1, which is

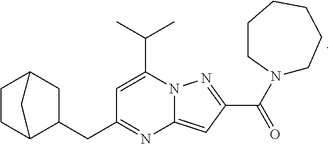 [SMILES: CC(C)c1cc(CC2CC3CCC2C3)nc2cc(C(=O)N3CCCCCC3)nn12]

90. The compound according to claim 1, which is

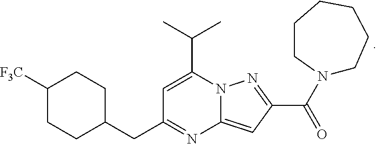 [SMILES: CC(C)c1cc(CC2CCC(C(F)(F)F)CC2)nc2cc(C(=O)N3CCCCCC3)nn12]

91. The compound according to claim 1, which is 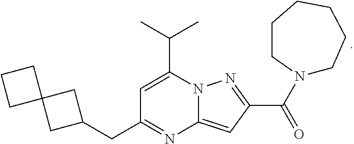 [SMILES: CC(C)c1cc(CC2CC3(CCC3)C2)nc2cc(C(=O)N3CCCCCC3)nn12]
